(19) United States
(12) Patent Application Publication
(10) Pub. No.: US 2025/0369036 A1
(43) Pub. Date: Dec. 4, 2025

(54) RIBOZYMES

(71) Applicant: Agency for Science, Technology and Research, Singapore (SG)

(72) Inventors: Shiying Sherry Aw, Singapore (SG); Yu Theng Mandy Lim, Singapore (SG); Shi Jie Teo, Singapore (SG)

(21) Appl. No.: 18/863,454

(22) PCT Filed: May 8, 2023
(86) PCT No.: PCT/SG2023/050318

§ 371 Date: Nov. 6, 2024

(30) Foreign Application Priority Data
May 6, 2022 (SG) 10202204811P

(51) Int. Cl. C12Q 1/6823; C12N 15/113

(52) U.S. Cl. CPC C12Q 1/6823; C12N 15/113; C12N 2310/121; C12N 2310/3519; C12N 2310/531

(57) ABSTRACT

There is provided a ribozyme comprising: a) one or more catalytic domains capable of switching between an active state and an inactive state; b) one or more releasable RNA segments, wherein each of said releasable RNA segments is flanked by two ribozyme cleavage sites, wherein cleavage at each cleavage site is catalysed by at least one of the one or more catalytic domains in an active state; c) one or more trigger-binding domains, each of which is for the binding of a trigger nucleic acid molecule; wherein each of the one or more catalytic domains is linked to one of the one or more trigger-binding domains; wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and wherein when both cleavage sites flanking a releasable RNA segment are cleaved when catalysed by the one or more catalytic domains, the one or more releasable RNA segment is released from the ribozyme. Also disclosed are methods of detecting presence of a trigger nucleic acid molecule in a sample, methods of detecting presence of a sequence or mutation of interest on an nucleic acid of interest in a sample, and kits comprising the ribozymes thereof.

### TECHNICAL FIELD

The present disclosure relates broadly to a ribozyme engineered to comprise one or more target/trigger-binding domains.

### BACKGROUND

The levels and profiles of coding and non-coding RNA in cells and in individuals present substantial information about biological and disease states. Accordingly, methods for RNA detection and quantification have been mainstays of molecular biology, and have continued to evolve with increasing technological sophistication. Traditional RT-qPCR and in situ hybridisation methods are still routinely used, supplemented with state-of-the-art single-cell RNA-sequencing and spatial transcriptomics methods to answer ever-more complex biological questions. The RNA content of most cells can now be determined with precision, allowing the probing of molecular and cellular functions of the transcriptome in normal and disease states. In addition, cellular and systemic RNA biomarkers are important for disease diagnosis and to guide clinical decision-making.

While in situ RNA detection has many important applications, there is currently no generalisable method that can directly sense and convert a specific RNA signal into a second functional, non-coding RNA readout. Such a method could be genetically encoded and act as a compact gene switch to transduce RNA context to functional outputs. RNA-sensing gene switches have been developed for some gene regulatory systems, most notably, CRISPR. For example, guide RNAs have been modified to respond to antisense blocking sequences at the guide spacer or other regions, so that they can be activated or deactivated in response to RNA triggers or ligands via toehold-mediated strand displacement, and CRISPR machinery have been modified to be conditionally activated upon microRNA function, e.g. microRNA-directed Ago2 cleavage and release of sgRNA, and miRNA-regulated Cas9 mRNA. Many of these systems have sequence constraints as their designs involve strand displacement of critical regions of the gRNA or require multiple RNA or protein components. When the guide RNA itself is modified in this way, careful design of the switching mechanism is required so that sgRNA function and specificity is not affected. They are also CRISPR-specific and are not generalisable for transducing RNA signals to other functional non-coding RNA pathways, e.g. shRNA, anti-sense or splice-switching oligonucleotides and RNA aptamers.

Therefore, there is a need for a molecular system that can transduce binding of an RNA or other nucleic acid signal, into release of a functional RNA. The present disclosure provides a solution in the form of a ribozyme engineered to be capable of releasing functional RNA upon binding to a sequence-complementary trigger.

### SUMMARY

In one aspect, there is provided a ribozyme comprising:
a) one or more catalytic domains capable of switching between an active state and an inactive state;
b) one or more releasable RNA segments, wherein each of said releasable RNA segments is flanked by two ribozyme cleavage sites, wherein cleavage at each cleavage site is catalysed by at least one of the one or more catalytic domains in an active state;
c) one or more trigger-binding domains, each of which is for the binding of a trigger nucleic acid molecule; wherein each of the one or more catalytic domains is linked to one of the one or more trigger-binding domains;
wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and
wherein when both cleavage sites flanking a releasable RNA segment are cleaved, the one or more releasable RNA segment is released from the ribozyme,
wherein the ribozyme comprises an RNA strand with
   motifs [A] and [a], wherein motifs [A] and [a] constitute the trigger-binding domain for binding the trigger nucleic acid molecule;
   motifs [B] and [b], wherein motifs [B] and [b] constitute a linker that functions as a communication module to stabilise the catalytic domain when the trigger nucleic acid is bound, wherein motif [B] and [b] are independently at least 1 nucleotide in length;
   motifs [C] and [c], wherein motifs [C] and [c] constitute the catalytic domain;
   motif [D], wherein motif [D] comprises the first cleavage site capable of being cleaved when catalysed by the catalytic domain;
   motif [D′], wherein motif [D′] comprises the second cleavage site capable of being cleaved when catalysed by the catalytic domain;
   motif [E], wherein motif [E] comprises the releasable RNA segment;
   motif [e], wherein motif [e] comprises a sequence that is partially or fully complementary to the sequence of motif [E];
   and wherein the motifs are connected by one or more optional linker region.

In some examples, the linker between motifs [C] and [D] is selected from the group consisting of two-way junction, three-way junction, four-way junction, a stem, single-nucleotide bulges, two-nucleotide bulges, three-nucleotide bulges, multi-nucleotide bulges and combinations thereof.

In some examples, the linker between motifs [C] and [D] comprises a three-way junction and a stem.

In some examples, the stem sequence connecting the junction to motif [D] is 4 to 12 nucleotides in length.

In some examples, the stem sequence connecting motif [C] and [c] to the junction is 4 to 12 nucleotides in length.

In some examples, the trigger nucleic acid molecule comprises a region that is complementary to the trigger-binding domain, wherein said region is more than 10 nucleotides in length, optionally the one or more trigger-binding domains are for binding the same trigger nucleic acid molecule.

In some examples, the releasable RNA segment is 6 to 150 nucleotides in length.

In some examples, the releasable RNA segment comprises a sequence that is identical to at least one of the one or more trigger RNA molecules.

In some examples, the releasable RNA segment is a functional RNA selected from the group consisting of single-guide RNA (sgRNA), guide RNA (gRNA), short hairpin RNA (shRNA), and RNA aptamer.

In some examples, motifs [B] and [b] are independently 1 or more nucleotides in length, optionally 3 or more nucleotides in length.

In some examples, motifs [B] and [b] has a sequence selected from the group consisting of SEQ ID NO: 1 (5′-ACG/CGU-3′), SEQ ID NO: 2 (5′-ACG/CGA-3′), SEQ ID NO: 449 (5′-ACG/UGA-3′), SEQ ID NO: 450 (5′-AUG/CGA-3′), SEQ ID NO: 451 (5′-AUG/UGA-3′), SEQ ID NO: 452 (5′-CG/CG-3′), SEQ ID NO: 453 (5′-UUG/UGG-3′), SEQ ID NO: 454 (5′-UAU/AUA-3′), SEQ ID NO: 455 (5′-ACU/AGA-3′), SEQ ID NO: 456 (5′-AUG/CAA-3′), SEQ ID NO: 457 (5′-CU/AG-3′), and SEQ ID NO: 458 (5′-UG/CA-3′).

In some examples, if motifs [e] and [E] are partially complementary to each other, the complementarity between motif [e] and [E] is characterised by alternating regions of complementarity and regions of non-complementarity.

In some examples, motif [D] comprises a mutation of nucleotide N₇ to pair with nucleotide N₊₃.

In some examples, the optional linker regions individually or collectively form one or more secondary structures, optionally the one or more secondary structures are selected from the group consisting of: single-nucleotide bulges, two-nucleotide bulges, three-nucleotide bulges, multi-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junctions), two-stem junctions (2-way junctions) or coaxial stacks or combinations thereof.

In some examples, the ribozyme complex comprises the sequences of any one or more of SEQ ID NOs: 3 to SEQ ID NOs: 448.

In some examples, the ribozyme further comprises one or more modification.

In some examples, the trigger nucleic acid comprises one or more modified nucleotide.

In another aspect, there is provided a method of detecting presence of a target/trigger nucleic acid molecule in a sample, wherein the method comprises:
incubating the sample with a ribozyme as disclosed herein at temperature T1 which allows the binding of the target/trigger nucleic acid molecule with one or more target/trigger-binding domains comprised in the ribozyme;
incubating the sample at temperature T2 which allows the nucleic acid molecule and the RNA segment to be released from the ribozyme;
detecting the release of the releasable RNA segment from the ribozyme.

In yet another aspect, there is provided a method of detecting presence of a sequence or mutation of interest on a nucleic acid of interest in a sample, wherein the method comprises:
incubating the sample with a ribozyme as disclosed herein, thereby allowing binding of the nucleic acid molecule of interest with one or more target/trigger-binding domains comprised in the ribozyme;
incubating the sample which allows the nucleic acid molecule and a releasable RNA segment to be released from the ribozyme;
detecting the release of the releasable RNA segment from the ribozyme;
wherein the releasable RNA segment is an sgRNA or shRNA; wherein detection of the sequence or mutation of interest in the sample results in a signal being generated.

In some examples, the trigger nucleic acid molecule is a genome of a virus, or a fragment thereof.

In yet another aspect, there is provided a kit comprising the ribozyme as disclosed herein.

### DESCRIPTION OF EMBODIMENTS

Detection of specific RNA or other nucleic acid sequences is integral to many applications in research, disease diagnosis, and therapeutics. Such applications will be further enabled if a detected nucleic acid signal can be directly functionally transduced via a second signal. This need is addressed by the present invention, which is a modular ribozyme whose self-cleavage is activated by binding of a specific complementary nucleic acid trigger sequence, leading to release of a second embedded RNA product without alteration of the original trigger. This reaction is entirely encoded within one single strand of RNA, and does not require any protein or DNA cofactors.

The inventors of the present disclosure show that the ribozymes disclosed herein are specific and sensitive. The inventors demonstrate that the ribozymes can be modularly designed for cell-free and in-cell applications. Thus, it is a versatile platform for which many potential applications can be envisioned.

The present disclosure describes a modular RNA signal transduction platform based on an altered self-cleaving ribozyme with one trigger-binding site and two cleavage sites, between which is embedded a releasable RNA cleavage product. The ribozyme's self-cleavage activity is dependent on complementary detection and binding of a specific trigger nucleic acid. Upon trigger-binding, ribozyme self-cleavage is activated to release the embedded RNA cleavage product. Hence, the ribozyme acts simultaneously as a direct RNA signal detector and transducer.

Accordingly, in one aspect of the present disclosure, there is provided a ribozyme comprising:
a) one or more catalytic domains capable of switching between an active state and an inactive state;
b) one or more releasable RNA segments, wherein each of said releasable RNA segments is flanked by two ribozyme cleavage sites, wherein cleavage at each cleavage site is catalysed by at least one of the one or more catalytic domains in an active state;
c) one or more trigger-binding domains, each of which is for the binding of a trigger nucleic acid molecule; wherein each of the one or more catalytic domains is linked to one of the one or more trigger-binding domains;
wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and wherein when both cleavage sites flanking a releasable RNA segment are cleaved, the one or more releasable RNA segment is released from the ribozyme,
wherein the ribozyme comprises an RNA strand with
   motifs [A] and [a], wherein motifs [A] and [a] constitute the trigger-binding domain for binding the trigger nucleic acid molecule;
   motifs [B] and [b], wherein motifs [B] and [b] constitute a linker that functions as a communication module to stabilise the catalytic domain when the trigger nucleic acid is bound, wherein motif [B] and [b] are independently at least 1 nucleotide in length;
   motifs [C] and [c], wherein motifs [C] and [c] constitute the catalytic domain;
   motif [D], wherein motif [D] comprises the first cleavage site capable of being cleaved when catalysed by the catalytic domain;
   motif [D′], wherein motif [D′] comprises the second cleavage site capable of being cleaved when catalysed by the catalytic domain;
   motif [E], wherein motif [E] comprises the releasable RNA segment;
   motif [e], wherein motif [e] comprises a sequence that is partially or fully complementary to the sequence of motif [E];
   and wherein the motifs are connected by one or more optional linker region. In some examples, the one or more motifs [A], [B], [C], [D], [E], [D′], [e], [c], [b], to [a] is in 5′ to 3′ directionality or 3′ to 5′ directionality. In some examples, the one or more motifs [A], [B], [D], [E], [D′], [e], [b], to [a] is in the 5′ to 3′ directionality or 3′ to 5′ directionality.

In some examples, there is provided a ribozyme comprising:
a) one or more catalytic domains capable of switching between an active state and an inactive state;
b) one or more releasable RNA segments, wherein each of said releasable RNA segments is flanked by two ribozyme cleavage sites, wherein cleavage at each cleavage site is catalysed by at least one of the one or more catalytic domains in an active state;
c) one or more trigger-binding domains, each of which is for the binding of a trigger nucleic acid molecule; wherein each of the one or more catalytic domains is linked to one of the one or more trigger-binding domains;
wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and wherein when both cleavage sites flanking a releasable RNA segment are cleaved, the one or more releasable RNA segment is released from the ribozyme,
wherein the ribozyme comprises an RNA strand with the following structure:

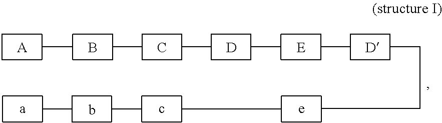

wherein [A] to [a] is in 5′ to 3′ directionality or 3′ to 5′ directionality, and wherein:
motifs [A] and [a] constitute the trigger-binding domain for binding the trigger nucleic acid molecule;
motifs [B] and [b] constitute a linker that functions as a communication module to stabilise the catalytic domain when the trigger nucleic acid is bound, wherein motif [B] and [b] are independently at least 1 nucleotide in length;
motifs [C] and [c] constitute the catalytic domain;
motif [D] comprises the first cleavage site capable of being cleaved when catalysed by the catalytic domain;
motif [D′] comprises the second cleavage site capable of being cleaved when catalysed by the catalytic domain;
motif [E] comprises the releasable RNA segment;
motif [e] comprises a sequence that is partially or fully complementary to the sequence of motif [E];
each of the horizontal lines connecting the motifs represents an optional linker region.

Whereby a catalytic domain refers to a domain or domains that comprise nucleotides that are required for or participate in ribozyme catalysis, or motifs that comprise nucleotides that together are required for or participate in catalysis. In some examples, there is no single catalytic domain that switches between an active state and an inactive state. In some examples, trigger-binding is required for a ribozyme to fold into a conformation that allows cleavage by nucleotides that participate in catalysis. In some examples, the cleavage at the cleavage site may be catalysed by the one or more catalytic nucleotides located within catalytic domains. As a result, the ribozyme is in an inactive state when the trigger-binding domain linked to said catalytic domain is not bound by the trigger nucleic acid molecule, and wherein the ribozyme is in an active state when the trigger-binding domain linked to said catalytic domain is bound by the trigger nucleic acid molecule

In some examples, the ribozyme may comprise:
a) one or more catalytic domains capable of switching between an active state and an inactive state,
b) one or more releasable RNA segments, wherein each of said releasable RNA segments is flanked by two ribozyme cleavage sites, wherein cleavage at each cleavage site is catalysed by at least one of the one or more catalytic domains in an active state;
c) one or more trigger-binding domains, each of which is for the binding of a trigger nucleic acid molecule; wherein each of the one or more catalytic domains is linked to one of the one or more trigger-binding domains;
wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and
wherein when both cleavage sites flanking a releasable RNA segment are cleaved when catalysed by the one or more catalytic domains, the one or more releasable RNA segment is released from the ribozyme,
wherein the ribozyme comprises an RNA strand with the following structure:

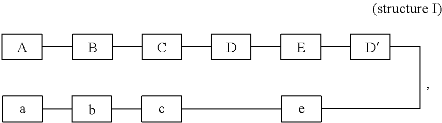

wherein [A] to [a] is in 5′ to 3′ directionality or 3′ to 5′ directionality, and wherein:
motifs [A] and [a] constitute the trigger-binding domain for binding the trigger nucleic acid molecule;
motifs [B] and [b] constitute a linker that functions as a communication module to stabilise the catalytic domain when the trigger nucleic acid is bound, wherein motif [B] and [b] are independently at least 1 nucleotide in length;
motifs [C] and [c] constitute the catalytic domain;
motif [D] comprises the first cleavage site capable of being cleaved when catalysed by the catalytic domain;
motif [D′] comprises the second cleavage site capable of being cleaved when catalysed by the catalytic domain;
motif [E] comprises the releasable RNA segment;
motif [e] comprises a sequence that is partially or fully complementary to the sequence of motif [E];
each of the horizontal lines connecting the motifs represents an optional linker region.

As used herein, the term “ribozyme” refers to an RNA molecule that is capable of catalysing specific biochemical reactions. Common examples of such reactions include the cleavage or ligation of RNA and DNA, and peptide bond formation. The term “ribozyme” as used herein includes both natural and artificial ribozymes. Artificial ribozymes include synthetic ribozymes and ribozymes modified or engineered from natural ribozymes. The term “ribozymes” also encompasses ribozyme fusions and ribozyme complexes derived from natural or artificial ribozymes.

In some examples, the term “ribozyme” may include ribozymes comprising one or more modifications to their phosphate backbone, sugar, or nucleobase, or with conjugations. That is, the ribozymes as disclosed herein may contain alterations to their phosphate backbone (e.g. phosphorothioate instead of phosphate linkages). They may contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2-guanidoethyl (2′-OGE), 2′-0,4′-C-methylene (LNA), 2′-O—(N-(methyl) acetamido) (2′-OMA), 2′-O-methyl, 2′-fluoro, 2′-O-(methoxyethyl) (2′-OME), and the like. They can also contain nucleobase modifications, e.g. 5-methylcytosine or pseudouridine. Such modifications are introduced to improve stability and reduce immunogenicity of the ribozymes. Methods of introducing such modification to an RNA (such as ribozymes) are common general knowledge in the art.

As used herein, the term “nucleic acid” or “polynucleotide” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “catalytic domain” refers to a domain or domains within a ribozyme that comprise nucleotides that are required for or participate in catalyzing the biochemical reactions as mentioned above, or motifs that together are required for or participate in catalysis. The domain may not be one contiguous segment or structure of the ribozyme and may instead comprise nucleotides located in different parts of the ribozyme sequence or structure. In some examples, trigger-binding is required for a ribozyme to fold into a conformation that allows cleavage by nucleotides that participate in catalysis. In some examples, the cleavage at the cleavage site may be catalysed by the one or more catalytic nucleotides, which are generally referred to as a catalytic domain. As a result, the ribozyme is in an inactive state when the trigger-binding domain linked to said catalytic domain is not bound by the trigger nucleic acid molecule, and wherein the ribozyme is in an active state when the trigger-binding domain linked to said catalytic domain is bound by the trigger nucleic acid molecule. In one example, in a ribozyme capable of cleaving RNA, the catalytic domain or domains are participate in catalyzing the cleavage of the RNA backbone at a ribozyme cleavage site.

The term “ribozyme cleavage site” refers to the sequences recognized and cleaved by a ribozyme catalytic domain or domains. Unless specified otherwise, the term “cleavage site” as used herein refers a ribozyme cleavage site. A ribozyme is in an “active state” when it is capable of catalyzing the biochemical reaction; whereas a ribozyme is in an “inactive state” when it is incapable of catalyzing the biochemical reaction. In a further example, a ribozyme is in an “active state” when it is capable of cleaving a ribozyme cleavage site; whereas a ribozyme is in an “inactive state” when it is incapable of cleaving a ribozyme cleavage site.

The term “target-binding domain” refers to a domain that is capable of binding a target nucleic acid molecule. In some examples, the term “target-binding domain” may be used interchangeably with the term “trigger-binding domain”. In some examples, the binding between the target/trigger nucleic acid molecule and the target/trigger-binding domain occurs through the annealing of complementary sequences between the two. Thus, it is possible to design or modify the sequence of the one or more target/trigger-binding domains so that they can bind to different target nucleic acid molecules with specific sequences. In some examples, the target nucleic acid molecules may be a target RNA molecule and/or a target DNA molecule.

The term “complementary” as used herein describes a relationship between two nucleotides or two polynucleotides. When referring to nucleic acid complementarity, the nucleotide A is complementary to the nucleotide U, and vice versa, and the nucleotide C is complementary to the nucleotide G, and vice versa. Complementary nucleotides include those that undergo Watson and Crick base pairing and those that base pair in alternative modes, for example the G: U wobble base-pair. It should be understood that, unless explicitly specified (e,g. by assigning a percentage or the term “fully” or “partially), the term “complementary” when used in relation to a nucleotide, includes varying degrees of complementarity. As used herein, the term “complementarity” refers to the degree and pattern by which one nucleic acid strand or segment is complementary to another nucleic acid strand of segment. When a percentage is assigned to a “complementarity” or a “degree of complementarity” between two polynucleotides (or segments thereof), the percentage refers to the percentage of nucleotides in one polynucleotide (or a segment thereof) that are complementary to the other polynucleotide (or a segment thereof). Therefore, a reference to two polynucleotide strands being “complementary” should be understood to cover both full and partial complementarity.

The term “target nucleic acid molecule” and the term “trigger nucleic acid molecule” may be used interchangeably in the present disclosure. Both terms as used herein refer to nucleic acid molecules of interest that are to be sensed and bound by the target-binding domain or trigger-binding domain. In some examples, the trigger nucleic acid when bound to the trigger-binding domain switches the ribozyme from an inactive to an active state. Without wishing to be bound by theory, the present disclosure also includes the possibility that trigger binding stabilises the ribozyme to enable the ribozyme to fold into the conformation required to cleave the cleavage site. Therefore, in some examples, the trigger binding may serve to stabilise the ribozyme to allow cleavage by nucleotides that participate in the catalysis. In some examples, trigger binding may include both scenarios where trigger binding switches catalytic domain from inactive to active state as well as trigger binding stabilising ribozyme to allow structural conformation required for cleavage.

The term “linked” refers to the relationship between two domains, and can refer to physical linkage, functional linkage, or both. In one example of the present invention, a catalytic domain is linked to a target-binding domain when the capability of the catalytic nucleotides to carry out catalysis is determined by the state of the target-binding domain, specifically whether the target-binding domain is bound to its corresponding target RNA molecule.

The term “flanked” refers to a polynucleotide sequence that is adjacent to another sequence or that is in between an upstream polynucleotide sequence and/or a downstream polynucleotide sequence, i.e., 5′ and/or 3′, relative to the sequence. For example, “a releasable RNA segment that is “flanked” by two cleavage sites” indicates that one cleavage site is located 5′ to the releasable RNA segment and the other cleavage site is located 3′ to the releasable RNA segment; however, there may be intervening sequences therebetween.

As the cleavage sites are comprised on the ribozyme itself, the ribozyme of the present disclosure is considered a self-cleaving ribozyme, and the “releasable RNA segment” can be considered a cleavage product of the self-cleaving activity. As the release of the releasable RNA segment from the ribozyme is a result of the ribozyme binding with its one or more target nucleic acid molecules, the ribozyme can be used to detect the presence of target nucleic acid molecules. As the target nucleic acid molecule(s) can be released from the ribozyme to activate more ribozymes and trigger the release of more “releasable RNA segments”, the presence of the target nucleic acid molecules can be amplified through the “releasable RNA segments” released in higher copies. The “releasable RNA segment” is variable and can be designed to comprise a large variety of sequences. In some examples where the “releasable RNA segment” comprises the same sequence as the target nucleic acid molecule, the target nucleic acid molecule (or the sequence thereof) is amplified using the ribozyme of the present disclosure. In some examples, the releasable RNA segment may comprise any one or more sequence(s) SEQ ID NO: 307 to 409.

In some examples, the binding of the target nucleic acid molecule to the target-binding domain enables the catalytic nucleotides to carry out catalysis, which results in the cleavage of both cleavage sites and the subsequent release of the releasable nucleic acid segment.

In view of the definition of “complementary” provided earlier in the present description, it would be understood by a person skilled in the art that the expression “optionally complementary” as used herein and the present description encompasses not only full complementarity (100% complementary) and partially complementary (between 0% and 100% complementarity), but also non-complementarity (0% complementarity). In some examples, the present disclosure includes about 5, 10, 15, 20, 25, 30, 35, 40, 50, 60, 70, 80, 90, 91, 92, 93, 94, 95, 96, 97, 98, 99, or 100% complementarity.

The term “directionality” as used herein refers to the end-to-end chemical orientation of a single strand of the RNA molecule. In a single strand of RNA, the chemical convention of naming carbon atoms in the nucleotide sugar-ring means that there will be a 5′-end, which contains a phosphate (or modified phosphate) group attached to the 5′ carbon of the ribose ring, and a 3′-end, which in natural RNA contains —OH at the 2′ position, but can also include various modifications, including, but is not limited to, 2′-fluoro, 2′-O-methyl, 2′ methoxyethyl, and the like. As an illustrative example, when [A] to [A′] of strand S1 is in 5′ to 3′ direction, [a] to [a′] of strand S2 will be in 3′ to 5′ direction.

As used herein, the term “motif” refers to a region on an RNA strand that has a specific structure or is involved with a specific function. The term “domain” as used herein refers to a region of the ribozyme that has a specific structure or is involved with a specific function. As used herein, the term “domain” is used when referring to a functional entity formed by more than one RNA strand or by more than one motif of one RNA strand. As an illustrative example, the target-binding domain comprises both motifs [A] and [a], and the target-binding domain is considered “bound” to a target RNA molecule only when both motifs are bound to the RNA molecule.

In some examples, the ribozyme as disclosed herein further comprises one or more inhibitory domains; wherein each of the one or more catalytic domains is functionally linked to one of the one or more inhibitory domains, wherein the catalytic domain is in an inactive state due to inhibition from the inhibitory domain, said inhibitory domain being linked to one of the one or more target-binding domains; wherein when one of the one or more target-binding domains is bound to the target nucleic acid molecule, the inhibitory domain linked to said target-binding domain ceases to inhibit the catalytic domain linked to said inhibitory domain, which results in the catalytic domain switching to an active state. In this example, the linkage between a target-binding domain and a catalytic domain is achieved by an inhibitory domain, which is linked to both the target-binding domain and the catalytic domain.

As is commonly known in the art, secondary structures are commonly formed within a ribozyme. In the examples of the present disclosure, one or more secondary structures are either formed individually by any of the motifs or the optional linker regions, or formed collectively by motifs, linker regions, or combinations thereof. In some examples, the optional linker regions individually or collectively form one or more secondary structures.

As used herein, the term “secondary structure” refers to structures formed by the interactions between nucleotides in one or more polynucleotides. Examples for secondary structures include, but are not limited to, single-nucleotide bulges, three-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junction), two-stem junctions (2-way junctions) or coaxial stacks or combinations thereof. Specific examples of secondary structures include stems, stem loops, t-RNA type structures, cloverleaves, tetraloops, pseudoknots or combinations thereof. As used herein, the term “stem loop”, also known as a “hairpin loop”, refers to a secondary nucleic acid structure that forms when two regions of the same strand, usually complementary in nucleotide sequence when read in opposite directions, base-pair to form a double helix that ends with an unpaired loop.

Therefore, in some examples, the optional linker regions in the ribozymes as disclosed herein may individually or collectively form one or more secondary structures. In some examples, the one or more secondary structures may include, but are not limited to, single-nucleotide bulges, two-nucleotide bulges, three-nucleotide bulges, multi-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junctions), two-stem junctions (2-way junctions) or coaxial stacks or combinations thereof.

As exemplified in the Experimental Section and Drawings, the ribozyme complex as disclosed herein may comprise a linker having a motif such as, but is not limited to, two-way junction, three-way junction, four-way junction, a stem, single-nucleotide bulges, two-nucleotide bulges, three-nucleotide bulges, multi-nucleotide bulges and combinations thereof. In some examples, the linker(s) between motifs [C] and [D] (i.e. Helices 2 and 3) may be, but is not limited to, a two-way junction, a three-way junction, a four-way junction, a stem, single-nucleotide bulges, two-nucleotide bulges, three-nucleotide bulges, multi-nucleotide bulges and combinations thereof. In some examples, the linker between motifs [C] and [D] may comprise a three-way junction and a stem (or may be referred to as a 4-way junction).

In some examples, the stem sequence connecting the junction to motif [D] is 4 to 12 nucleotides in length.

In some examples, the stem sequence connecting the junction to motif [D] is 4 or more, 5 or more, 6 or more, 7 or more, 8 or more, 9 or more, 10 or more, 11 or more, 12 or more nucleotides in length. In some examples, the stem sequence connecting the junction to motif [D] is about 4, 5, 6, 7, 8, 9, 10, 11, or 12 nucleotides in length. In some examples, the stem sequence connecting to the junction to motif [D] is about 5, 6, 7, 8, 10, 11, or 12 nucleotides in length.

In some examples, the junction is selected from the group consisting of helix-helix-helix (HHH), helix [minus 1 nucleotide]-strand [7 nucleotides long]-helix (H 1S7H), helix-helix-strand [4 nucleotides long]-helix (HHS4H), and helix-helix-strand [2 nucleotides long]-helix (HHHS2H), where strand indicates unpaired nucleotides.

In some examples, the stem sequence connecting motif [C] and [c] to the junction to 4 to 15 nucleotides in length. In some examples, the stem sequence connecting the junction to motif [C] and [c] is 4 or more, 5 or more, 6 or more, 7 or more, 8 or more, 9 or more, 10 or more, 11 or more, 12 or more, 13 or more, 14 or more, 15 or more nucleotides in length. In some examples, the stem sequence connecting the junction to motif [C] and [c] is about 4, 5, 6, 7, 8, 9, 10, 11, or 12 nucleotides in length. In some examples, the stem sequence connecting motif [C] and [c] to the junction is about 4, 5, 6, 7, 8, 10, 11, 12, 13, 14 or 15 nucleotides in length.

In some examples, the junction is selected from the group consisting of helix-helix-helix (HHH), helix [minus 1 nucleotide]-strand [7 nucleotides long]-helix (H 1S7H), helix-helix-strand [4 nucleotides long]-helix (HHS4H), and helix-helix-strand [2 nucleotides long]-helix (HHHS2H), where strand indicates unpaired nucleotides

In some examples, the trigger nucleic acid molecule comprises a region that is complementary to the trigger-binding domain, wherein said region is more than 10, more than 20, more than 30, more than 40, more than 50, more than 60, more than 70, more than 80, more than 90, more than 100, more than 110, more than 120, more than 130, more than 140, more than 150, more than 160, more than 170, more than 180, more than 190, or 200 nucleotides in length. In some examples, the region is no more than 200, no more than 190, no more than 180, no more than 170, no more than 160, no more than 150, no more than 140, no more than 130, no more than 120, no more than 110, no more than 100, no more than 90, no more than 80, no more than 70, no more than 60, no more than 50, no more than 40, no more than 30, no more than 20, or no more than 10 nucleotides in length. In some examples, the trigger nucleic acid molecule may be of any size from no more than 5 nucleotides in length up to many thousands or tens of thousands of nucleotides in length or more. Without wishing to be bound by theory, it would be understood by the person skilled in the art that the ribozymes as described herein is capable of binding to a trigger nucleic acid of any size provided the ribozymes can bind to a binding site of the trigger nucleic acid.

In some examples, the nucleotides of motifs [A] and [a] may bind to any site of the target/trigger nucleic acid molecule that the motifs can bind to. For example, where the nucleotides of motifs [A] and [a] are 10 nucleotides each and the trigger nucleic acid is 2000 nucleotides in length, [A] and [a] can bind to any two 10 nucleotides regions that the motifs can bind to. In some examples, motifs [A] and [a] may be able to bind to any two regions of the target/trigger nucleic acid that are not sequentially adjacent to one another.

In some examples, the nucleotides of motifs [A] and [a] may complementarily bind to the opposite ends of the target nucleic acid molecule respectively. For example, motif [A] may complementarily bind to 5′ end of the target nucleic acid molecule, while motif [a] may complementarily bind to the 3′ end of the target nucleic acid molecule, and vice versa.

The lengths of motifs [A] and [a] are independent from each other, and can be the same or different. For example, each of motifs [A] and [a] can be between 3 to 5, or between 5 to 10, or between 10 to 15, or between 15 to 20, or between 20 to 30, or between 30 to 40, or between 40 to 50, or 3, 4, 5, 6, 7, 8, 9, 10, 11, 12, 13, 14, 15 nucleotides in length. In some other examples, each of motifs [A] or [a] is between 1 to 5, or between 5 to 10, or between 10 to 15, or between 15 to 20, or between 20 to 30, or between 30 to 40, or between 40 to 50, between 50 to 60, between 60 to 70, between 70 to 80, between 80 to 90, between 90 to 100, between 100 to 110, between 110 to 120, between 130 to 140, between 140 to 150, between 150 to 160 nucleotides in length. In some examples, motif [a] is 11 nucleotides long. In some examples, the above descriptions for motifs [A] and [a] also apply to motifs [A′] and [a′].

The complementary binding is either partially complementary or fully complementary. For example, motif [A] is between about 70 to about 80%, or between about 80% to about 90%, or between about 90% to about 100%, or between about 75% to about 85%, or between about 85% to about 95%, or between about 95% to about 100%, or between about 88% to about 98%, or about 90%, about 91%, about 92%, about 93%, about 94%, about 95%, about 96%, about 97%, about 98%, about 99% or about 100% complementary to the a region of the target/trigger nucleic acid molecule; and motif [a] is between about 70 to about 80%, or between about 80% to about 90%, or between about 90% to about 100%, or between about 75% to about 85%, or between about 85% to about 95%, or between about 95% to about 100%, or between about 88% to about 98%, or about 90%, about 91%, about 92%, about 93%, about 94%, about 95%, about 96%, about 97%, about 98%, about 99% or about 100% complementary to another region of the target/trigger RNA molecule, and vice versa.

In some examples, the one or more trigger-binding domains are for binding the same target/trigger nucleic acid molecule. Examples of target or trigger nucleic acid molecules may include, but are not limited to, viral nucleic acid, bacterial nucleic acid, modified nucleic acid (such as mutation in a nucleic acid), messenger nucleic acid, coding nucleic acid, genomic nucleic acid, and the like.

Examples of target or trigger DNA molecules may include, but are not limited to viral DNA, cDNA, circulating DNA, cell free DNA (cfDNA), foetal DNA, modified DNA (e.g. mutation), single stranded DNA, double stranded DNA, mitochondrial DNA, and the like.

In some examples, the target/trigger nucleic acid may include, but is not limited to, viral RNA, a microRNA (miRNA), short interfering RNA (siRNA), small RNA (sRNA), messenger RNA (mRNA), non-coding RNA (ncRNA), short non-coding RNA, transfer RNA (tRNA), ribsomal RNA (rRNA), transfer-messenger RNA (tmRNA), clustered regularly interspaced short palindromic repeats RNA (CRISPR RNA), antisense RNA, pre-mRNA, circular RNA or pre-miRNA, or fragment thereof. In some examples, the trigger RNA molecule is a micro-RNA, or a precursor thereof, or a fragment thereof. In some examples, the trigger RNA may be a Let-7 microRNA precursor. In some examples, the trigger nucleic acid may comprise any one or more sequence(s) SEQ ID NO: 221 to 306.

In some examples, the target/trigger nucleic acid molecule may include one or more modified nucleotide. In some examples, the target/trigger nucleic acid molecule may include nucleotides with modified phosphate backbone, modified sugar moieties or sugar moiety analogs, or modified nucleobases. Sugar moiety modifications include, but are not limited to, 2′-O-aminoetoxy, 2′-O-amonioethyl (2′-OAE), 2′-O-methoxy, 2-guanidoethyl (2′-OGE), 2′-0,4′-C-methylene (LNA), 2′-O—(N-(methyl) acetamido) (2′-OMA), 2′-O-methyl, 2′-fluoro, 2′-O-(methoxyethyl) (2′-OME), and the like. Modified phosphate linkages include phosphorothioate and phosphorothiolate linkages. Modified nucleobases can include hundreds of naturally occurring and synthetic modified examples, e.g. 5-methylcytosine, methyladenosine, and pseudouridine, to cite a few of the most common

The term “micro-RNA” (abbreviated miRNA) as used herein refers to a small non-coding RNA molecule. It generally functions in RNA silencing and post-transcription regulation of gene expression. While the majority of miRNAs are located within the cell, some miRNAs, commonly known as circulating miRNAs or extracellular miRNAs, have also been found in the extracellular environment, including various biological fluids and cell culture media.

In some examples, the releasable RNA segment is more than 5, more than 10, more than 15, more than 20, more than 30, more than 40, more than 50, more than 60, more than 70, more than 80, more than 90, more than 100, more than 110, more than 120, more than 130, more than 140, or 150 nucleotides in length. In some examples, the releasable RNA segment is no more than 150, no more than 140, no more than 130, no more than 120, no more than 110, no more than 100, no more than 90, no more than 80, no more than 70, no more than 60, no more than 50, no more than 40, no more than 30, no more than 20, or no more than 10 nucleotides in length. In some examples, the releasable RNA segment may be about 1 to about 200, 1 to 20, 21 to 30, 31 to 40, 41 to 50, 51 to 60, 61 to 70, 71 to 80, 81 to 90, 91 to 100, 101 to 110, 111 to 120, 121 to 130, 131 to 140, or 141 to 150 nucleotides in length. In some examples, the releasable RNA segment is 6 to 150 nucleotides in length.

Some of the ribozymes as disclosed herein can function with full or partial complementarity between the releasable cleavage product and its complementary strand. In some examples, the releasable RNA segment comprises a sequence that is identical to at least one of the one or more target/trigger nucleic acid molecules.

In some examples, each of motifs [E] and [e] is between 6 to 160 nucleotides in length. In some examples, each of motifs [E] and [e] is between 5 to 10, or between 10 to 15, or between 15 to 20, or between 20 to 30, or between 30 to 40, or between 40 to 50, between 50 to 60, between 60 to 70, between 70 to 80, between 80 to 90, between 90 to 100, between 100 to 110, between 110 to 120, between 120 to 130, between 130 to 140, between 140 to 150, or 5, 6, 7, 8, 9, 10, 15, 20, 25, 30, 40, 50, 60, 70, 80, 90, 100, 110, 120, 130, 140, or 150 nucleotides in length.

In some examples, if motifs [e] and [E] are partially complementary to each other, the complementarity between motif [e] and [E] is characterised by alternating regions of complementarity and regions of non-complementarity.

In some examples, motifs [E] and [e] is less than 90%, less than 80%, less than 70%, less than 60%, less than 50%, less than 40%, or less than 30%, or about 10%, or about 20%, or about 30%, or about 40%, or about 50%, or about 60%, or about 70%, or about 80%, or about 90% complementary to each other. In some examples, the total complementarity between motifs [E] and [e] is at least 20%.

In some examples, the complementarity between motifs [E] and [e] is characterized by alternating regions of complementarity and regions of non-complementarity. The inventors envisage the complementarity of the nucleotides may include limitless patterns of complementarity. A non-exhaustive list of possible complementarity patterns include, but is not limited to, 5′-[1 nucleotide complementary-1 nucleotide non-complementary]n-3′; 5′-[1 nucleotide complementary-2 nucleotide non-complementary]n-3′; 5′-[1 nucleotide complementary-3 nucleotide non-complementary]n-3′; 5′-[1 nucleotide complementary-4 nucleotide non-complementary]n-3′; 5′-[2 nucleotide complementary-1 nucleotide non-complementary]n-3′; 5′ [2 nucleotide complementary-2 nucleotide non-complementary]n-3′; 5′-[2 nucleotide complementary-3 nucleotide non-complementary]n-3′; 5′-[2 nucleotide complementary-4 nucleotide non-complementary]n-3′; 5′-[3 nucleotide complementary-1 nucleotide non-complementary]n-3′; 5′-[3 nucleotide complementary-2 nucleotide non-complementary]n-3′; 5′-[3 nucleotide complementary-3 nucleotide non-complementary]n-3′; 5′-[3 nucleotide complementary-4 nucleotide non-complementary]n-3′; 5′-[4 nucleotide complementary-1 nucleotide non-complementary]n-3′; 5′-[4 nucleotide complementary-2 nucleotide non-complementary]n-3′; 5′-[4 nucleotide complementary-3 nucleotide non-complementary]n-3′; 5′-[4 nucleotide complementary-4 nucleotide non-complementary]n-3′; 5′-[2 nucleotide complementary-2 nucleotide non-complementary-1 nucleotide complementary-1 nucleotide non-complementary]n-3′; 5′-[3 nucleotide complementary-3 nucleotide non-complementary-1 nucleotide complementary-1 nucleotide non-complementary]n-3′; 5′-[4 nucleotide complementary-4 nucleotide non-complementary-1 nucleotide complementary-1 nucleotide non-complementary]n-3′; and the like. In some examples, n may be an integer from 1 to 200.

Examples of 5′-[3 nucleotide complementary-2 nucleotide non-complementary]n-3′ are SEQ ID NO: 66, SEQ ID NO: 106, and the like; Example of 5′-[3 nucleotide complementary-3 nucleotide non-complementary]n-3′ is SEQ ID NO: 99; Example of 5′-[4 nucleotide complementary-1 nucleotide non-complementary]n-3′ is SEQ ID NO: 108; Example of full complementarity is SEQ ID NO: 130.

In some examples, the alternating regions of complementarity and regions of non-complementarity may not have a pattern (or random in nature). The possible combinations can be determined as required when designing a ribozyme of interest. In some examples, the complementarity pattern may be determined by the target/trigger nucleic acid of interest. In some examples, the complementarity pattern may be determined by the cleaved product.

In some examples, each region of complementarity is not more than 3 consecutive nucleotides in length, and each region of non-complementarity is at least 3 consecutive nucleotides in length. In some examples, each region of complementarity between motifs [E] and [e] is not more than 3 consecutive nucleotides in length, wherein each region of non-complementarity between motifs [E] and [e] is at least 2 consecutive nucleotides long.

As used herein, a “region of complementarity” refers to a region of the ribozyme in which the first and second RNA strand are fully complementary to each other; and a “region of non-complementarity” refers to a region of the ribozyme in which the first and second RNA strand are not complementary to each other.

In some examples, the complementarity follows the pattern of 5′-3 nucleotides complementary-3 nucleotides non-complementary [2 nucleotides complementary-3 nucleotides non-complementary-2 nucleotides complementary]n-3 nucleotides non complementary-3 nucleotides complementary-3′; wherein n is the number of repeats of the pattern required to cover the length of motifs [E] and [e]. In some examples, n can be a number between 1 to 50, or 1 to 10, 10 to 20, 20 to 30, 30 to 40, 40 to 50, or 1, 2, 3, 4, 5, 6, 7, 8, 9, 10, 20, 30, 40, 50, 60, 70, 80, 90, 100, 110, 120, 130, 140, 150, 160, 170, 180, 190, or 200.

In some examples, the releasable RNA segment may be a functional RNA, such as, but is not limited to, single-guide RNA (sgRNA), guide RNA (gRNA), short hairpin RNA (shRNA), RNA aptamer, and the like.

Without wishing to be bound by theory, it is observed that various forms of motifs [B] and [b] as described herein (i.e. the Helix 4 communication module) advantageously confers properties of very low background cleavage, low leakiness and high cleavage signal, which is ideal for various applications.

Without wishing to be bound by theory, it is observed that modified RNA (such as locked nucleic acid modification as disclosed herein) can bind more strongly to the ribozyme than unmodified RNA. In some examples, modified RNA can bind more strongly to modified ribozymes (such as ribozymes modified with locked nucleic acid nucleotides as disclosed herein). As such, in some examples, motif [B] and [b] may be as short as 1 nucleotide in length. In some examples, motifs [B] and [b] are independently 1 or more nucleotides in length, optionally 2 or more nucleotides in length, optionally 3 or more nucleotides in length. In some examples, the motif [B] and [b] are independently 1 or more, 2 or more, 3 or more, or 4 or no more than 4 nucleotides in length. In some examples, the motif [B] and [b] are independently 1, 2, 3, or 4 nucleotides in length.

In some examples, motifs [B] and [b] has a sequence selected from the group consisting of SEQ ID NO: 1 (5′-ACG/CGU-3′), SEQ ID NO: 2 (5′-ACG/CGA-3′), SEQ ID NO: 449 (5′-ACG/UGA-3′), SEQ ID NO: 450 (5′-AUG/CGA-3′), SEQ ID NO: 451 (5′-AUG/UGA-3′), SEQ ID NO: 452 (5′-CG/CG-3′), SEQ ID NO: 453 (5′-UUG/UGG-3′), SEQ ID NO: 454 (5′-UAU/AUA-3′), SEQ ID NO: 455 (5′-ACU/AGA-3′), SEQ ID NO: 456 (5′-AUG/CAA-3′), SEQ ID NO: 457 (5′-CU/AG-3′), and SEQ ID NO: 458 (5′-UG/CA-3′).

In some examples, each of motifs [C], [c], [D] and [d] is independently between 1 to 100 nucleotides in length. In some examples, each of [C], [c], [D] and [d] is between 1 to 5, or between 5 to 10, or between 10 to 15, or between 15 to 20, or between 20 to 30, or between 30 to 40, or between 40 to 50, between 50 to 60, between 60 to 70, between 70 to 80 or 3, 4, 5, 6, 7, 8, 9, 10, 11, 12, 13, 14, 15, 16 nucleotides in length. In some specific examples, motif [C] has the same length as motif [c].

As illustrated in the Experimental Section, the inventors of the present disclosure found that a mutation in a specific site of a motif advantageously increase cleavage of the ribozymes. Thus, the ribozymes as disclosed herein may comprise a mutation at nucleotide N₇ to pair with nucleotide N₊₃, wherein the mutation increases cleavage when the sequence at the cleavage site deviates from the canonical sequences. In some examples, motif [D] comprises a mutation of nucleotide N7 to pair with nucleotide N+3.

Without wishing to be bound by theory, it is believed that one or more or all of the features mentioned above allows for the formation of a trigger-activated ribozyme (or dual ribozyme). That is, in some examples, the present disclosure provides a (dual) ribozyme comprising one or more features such as a circularly permuted (dual) ribozyme, reduced complementarity of the cleavage product, a 4-way junction, a shortened Helix 4, and an altered Helix 4 to encompass a branched nucleic acid-binding trigger region. In some examples, the present disclosure comprises a (dual) ribozyme comprising two or more features, or three or more, or all four features as disclosed herein. As disclosed herein, each element of the ribozyme as disclosed herein may be combined with the others in different combinations.

Thus, as disclosed herein are examples of ribozymes with one or more of the motifs as disclosed herein:
1) Sequence motifs in the Helix 4 communication module of the ribozyme that result in ribozymes with very low background cleavage in absence of the target/trigger RNA, and high cleavage rates in presence of the target/trigger nucleic acid, applicable over a series of ribozymes.
2) Altered and lengthened Helix 2 domains, which reduce the background cleavage of the cleavage site proximal to the catalytic domain.
3) Ribozymes with increased and full complementarity between the releasable cleavage product and its complementary strand, to reduce background cleavage product release.
4) A mutation of nucleotide N₇ to pair with nucleotide N₊₃, to increase cleavage when the sequence at or near the cleavage site deviates from canonical cleavage site sequences.
5) Altered and lengthened Helix 3 domains, which reduce the background cleavage of the cleavage site proximal to the catalytic domain.

In some examples, the ribozymes as disclosed herein may comprise two or more, three or more, or all four of the motifs as disclosed herein.

In some examples, the ribozymes may comprise a sequence as disclosed herein. In some examples, the ribozymes may comprise a sequence as disclosed in Table 1. In some examples, the ribozyme may comprise a sequence of one or more of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170. SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, and SEQ ID NO: 448. In some examples, the ribozyme complex comprises the sequences of any one or more of SEQ ID NOs: 3 to SEQ ID NOs: 448.

As would be appreciated by the person skilled in the art, the ribozyme as disclosed herein can be used for detecting and/or amplifying a trigger/target nucleic acid molecule in a sample. Therefore, in a second aspect of the present disclosure, there is provided a method of detecting presence of a trigger nucleic acid molecule in a sample, wherein the method comprises:
incubating the sample with a ribozyme as disclosed herein at temperature T1 which allows the binding of the trigger nucleic acid molecule with one or more trigger-binding domains comprised in the ribozyme;
incubating the sample at temperature T2 which allows the nucleic acid molecule and a RNA segment to be released from the ribozyme;
detecting the release of the releasable RNA segment from the ribozyme.

One exemplary method for using the ribozymes disclosed herein includes a method of detecting presence of a trigger nucleic acid molecule in a sample, wherein the method comprises:
a) incubating the sample with a ribozyme as disclosed herein at temperature T1 which allows the binding of the trigger RNA molecule with one or more trigger-binding domains comprised in the ribozyme;
b) incubating the sample at temperature T2 which allows the nucleic acid molecule and a releasable RNA segment to be released from the ribozyme;
c) detecting the release of the releasable RNA segment from the ribozyme.

In some examples, step c) is carried out by detecting the presence of the releasable RNA segment in the sample. Any RNA detection method or RNA detection systems known in the art can be used. Exemplary and non-exhaustive examples of RNA detection methods include: Reverse transcription polymerase chain reaction (RT-PCR), quantitative RT-PCR (RT-qPCR), probe-based RNA detection (such as northern blotting, microarrays and molecular beacons). Exemplary and non-exhaustive examples of RNA detection systems include: NanoString Technologies' nCounter@ miRNA expression assay and Exiqon's Smart Flares), RNA-activated fluorescent sensors such as the Pandan fluorescent sensor (PCT patent PCT/SG2017/050086; Aw et. al., Nucleic Acids Research 2016), and CRISPR-Cas based nucleic acid detection systems such as DETECTR (Chen, J. S. et al. CRISPR-Cas12a target-binding unleashes indiscriminate single-stranded DNase activity. Science 360, 436-439, doi: 10.1126/science.aar6245 (2018)) and SHERLOCK (Gootenberg, J. S. et al. Multiplexed and portable nucleic acid detection platform with Cas13, Cas12a, and Csm6. Science 360, 439-444, doi: 10.1126/science.aaq0179 (2018)).

In some examples, steps a) to b) are repeated for one or more times before step c) is carried out, wherein the RNA molecules released from step b) are for binding to another copy of the ribozyme. In this example, the sample can be incubated with an excess amount of the ribozyme when performing step a) for the first time, so that when performing a) for the second or subsequent time, additional ribozymes may not be supplemented to the sample. As the target nucleic acid molecules released from the ribozymes in step b) can further bind to new ribozymes, the copy number of released cleavage products (the “releasable RNA segment”) can be many folds higher than the copy number of target/trigger molecules in the sample. Therefore, the presence or the amount of the released “releasable RNA segment” serve as an amplified signal for the presence or the amount of the target/trigger nucleic acid molecules in the sample. Thus, this method can improve the sensitivity of existing nucleic acid detection technologies when used in combination.

The releasable RNA segment can comprise a sequence identical to the target/trigger nucleic acid, in which case the released “releasable RNA segments” can further bind to new ribozymes as target nucleic acid molecules themselves. By repeating steps a) to b) as above, substantial amplification of the target/trigger nucleic acid molecule (or the sequence the target/trigger nucleic acid molecule) can be achieved. Therefore, a method of amplifying a target/trigger nucleic acid molecule is also considered to be part of the present disclosure. Such methods may comprise the steps of a) incubating the target/trigger nucleic acid molecule with an ribozyme of the present disclosure at temperature T1 which allows the binding of the target/trigger nucleic acid molecule with one or more target-binding domains comprised in the ribozyme, and wherein the releasable RNA segment comprises a sequence identical to the target/trigger nucleic acid molecule; b) incubating the ribozyme bound to the target/trigger nucleic acid molecule at temperature T2 which allows the target/trigger nucleic acid molecule and the RNA segment to be released from the ribozyme. In some examples, steps a) to b) are repeated for one or more times, wherein the target/trigger nucleic acid molecules and the releasable RNA segment released from step b) are for binding to another copy of the ribozyme.

In some examples, the releasable RNA segment comprises a sequence that is identical to at least one of the one or or more trigger nucleic acid molecules. In some examples, the ribozyme comprises two trigger-binding domains for binding a specific trigger nucleic acid molecule, wherein the trigger nucleic acid molecule comprises a sequence that is identical to the trigger nucleic acid molecule. In some examples, the binding of trigger nucleic acid molecules leads to the release of an RNA molecule comprising the same sequence as the trigger nucleic acid molecule.

In some examples, the binding of the trigger nucleic acid molecule with the one or more trigger-binding domains of the ribozyme occurs at a temperature T1. In some examples, T1 is a temperature not more than 50° C. In some examples, T1 is a temperature between 0° C. to 50° C., a temperature between 15° C. to 45° C. a temperature between 25° C. to 45° C., a temperature between 30° C. to 40° C., a temperature between 35° C. to 38° C., a temperature between 36.5° C. to 37.5° C. In a specific example, T1 is a temperature of about 37° C.

In some examples, the temperatures T1 and T2 are identical. In another example, temperature cycling does not occur and/or is not required. Instead, the method requires sample incubation at one specific temperature. In some examples, the ribozymes are incubated at 37° C. for 4 hours. In one example, such a method is a cleavage assay.

In some examples, the trigger nucleic acid molecule bound to a trigger-binding domain of the ribozyme is released from said target-binding domain at a temperature T2. In some examples, wherein the two cleavage sites flanking the releasable RNA segment are cleaved, the releasable RNA segment is released at a preferred temperature T2. In some examples, T2 is a temperature between 20° C. to 100° C., a temperature between 25° C. to 80° C., a temperature between 30° C. to 80° C., a temperature between 35° C. to 80° C., a temperature between 40° C. to 80° C., a temperature between 45° C. to 80° C., a temperature between 50° C. to 80° C., a temperature between 55° C. to 75° C., or a temperature between 57° C. to 63° C. In a specific example, T2 is a temperature of about 60° C.

It should be understood that T1 and T2 as described above refer to temperatures which allow the binding (of the targeting RNA molecule) and the release (of the target nucleic acid molecules and the releasable RNA segment) respectively. T1 should not be taken to mean a temperature under which no trigger nucleic acid molecules or releasable RNA segments can be released; and T2 should not be taken to mean a temperature under which no target nucleic acid molecule can bind with the trigger-binding domain. It is generally understood in the art that the binding (also known as “annealing”) and release (also known as “melting”) of complementary RNA strands can occur simultaneously, albeit with differing kinetics, across a wide range of temperatures, therefore T1 and T2 can be the same or different. In some examples, the trigger nucleic acid molecules can bind to the trigger-binding domains of the ribozyme, triggering the cleavage and release of the releasable RNA segment, and release from the ribozyme, all at a temperature between 35° C. to 38° C. However, when the ribozyme is used to detect and amplify trigger molecules, the implementation of annealing (under T1) and melting (under T2), wherein T2 is higher than T1, drives the reaction forward and can result in increased number of released releasable RNA products.

In yet another aspect, there is provided a method of detecting presence of a sequence or mutation of interest on an nucleic acid of interest in a sample, wherein the method comprises:
incubating the sample with a ribozyme as disclosed herein, thereby allowing binding of the trigger nucleic acid molecule with one or more trigger-binding domains comprised in the ribozyme;
incubating the sample which allows the nucleic acid molecule and a releasable RNA segment to be released from the ribozyme;
detecting the release of the releasable RNA segment from the ribozyme;
wherein the releasable RNA segment is an sgRNA or shRNA; wherein detection of the sequence or mutation of interest in the sample results in a signal being generated.

In some examples, the trigger nucleic acid molecule is an nucleic acid molecule obtained from animals, viruses, bacteria, yeast or plants. Therefore, in some examples, the target or trigger nucleic acid molecules may include, but are not limited to, viral nucleic acid, bacterial nucleic acid, modified nucleic acid (such as mutation in a nucleic acid), messenger nucleic acid, coding nucleic acid, genomic nucleic acid, and the like.

Examples of target or trigger DNA molecules may include, but are not limited to viral DNA, cDNA, circulating DNA, cell free DNA (cfDNA), foetal DNA, modified DNA (e.g. mutation), single stranded DNA, double stranded DNA, mitochondrial DNA, and the like.

In some examples, the target/trigger nucleic acid may include, but is not limited to, viral RNA, a microRNA (miRNA), short interfering RNA (siRNA), small RNA (sRNA), messenger RNA (mRNA), non-coding RNA (ncRNA), short non-coding RNA, transfer RNA (tRNA), ribsomal RNA (rRNA), transfer-messenger RNA (tmRNA), clustered regularly interspaced short palindromic repeats RNA (CRISPR RNA), antisense RNA, pre-mRNA, circular RNA or pre-miRNA, or fragment thereof. In some examples, the trigger RNA molecule is a micro-RNA, or a precursor thereof, or a fragment thereof, the trigger nucleic acid molecule is an RNA or DNA molecule, or modified RNA or DNA molecule.

In some examples, the trigger nucleic acid molecule is a genome of a virus, or a fragment thereof. In some examples, the virus may include, but is not limited to, a Retroviridae virus, a Lentiviridae virus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, a Deltavirus.

In some examples, the virus may include, but is not limited to, Lymphocytic choriomeningitis virus, Coronavirus, human immunodeficiency virus (HIV), Severe acute respiratory syndrome virus (SARS), Severe acute respiratory syndrome coronavirus 2 (SARS-CoV-2), Poliovirus, Rhinovirus, Hepatitis A virus, Hepatitis B virus, Hepatitis C virus, Hepatitis D virus, Norwalk virus, Yellow fever virus, West Nile virus, Dengue fever virus, Zika virus, Rubella virus, Ross River virus, Rhinovirus C, Sindbis virus, Chikungunya virus, Coxsackievirus virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza virus (such as influenza A virus), smallpox virus (variola virus), Japanese encephalitis virus, Seoul virus, adenovirus, and Herpes simplex virus.

The inventors have exemplified in the Experimental Section, potential applications for the ribozyme for cell-free RNA signal amplification and as an in vitro and in vivo gene reporter and signal transducer, in fish and human cells.

Therefore, in some examples, the methods or ribozymes as disclosed herein may be used in any one of mammalian cells, mammalian cell lines, human cell, human cell lines, zebrafish cells, and zebrafish embryos. In some examples, the methods or ribozymes as disclosed herein may be used either in vitro or in vivo.

In some examples, the ribozymes as disclosed herein may be used in any one of mammalian cells, mammalian cell lines, human cell, human cell lines, zebrafish cells, and zebrafish embryos.

In yet another aspect, there is provided a polynucleotide encoding the ribozymes as disclosed herein. In some examples where the ribozyme is comprised of more than one RNA strand, the RNA strands can be encoded together on one polynucleotide or separately on several polynucleotides.

The isolated nucleotide sequence as disclosed herein can be used to transduce the binding signal of a target trigger nucleic acid sequence into release of a second functional nucleic acid sequence. The isolated nucleic acid sequence as disclosed herein contains a trigger-binding domain comprised of two trigger-binding arms that are sequence-complementary to an nucleic acid trigger of interest and is capable of forming bimolecular interactions with the target trigger nucleic acid sequence. The isolated nucleic acid sequence as disclosed herein also forms a ternary complex allowing self-cleavage of itself when binding the target nucleic acid sequence, releasing an RNA cleavage product fragment that can take the form of a functional RNA with tertiary structure, e.g. CRISPR guide RNA (gRNA or sgRNA), short hairpin RNA (shRNA), RNA aptamer and the like. In some examples, the ribozymes as disclosed herein release CRISPR gRNA, shRNA or an RNA aptamer in presence of the trigger nucleic acid. Therefore, the ribozymes as disclosed herein has the ability to function in vitro and in vivo for gene regulation. For example, the Experimental Section has demonstrated functional gene regulation by the ribozyme in vitro and in vivo, in fish and mammalian cells.

This isolated nucleotide sequence also comprises one or both distinct features of: A) A catalytic domain that is stabilised by binding of the trigger nucleic acid, and B) A communication module between the catalytic domain and the trigger arms. While a range of sequence motifs can be envisioned to work, and indeed the best module for any particular ribozyme may need to be empirically optimised, 5-10 sequence motifs were identified in Helix 4 that allow for optimised low background/high-cleavage activity in absence and presence of the trigger nucleic acid respectively. It was also found that lengthening of Helix 2 or Helix 3 decreases background cleavage of the cleavage site proximal to the catalytic domain in absence of the trigger.

In yet another aspect, there is provided a kit comprising the ribozymes as disclosed herein.

In some examples, the kit further comprises a nucleic acid detection system. In some examples, the nucleic acid detection system comprises an RNA-activated fluorescent sensor. In some examples, the sensor is a Pandan fluorescent sensor or detection system (PCT patent PCT/SG2017/050086; Aw et. al., Nucleic Acids Research 2016). In some examples the nucleic acid detection system is a CRISPR-Cas based nucleic acid detection system. CRISPR Cas-based nucleic acid detection methods and systems are known in the art, and are disclosed for example in Chen, J. S. et al. CRISPR-Cas12a target-binding unleashes indiscriminate single-stranded DNase activity. Science 360, 436-439, doi: 10.1126/science.aar6245 (2018), and Gootenberg, J. S. et al. Multiplexed and portable nucleic acid detection platform with Cas13, Cas12a, and Csm6. Science 360, 439-444, doi: 10.1126/science.aaq0179 (2018).

In some examples, the ribozymes as disclosed herein may also act as an nucleic acid signal transducer and amplifier.

In some examples, the ribozyme is modified from a naturally existing ribozyme. In some examples, the ribozyme is modified from an artificial ribozyme, fusion ribozyme, fragments and derivatives thereof. In some examples, the ribozyme is characteristic of a hairpin ribozyme or a hammerhead ribozyme, or fragments and fusions thereof. In some examples, the ribozyme is a twin ribozyme or duplex ribozyme. In some examples, the ribozyme comprises a twin-hairpin ribozyme structure. Design of ribozymes as described herein is illustrated in an earlier application, PCT/SG2020/050226, the content of which is incorporated herein and described briefly below.

As is commonly known in the art, a ribozyme can comprise one or more RNA strands. In a specific example, the ribozyme comprises a first RNA strand and a second RNA strand. When a ribozyme is comprised of two RNA strands, the two RNA strands have sufficient complementarity so that they are bound to each other. Typically, the two RNA strands are not fully complementary across their entire lengths. Each RNA strand can form secondary structures independently, as is generally known in the art. In one example, the ribozyme comprises the following structure:

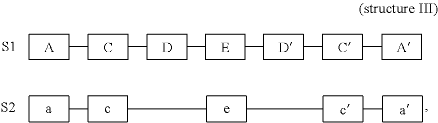

wherein: S1 is the first RNA strand and S2 is the second RNA strand, wherein [A] to [A′] and [a] to [a′] represent opposite directionalities; and motifs [A] and [a] constitute a first trigger-binding domain for binding a first trigger nucleic acid molecule, motifs [C] and [c] constitute a first catalytic domain, motif [D] comprises a first cleavage site capable of being cleaved, motifs [A′] and [a′] constitute a second trigger-binding domain for binding a second trigger nucleic acid molecule, motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region; and wherein the first catalytic domain is in an active state when the first target-binding domain is bound to first target RNA molecule; and wherein the second catalytic domain is in an active state when the second target-binding domain is bound to the second target nucleic acid molecule.

In another example, the ribozyme comprises the following structure:

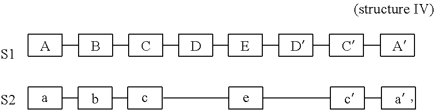

wherein: S1 is the first RNA strand and S2 is the second RNA strand, wherein [A] to [A′] and [a] to [a′] represent opposite directionalities; wherein motifs [A] and [a] constitute a first trigger-binding domain for binding a first trigger nucleic acid molecule, motifs [B] and [b] constitute a linker that functions as a communication module to stabilise the catalytic domain when the trigger nucleic acid is bound, wherein motif [B] and [b] are independently at least 1 nucleotide in length;
or, wherein motifs [B] and [b] constitute a first inhibitory domain, motifs [C] and [c] constitute a first catalytic domain, motif [D] comprises a first cleavage site capable of being cleaved by the first catalytic domain, motifs [A′] and [a′] constitute a second trigger-binding domain for binding a second trigger nucleic acid molecule; motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region, wherein the first inhibitory domain is characterized by i) or ii) below:
   i) motif [b] anneals with [C] when the first target-binding domain is not bound by the first target nucleic acid molecule, but anneals with [B] when the first target-binding domain is bound by the first target nucleic acid molecule,
   ii) motif [B] anneals with motif [c] when the first target-binding domain is not bound by the first target nucleic acid molecule, but anneals with [b] when the first target-binding domain is bound by the first target nucleic acid molecule,
   wherein the first catalytic domain is in an active state when motif [b] is annealed with motif [B].

In a further example, the first inhibitory domain is further characterized by i) or ii) below: i) motif [b] is at least 50% complementary to motif [C], and at least 20% complementary to motif [B], and ii) motif [B] is at least 50% complementary to motif [c], and at least 20% complementary to motif [b].

In a further example, the first inhibitory domain is further characterized by i) or ii) below:
i) motif [b] is at least 60%, at least 70%, at least 80% or at least 90% 6complementary to motif [C], and at least 20% complementary to motif [B], or
   ii) motif [B] is at least 60%, at least 70%, at least 80% or at least 90% complementary to motif [c], and at least 20% complementary to motif [b].

In another example, the ribozyme comprises the following structure:

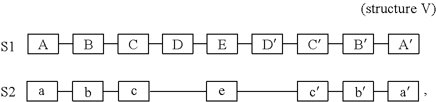

wherein:
S1 is the first RNA strand and S2 is the second RNA strand, wherein [A] to [A′] and [a] to [a′] represent opposite directionalities; wherein motifs [A] and [a] constitute a first trigger-binding domain for binding a first trigger nucleic acid molecule, motifs [B] and [b] constitute a linker that functions as a communication module to stabilise the catalytic domain when the trigger nucleic acid is bound, wherein motif [B] and [b] are independently at least 1 nucleotide in length;
or, wherein motifs [B] and [b] constitute a first inhibitory domain, motifs [C] and [c] constitute a first catalytic domain, motif [D] comprises a first cleavage site capable of being cleaved by the first catalytic domain, motifs [A′] and [a′] constitute a second trigger-binding domain for binding a second target RNA molecule; motifs [B′] and [b′] constitute a second linker that functions as a communication module to stabilise the catalytic domain when the trigger nucleic acid is bound, wherein motif [B′] and [b′] are independently at least 1 nucleotide in length;
or, wherein motifs [B′] and [b′] constitute a second inhibitory domain, motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region; wherein the first inhibitory domain is characterized by i) or ii) below:
   i) motif [b] anneals with [C] when the first target-binding domain is not bound by the first target nucleic acid molecule, but anneals with [B] when the first target-binding domain is bound by the first target nucleic acid molecule,
   ii) motif [B] anneals with motif [c] when the first target-binding domain is not bound by the first target nucleic acid molecule, but anneals with [b] when the first target-binding domain is bound by the first target nucleic acid molecule,
   wherein the second inhibitory domain is characterized by i) or ii) below:
      i) motif [b′] anneals with motif [C′] when the second target-binding domain is not bound by the first target nucleic acid molecule, but anneals with [B′] when the second target-binding domain is bound by the second target nucleic acid molecule,
      ii) motif [B′] anneals with motif [c′] when the second target-binding domain is not bound by the second target RNA molecule, but anneals with [b′] when the second target-binding domain is bound by the second target RNA molecule,
   wherein the first catalytic domain is in an active state when motif [b] is annealed with motif [B], and the second catalytic domain is in an active state when motif [b′] is annealed with motif [B′].

One or more secondary structures can be formed either individually by any of the motifs or optional linker regions, or formed collectively by motifs, linker regions, or combinations thereof. In an example, the optional linker regions individually or collectively form one or more secondary structures. In some examples of the ribozyme where the ribozymes comprise two target-binding domains and two catalytic domains, the ribozyme comprises one of the following structures:

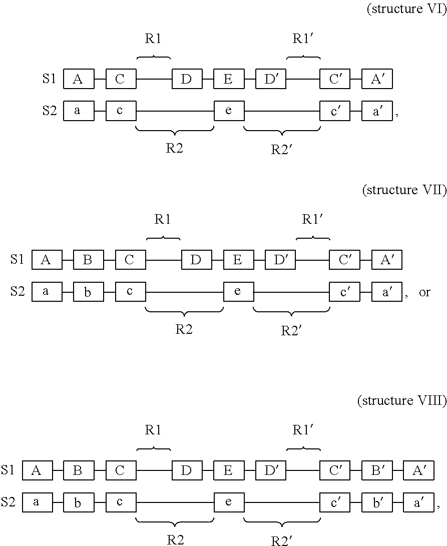

wherein linker regions R1 and R2 individually or collectively form one or more secondary structures, and linker regions R1′ and R2′ individually or collectively form one or more secondary structures.

TABLE 1
Sequence Listing table

| | SEQ | Trigger | Cleavage | SEQ | Cleavage | SEQ | Full | SEQ | Full |
|---|---|---|---|---|---|---|---|---|---|
| Ribozyme | ID | Se- | Product | ID | Product | ID | Sequence | ID | Sequence |
| Description | NO: | quence | Name | NO: | Sequence | NO: | (S1) | NO: | (S2) |
| Chong | | — | 29nt- | 307 | GUCCU | 3 | GGGAAGUGGU | 410 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 8-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CACACUUCC |
| | | | | | | | AUUGAGGUCG | | C |
| | | | | | | | UGAGUCCACG | | |
| | | | | | | | ACCUCCGUGG | | |
| | | | | | | | UAUAUUACCU | | |
| | | | | | | | GGUCCC | | |
| Chong | | — | 29nt- | 308 | GUCCU | 4 | GGGAAGUGGU | 411 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 6-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CACACUUC |
| | | | | | | | AUUGAGGUCG | | |
| | | | | | | | UGAGUCCACG | | |
| | | | | | | | ACCUCCGUGG | | |
| | | | | | | | UAUAUUACCU | | |
| | | | | | | | GGUU | | |
| Chong | | — | 29nt- | 309 | GUCCU | 5 | GGGAAGUGGU | 412 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 4-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CACACU |
| | | | | | | | AUUGAGGUCG | | |
| | | | | | | | UGAGUCCACG | | |
| | | | | | | | ACCUCCGUGG | | |
| | | | | | | | UAUAUUACCU | | |
| | | | | | | | GG | | |
| Chong | | — | 29nt- | 310 | GUCCU | 6 | GGGAAGUGGU | 413 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 3-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CACAC |
| | | | | | | | AUUGAGGUCG | | |
| | | | | | | | UGAGUCCACG | | |
| | | | | | | | ACCUCCGUGG | | |
| | | | | | | | UAUAUUACCU | | |
| | | | | | | | G | | |
| Chong | | — | 29nt- | 311 | GUCCU | 7 | GGGAAGUGGU | 414 | GGGAACCAG |
| Hui | | | clvR | | UAGUC | | AUAUUACCUG | | AGAAACACA |
| Circularly | | | NA | | GAAAGU | | GUACGCGUUC | | CGAAUCAGA |
| permutated | | | | | UUUACU | | ACGGUUCGCC | | GAAGACUCU |
| ribozyme | | | | | AGAGU | | GUGAACGCGU | | AGUAAAACU |
| 2-nt Helix | | | | | CA | | UGACAGUCCU | | UUCGACUAA |
| 4 | | | | | | | UAGUCGAAAG | | GAGAAGUCA |
| | | | | | | | UUUUACUAGA | | ACCAGAGAA |
| | | | | | | | GUCAGUCCUG | | ACACA |
| | | | | | | | AUUGAGGUCG | | |
| | | | | | | | UGAGUCCACG | | |
| | | | | | | | ACCUCCGUGG | | |
| | | | | | | | UAUAUUACCU | | |
| Chong | | — | 29nt- | 312 | GUCCU | 8 | GGGAAGUGGU | 415 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 1-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CAC |
| | | | | | | | AUUGAGGUCG | | |
| | | | | | | | UGAGUCCACG | | |
| | | | | | | | ACCUCCGUGG | | |
| | | | | | | | UAUAUUACC | | |
| Chong | | — | 29nt- | 313 | GUCCU | 9 | GGGAAGUGGU | 416 | GGGACCAGA |
| Hui | | | clvR | | UAGUC | | AUAUUACCUG | | GAAACACAC |
| Circularly | | | NA | | GAAAGU | | GUACGCGUUC | | GAAUCAGAG |
| permutated | | | | | UUUACU | | ACGGUUCGCC | | AAGACUCUA |
| ribozyme | | | | | AGAGU | | GUGAACGCGU | | GUAAAACUU |
| 0-nt Helix | | | | | CA | | UGACAGUCCU | | UCGACUAAG |
| 4 | | | | | | | UAGUCGAAAG | | AGAAGUCAA |
| | | | | | | | UUUUACUAGA | | CCAGAGAAA |
| | | | | | | | GUCAGUCCUG | | CA |
| | | | | | | | AUUGAGGUCG | | |
| | | | | | | | UGAGUCCACG | | |
| | | | | | | | ACCUCCGUGG | | |
| | | | | | | | UAUAUUAC | | |
| Chong | 221 | CCGG | 29nt- | 314 | GUCCU | 10 | GGGAAGUCAA | 417 | GGGACCGG |
| Hui IN- | | UUUU | clvR | | UAGUC | | ACCAAAGUGG | | UUUUCGAUU |
| form | | CGAU | NA | | GAAAGU | | UAUAUUACCU | | UGGUUUGAC |
| ribozyme | | UUGG | | | UUUACU | | GGUUUUCGAU | | UUUCGAGUC |
| 4WJ | | UUUG | | | AGAGU | | CGAAAGAUCG | | AAACCAACC |
| (HHHS2H) 2 x | | ACU | | | CA | | ACGAGGUGAA | | AGAGAAACA |
| 3/4 4-nt | | | | | | | AACCUCGUGA | | CACGAAUCA |
| Helix 4 | | | | | | | CAGUCCUUAG | | GAGAAGACG |
| | | | | | | | UCGAAAGUUU | | AGCGUCCCA |
| | | | | | | | UACUAGAGUC | | CGGGCGCA |
| | | | | | | | AGUCCUGAUU | | GAAGAGAAG |
| | | | | | | | GUGCUCGAAA | | UCAACCAGA |
| | | | | | | | GAGCACAGAC | | GAAACACAC |
| | | | | | | | CUGAAAAGGU | | UAUCGAAAA |
| | | | | | | | CUUUCGUGGU | | CCGGUUCGC |
| | | | | | | | AUAUUACCUG | | CGGUUUUCG |
| | | | | | | | GAUCGAAAAC | | AUUUGGUUU |
| | | | | | | | CGG | | GACU |
| Chong | 222 | CCGG | 29nt- | 315 | GUCCU | 11 | GGGAAGUCAA | 418 | GGGACCGG |
| Hui IN- | | UUUU | clvR | | UAGUC | | ACCAAAGUGU | | UUUUCGAUU |
| form | | CGAU | NA | | GAAAGU | | AUAUUACCUG | | UGGUUUGAC |
| ribozyme | | UUGG | | | UUUACU | | GUUUUCGAUC | | UUUCGAGUC |
| 4WJ | | UUUG | | | AGAGU | | GAAAGAUCGA | | AAACCAACA |
| (HHHS2H) 2 x | | ACU | | | CA | | CGAGGUGAAA | | GAGAAACAC |
| 3/4 3-nt | | | | | | | ACCUCGUGAC | | ACGAAUCAG |
| Helix 4 | | | | | | | AGUCCUUAGU | | AGAAGACGA |
| | | | | | | | CGAAAGUUUU | | GCGUCCCAC |
| | | | | | | | ACUAGAGUCA | | GGGCGCAGA |
| | | | | | | | GUCCUGAUUG | | AGAGAAGUC |
| | | | | | | | UGCUCGAAAG | | AACCAGAGA |
| | | | | | | | AGCACAGACC | | AACAACUAU |
| | | | | | | | UGAAAAGGUC | | CGAAAACCG |
| | | | | | | | UUUCGUGGUA | | GUUCGCCG |
| | | | | | | | UAUUACCUGA | | GUUUUCGAU |
| | | | | | | | UCGAAAACCG | | UUGGUUUGA |
| | | | | | | | G | | CU |
| Chong | 223 | CCGG | 29nt- | 316 | GUCCU | 12 | GGGAAGUCAA | 419 | GGGACCGG |
| Hui IN- | | UUUU | clvR | | UAGUC | | ACCAAAGGUA | | UUUUCGAUU |
| form | | CGAU | NA | | GAAAGU | | UAUUACCUGG | | UGGUUUGAC |
| ribozyme | | UUGG | | | UUUACU | | UUUUCGAUCG | | UUUCGAGUC |
| 4WJ | | UUUG | | | AGAGU | | AAAGAUCGAC | | AAACCAAAG |
| (HHHS2H) 2 x | | ACU | | | CA | | GAGGUGAAAA | | AGAAACACA |
| 3/4 2-nt | | | | | | | CCUCGUGACA | | CGAAUCAGA |
| Helix 4 | | | | | | | GUCCUUAGUC | | GAAGACGAG |
| | | | | | | | GAAAGUUUUA | | CGUCCCACG |
| | | | | | | | CUAGAGUCAG | | GGCGCAGAA |
| | | | | | | | UCCUGAUUGU | | GAGAAGUCA |
| | | | | | | | GCUCGAAAGA | | ACCAGAGAA |
| | | | | | | | GCACAGACCU | | ACACUAUCG |
| | | | | | | | GAAAAGGUCU | | AAAACCGGU |
| | | | | | | | UUCGUGGUAU | | UCGCCGGUU |
| | | | | | | | AUUACCUAUC | | UUCGAUUUG |
| | | | | | | | GAAAACCGG | | GUUUGACU |
| Chong | 224 | CCGG | 29nt- | 317 | GUCCU | 13 | GGGAAGUCAA | 420 | GGGACCGG |
| Hui IN- | | UUUU | clvR | | UAGUC | | ACCAAAGUAU | | UUUUCGAUU |
| form | | CGAU | NA | | GAAAGU | | AUUACCUGGU | | UGGUUUGAC |
| ribozyme | | UUGG | | | UUUACU | | UUUCGAUCGA | | UUUCGAGUC |
| 4WJ | | UUUG | | | AGAGU | | AAGAUCGACG | | AAACCAAGA |
| (HHHS2H) 2 x | | ACU | | | CA | | AGGUGAAAAC | | GAAACACAC |
| 3/4 1-nt | | | | | | | CUCGUGACAG | | GAAUCAGAG |
| Helix 4 | | | | | | | UCCUUAGUCG | | AAGACGAGC |
| | | | | | | | AAAGUUUUAC | | GUCCCACGG |
| | | | | | | | UAGAGUCAGU | | GCGCAGAAG |
| | | | | | | | CCUGAUUGUG | | AGAAGUCAA |
| | | | | | | | CUCGAAAGAG | | CCAGAGAAA |
| | | | | | | | CACAGACCUG | | CAUAUCGAA |
| | | | | | | | AAAAGGUCUU | | AACCGGUUC |
| | | | | | | | UCGUGGUAUA | | GCCGGUUUU |
| | | | | | | | UUACCAUCGA | | CGAUUUGGU |
| | | | | | | | AAACCGG | | UUGACU |
| Chong | 225 | CCGG | 29nt- | 318 | GUCCU | 14 | GGGAAGUCAA | 421 | GGGAAGUCA |
| Hui OUT- | | UUUU | clvR | | UAGUC | | ACCAAGGGAA | | AACCAAGGG |
| form | | CGAU | NA | | GAAAGU | | GUGGUAUAUU | | ACCAGAGAA |
| ribozyme | | UUGG | | | UUUACU | | ACCUGGUUUU | | ACACACGAA |
| 4WJ | | UUUG | | | AGAGU | | CGAUCGAAAG | | UCAGAGAAG |
| (HHHS2H) 2 x | | ACU | | | CA | | AUCGACGAGG | | ACGAGCGUC |
| 3/4 8-nt | | | | | | | UGAAAACCUC | | CCACGGGCG |
| Helix 4 | | | | | | | GUGACAGUCC | | CAGAAGAGA |
| | | | | | | | UUAGUCGAAA | | AGUCAACCA |
| | | | | | | | GUUUUACUAG | | GAGAAACAC |
| | | | | | | | AGUCAGUCCU | | ACUUCCCAU |
| | | | | | | | GAUUGUGCUC | | CGAAAACCG |
| | | | | | | | GAAAGAGCAC | | G |
| | | | | | | | AGACCUGAAA | | |
| | | | | | | | AGGUCUUUCG | | |
| | | | | | | | UGGUAUAUUA | | |
| | | | | | | | CCUGGUCCCA | | |
| | | | | | | | UCGAAAACCG | | |
| | | | | | | | G | | |
| Chong | 226 | CCGG | 29nt- | 319 | GUCCU | 15 | GGGAAGUCAA | 422 | GGGAAGUCA |
| Hui OUT- | | UUUU | clvR | | UAGUC | | ACCAAAGUGG | | AACCAACCA |
| form | | CGAU | NA | | GAAAGU | | UAUAUUACCU | | GAGAAACAC |
| ribozyme | | UUGG | | | UUUACU | | GGUUUUCGAU | | ACGAAUCAG |
| 4WJ | | UUUG | | | AGAGU | | CGAAAGAUCG | | AGAAGACGA |
| (HHHS2H) 2 x | | ACU | | | CA | | ACGAGGUGAA | | GCGUCCCAC |
| 3/4 4-nt | | | | | | | AACCUCGUGA | | GGGCGCAGA |
| Helix 4 | | | | | | | CAGUCCUUAG | | AGAGAAGUC |
| | | | | | | | UCGAAAGUUU | | AACCAGAGA |
| | | | | | | | UACUAGAGUC | | AACACACUA |
| | | | | | | | AGUCCUGAUU | | UCGAAAACC |
| | | | | | | | GUGCUCGAAA | | GG |
| | | | | | | | GAGCACAGAC | | |
| | | | | | | | CUGAAAAGGU | | |
| | | | | | | | CUUUCGUGGU | | |
| | | | | | | | AUAUUACCUG | | |
| | | | | | | | GAUCGAAAAC | | |
| | | | | | | | CGG | | |
| Chong | 227 | CCGG | 29nt- | 320 | GUCCU | 16 | GGGAAGUCAA | 423 | GGGAAGUCA |
| Hui OUT- | | UUUU | clvR | | UAGUC | | ACCAAGUGGU | | AACCAACAG |
| form | | CGAU | NA | | GAAAGU | | AUAUUACCUG | | AGAAACACA |
| ribozyme | | UUGG | | | UUUACU | | GUUUUCGAUC | | CGAAUCAGA |
| 4WJ | | UUUG | | | AGAGU | | GAAAGAUCGA | | GAAGACGAG |
| (HHHS2H) 2 x | | ACU | | | CA | | CGAGGUGAAA | | CGUCCCACG |
| 3/4 3-nt | | | | | | | ACCUCGUGAC | | GGCGCAGAA |
| Helix 4 | | | | | | | AGUCCUUAGU | | GAGAAGUCA |
| | | | | | | | CGAAAGUUUU | | ACCAGAGAA |
| | | | | | | | ACUAGAGUCA | | ACACACAUC |
| | | | | | | | GUCCUGAUUG | | GAAAACCGG |
| | | | | | | | UGCUCGAAAG | | |
| | | | | | | | AGCACAGACC | | |
| | | | | | | | UGAAAAGGUC | | |
| | | | | | | | UUUCGUGGUA | | |
| | | | | | | | UAUUACCUGA | | |
| | | | | | | | UCGAAAACCG | | |
| | | | | | | | G | | |
| Chong | 228 | CCGG | 29nt- | 321 | GUCCU | 17 | GGGAAGUCAA | 424 | GGGAAGUCA |
| Hui OUT- | | UUUU | clvR | | UAGUC | | ACCAAUGGUA | | AACCAAAGA |
| form | | CGAU | NA | | GAAAGU | | UAUUACCUGG | | GAAACACAC |
| ribozyme | | UUGG | | | UUUACU | | UUUUCGAUCG | | GAAUCAGAG |
| 4WJ | | UUUG | | | AGAGU | | AAAGAUCGAC | | AAGACGAGC |
| (HHHS2H) 2 x | | ACU | | | CA | | GAGGUGAAAA | | GUCCCACGG |
| 3/4 2-nt | | | | | | | CCUCGUGACA | | GCGCAGAAG |
| Helix 4 | | | | | | | GUCCUUAGUC | | AGAAGUCAA |
| | | | | | | | GAAAGUUUUA | | CCAGAGAAA |
| | | | | | | | CUAGAGUCAG | | CACAAUCGA |
| | | | | | | | UCCUGAUUGU | | AAACCGG |
| | | | | | | | GCUCGAAAGA | | |
| | | | | | | | GCACAGACCU | | |
| | | | | | | | GAAAAGGUCU | | |
| | | | | | | | UUCGUGGUAU | | |
| | | | | | | | AUUACCUAUC | | |
| | | | | | | | GAAAACCGG | | |
| Chong | 229 | CCGG | 29nt- | 322 | GUCCU | 18 | GGGAAGUCAA | 425 | GGGAAGUCA |
| Hui OUT- | | UUUU | clvR | | UAGUC | | ACCAAGGUAU | | AACCAAGAG |
| form | | CGAU | NA | | GAAAGU | | AUUACCUGGU | | AAACACACG |
| ribozyme | | UUGG | | | UUUACU | | UUUCGAUCGA | | AAUCAGAGA |
| 4WJ | | UUUG | | | AGAGU | | AAGAUCGACG | | AGACGAGCG |
| (HHHS2H) 2 x | | ACU | | | CA | | AGGUGAAAAC | | UCCCACGGG |
| 3/4 1-nt | | | | | | | CUCGUGACAG | | CGCAGAAGA |
| Helix 4 | | | | | | | UCCUUAGUCG | | GAAGUCAAC |
| | | | | | | | AAAGUUUUAC | | CAGAGAAAC |
| | | | | | | | UAGAGUCAGU | | ACAUCGAAA |
| | | | | | | | CCUGAUUGUG | | ACCGG |
| | | | | | | | CUCGAAAGAG | | |
| | | | | | | | CACAGACCUG | | |
| | | | | | | | AAAAGGUCUU | | |
| | | | | | | | UCGUGGUAUA | | |
| | | | | | | | UUACCAUCGA | | |
| | | | | | | | AAACCGG | | |
| Dual | | — | 29nt- | 323 | GUCCU | 19 | gggaagtgGTAT | 426 | gggaccagAGA |
| ribozyme | | | clvR | | UAGUC | | ATTACctggtTCT | | AACAcacgatc |
| 2WJ | | | NA | | GAAAGU | | TTCTgacAGTCc | | agAGAAgACT |
| Paired | | | | | UUUACU | | TTAGTCGAAA | | CTAGTAAAA |
| | | | | | AGAGU | | GTTTTACTAGA | | CTTTCGACT |
| | | | | | CA | | GTcAGTCctgatT | | AAgAGAAgtca |
| | | | | | | | CTTTCTcgtgGT | | ccagAGAAAC |
| | | | | | | | ATATTACctggtc | | Acacttccc |
| | | | | | | | CC | | |
| Dual | | — | 29nt- | 324 | GUCCU | 20 | gggaagtgGTAT | 427 | gggaccagAGA |
| ribozyme | | | clvR | | UAGUC | | ATTACctggtacg | | AACAcacgaat |
| 3WJ | | | NA | | GAAAGU | | cgttcacggTTCG | | cagAGAAgAC |
| (HHH) | | | | | UUUACU | | ccgtgaacgcgttga | | TCTAGTAAAA |
| Paired | | | | | AGAGU | | CAGTCcTTAGT | | CTTTCGACT |
| | | | | | CA | | CGAAAGTTTTA | | AAgAGAAgtca |
| | | | | | | | CTAGAGTcAGT | | accagAGAAA |
| | | | | | | | Cctgattgaggtcgt | | CAcacttccc |
| | | | | | | | gAGTCcacgacct | | |
| | | | | | | | ccgtgGTATATT | | |
| | | | | | | | ACctggtccc | | |
| Dual | | — | 29nt- | 325 | GUCCU | 21 | gggaagtgGTAT | 428 | gggaccagAGA |
| ribozyme | | | clvR | | UAGUC | | ATTACctggtacg | | AACAcacgaat |
| 3WJ | | | NA | | GAAAGU | | cgttcacggTTCG | | cagAGAAgAC |
| (HHH) | | | | | UUUACU | | ccgtgaacgcgttga | | AGATCATTTT |
| Unpaired | | | | | AGAGU | | CAGTCcTTAGT | | GAAAGCTGA |
| | | | | | CA | | CGAAAGTTTTA | | AAgAGAAgtca |
| | | | | | | | CTAGAGTcAGT | | accagAGAAA |
| | | | | | | | Cctgattgaggtcgt | | CAcacttccc |
| | | | | | | | gAGTCcacgacct | | |
| | | | | | | | ccgtgGTATATT | | |
| | | | | | | | ACctggtccc | | |
| Dual | | — | 29nt- | 326 | GUCCU | 22 | gggaagtgGTAT | 429 | gggaccagAGA |
| ribozyme | | | clvR | | UAGUC | | ATTACctggtCTT | | AACAcacgaat |
| 3WJ | | | NA | | GAAAGU | | TacgcgttcacggT | | cagAGAAgAC |
| (HHS4H) | | | | | UUUACU | | TCGccgtgaacgc | | TCTAGTAAAA |
| Paired | | | | | AGAGU | | gttgacAGTCcTT | | CTTTCGACT |
| | | | | | CA | | AGTCGAAAGT | | AAgAGAAgtca |
| | | | | | | | TTTACTAGAGT | | accagAGAAA |
| | | | | | | | CAGTCctgattgag | | CAcacttccc |
| | | | | | | | gtcgtgAGTCcac | | |
| | | | | | | | gacctcCTTTcgtg | | |
| | | | | | | | GTATATTACctg | | |
| | | | | | | | gtccc | | |
| Dual | | — | 29nt- | 327 | GUCCU | 23 | gggaagtgGTAT | 430 | gggaccagAGA |
| ribozyme | | | clvR | | UAGUC | | ATTACctggtCTT | | AACAcacgaat |
| 3WJ | | | NA | | GAAAGU | | TacgcgttcacggT | | cagAGAAgAC |
| (HHS4H) | | | | | UUUACU | | TCGccgtgaacgc | | AGATCATTTT |
| Unpaired | | | | | AGAGU | | gttgacAGTCcTT | | GAAAGCTGA |
| | | | | | CA | | AGTCGAAAGT | | AAgAGAAgtca |
| | | | | | | | TTTACTAGAGT | | accagAGAAA |
| | | | | | | | CAGTCctgattgag | | CAcacttccc |
| | | | | | | | gtcgtgAGTCcac | | |
| | | | | | | | gacctcCTTTcgtg | | |
| | | | | | | | GTATATTACctg | | |
| | | | | | | | gtccc | | |
| Dual | | — | 29nt- | 328 | GUCCU | 24 | gggaagtgGTAT | 431 | gggaccagAGA |
| ribozyme | | | clvR | | UAGUC | | ATTACctggtTTtc | | AACAcacgaat |
| 4WJ | | | NA | | GAAAGU | | gatcGAAAgatcg | | cagAGAAgAC |
| (HHHS2H) Paired | | | | | UUUACU | | acgaggtGAAAac | | TCTAGTAAAA |
| | | | | | AGAGU | | ctcgtgacAGTCc | | CTTTCGACT |
| | | | | | CA | | TTAGTCGAAA | | AAgAGAAgtca |
| | | | | | | | GTTTTACTAGA | | accagAGAAA |
| | | | | | | | GTcAGTCctgatt | | CAcacttccc |
| | | | | | | | gtgctcGAAAgag | | |
| | | | | | | | cacagacctGAAA | | |
| | | | | | | | aggtctTTcgtgGT | | |
| | | | | | | | ATATTACctggtc | | |
| | | | | | | | CC | | |
| Dual | | — | 29nt- | 329 | GUCCU | 25 | gggaagtgGTAT | 432 | gggaccagAGA |
| ribozyme | | | clvR | | UAGUC | | ATTACctggtTTtc | | AACAcacgaat |
| 4WJ | | | NA | | GAAAGU | | gatcGAAAgatcg | | cagAGAAgAC |
| (HHHS2H) | | | | | UUUACU | | acgaggtGAAAac | | AGATCATTTT |
| Unpaired | | | | | AGAGU | | ctcgtgacAGTCc | | GAAAGCTGA |
| | | | | | CA | | TTAGTCGAAA | | AAgAGAAgtca |
| | | | | | | | GTTTTACTAGA | | accagAGAAA |
| | | | | | | | GTcAGTCctgatt | | CAcacttccc |
| | | | | | | | gtgctcGAAAgag | | |
| | | | | | | | cacagacctGAAA | | |
| | | | | | | | aggtctTTcgtgGT | | |
| | | | | | | | ATATTACctggtc | | |
| | | | | | | | CC | | |
| Dual | | — | 29nt- | 330 | GUCCU | 26 | gggaagtgGTAT | 433 | gggaccagAGA |
| ribozyme | | | clvR | | UAGUC | | ATTACctggtTTtc | | AACAcacgaat |
| 4WJ | | | NA | | GAAAGU | | gatcGAAAgatcg | | cagAGAAgAC |
| (HHHS2H) | | | | | UUUACU | | acgaggtGAAAac | | GAGCTGCCC |
| Unpaired | | | | | AGAGU | | ctcgtgacAGTCc | | CAGGGATCA |
| #3 | | | | | CA | | TTAGTCGAAA | | GAAgAGAAgt |
| | | | | | | | GTTTTACTAGA | | caaccagAGAA |
| | | | | | | | GTcAGTCctgatt | | ACAcacttccc |
| | | | | | | | gtgctcGAAAgag | | |
| | | | | | | | cacagacctGAAA | | |
| | | | | | | | aggtctTTcgtgGT | | |
| | | | | | | | ATATTACctggtc | | |
| | | | | | | | CC | | |
| Dual | | — | 29nt- | 331 | GUCCU | 27 | gggaagtgGTAT | 434 | gggaccagAGA |
| ribozyme | | | clvR | | UAGUC | | ATTACctggtTTtc | | AACAcacgaat |
| 4WJ | | | NA | | GAAAGU | | gatcGAAAgatcg | | cagAGAAgAC |
| (HHHS2H) | | | | | UUUACU | | acgaggtGAAAac | | GAGAGTCCA |
| 3 x 2 | | | | | AGAGU | | ctcgtgacAGTCc | | ACGGTCGCA |
| #4 | | | | | CA | | TTAGTCGAAA | | GAAgAGAAgt |
| | | | | | | | GTTTTACTAGA | | caaccagAGAA |
| | | | | | | | GTcAGTCctgatt | | ACAcacttccc |
| | | | | | | | gtgctcGAAAgag | | |
| | | | | | | | cacagacctGAAA | | |
| | | | | | | | aggtctTTcgtgGT | | |
| | | | | | | | ATATTACctggtc | | |
| | | | | | | | CC | | |
| Dual | | — | 29nt- | 332 | GUCCU | 28 | gggaagtgGTAT | 435 | gggaccagAGA |
| ribozyme | | | clvR | | UAGUC | | ATTACctggtTTtc | | AACAcacgaat |
| 4WJ | | | NA | | GAAAGU | | gatcGAAAgatcg | | cagAGAAgAC |
| (HHHS2H) | | | | | UUUACU | | acgaggtGAAAac | | GAGCGTCCC |
| 2 x | | | | | AGAGU | | ctcgtgacAGTCc | | ACGGGCGCA |
| 3/4 #5 | | | | | CA | | TTAGTCGAAA | | GAAgAGAAgt |
| | | | | | | | GTTTTACTAGA | | caaccagAGAA |
| | | | | | | | GTcAGTCctgatt | | ACAcacttccc |
| | | | | | | | gtgctcGAAAgag | | |
| | | | | | | | cacagacctGAAA | | |
| | | | | | | | aggtctTTcgtgGT | | |
| | | | | | | | ATATTACctggtc | | |
| | | | | | | | CC | | |
| Dual | 230 | UGAG | 29nt- | 333 | GUCCU | 29 | gggaAACUAUA | 436 | gggaAACUAU |
| ribozyme | | GUAG | clvR | | UAGUC | | CAAUGUAUAU | | ACAAUGUAA |
| T-let- | | UAGA | NA | | GAAAGU | | UAGUAUAUUA | | UAUAAGAAA |
| 7f Cl | | UUGU | | | UUUACU | | CCUGGUUUUC | | CACACGAAU |
| 29nt- | | AUAG | | | AGAGU | | GAUCGAAAGA | | CAGAGAAGA |
| clvRNA | | UU | | | CA | | UCGACGAGGU | | CGAGCGUCC |
| | | | | | | | GAAAACCUCG | | CACGGGCGC |
| | | | | | | | UGACAGUCCU | | AGAAGAGAA |
| | | | | | | | UAGUCGAAAG | | GUCAACCAG |
| | | | | | | | UUUUACUAGA | | AGAAACAUA |
| | | | | | | | GUCAGUCCUG | | AUAUACCUA |
| | | | | | | | AUUGUGCUCG | | CUACCUCA |
| | | | | | | | AAAGAGCACA | | |
| | | | | | | | GACCUGAAAA | | |
| | | | | | | | GGUCUUUCGU | | |
| | | | | | | | GGUAUAUUAC | | |
| | | | | | | | UCUCUGAACU | | |
| | | | | | | | ACUACCUCA | | |
| Dual | 231 | CCGG | 29nt- | 334 | GUCCU | 30 | GGGAAGUCAA | 437 | GGGAAGUCA |
| ribozyme | | UUUU | clvR | | UAGUC | | ACCAAGUAUA | | AACCAAGUA |
| T-ban- | | CGAU | NA | | GAAAGU | | UUAGUAUAUU | | AUAUAAGAA |
| 5p_Cl- | | UUGG | | | UUUACU | | ACCUGGUUUU | | ACACACGAA |
| 29nt- | | UUUG | | | AGAGU | | CGAUCGAAAG | | UCAGAGAAG |
| clvRNA | | ACU | | | CA | | AUCGACGAGG | | ACGAGCGUC |
| | | | | | | | UGAAAACCUC | | CCACGGGCG |
| | | | | | | | GUGACAGUCC | | CAGAAGAGA |
| | | | | | | | UUAGUCGAAA | | AGUCAACCA |
| | | | | | | | GUUUUACUAG | | GAGAAACAU |
| | | | | | | | AGUCAGUCCU | | AAUAUACAU |
| | | | | | | | GAUUGUGCUC | | CGAAAACCG |
| | | | | | | | GAAAGAGCAC | | G |
| | | | | | | | AGACCUGAAA | | |
| | | | | | | | AGGUCUUUCG | | |
| | | | | | | | UGGUAUAUUA | | |
| | | | | | | | CUCUCUGAAA | | |
| | | | | | | | UCGAAAACCG | | |
| | | | | | | | G | | |
| ban-5p | 232 | CCGG | 29nt- | 335 | GUCCU | 31 | GGGAAGUCAA | 438 | GGGAAGUCA |
| double | | UUUU | clvR | | UAGUC | | ACCAAGUAUA | | AACCAAGUA |
| catalytic | | CGAU | NA | | GAAAGU | | UUAGUuUAUU | | AUAUAAGAA |
| mutant | | UUGG | | | UUUACU | | ACCUGGUUUU | | AgACACGAA |
| | | UUUG | | | AGAGU | | CGAUCGAAAG | | UCAGAGAAG |
| | | ACU | | | CA | | AUCGACGAGG | | ACGAGCGUC |
| | | | | | | | UGAAAACCUC | | CCACGGGCG |
| | | | | | | | GUGACAGUCC | | CAGAAGAGA |
| | | | | | | | UUAGUCGAAA | | AGUCAACCA |
| | | | | | | | GUUUUACUAG | | GAGAAAgAU |
| | | | | | | | AGUCAGUCCU | | AAUAUACAU |
| | | | | | | | GAUUGUGCUC | | CGAAAACCG |
| | | | | | | | GAAAGAGCAC | | G |
| | | | | | | | AGACCUGAAA | | |
| | | | | | | | AGGUCUUUCG | | |
| | | | | | | | UGGUuUAUUA | | |
| | | | | | | | CUCUCUGAAA | | |
| | | | | | | | UCGAAAACCG | | |
| | | | | | | | G | | |
| ban-5p | 233 | CCGG | 29nt- | 336 | GUCCU | 32 | GGGAAGUCAA | 439 | GGGAAGUCA |
| distal | | UUUU | clvR | | UAGUC | | ACCAAGUAUA | | AACCAAGUA |
| catalytic | | CGAU | NA | | GAAAGU | | UUAGUAUAUU | | AUAUAAGAA |
| mutant | | UUGG | | | UUUACU | | ACCUGGUUUU | | AgACACGAA |
| | | UUUG | | | AGAGU | | CGAUCGAAAG | | UCAGAGAAG |
| | | ACU | | | CA | | AUCGACGAGG | | ACGAGCGUC |
| | | | | | | | UGAAAACCUC | | CCACGGGCG |
| | | | | | | | GUGACAGUCC | | CAGAAGAGA |
| | | | | | | | UUAGUCGAAA | | AGUCAACCA |
| | | | | | | | GUUUUACUAG | | GAGAAACAU |
| | | | | | | | AGUCAGUCCU | | AAUAUACAU |
| | | | | | | | GAUUGUGCUC | | CGAAAACCG |
| | | | | | | | GAAAGAGCAC | | G |
| | | | | | | | AGACCUGAAA | | |
| | | | | | | | AGGUCUUUCG | | |
| | | | | | | | UGGUuUAUUA | | |
| | | | | | | | CUCUCUGAAA | | |
| | | | | | | | UCGAAAACCG | | |
| | | | | | | | G | | |
| ban-5p | 234 | CCGG | 29nt- | 337 | GUCCU | 33 | GGGAAGUCAA | 440 | GGGAAGUCA |
| proximal | | UUUU | clvR | | UAGUC | | ACCAAGUAUA | | AACCAAGUA |
| catalytic | | CGAU | NA | | GAAAGU | | UUAGUuUAUU | | AUAUAAGAA |
| mutant | | UUGG | | | UUUACU | | ACCUGGUUUU | | ACACACGAA |
| | | UUUG | | | AGAGU | | CGAUCGAAAG | | UCAGAGAAG |
| | | ACU | | | CA | | AUCGACGAGG | | ACGAGCGUC |
| | | | | | | | UGAAAACCUC | | CCACGGGCG |
| | | | | | | | GUGACAGUCC | | CAGAAGAGA |
| | | | | | | | UUAGUCGAAA | | AGUCAACCA |
| | | | | | | | GUUUUACUAG | | GAGAAAgAU |
| | | | | | | | AGUCAGUCCU | | AAUAUACAU |
| | | | | | | | GAUUGUGCUC | | CGAAAACCG |
| | | | | | | | GAAAGAGCAC | | G |
| | | | | | | | AGACCUGAAA | | |
| | | | | | | | AGGUCUUUCG | | |
| | | | | | | | UGGUAUAUUA | | |
| | | | | | | | CUCUCUGAAA | | |
| | | | | | | | UCGAAAACCG | | |
| | | | | | | | G | | |
| Dual | 235 | AAAC | 29nt- | 338 | GUCCU | 34 | GGGAUUUGGC | 441 | GGGAUUUG |
| ribozyme | | CGUU | clvR | | UAGUC | | AAUGGGUAUA | | GCAAUGGGU |
| T-mir- | | ACCA | NA | | GAAAGU | | UUAGUAUAUU | | AAUAUAAGA |
| 451a_Cl- | | UUAC | | | UUUACU | | ACCUGGUUUU | | AACACACGA |
| 29nt- | | UGAG | | | AGAGU | | CGAUCGAAAG | | AUCAGAGAA |
| clvRNA | | UU | | | CA | | AUCGACGAGG | | GACGAGCGU |
| | | | | | | | UGAAAACCUC | | CCCACGGGC |
| | | | | | | | GUGACAGUCC | | GCAGAAGAG |
| | | | | | | | UUAGUCGAAA | | AAGUCAACC |
| | | | | | | | GUUUUACUAG | | AGAGAAACA |
| | | | | | | | AGUCAGUCCU | | UAAUAUACU |
| | | | | | | | GAUUGUGCUC | | AAUGACUCA |
| | | | | | | | GAAAGAGCAC | | A |
| | | | | | | | AGACCUGAAA | | |
| | | | | | | | AGGUCUUUCG | | |
| | | | | | | | UGGUAUAUUA | | |
| | | | | | | | CUCUCUGAAU | | |
| | | | | | | | AAUGACUCAA | | |
| mir-451a | 236 | AAAC | 29nt- | 339 | GUCCU | 35 | GGGAUUUGGC | 442 | GGGAUUUG |
| double | | CGUU | clvR | | UAGUC | | AAUGGGUAUA | | GCAAUGGGU |
| catalytic | | ACCA | NA | | GAAAGU | | UUAGUuUAUU | | AAUAUAAGA |
| mutant | | UUAC | | | UUUACU | | ACCUGGUUUU | | AAgACACGA |
| | | UGAG | | | AGAGU | | CGAUCGAAAG | | AUCAGAGAA |
| | | UU | | | CA | | AUCGACGAGG | | GACGAGCGU |
| | | | | | | | UGAAAACCUC | | CCCACGGGC |
| | | | | | | | GUGACAGUCC | | GCAGAAGAG |
| | | | | | | | UUAGUCGAAA | | AAGUCAACC |
| | | | | | | | GUUUUACUAG | | AGAGAAAgA |
| | | | | | | | AGUCAGUCCU | | UAAUAUACU |
| | | | | | | | GAUUGUGCUC | | AAUGACUCA |
| | | | | | | | GAAAGAGCAC | | A |
| | | | | | | | AGACCUGAAA | | |
| | | | | | | | AGGUCUUUCG | | |
| | | | | | | | UGGUuUAUUA | | |
| | | | | | | | CUCUCUGAAU | | |
| | | | | | | | AAUGACUCAA | | |
| mir-451a | 237 | AAAC | 29nt- | 340 | GUCCU | 36 | GGGAUUUGGC | 443 | GGGAUUUG |
| distal | | CGUU | clvR | | UAGUC | | AAUGGGUAUA | | GCAAUGGGU |
| catalytic | | ACCA | NA | | GAAAGU | | UUAGUAUAUU | | AAUAUAAGA |
| mutant | | UUAC | | | UUUACU | | ACCUGGUUUU | | AAgACACGA |
| | | UGAG | | | AGAGU | | CGAUCGAAAG | | AUCAGAGAA |
| | | UU | | | CA | | AUCGACGAGG | | GACGAGCGU |
| | | | | | | | UGAAAACCUC | | CCCACGGGC |
| | | | | | | | GUGACAGUCC | | GCAGAAGAG |
| | | | | | | | UUAGUCGAAA | | AAGUCAACC |
| | | | | | | | GUUUUACUAG | | AGAGAAACA |
| | | | | | | | AGUCAGUCCU | | UAAUAUACU |
| | | | | | | | GAUUGUGCUC | | AAUGACUCA |
| | | | | | | | GAAAGAGCAC | | A |
| | | | | | | | AGACCUGAAA | | |
| | | | | | | | AGGUCUUUCG | | |
| | | | | | | | UGGUuUAUUA | | |
| | | | | | | | CUCUCUGAAU | | |
| | | | | | | | AAUGACUCAA | | |
| mir-451a | 238 | AAAC | 29nt- | 341 | GUCCU | 37 | GGGAUUUGGC | 444 | GGGAUUUG |
| proximal | | CGUU | clvR | | UAGUC | | AAUGGGUAUA | | GCAAUGGGU |
| catalytic | | ACCA | NA | | GAAAGU | | UUAGUuUAUU | | AAUAUAAGA |
| mutant | | UUAC | | | UUUACU | | ACCUGGUUUU | | AACACACGA |
| | | UGAG | | | AGAGU | | CGAUCGAAAG | | AUCAGAGAA |
| | | UU | | | CA | | AUCGACGAGG | | GACGAGCGU |
| | | | | | | | UGAAAACCUC | | CCCACGGGC |
| | | | | | | | GUGACAGUCC | | GCAGAAGAG |
| | | | | | | | UUAGUCGAAA | | AAGUCAACC |
| | | | | | | | GUUUUACUAG | | AGAGAAAgA |
| | | | | | | | AGUCAGUCCU | | UAAUAUACU |
| | | | | | | | GAUUGUGCUC | | AAUGACUCA |
| | | | | | | | GAAAGAGCAC | | A |
| | | | | | | | AGACCUGAAA | | |
| | | | | | | | AGGUCUUUCG | | |
| | | | | | | | UGGUAUAUUA | | |
| | | | | | | | CUCUCUGAAU | | |
| | | | | | | | AAUGACUCAA | | |
| Dual | 239 | UGAG | 29nt- | 342 | GUCCU | 38 | gggaAACUAUA | 445 | gggaAACUAU |
| ribozyme | | GUAG | clvR | | UAGUC | | CAAUGUAUAU | | ACAAUGUAA |
| T-let- | | UAGA | NA | | GAAAGU | | UAGUAUAUUA | | UAUAAGAAA |
| 7f_Cl | | UUGU | | | UUUACU | | CCUGGUUUUC | | CACACGAAU |
| 29nt- | | AUAG | | | AGAGU | | GAUCGAAAGA | | CAGAGAAGA |
| clvRNA | | UU | | | CA | | UCGACGAGGU | | CGAGCGUCC |
| | | | | | | | GAAAACCUCG | | CACGGGCGC |
| | | | | | | | UGACAGUCCU | | AGAAGAGAA |
| | | | | | | | UAGUCGAAAG | | GUCAACCAG |
| | | | | | | | UUUUACUAGA | | AGAAACAUA |
| | | | | | | | GUCAGUCCUG | | AUAUACCUA |
| | | | | | | | AUUGUGCUCG | | CUACCUCA |
| | | | | | | | AAAGAGCACA | | |
| | | | | | | | GACCUGAAAA | | |
| | | | | | | | GGUCUUUCGU | | |
| | | | | | | | GGUAUAUUAC | | |
| | | | | | | | UCUCUGAACU | | |
| | | | | | | | ACUACCUCA | | |
| let-7f | 240 | UGAG | 29nt- | 343 | GUCCU | 39 | gggaAACUAUA | 446 | gggaAACUAU |
| double | | GUAG | clvR | | UAGUC | | CAAUGUAUAU | | ACAAUGUAA |
| catalytic | | UAGA | NA | | GAAAGU | | UAGUuUAUUA | | UAUAAGAAA |
| mutant | | UUGU | | | UUUACU | | CCUGGUUUUC | | gACACGAAU |
| | | AUAG | | | AGAGU | | GAUCGAAAGA | | CAGAGAAGA |
| | | UU | | | CA | | UCGACGAGGU | | CGAGCGUCC |
| | | | | | | | GAAAACCUCG | | CACGGGCGC |
| | | | | | | | UGACAGUCCU | | AGAAGAGAA |
| | | | | | | | UAGUCGAAAG | | GUCAACCAG |
| | | | | | | | UUUUACUAGA | | AGAAAgAUA |
| | | | | | | | GUCAGUCCUG | | AUAUACCUA |
| | | | | | | | AUUGUGCUCG | | CUACCUCA |
| | | | | | | | AAAGAGCACA | | |
| | | | | | | | GACCUGAAAA | | |
| | | | | | | | GGUCUUUCGU | | |
| | | | | | | | GGUuUAUUAC | | |
| | | | | | | | UCUCUGAACU | | |
| | | | | | | | ACUACCUCA | | |
| let-7f | 241 | UGAG | 29nt- | 344 | GUCCU | 40 | gggaAACUAUA | 447 | gggaAACUAU |
| distal | | GUAG | clvR | | UAGUC | | CAAUGUAUAU | | ACAAUGUAA |
| catalytic | | UAGA | NA | | GAAAGU | | UAGUAUAUUA | | UAUAAGAAA |
| mutant | | UUGU | | | UUUACU | | CCUGGUUUUC | | gACACGAAU |
| | | AUAG | | | AGAGU | | GAUCGAAAGA | | CAGAGAAGA |
| | | UU | | | CA | | UCGACGAGGU | | CGAGCGUCC |
| | | | | | | | GAAAACCUCG | | CACGGGCGC |
| | | | | | | | UGACAGUCCU | | AGAAGAGAA |
| | | | | | | | UAGUCGAAAG | | GUCAACCAG |
| | | | | | | | UUUUACUAGA | | AGAAACAUA |
| | | | | | | | GUCAGUCCUG | | AUAUACCUA |
| | | | | | | | AUUGUGCUCG | | CUACCUCA |
| | | | | | | | AAAGAGCACA | | |
| | | | | | | | GACCUGAAAA | | |
| | | | | | | | GGUCUUUCGU | | |
| | | | | | | | GGUuUAUUAC | | |
| | | | | | | | UCUCUGAACU | | |
| | | | | | | | ACUACCUCA | | |
| let-7f | 242 | UGAG | 29nt- | 345 | GUCCU | 41 | gggaAACUAUA | 448 | gggaAACUAU |
| proximal | | GUAG | clvR | | UAGUC | | CAAUGUAUAU | | ACAAUGUAA |
| catalytic | | UAGA | NA | | GAAAGU | | UAGUuUAUUA | | UAUAAGAAA |
| mutant | | UUGU | | | UUUACU | | CCUGGUUUUC | | CACACGAAU |
| | | AUAG | | | AGAGU | | GAUCGAAAGA | | CAGAGAAGA |
| | | UU | | | CA | | UCGACGAGGU | | CGAGCGUCC |
| | | | | | | | GAAAACCUCG | | CACGGGCGC |
| | | | | | | | UGACAGUCCU | | AGAAGAGAA |
| | | | | | | | UAGUCGAAAG | | GUCAACCAG |
| | | | | | | | UUUUACUAGA | | AGAAAgAUA |
| | | | | | | | GUCAGUCCUG | | AUAUACCUA |
| | | | | | | | AUUGUGCUCG | | CUACCUCA |
| | | | | | | | AAAGAGCACA | | |
| | | | | | | | GACCUGAAAA | | |
| | | | | | | | GGUCUUUCGU | | |
| | | | | | | | GGUAUAUUAC | | |
| | | | | | | | UCUCUGAACU | | |
| | | | | | | | ACUACCUCA | | |
| One- | 243 | CCGG | 29nt- | 346 | GUCCU | 42 | gggaAGUCAAA | | |
| sided T- | | UUUU | clvR | | UAGUC | | CCAAGUAUAU | | |
| ban5p_Cl | | CGAU | NA | | GAAAGU | | UAGUAUAUUA | | |
| -29nt- | | UUGG | | | UUUACU | | CCUGGUUUUC | | |
| clvRNA | | UUUG | | | AGAGU | | GAUCGAAAGA | | |
| | | ACU | | | CA | | UCGACGAGGU | | |
| | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | AUCGAAAACC | | |
| | | | | | | | GG | | |
| One- | 244 | AAAC | 29nt- | 347 | GUCCU | 43 | gggaAACUCAG | | |
| sided T- | | CGUU | clvR | | UAGUC | | UAAUGUAUAU | | |
| mir- | | ACCA | NA | | GAAAGU | | UAGUAUAUUA | | |
| 451a_Cl | | UUAC | | | UUUACU | | CCUGGUUUUC | | |
| 29nt- | | UGAG | | | AGAGU | | GAUCGAAAGA | | |
| clvRNA | | UU | | | CA | | UCGACGAGGU | | |
| | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | GGUAACGGUU | | |
| | | | | | | | U | | |
| One- | 245 | UUCG | 29nt- | 348 | GUCCU | 44 | gggaACGUACC | | |
| sided T- | | GAAG | clvR | | UAGUC | | UGUGUAUAUU | | |
| E- | | AGAC | NA | | GAAAGU | | AGUAUAUUAC | | |
| gene_Cl | | AGGU | | | UUUACU | | CUGGUUUUCG | | |
| 29nt- | | ACGU | | | AGAGU | | AUCGAAAGAU | | |
| clvRNA | | | | | CA | | CGACGAGGUG | | |
| | | | | | | | AAAACCUCGU | | |
| | | | | | | | GACAGUCCUU | | |
| | | | | | | | AGUCGAAAGU | | |
| | | | | | | | UUUACUAGAG | | |
| | | | | | | | UCAGUCCUGA | | |
| | | | | | | | UUUUCGAAUC | | |
| | | | | | | | AGAGAAGACG | | |
| | | | | | | | AGCGUCCCAC | | |
| | | | | | | | GGGCGCAGAA | | |
| | | | | | | | GAGAAGUCAA | | |
| | | | | | | | CCAGAGAAAC | | |
| | | | | | | | AUAAUAUACC | | |
| | | | | | | | UCUUCCGAA | | |
| One- | 246 | UGGA | 29nt- | 349 | GUCCU | 45 | gggaGCCCUUA | | |
| sided T- | | CGGA | clvR | | UAGUC | | UCAGGUAUAU | | |
| mir- | | GAAC | NA | | GAAAGU | | UAGUAUAUUA | | |
| 184_Cl | | UGAU | | | UUUACU | | CCUGGUUUUC | | |
| 29nt- | | AAGG | | | AGAGU | | GAUCGAAAGA | | |
| clvRNA | | GC | | | CA | | UCGACGAGGU | | |
| | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | UUCUCCGUCC | | |
| | | | | | | | A | | |
| One- | 247 | CUAA | 29nt- | 350 | GUCCU | 46 | gggaCUCCUGC | | |
| sided T- | | GUAC | clvR | | UAGUC | | GGCAGUAUAU | | |
| mir- | | UAGU | NA | | GAAAGU | | UAGUAUAUUA | | |
| 252_Cl | | GCCG | | | UUUACU | | CCUGGUUUUC | | |
| 29nt- | | CAGG | | | AGAGU | | GAUCGAAAGA | | |
| clvRNA | | AG | | | CA | | UCGACGAGGU | | |
| | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | CUAGUACUUA | | |
| | | | | | | | G | | |
| One- | 248 | AAUG | 29nt- | 351 | GUCCU | 47 | gggaCCCGUGA | | |
| sided T- | | GCAC | clvR | | UAGUC | | AUUCUGUAUA | | |
| mir- | | UGGA | NA | | GAAAGU | | UUAGUAUAUU | | |
| 263a_Cl- | | AGAA | | | UUUACU | | ACCUGGUUUU | | |
| 29nt- | | UUCA | | | AGAGU | | CGAUCGAAAG | | |
| clvRNA | | CGGG | | | CA | | AUCGACGAGG | | |
| | | | | | | | UGAAAACCUC | | |
| | | | | | | | GUGACAGUCC | | |
| | | | | | | | UUAGUCGAAA | | |
| | | | | | | | GUUUUACUAG | | |
| | | | | | | | AGUCAGUCCU | | |
| | | | | | | | GAUUUUCGAA | | |
| | | | | | | | UCAGAGAAGA | | |
| | | | | | | | CGAGCGUCCC | | |
| | | | | | | | ACGGGCGCAG | | |
| | | | | | | | AAGAGAAGUC | | |
| | | | | | | | AACCAGAGAA | | |
| | | | | | | | ACAUAAUAUA | | |
| | | | | | | | CUCCAGUGCC | | |
| | | | | | | | AUU | | |
| One- | 249 | GGGA | 29nt- | 352 | GUCCU | 48 | gggaGCAGCCA | | |
| sided T- | | CAGA | clvR | | UAGUC | | UUAGUAUAUU | | |
| Orf1ab- | | TCTAA | NA | | GAAAGU | | AGUAUAUUAC | | |
| 20nt_Cl- | | TGGC | | | UUUACU | | CUGGUUUUCG | | |
| 29nt- | | TGC | | | AGAGU | | AUCGAAAGAU | | |
| clvRNA | | | | | CA | | CGACGAGGUG | | |
| | | | | | | | AAAACCUCGU | | |
| | | | | | | | GACAGUCCUU | | |
| | | | | | | | AGUCGAAAGU | | |
| | | | | | | | UUUACUAGAG | | |
| | | | | | | | UCAGUCCUGA | | |
| | | | | | | | UUUUCGAAUC | | |
| | | | | | | | AGAGAAGACG | | |
| | | | | | | | AGCGUCCCAC | | |
| | | | | | | | GGGCGCAGAA | | |
| | | | | | | | GAGAAGUCAA | | |
| | | | | | | | CCAGAGAAAC | | |
| | | | | | | | AUAAUAUACG | | |
| | | | | | | | AUCUGUCCC | | |
| One- | 250 | GGGA | 29nt- | 353 | GUCCU | 49 | gggaAUUAGGU | | |
| sided T- | | GTAG | clvR | | UAGUC | | UUCUUAAUAG | | |
| Orf1ab- | | ACAAT | NA | | GAAAGU | | UAAGUAUAUU | | |
| 40nt_Cl- | | TCTAG | | | UUUACU | | AGUAUAUUAC | | |
| 29nt- | | TCTTA | | | AGAGU | | CUGGUUUUCG | | |
| clvRNA | | CTATT | | | CA | | AUCGAAAGAU | | |
| | | AAGA | | | | | CGACGAGGUG | | |
| | | AACCT | | | | | AAAACCUCGU | | |
| | | AAT | | | | | GACAGUCCUU | | |
| | | | | | | | AGUCGAAAGU | | |
| | | | | | | | UUUACUAGAG | | |
| | | | | | | | UCAGUCCUGA | | |
| | | | | | | | UUUUCGAAUC | | |
| | | | | | | | AGAGAAGACG | | |
| | | | | | | | AGCGUCCCAC | | |
| | | | | | | | GGGCGCAGAA | | |
| | | | | | | | GAGAAGUCAA | | |
| | | | | | | | CCAGAGAAAC | | |
| | | | | | | | AUAAUAUACG | | |
| | | | | | | | ACUAGAAUUG | | |
| | | | | | | | UCUACUCCC | | |
| One- | 251 | UGAG | 29nt- | 354 | GUCCU | 50 | gggaAACUAUA | | |
| sided T- | | GUAG | clvR | | UAGUC | | CAAUGUAUAU | | |
| let-7f_Cl | | UAGA | NA | | GAAAGU | | UAGUAUAUUA | | |
| 29nt- | | UUGU | | | UUUACU | | CCUGGUUUUC | | |
| clvRNA | | AUAG | | | AGAGU | | GAUCGAAAGA | | |
| | | UU | | | CA | | UCGACGAGGU | | |
| | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | CUACUACCUC | | |
| | | | | | | | A | | |
| One- | 252 | UUCG | 29nt- | 355 | GUCCU | 51 | gggaACGUACC | | |
| sided T- | | GAAG | clvR | | UAGUC | | UGUGUAUAUU | | |
| Egene_C | | AGAC | NA | | GAAAGU | | AGUAUAUUAC | | |
| I-29nt- | | AGGU | | | UUUACU | | CUGGUUUUCG | | |
| clvRNA | | ACGU | | | AGAGU | | AUCGAAAGAU | | |
| 8-nt Helix | | | | | CA | | CGACGAGGUG | | |
| 4 | | | | | | | AAAACCUCGU | | |
| | | | | | | | GACAGUCCUU | | |
| | | | | | | | AGUCGAAAGU | | |
| | | | | | | | UUUACUAGAG | | |
| | | | | | | | UCAGUCCUGA | | |
| | | | | | | | UUUUCGAAUC | | |
| | | | | | | | AGAGAAGACG | | |
| | | | | | | | AGCGUCCCAC | | |
| | | | | | | | GGGCGCAGAA | | |
| | | | | | | | GAGAAGUCAA | | |
| | | | | | | | CCAGAGAAAC | | |
| | | | | | | | AUAAUAUACC | | |
| | | | | | | | UCUUCCGAA | | |
| One- | 253 | UUCG | 29nt- | 356 | GUCCU | 52 | gggaACGUACC | | |
| sided T- | | GAAG | clvR | | UAGUC | | UGUUAUUAGU | | |
| Egene_C | | AGAC | NA | | GAAAGU | | AUAUUACCUG | | |
| I-29nt- | | AGGU | | | UUUACU | | GUUUUCGAUC | | |
| clvRNA | | ACGU | | | AGAGU | | GAAAGAUCGA | | |
| 5-nt Helix | | | | | CA | | CGAGGUGAAA | | |
| 4 | | | | | | | ACCUCGUGAC | | |
| | | | | | | | AGUCCUUAGU | | |
| | | | | | | | CGAAAGUUUU | | |
| | | | | | | | ACUAGAGUCA | | |
| | | | | | | | GUCCUGAUUU | | |
| | | | | | | | UCGAAUCAGA | | |
| | | | | | | | GAAGACGAGC | | |
| | | | | | | | GUCCCACGGG | | |
| | | | | | | | CGCAGAAGAG | | |
| | | | | | | | AAGUCAACCA | | |
| | | | | | | | GAGAAACAUA | | |
| | | | | | | | AUACUCUUCC | | |
| | | | | | | | GAA | | |
| One- | 254 | UUCG | 29nt- | 357 | GUCCU | 53 | gggaACGUACC | | |
| sided T- | | GAAG | clvR | | UAGUC | | UGUACGGUAU | | |
| Egene_C | | AGAC | NA | | GAAAGU | | AUUACCUGGU | | |
| I-29nt- | | AGGU | | | UUUACU | | UUUCGAUCGA | | |
| clvRNA | | ACGU | | | AGAGU | | AAGAUCGACG | | |
| 3-nt Helix | | | | | CA | | AGGUGAAAAC | | |
| 4 v1 | | | | | | | CUCGUGACAG | | |
| | | | | | | | UCCUUAGUCG | | |
| | | | | | | | AAAGUUUUAC | | |
| | | | | | | | UAGAGUCAGU | | |
| | | | | | | | CCUGAUUUUC | | |
| | | | | | | | GAAUCAGAGA | | |
| | | | | | | | AGACGAGCGU | | |
| | | | | | | | CCCACGGGCG | | |
| | | | | | | | CAGAAGAGAA | | |
| | | | | | | | GUCAACCAGA | | |
| | | | | | | | GAAACACGUC | | |
| | | | | | | | UCUUCCGAA | | |
| One- | 255 | UUCG | 29nt- | 358 | GUCCU | 54 | gggaACGUACC | | |
| sided T- | | GAAG | clvR | | UAGUC | | UGUACGGUAU | | |
| Egene_C | | AGAC | NA | | GAAAGU | | AUUACCUGGU | | |
| I-29nt- | | AGGU | | | UUUACU | | UUUCGAUCGA | | |
| clvRNA | | ACGU | | | AGAGU | | AAGAUCGACG | | |
| 3-nt Helix | | | | | CA | | AGGUGAAAAC | | |
| 4 v2 | | | | | | | CUCGUGACAG | | |
| | | | | | | | UCCUUAGUCG | | |
| | | | | | | | AAAGUUUUAC | | |
| | | | | | | | UAGAGUCAGU | | |
| | | | | | | | CCUGAUUUUC | | |
| | | | | | | | GAAUCAGAGA | | |
| | | | | | | | AGACGAGCGU | | |
| | | | | | | | CCCACGGGCG | | |
| | | | | | | | CAGAAGAGAA | | |
| | | | | | | | GUCAACCAGA | | |
| | | | | | | | GAAACACGAC | | |
| | | | | | | | UCUUCCGAA | | |
| One- | 256 | UUCG | 29nt- | 359 | GUCCU | 55 | gggaACGUACC | | |
| sided T- | | GAAG | clvR | | UAGUC | | UGUACGUAUA | | |
| Egene_C | | AGAC | NA | | GAAAGU | | UUACCUGGUU | | |
| I-29nt- | | AGGU | | | UUUACU | | UUCGAUCGAA | | |
| clvRNA | | ACGU | | | AGAGU | | AGAUCGACGA | | |
| 2-nt Helix | | | | | CA | | GGUGAAAACC | | |
| 4 | | | | | | | UCGUGACAGU | | |
| | | | | | | | CCUUAGUCGA | | |
| | | | | | | | AAGUUUUACU | | |
| | | | | | | | AGAGUCAGUC | | |
| | | | | | | | CUGAUUUUCG | | |
| | | | | | | | AAUCAGAGAA | | |
| | | | | | | | GACGAGCGUC | | |
| | | | | | | | CCACGGGCGC | | |
| | | | | | | | AGAAGAGAAG | | |
| | | | | | | | UCAACCAGAG | | |
| | | | | | | | AAACAGUCUC | | |
| | | | | | | | UUCCGAA | | |
| One- | 257 | AAAC | 29nt- | 360 | GUCCU | 56 | gggaAACUCAG | | |
| sided T- | | CGUU | clvR | | UAGUC | | UAAUGUAUAU | | |
| mir451a | | ACCA | NA | | GAAAGU | | UAGUAUAUUA | | |
| Cl29nt- | | UUAC | | | UUUACU | | CCUGGUUUUC | | |
| clvRNA | | UGAG | | | AGAGU | | GAUCGAAAGA | | |
| 8-nt Helix | | UU | | | CA | | UCGACGAGGU | | |
| 4 | | | | | | | GAAAACCUCG | | |
| | | | | | | | UGACAGUCCU | | |
| | | | | | | | UAGUCGAAAG | | |
| | | | | | | | UUUUACUAGA | | |
| | | | | | | | GUCAGUCCUG | | |
| | | | | | | | AUUUUCGAAU | | |
| | | | | | | | CAGAGAAGAC | | |
| | | | | | | | GAGCGUCCCA | | |
| | | | | | | | CGGGCGCAGA | | |
| | | | | | | | AGAGAAGUCA | | |
| | | | | | | | ACCAGAGAAA | | |
| | | | | | | | CAUAAUAUAC | | |
| | | | | | | | GGUAACGGUU | | |
| | | | | | | | U | | |
| One- | 258 | AAAC | 29nt- | 361 | GUCCU | 57 | gggaAACUCAG | | |
| sided T- | | CGUU | clvR | | UAGUC | | UAAUUAUUAG | | |
| mir451a | | ACCA | NA | | GAAAGU | | UAUAUUACCU | | |
| Cl29nt- | | UUAC | | | UUUACU | | GGUUUUCGAU | | |
| clvRNA | | UGAG | | | AGAGU | | CGAAAGAUCG | | |
| 5-nt Helix | | UU | | | CA | | ACGAGGUGAA | | |
| 4 | | | | | | | AACCUCGUGA | | |
| | | | | | | | CAGUCCUUAG | | |
| | | | | | | | UCGAAAGUUU | | |
| | | | | | | | UACUAGAGUC | | |
| | | | | | | | AGUCCUGAUU | | |
| | | | | | | | UUCGAAUCAG | | |
| | | | | | | | AGAAGACGAG | | |
| | | | | | | | CGUCCCACGG | | |
| | | | | | | | GCGCAGAAGA | | |
| | | | | | | | GAAGUCAACC | | |
| | | | | | | | AGAGAAACAU | | |
| | | | | | | | AAUAGGUAAC | | |
| | | | | | | | GGUUU | | |
| One- | 259 | AAAC | 29nt- | 362 | GUCCU | 58 | gggaAACUCAG | | |
| sided T- | | CGUU | clvR | | UAGUC | | UAAUACGGUA | | |
| mir451a | | ACCA | NA | | GAAAGU | | UAUUACCUGG | | |
| Cl29nt- | | UUAC | | | UUUACU | | UUUUCGAUCG | | |
| clvRNA | | UGAG | | | AGAGU | | AAAGAUCGAC | | |
| 3-nt Helix | | UU | | | CA | | GAGGUGAAAA | | |
| 4 v1 | | | | | | | CCUCGUGACA | | |
| | | | | | | | GUCCUUAGUC | | |
| | | | | | | | GAAAGUUUUA | | |
| | | | | | | | CUAGAGUCAG | | |
| | | | | | | | UCCUGAUUUU | | |
| | | | | | | | CGAAUCAGAG | | |
| | | | | | | | AAGACGAGCG | | |
| | | | | | | | UCCCACGGGC | | |
| | | | | | | | GCAGAAGAGA | | |
| | | | | | | | AGUCAACCAG | | |
| | | | | | | | AGAAACACGU | | |
| | | | | | | | GGUAACGGUU | | |
| | | | | | | | U | | |
| One- | 260 | AAAC | 29nt- | 363 | GUCCU | 59 | gggaAACUCAG | | |
| sided T- | | CGUU | clvR | | UAGUC | | UAAUACGGUA | | |
| mir451a | | ACCA | NA | | GAAAGU | | UAUUACCUGG | | |
| Cl29nt- | | UUAC | | | UUUACU | | UUUUCGAUCG | | |
| clvRNA | | UGAG | | | AGAGU | | AAAGAUCGAC | | |
| 3-nt Helix | | UU | | | CA | | GAGGUGAAAA | | |
| 4 v2 | | | | | | | CCUCGUGACA | | |
| | | | | | | | GUCCUUAGUC | | |
| | | | | | | | GAAAGUUUUA | | |
| | | | | | | | CUAGAGUCAG | | |
| | | | | | | | UCCUGAUUUU | | |
| | | | | | | | CGAAUCAGAG | | |
| | | | | | | | AAGACGAGCG | | |
| | | | | | | | UCCCACGGGC | | |
| | | | | | | | GCAGAAGAGA | | |
| | | | | | | | AGUCAACCAG | | |
| | | | | | | | AGAAACACGA | | |
| | | | | | | | GGUAACGGUU | | |
| | | | | | | | U | | |
| One- | 261 | AAAC | 29nt- | 364 | GUCCU | 60 | gggaAACUCAG | | |
| sided T- | | CGUU | clvR | | UAGUC | | UAAUACGUAU | | |
| mir451a | | ACCA | NA | | GAAAGU | | AUUACCUGGU | | |
| Cl29nt- | | UUAC | | | UUUACU | | UUUCGAUCGA | | |
| clvRNA | | UGAG | | | AGAGU | | AAGAUCGACG | | |
| 2-nt Helix | | UU | | | CA | | AGGUGAAAAC | | |
| 4 | | | | | | | CUCGUGACAG | | |
| | | | | | | | UCCUUAGUCG | | |
| | | | | | | | AAAGUUUUAC | | |
| | | | | | | | UAGAGUCAGU | | |
| | | | | | | | CCUGAUUUUC | | |
| | | | | | | | GAAUCAGAGA | | |
| | | | | | | | AGACGAGCGU | | |
| | | | | | | | CCCACGGGCG | | |
| | | | | | | | CAGAAGAGAA | | |
| | | | | | | | GUCAACCAGA | | |
| | | | | | | | GAAACAGUGG | | |
| | | | | | | | UAACGGUUU | | |
| One- | 262 | CUUC | 29nt- | 365 | GUCCU | 61 | gggaAGCUGUC | | |
| sided T- | | UUCA | clvR | | UAGUC | | CAAGUAUAUU | | |
| Sgene_C | | GGUU | NA | | GAAAGU | | AGUAUAUUAC | | |
| I-29nt- | | GGAC | | | UUUACU | | CUGGUUUUCG | | |
| clvRNA | | AGCU | | | AGAGU | | AUCGAAAGAU | | |
| 8-nt Helix | | | | | CA | | CGACGAGGUG | | |
| 4 | | | | | | | AAAACCUCGU | | |
| | | | | | | | GACAGUCCUU | | |
| | | | | | | | AGUCGAAAGU | | |
| | | | | | | | UUUACUAGAG | | |
| | | | | | | | UCAGUCCUGA | | |
| | | | | | | | UUUUCGAAUC | | |
| | | | | | | | AGAGAAGACG | | |
| | | | | | | | AGCGUCCCAC | | |
| | | | | | | | GGGCGCAGAA | | |
| | | | | | | | GAGAAGUCAA | | |
| | | | | | | | CCAGAGAAAC | | |
| | | | | | | | AUAAUAUACC | | |
| | | | | | | | CUGAAGAAG | | |
| One- | 263 | CUUC | 29nt- | 366 | GUCCU | 62 | gggaAGCUGUC | | |
| sided T- | | UUCA | clvR | | UAGUC | | CAAUAUUAGU | | |
| Sgene_C | | GGUU | NA | | GAAAGU | | AUAUUACCUG | | |
| I-29nt- | | GGAC | | | UUUACU | | GUUUUCGAUC | | |
| clvRNA | | AGCU | | | AGAGU | | GAAAGAUCGA | | |
| 5-nt Helix | | | | | CA | | CGAGGUGAAA | | |
| 4 | | | | | | | ACCUCGUGAC | | |
| | | | | | | | AGUCCUUAGU | | |
| | | | | | | | CGAAAGUUUU | | |
| | | | | | | | ACUAGAGUCA | | |
| | | | | | | | GUCCUGAUUU | | |
| | | | | | | | UCGAAUCAGA | | |
| | | | | | | | GAAGACGAGC | | |
| | | | | | | | GUCCCACGGG | | |
| | | | | | | | CGCAGAAGAG | | |
| | | | | | | | AAGUCAACCA | | |
| | | | | | | | GAGAAACAUA | | |
| | | | | | | | AUACCUGAAG | | |
| | | | | | | | AAG | | |
| One- | 264 | CUUC | 29nt- | 367 | GUCCU | 63 | gggaAGCUGUC | | |
| sided T- | | UUCA | clvR | | UAGUC | | CAAACGGUAU | | |
| Sgene_C | | GGUU | NA | | GAAAGU | | AUUACCUGGU | | |
| I-29nt- | | GGAC | | | UUUACU | | UUUCGAUCGA | | |
| clvRNA | | AGCU | | | AGAGU | | AAGAUCGACG | | |
| 3-nt Helix | | | | | CA | | AGGUGAAAAC | | |
| 4 v1 | | | | | | | CUCGUGACAG | | |
| | | | | | | | UCCUUAGUCG | | |
| | | | | | | | AAAGUUUUAC | | |
| | | | | | | | UAGAGUCAGU | | |
| | | | | | | | CCUGAUUUUC | | |
| | | | | | | | GAAUCAGAGA | | |
| | | | | | | | AGACGAGCGU | | |
| | | | | | | | CCCACGGGCG | | |
| | | | | | | | CAGAAGAGAA | | |
| | | | | | | | GUCAACCAGA | | |
| | | | | | | | GAAACACGUC | | |
| | | | | | | | CUGAAGAAG | | |
| One- | 265 | CUUC | 29nt- | 368 | GUCCU | 64 | gggaAGCUGUC | | |
| sided T- | | UUCA | clvR | | UAGUC | | CAAACGGUAU | | |
| Sgene_C | | GGUU | NA | | GAAAGU | | AUUACCUGGU | | |
| I-29nt- | | GGAC | | | UUUACU | | UUUCGAUCGA | | |
| clvRNA | | AGCU | | | AGAGU | | AAGAUCGACG | | |
| 3-nt Helix | | | | | CA | | AGGUGAAAAC | | |
| 4 v2 | | | | | | | CUCGUGACAG | | |
| | | | | | | | UCCUUAGUCG | | |
| | | | | | | | AAAGUUUUAC | | |
| | | | | | | | UAGAGUCAGU | | |
| | | | | | | | CCUGAUUUUC | | |
| | | | | | | | GAAUCAGAGA | | |
| | | | | | | | AGACGAGCGU | | |
| | | | | | | | CCCACGGGCG | | |
| | | | | | | | CAGAAGAGAA | | |
| | | | | | | | GUCAACCAGA | | |
| | | | | | | | GAAACACGAC | | |
| | | | | | | | CUGAAGAAG | | |
| One- | 266 | CUUC | 29nt- | 369 | GUCCU | 65 | gggaAGCUGUC | | |
| sided T- | | UUCA | clvR | | UAGUC | | CAAACGUAUA | | |
| Sgene_C | | GGUU | NA | | GAAAGU | | UUACCUGGUU | | |
| I-29nt- | | GGAC | | | UUUACU | | UUCGAUCGAA | | |
| clvRNA | | AGCU | | | AGAGU | | AGAUCGACGA | | |
| 2-nt Helix | | | | | CA | | GGUGAAAACC | | |
| 4 | | | | | | | UCGUGACAGU | | |
| | | | | | | | CCUUAGUCGA | | |
| | | | | | | | AAGUUUUACU | | |
| | | | | | | | AGAGUCAGUC | | |
| | | | | | | | CUGAUUUUCG | | |
| | | | | | | | AAUCAGAGAA | | |
| | | | | | | | GACGAGCGUC | | |
| | | | | | | | CCACGGGCGC | | |
| | | | | | | | AGAAGAGAAG | | |
| | | | | | | | UCAACCAGAG | | |
| | | | | | | | AAACAGUCCU | | |
| | | | | | | | GAAGAAG | | |
| T-let- | 267 | UGAG | sgRN | 370 | GUCCU | 66 | gggaAACUAUA | | |
| 7f_Cl | | GUAG | A- | | UGGGC | | CAAUACGGUA | | |
| sgRNA- | | UAGA | GFP | | ACGGG | | UAUUACCUGG | | |
| GFP | | UUGU | | | CAGCU | | UUUUCGAUCG | | |
| | | AUAG | | | UGCCG | | AAAGAUCGAC | | |
| | | UU | | | GGUUU | | GAGGUGAAAA | | |
| | | | | | UAGAG | | CCUCGUGACA | | |
| | | | | | CUAGAA | | GUCCUUGGGC | | |
| | | | | | AUAGCA | | ACGGGCAGCU | | |
| | | | | | AGUUAA | | UGCCGGGUUU | | |
| | | | | | AAUAAG | | UAGAGCUAGA | | |
| | | | | | GCUAG | | AAUAGCAAGU | | |
| | | | | | UCCGU | | UAAAAUAAGG | | |
| | | | | | UAUCAA | | CUAGUCCGUU | | |
| | | | | | CUUGAA | | AUCAACUUGA | | |
| | | | | | AAAGUG | | AAAAGUGGCA | | |
| | | | | | GCACC | | CCGAGUCGGU | | |
| | | | | | GAGUC | | GCUUUUUUUG | | |
| | | | | | GGUGC | | UCAGUCCUGA | | |
| | | | | | UUUUU | | UUUUCGAAUC | | |
| | | | | | UUGUC | | AGAGAAGACC | | |
| | | | | | A | | CCAACCUAUC | | |
| | | | | | | | CCCUUAAAUA | | |
| | | | | | | | GGCAAUUGAA | | |
| | | | | | | | AAAGAGAAGU | | |
| | | | | | | | CAACCAGAGA | | |
| | | | | | | | AACACGACUA | | |
| | | | | | | | CUACCUCA | | |
| T-let- | 268 | UGAG | sgRN | 371 | GGGCA | 67 | gggaAACUAUA | | |
| 7f_Cl | | GUAG | A- | | CGGGC | | CAAUACGGUA | | |
| sgRNAG | | UAGA | GFP | | AGCUU | | UAUUACCUGG | | |
| FP_A7 | | UUGU | | | GCCGG | | UUUUCGAUCG | | |
| | | AUAG | | | GUUUU | | AAAGAUCGAC | | |
| | | UU | | | AGAGC | | GAGGUGAAAA | | |
| | | | | | UAGAAA | | CCUCGUGACA | | |
| | | | | | UAGCAA | | GGGCACGGG | | |
| | | | | | GUUAAA | | CAGCUUGCCG | | |
| | | | | | AUAAGG | | GGUUUUAGAG | | |
| | | | | | CUAGU | | CUAGAAAUAG | | |
| | | | | | CCGUU | | CAAGUUAAAA | | |
| | | | | | AUCAAC | | UAAGGCUAGU | | |
| | | | | | UUGAAA | | CCGUUAUCAA | | |
| | | | | | AAGUG | | CUUGAAAAAG | | |
| | | | | | GCACC | | UGGCACCGAG | | |
| | | | | | GAGUC | | UCGGUGCGUC | | |
| | | | | | GGUGC | | AGUCCUGAUU | | |
| | | | | | GUCA | | UUCGAAUCAG | | |
| | | | | | | | AGAAGACUAU | | |
| | | | | | | | CCACCUUAAA | | |
| | | | | | | | UAGGCAAGUG | | |
| | | | | | | | AGAAGUCAAC | | |
| | | | | | | | CAGAGAAACA | | |
| | | | | | | | CGACUACUAC | | |
| | | | | | | | CUCA | | |
| T-let- | 269 | UGAG | sgRN | 372 | GGGCA | 68 | gggaAACUAUA | | |
| 7f_Cl | | GUAG | A- | | CGGGC | | CAAUACGGUA | | |
| sgRNAG | | UAGA | GFP | | AGCUU | | UAUUACCUGG | | |
| FP_C7 | | UUGU | | | GCCGG | | UUUUCGAUCG | | |
| | | AUAG | | | GUUUU | | AAAGAUCGAC | | |
| | | UU | | | AGAGC | | GAGGUGAAAA | | |
| | | | | | UAGAAA | | CCUCGUGACA | | |
| | | | | | UAGCAA | | GGGCACGGG | | |
| | | | | | GUUAAA | | CAGCUUGCCG | | |
| | | | | | AUAAGG | | GGUUUUAGAG | | |
| | | | | | CUAGU | | CUAGAAAUAG | | |
| | | | | | CCGUU | | CAAGUUAAAA | | |
| | | | | | AUCAAC | | UAAGGCUAGU | | |
| | | | | | UUGAAA | | CCGUUAUCAA | | |
| | | | | | AAGUG | | CUUGAAAAAG | | |
| | | | | | GCACC | | UGGCACCGAG | | |
| | | | | | GAGUC | | UCGGUGCGUC | | |
| | | | | | GGUGC | | AGUCCUGAUU | | |
| | | | | | GUCA | | UUCGAAUCAG | | |
| | | | | | | | AGAAGACUAU | | |
| | | | | | | | CCACCUUAAA | | |
| | | | | | | | UAGGCAAGUG | | |
| | | | | | | | AGAAGUCAAC | | |
| | | | | | | | CAGAGAAACA | | |
| | | | | | | | CGACUACUAC | | |
| | | | | | | | CUCA | | |
| T-let- | 270 | UGAG | sgRN | 373 | GGGCA | 69 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC1_8R | | AUAG | | | GUUUU | | AAAGAUCGAC | | |
| H2_A7R | | UU | | | AGAGC | | GAGGUGAAAA | | |
| _A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGACUACUA | | |
| | | | | | | | CCUCA | | |
| T-let- | 271 | UGAG | sgRN | 374 | GGGCA | 70 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC2_8R | | AUAG | | | GUUUU | | AAAGAUCGAC | | |
| H2_A7R | | UU | | | AGAGC | | GAGGUGAAAA | | |
| A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGACUAC | | |
| | | | | | | | UACCUCA | | |
| T-let- | 272 | UGAG | sgRN | 375 | GGGCA | 71 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC3_8R | | AUAG | | | GUUUU | | AAAGAUCGAC | | |
| H2_A7R | | UU | | | AGAGC | | GAGGUGAAAA | | |
| A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | GGACUAGCCU | | |
| | | | | | | | UAUUUUAACU | | |
| | | | | | | | UGCUAUUUCU | | |
| | | | | | | | AGCUCUAAAA | | |
| | | | | | | | CCCGGCAAGC | | |
| | | | | | | | UGCCCGUGAG | | |
| | | | | | | | AAGUCAUACG | | |
| | | | | | | | ACCAGAGAAA | | |
| | | | | | | | CACGACUACU | | |
| | | | | | | | ACCUCA | | |
| T-let- | 273 | UGAG | sgRN | 376 | GGGCA | 72 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC4_8R | | AUAG | | | GUUUU | | AAAGAUCGAC | | |
| H2_A7R | | UU | | | AGAGC | | GAGGUGAAAA | | |
| A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | GCACCGACUC | | |
| | | | | | | | GGUGCCACUU | | |
| | | | | | | | UUUCAAGUUG | | |
| | | | | | | | AUAACGGACU | | |
| | | | | | | | AGCCUUAUUU | | |
| | | | | | | | UAACUUGCUA | | |
| | | | | | | | UUUCUAGCUC | | |
| | | | | | | | UAAAACCCGG | | |
| | | | | | | | CAAGCUGCCC | | |
| | | | | | | | GUGAGAAGUC | | |
| | | | | | | | AUACGACCAG | | |
| | | | | | | | AGAAACACGA | | |
| | | | | | | | CUACUACCUC | | |
| | | | | | | | A | | |
| T-cel-mir- | 274 | UUUG | sgRN | 377 | GGGCA | 73 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC1_8R | | AUUC | | | GUUUU | | AAAGAUCGAC | | |
| H2_A7R | | AGA | | | AGAGC | | GAGGUGAAAA | | |
| A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGAUCGGAG | | |
| | | | | | | | UACAAA | | |
| T-cel-mir- | 275 | UUUG | sgRN | 378 | GGGCA | 74 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC2_8R | | AUUC | | | GUUUU | | AAAGAUCGAC | | |
| H2_A7R | | AGA | | | AGAGC | | GAGGUGAAAA | | |
| _A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGAUCGG | | |
| | | | | | | | AGUACAAA | | |
| T-cel-mir- | 276 | UUUG | sgRN | 379 | GGGCA | 75 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC3_8R | | AUUC | | | GUUUU | | AAAGAUCGAC | | |
| H2_A7R | | AGA | | | AGAGC | | GAGGUGAAAA | | |
| _A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | CCAUGAUAAC | | |
| | | | | | | | GGACUAGCCU | | |
| | | | | | | | UAUUUUAACU | | |
| | | | | | | | UGCUAUUUCU | | |
| | | | | | | | AGCUCUAAAA | | |
| | | | | | | | CCCGGCAAGC | | |
| | | | | | | | UGCCCGUGAG | | |
| | | | | | | | AAGUCAUACG | | |
| | | | | | | | ACCAGAGAAA | | |
| | | | | | | | CACGAUCGGA | | |
| | | | | | | | GUACAAA | | |
| T-cel-mir- | 277 | UUUG | sgRN | 380 | GGGCA | 76 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC4_8R | | AUUC | | | GUUUU | | AAAGAUCGAC | | |
| H2_A7R | | AGA | | | AGAGC | | GAGGUGAAAA | | |
| A7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGAGAAGAC | | |
| | | | | | | | GCACCGACUC | | |
| | | | | | | | GGUGCCACUU | | |
| | | | | | | | UUUCAAGUUG | | |
| | | | | | | | AUAACGGACU | | |
| | | | | | | | AGCCUUAUUU | | |
| | | | | | | | UAACUUGCUA | | |
| | | | | | | | UUUCUAGCUC | | |
| | | | | | | | UAAAACCCGG | | |
| | | | | | | | CAAGCUGCCC | | |
| | | | | | | | GUGAGAAGUC | | |
| | | | | | | | AUACGACCAG | | |
| | | | | | | | AGAAACACGA | | |
| | | | | | | | UCGGAGUACA | | |
| | | | | | | | AA | | |
| T-let- | 278 | UGAG | sgRN | 381 | GGGCA | 77 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC1_8R | | AUAG | | | GUUUU | | AAAGAUCGAC | | |
| H2_A7R | | UU | | | AGAGC | | GAGGUGAAAA | | |
| C7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGCGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGACUACUA | | |
| | | | | | | | CCUCA | | |
| T-let- | 279 | UGAG | sgRN | 382 | GGGCA | 78 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC2_8R | | AUAG | | | GUUUU | | AAAGAUCGAC | | |
| H2_A7R | | UU | | | AGAGC | | GAGGUGAAAA | | |
| _C7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | | | |
| | | | | | GGUGC | | GAGUCGGUGC | | |
| | | | | | GUCA | | GUCAGUCCUG | | |
| | | | | | | | AUUUUCGAAU | | |
| | | | | | | | CAGCGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGACUAC | | |
| | | | | | | | UACCUCA | | |
| T-let- | 280 | UGAG | sgRN | 383 | GGGCA | 79 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC1_8R | | AUAG | | | GUUUU | | AAAGAUCGAC | | |
| H2_A7R | | UU | | | AGAGC | | GAGGUGAAAA | | |
| _G7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGGGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGACUACUA | | |
| | | | | | | | CCUCA | | |
| T-let- | 281 | UGAG | sgRN | 384 | GGGCA | 80 | gggaAACUAUA | | |
| 7f_Cl | | GUAG | A- | | CGGGC | | CAAUACGGUA | | |
| sgRNA- | | UAGA | GFP | | AGCUU | | UAUUACCUGG | | |
| GFP_mo | | UUGU | | | GCCGG | | UUUUCGAUCG | | |
| dC2_8R | | AUAG | | | GUUUU | | AAAGAUCGAC | | |
| H2_A7R | | UU | | | AGAGC | | GAGGUGAAAA | | |
| _G7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | | | GUCAGUCCUG | | |
| | | | | | GGUGC | | AUUUUCGAAU | | |
| | | | | | GUCA | | CAGGGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGACUAC | | |
| | | | | | | | UACCUCA | | |
| T-cel-mir- | 282 | UUUG | sgRN | 385 | GGGCA | 81 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC1_8R | | AUUC | | | GUUUU | | AAAGAUCGAC | | |
| H2_A7R | | AGA | | | AGAGC | | GAGGUGAAAA | | |
| C7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGCGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGAUCGGAG | | |
| | | | | | | | UACAAA | | |
| T-cel-mir- | 283 | UUUG | sgRN | 386 | GGGCA | 82 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC2_8R | | AUUC | | | GUUUU | | AAAGAUCGAC | | |
| H2_A7R | | AGA | | | AGAGC | | GAGGUGAAAA | | |
| C7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGCGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGAUCGG | | |
| | | | | | | | AGUACAAA | | |
| T-cel-mir- | 284 | UUUG | sgRN | 387 | GGGCA | 83 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC1_8R | | AUUC | | | GUUUU | | AAAGAUCGAC | | |
| H2_A7R | | AGA | | | AGAGC | | GAGGUGAAAA | | |
| _G7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGGGAAGAC | | |
| | | | | | | | CCAUGAUAAC | | |
| | | | | | | | UUAUUUUAAC | | |
| | | | | | | | UUUCUAAAAC | | |
| | | | | | | | CCGGCAAGCU | | |
| | | | | | | | GCCCGUGAGA | | |
| | | | | | | | AGUCAUACGA | | |
| | | | | | | | CCAGAGAAAC | | |
| | | | | | | | ACGAUCGGAG | | |
| | | | | | | | UACAAA | | |
| T-cel-mir- | 285 | UUUG | sgRN | 388 | GGGCA | 84 | gggaUCUGAAU | | |
| 238_Cl | | UACU | A- | | CGGGC | | GGCAACGGUA | | |
| sgRNA- | | CCGA | GFP | | AGCUU | | UAUUACCUGG | | |
| GFP_mo | | UGCC | | | GCCGG | | UUUUCGAUCG | | |
| dC2_8R | | AUUC | | | GUUUU | | AAAGAUCGAC | | |
| H2_A7R | | AGA | | | AGAGC | | GAGGUGAAAA | | |
| _G7L | | | | | UAGAAA | | CCUCGCGUAU | | |
| | | | | | UAGCAA | | GACAGGGCAC | | |
| | | | | | GUUAAA | | GGGCAGCUUG | | |
| | | | | | AUAAGG | | CCGGGUUUUA | | |
| | | | | | CUAGU | | GAGCUAGAAA | | |
| | | | | | CCGUU | | UAGCAAGUUA | | |
| | | | | | AUCAAC | | AAAUAAGGCU | | |
| | | | | | UUGAAA | | AGUCCGUUAU | | |
| | | | | | AAGUG | | CAACUUGAAA | | |
| | | | | | GCACC | | AAGUGGCACC | | |
| | | | | | GAGUC | | GAGUCGGUGC | | |
| | | | | | GGUGC | | GUCAGUCCUG | | |
| | | | | | GUCA | | AUUUUCGAAU | | |
| | | | | | | | CAGGGAAGAC | | |
| | | | | | | | CCAUGAUAAC | | |
| | | | | | | | UUAUUUUAAC | | |
| | | | | | | | UUGCUAUUUC | | |
| | | | | | | | UAGCUCUAAA | | |
| | | | | | | | ACCCGGCAAG | | |
| | | | | | | | CUGCCCGUGA | | |
| | | | | | | | GAAGUCAUAC | | |
| | | | | | | | GACCAGAGAA | | |
| | | | | | | | ACACGAUCGG | | |
| | | | | | | | AGUACAAA | | |
| T-let- | 286 | UGAG | shRN | 389 | GUCUG | 85 | gggaAACUAUA | | |
| 7f_Cl | | GUAG | A- | | AUAGCA | | CAAUACGGUA | | |
| shRNA- | | UAGA | GFP | | AUGUCA | | UAUUACCUGG | | |
| GFP | | UUGU | | | GCAGU | | UUUUCGAUCG | | |
| | | AUAG | | | GCCUG | | AAAGAUCGAC | | |
| | | UU | | | CAAGCU | | GAGGUGAAAA | | |
| | | | | | GACCC | | CCUCGUGACA | | |
| | | | | | UGAAG | | GUCUGAUAGC | | |
| | | | | | UUCACC | | AAUGUCAGCA | | |
| | | | | | ACCUGA | | GUGCCUGCAA | | |
| | | | | | ACUUCA | | GCUGACCCUG | | |
| | | | | | GGGUC | | AAGUUCACCA | | |
| | | | | | AGCUU | | CCUGAACUUC | | |
| | | | | | GCAAG | | AGGGUCAGCU | | |
| | | | | | UAAGG | | UGCAAGUAAG | | |
| | | | | | UUGAC | | GUUGACCAUA | | |
| | | | | | CAUACU | | CUCUACAGUC | | |
| | | | | | CUACA | | CUGAUUUUCG | | |
| | | | | | | | AAUCAGAGAA | | |
| | | | | | | | GUAUCUGAUG | | |
| | | | | | | | AUUUAGCUCA | | |
| | | | | | | | AGAAGUCAAC | | |
| | | | | | | | CAGAGAAACA | | |
| | | | | | | | CGACUACUAC | | |
| | | | | | | | CUCA | | |
| T-let- | 287 | UGAG | shRN | 390 | GUCUG | 86 | gggaAACUAUA | | |
| 7f_Cl | | GUAG | A- | | AUAGCA | | CAAUACGGUA | | |
| shRNA- | | UAGA | GFP | | AUGUCA | | UAUUACCUGG | | |
| GFP_8R | | UUGU | | | GCAGU | | UUUUCGAUCG | | |
| H2 | | AUAG | | | GCCUG | | AAAGAUCGAC | | |
| | | UU | | | CAAGCU | | GAGGUGAAAA | | |
| | | | | | GACCC | | CCUCGCGUAU | | |
| | | | | | UGAAG | | GACAGUCUGA | | |
| | | | | | UUCACC | | UAGCAAUGUC | | |
| | | | | | ACCUGA | | AGCAGUGCCU | | |
| | | | | | ACUUCA | | GCAAGCUGAC | | |
| | | | | | GGGUC | | CCUGAAGUUC | | |
| | | | | | AGCUU | | ACCACCUGAA | | |
| | | | | | GCAAG | | CUUCAGGGUC | | |
| | | | | | UAAGG | | AGCUUGCAAG | | |
| | | | | | UUGAC | | UAAGGUUGAC | | |
| | | | | | CAUACU | | CAUACUCUAC | | |
| | | | | | CUACA | | AGUCCUGAUU | | |
| | | | | | | | UUCGAAUCAG | | |
| | | | | | | | AGAAGUAUCU | | |
| | | | | | | | GAUGAUUUAG | | |
| | | | | | | | CUCAAGAAGU | | |
| | | | | | | | CAUACGACCA | | |
| | | | | | | | GAGAAACACG | | |
| | | | | | | | ACUACUACCU | | |
| | | | | | | | CA | | |
| T-let- | 288 | UGAG | shRN | 391 | GUCUG | 87 | GGGAAACUAU | | |
| 7f_Cl | | GUAG | A- | | AUAGCA | | ACAAUACGGU | | |
| shRNA- | | UAGA | GFP | | AUGUCA | | UUAUUACCUG | | |
| GFP_ | | UUGU | | | GCAGU | | GUUUUCGAUC | | |
| A38mutant | | AUAG | | | GCCUG | | GAAAGAUCGA | | |
| | | UU | | | CAAGCU | | CGAGGUGAAA | | |
| | | | | | GACCC | | ACCUCGUGAC | | |
| | | | | | UGAAG | | AGUCUGAUAG | | |
| | | | | | UUCACC | | CAAUGUCAGC | | |
| | | | | | ACCUGA | | AGUGCCUGCA | | |
| | | | | | ACUUCA | | AGCUGACCCU | | |
| | | | | | GGGUC | | GAAGUUCACC | | |
| | | | | | AGCUU | | ACCUGAACUU | | |
| | | | | | GCAAG | | CAGGGUCAGC | | |
| | | | | | UAAGG | | UUGCAAGUAA | | |
| | | | | | UUGAC | | GGUUGACCAU | | |
| | | | | | CAUACU | | ACUCUACAGU | | |
| | | | | | CUACA | | CCUGAUUUUC | | |
| | | | | | | | GAAUCAGAGA | | |
| | | | | | | | AGUAUCUGAU | | |
| | | | | | | | GAUUUAGCUC | | |
| | | | | | | | AAGAAGUCAA | | |
| | | | | | | | CCAGAGAAAC | | |
| | | | | | | | ACGACUACUA | | |
| | | | | | | | CCUCA | | |
| T-let- | 289 | UGAG | shRN | 392 | GUCUG | 88 | GGGAAACUAU | | |
| 7f_Cl | | GUAG | A- | | AUAGCA | | ACAAUACGGU | | |
| shRNA- | | UAGA | GFP- | | AUGUCA | | AUAUUACCUG | | |
| GFP_ | | UUGU | targe | | GCAGU | | GUUUUCGAUC | | |
| targetting | | AUAG | tingm | | GCCUG | | GAAAGAUCGA | | |
| mutant | | UU | utant | | CAAGCU | | CGAGGUGAAA | | |
| | | | | | GCAACU | | ACCUCGUGAC | | |
| | | | | | GAAGU | | AGUCUGAUAG | | |
| | | | | | UCACCA | | CAAUGUCAGC | | |
| | | | | | CCUGAA | | AGUGCCUGCA | | |
| | | | | | CUUCA | | AGCUGCAACU | | |
| | | | | | GUUGC | | GAAGUUCACC | | |
| | | | | | AGCUU | | ACCUGAACUU | | |
| | | | | | GCAAG | | CAGUUGCAGC | | |
| | | | | | UAAGG | | UUGCAAGUAA | | |
| | | | | | UUGAC | | GGUUGACCAU | | |
| | | | | | CAUACU | | ACUCUACAGU | | |
| | | | | | CUACA | | CCUGAUUUUC | | |
| | | | | | | | GAAUCAGAGA | | |
| | | | | | | | AGUAUCUGAU | | |
| | | | | | | | GAUUUAGCUC | | |
| | | | | | | | AAGAAGUCAA | | |
| | | | | | | | CCAGAGAAAC | | |
| | | | | | | | ACGACUACUA | | |
| | | | | | | | CCUCA | | |
| T-cel-mir- | 290 | UUUG | shRN | 393 | GUCUG | 89 | gggaUCUGAAU | | |
| 238_Cl | | UACU | A- | | AUAGCA | | GGCAACGGUA | | |
| shRNA- | | CCGA | GFP | | AUGUCA | | UAUUACCUGG | | |
| GFP | | UGCC | | | GCAGU | | UUUUCGAUCG | | |
| | | AUUC | | | GCCUG | | AAAGAUCGAC | | |
| | | AGA | | | CAAGCU | | GAGGUGAAAA | | |
| | | | | | GACCC | | CCUCGUGACA | | |
| | | | | | UGAAG | | GUCUGAUAGC | | |
| | | | | | UUCACC | | AAUGUCAGCA | | |
| | | | | | ACCUGA | | GUGCCUGCAA | | |
| | | | | | ACUUCA | | GCUGACCCUG | | |
| | | | | | GGGUC | | AAGUUCACCA | | |
| | | | | | AGCUU | | CCUGAACUUC | | |
| | | | | | GCAAG | | AGGGUCAGCU | | |
| | | | | | UAAGG | | UGCAAGUAAG | | |
| | | | | | UUGAC | | GUUGACCAUA | | |
| | | | | | CAUACU | | CUCUACAGUC | | |
| | | | | | CUACA | | CUGAUUUUCG | | |
| | | | | | | | AAUCAGAGAA | | |
| | | | | | | | GUAUCUGAUG | | |
| | | | | | | | AUUUAGCUCA | | |
| | | | | | | | AGAAGUCAAC | | |
| | | | | | | | CAGAGAAACA | | |
| | | | | | | | CGAUCGGAGU | | |
| | | | | | | | ACAAA | | |
| T-cel-mir- | 291 | UUUG | shRN | 394 | GUCUG | 90 | gggaUCUGAAU | | |
| 238_Cl | | UACU | A- | | AUAGCA | | GGCAACGGUU | | |
| shRNA- | | CCGA | GFP | | AUGUCA | | UAUUACCUGG | | |
| GFP_ | | UGCC | | | GCAGU | | UUUUCGAUCG | | |
| A38mutant | | AUUC | | | GCCUG | | AAAGAUCGAC | | |
| | | AGA | | | CAAGCU | | GAGGUGAAAA | | |
| | | | | | GACCC | | CCUCGUGACA | | |
| | | | | | UGAAG | | GUCUGAUAGC | | |
| | | | | | UUCACC | | AAUGUCAGCA | | |
| | | | | | ACCUGA | | GUGCCUGCAA | | |
| | | | | | ACUUCA | | GCUGACCCUG | | |
| | | | | | GGGUC | | AAGUUCACCA | | |
| | | | | | AGCUU | | CCUGAACUUC | | |
| | | | | | GCAAG | | AGGGUCAGCU | | |
| | | | | | UAAGG | | UGCAAGUAAG | | |
| | | | | | UUGAC | | GUUGACCAUA | | |
| | | | | | CAUACU | | CUCUACAGUC | | |
| | | | | | CUACA | | CUGAUUUUCG | | |
| | | | | | | | AAUCAGAGAA | | |
| | | | | | | | GUAUCUGAUG | | |
| | | | | | | | AUUUAGCUCA | | |
| | | | | | | | AGAAGUCAAC | | |
| | | | | | | | CAGAGAAACA | | |
| | | | | | | | CGAUCGGAGU | | |
| | | | | | | | ACAAA | | |
| T-cel-mir- | 292 | UUUG | shRN | 395 | GUCUG | 91 | gggaUCUGAAU | | |
| 238_Cl | | UACU | A- | | AUAGCA | | GGCAACGGUA | | |
| shRNA- | | CCGA | GFP- | | AUGUCA | | UAUUACCUGG | | |
| GFP_ | | UGCC | targe | | GCAGU | | UUUUCGAUCG | | |
| targetting | | AUUC | tingm | | GCCUG | | AAAGAUCGAC | | |
| mutant | | AGA | utant | | CAAGCU | | GAGGUGAAAA | | |
| | | | | | GCAACU | | CCUCGUGACA | | |
| | | | | | GAAGU | | GUCUGAUAGC | | |
| | | | | | UCACCA | | AAUGUCAGCA | | |
| | | | | | CCUGAA | | GUGCCUGCAA | | |
| | | | | | CUUCA | | GCUGCAACUG | | |
| | | | | | GUUGC | | AAGUUCACCA | | |
| | | | | | AGCUU | | CCUGAACUUC | | |
| | | | | | GCAAG | | AGUUGCAGCU | | |
| | | | | | UAAGG | | UGCAAGUAAG | | |
| | | | | | UUGAC | | GUUGACCAUA | | |
| | | | | | CAUACU | | CUCUACAGUC | | |
| | | | | | CUACA | | CUGAUUUUCG | | |
| | | | | | | | AAUCAGAGAA | | |
| | | | | | | | GUAUCUGAUG | | |
| | | | | | | | AUUUAGCUCA | | |
| | | | | | | | AGAAGUCAAC | | |
| | | | | | | | CAGAGAAACA | | |
| | | | | | | | CGAUCGGAGU | | |
| | | | | | | | ACAAA | | |
| T-let- | 293 | UGAG | Broc | 396 | GUCCG | 92 | gggaAACUAUA | | |
| 7f Cl- | | GUAG | coli | | AGACG | | CAAUACGGUA | | |
| Red | | UAGA | RNA | | GUCGG | | UAUUACCUGG | | |
| Broccoli_ | | UUGU | apta | | GUCCA | | UUUUCGAUCG | | |
| ML | | AUAG | mer | | GUCCC | | AAAGAUCGAC | | |
| | | UU | | | AACGAU | | GAGGUGAAAA | | |
| | | | | | GUUGG | | CCUCGUGACA | | |
| | | | | | CUGUU | | GUCCGAGACG | | |
| | | | | | GAGUA | | GUCGGGUCCA | | |
| | | | | | GUGUG | | GUCCCAACGA | | |
| | | | | | UGGGC | | UGUUGGCUGU | | |
| | | | | | UCCA | | UGAGUAGUGU | | |
| | | | | | | | GUGGGCUCCA | | |
| | | | | | | | GUCCUGAUUU | | |
| | | | | | | | UCGAAUCAGA | | |
| | | | | | | | GAAGACCCCA | | |
| | | | | | | | ACCUAUCCCC | | |
| | | | | | | | UUAAAUAGGC | | |
| | | | | | | | AAUUGAAAAA | | |
| | | | | | | | GAGAAGUCAA | | |
| | | | | | | | CCAGAGAAAC | | |
| | | | | | | | ACGACUACUA | | |
| | | | | | | | CCUCA | | |
| tSJ057_sh | 294 | UGAG | shGF | 397 | GUCUG | 93 | GGGAAACUAU | | |
| GFP1Rz | | GUAG | P1 | | AUAGCA | | ACAAUACGGU | | |
| | | UAGA | | | AUGUCA | | AUAUUACCUG | | |
| | | UUGU | | | GCAGU | | GUUUUCGAUC | | |
| | | AUAG | | | GCCUG | | GAAAGAUCGA | | |
| | | UU | | | CAAGCU | | CGAGGUGAAA | | |
| | | | | | GACCC | | ACCUCGUGAC | | |
| | | | | | UGAAG | | AGUCUGAUAG | | |
| | | | | | UUCAUC | | CAAUGUCAGC | | |
| | | | | | UGCAG | | AGUGCCUGCA | | |
| | | | | | AUGAAC | | AGCUGACCCU | | |
| | | | | | UUCAG | | GAAGUUCAUC | | |
| | | | | | GGUCA | | UGCAGAUGAA | | |
| | | | | | GCUUG | | CUUCAGGGUC | | |
| | | | | | CAAGUA | | AGCUUGCAAG | | |
| | | | | | AGGUU | | UAAGGUUGAC | | |
| | | | | | GACCAU | | CAUACUCUAC | | |
| | | | | | | | AGUCCUGAUU | | |
| | | | | | ACUCUA | | UUCGAAUCAG | | |
| | | | | | CA | | AGAAGUAUCU | | |
| | | | | | | | GAUGUAAUUU | | |
| | | | | | | | AGCUCAAGAA | | |
| | | | | | | | GUCAACCAGA | | |
| | | | | | | | GAAACACGAC | | |
| | | | | | | | UACUACCUCA | | |
| tSJ058_sh | 295 | UGAG | shGF | 398 | GUCUG | 94 | GGGAAACUAU | | |
| GFP2Rz | | GUAG | P2 | | AUAGCA | | ACAAUACGGU | | |
| | | UAGA | | | AUGUCA | | AUAUUACCUG | | |
| | | UUGU | | | GCAGU | | GUUUUCGAUC | | |
| | | AUAG | | | GCCUG | | GAAAGAUCGA | | |
| | | UU | | | CAAGCT | | CGAGGUGAAA | | |
| | | | | | GACCCT | | ACCUCGUGAC | | |
| | | | | | GAAGTT | | AGUCUGAUAG | | |
| | | | | | CAccacc | | CAAUGUCAGC | | |
| | | | | | TGAACT | | AGUGCCUGCA | | |
| | | | | | TCAGG | | AGCUGACCCU | | |
| | | | | | GTCAGC | | GAAGUUCACC | | |
| | | | | | TTGCaa | | ACCUGAACUU | | |
| | | | | | GUAAG | | CAGGGUCAGC | | |
| | | | | | GUUGA | | UUGCAAGUAA | | |
| | | | | | CCAUAC | | GGUUGACCAU | | |
| | | | | | UCUACA | | ACUCUACAGU | | |
| | | | | | | | CCUGAUUUUC | | |
| | | | | | | | GAAUCAGAGA | | |
| | | | | | | | AGUAUCUGAU | | |
| | | | | | | | GUAAUUUAGC | | |
| | | | | | | | UCAAGAAGUC | | |
| | | | | | | | AACCAGAGAA | | |
| | | | | | | | ACACGACUAC | | |
| | | | | | | | UACCUCA | | |
| tSJ059_sh | 296 | UGAG | shGF | 399 | GUCUG | 95 | GGGAAACUAU | | |
| GFP3 | | GUAG | P3 | | AUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUG | | GAAAGAUCGA | | |
| | | UU | | | CAAGCT | | CGAGGUGAAA | | |
| | | | | | GACCCT | | ACCUCGUGAC | | |
| | | | | | GAAGTT | | AGUCUGAUAG | | |
| | | | | | CAccacc | | CAAUGUCAAC | | |
| | | | | | TGAACT | | CUCGCCUGCA | | |
| | | | | | TCAGG | | AGCUGACCCU | | |
| | | | | | GTCAGC | | GAAGUUCACC | | |
| | | | | | TTGCag | | ACCUGAACUU | | |
| | | | | | GCGCG | | CAGGGUCAGC | | |
| | | | | | GUUGA | | UUGCAGGCGC | | |
| | | | | | CCAUAC | | GGUUGACCAU | | |
| | | | | | CGUCC | | ACCGUCCAGU | | |
| | | | | | A | | CCUGAUUUUC | | |
| | | | | | | | GAAUCAGAGA | | |
| | | | | | | | AGGAAUUGAU | | |
| | | | | | | | GUAAUUUAGC | | |
| | | | | | | | UCAAGAAGUC | | |
| | | | | | | | AACCAGAGAA | | |
| | | | | | | | ACACGACUAC | | |
| | | | | | | | UACCUCA | | |
| tSJ060_sh | 297 | UGAG | shGF | 400 | GUCUG | 96 | GGGAAACUAU | | |
| GFP3mut | | GUAG | P3mut | | AUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUG | | GAAAGAUCGA | | |
| | | UU | | | CAAGCT | | CGAGGUGAAA | | |
| | | | | | GCAACT | | ACCUCGUGAC | | |
| | | | | | GAAGTT | | AGUCUGAUAG | | |
| | | | | | CAccacc | | CAAUGUCAAC | | |
| | | | | | TGAACT | | CUCGCCUGCA | | |
| | | | | | TCAGG | | AGCUGCAACU | | |
| | | | | | GTCAGC | | GAAGUUCACC | | |
| | | | | | TTGCag | | ACCUGAACUU | | |
| | | | | | GCGCG | | CAGGGUCAGC | | |
| | | | | | GUUGA | | UUGCAGGCGC | | |
| | | | | | CCAUAC | | GGUUGACCAU | | |
| | | | | | CGUCC | | ACCGUCCAGU | | |
| | | | | | A | | CCUGAUUUUC | | |
| | | | | | | | GAAUCAGAGA | | |
| | | | | | | | AGGAAUUGAU | | |
| | | | | | | | GUAAUUUAGC | | |
| | | | | | | | UCAAGAAGUC | | |
| | | | | | | | AACCAGAGAA | | |
| | | | | | | | ACACGACUAC | | |
| | | | | | | | UACCUCA | | |
| tSJ061_sh | 28 | UGAG | shGF | 401 | GUCUG | 97 | GGGAAACUAU | | |
| GFP4Rz | | GUAG | P4 | | CUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUG | | GAAAGAUCGA | | |
| | | UU | | | CAAGCT | | CGAGGUGAAA | | |
| | | | | | GACCCT | | ACCUCGUGAC | | |
| | | | | | GAAGTT | | AGUCUGCUAG | | |
| | | | | | CATgtgc | | CAAUGUCAAC | | |
| | | | | | caccucua | | CUCGCCUGCA | | |
| | | | | | ATGAAC | | AGCUGACCCU | | |
| | | | | | TTCAGG | | GAAGUUCAUG | | |
| | | | | | GTCAGC | | UGCCACCUCU | | |
| | | | | | TTGCag | | AAUGAACUUC | | |
| | | | | | GCGCG | | AGGGUCAGCU | | |
| | | | | | GUUGA | | UGCAGGCGCG | | |
| | | | | | CCAUAC | | GUUGACCAUA | | |
| | | | | | CGUCC | | CCGUCCAGUC | | |
| | | | | | A | | CUGAUUUUCG | | |
| | | | | | | | AAUCAGAGAA | | |
| | | | | | | | GGAAUUGAUG | | |
| | | | | | | | UAAUUUAGCG | | |
| | | | | | | | CAAGAAGUCA | | |
| | | | | | | | ACCAGAGAAA | | |
| | | | | | | | CACGACUACU | | |
| | | | | | | | ACCUCA | | |
| tSJ062_sh | 299 | UGAG | shGF | 402 | GUCUG | 98 | GGGAAACUAU | | |
| GFP5Rz | | GUAG | P5 | | CUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUG | | GAAAGAUCGA | | |
| | | UU | | | CAAGCT | | CGAGGUGAAA | | |
| | | | | | GACCCT | | ACCUCGUGAC | | |
| | | | | | GAAGTT | | AGUCUGCUAG | | |
| | | | | | CATgtgtt | | CAAUGUCAAC | | |
| | | | | | attcttgAT | | CUCGCCUGCA | | |
| | | | | | GAACTT | | AGCUGACCCU | | |
| | | | | | CAGGG | | GAAGUUCAUG | | |
| | | | | | TCAGCT | | UGUUAUUCUU | | |
| | | | | | TGCagG | | GAUGAACUUC | | |
| | | | | | CGCGG | | AGGGUCAGCU | | |
| | | | | | UUGAC | | UGCAGGCGCG | | |
| | | | | | CAUACC | | GUUGACCAUA | | |
| | | | | | GUCCA | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCG | | |
| | | | | | | | CAAGAAGUCA | | |
| | | | | | | | ACCAGAGAAA | | |
| | | | | | | | CACGACUACU | | |
| | | | | | | | ACCUCA | | |
| tSJ063_sh | 300 | UGAG | shGF | 403 | GUCUG | 99 | GGGAAACUAU | | |
| GFP6Rz | | GUAG | P6 | | AUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUAT | | GAAAGAUCGA | | |
| | | UU | | | GAACTT | | CGAGGUGAAA | | |
| | | | | | CAGGG | | ACCUCGUGAC | | |
| | | | | | TCAGCT | | AGUCUGAUAG | | |
| | | | | | TGCgtgtt | | CAAUGUCAAC | | |
| | | | | | attcttgGC | | CUCGCCUAUG | | |
| | | | | | AAGCTG | | AACUUCAGGG | | |
| | | | | | ACCCTG | | UCAGCUUGCG | | |
| | | | | | AAGTTC | | UGUUAUUCUU | | |
| | | | | | ATagGC | | GGCAAGCUGA | | |
| | | | | | GCGGU | | CCCUGAAGUU | | |
| | | | | | UGACCA | | CAUAGGCGCG | | |
| | | | | | UACCG | | GUUGACCAUA | | |
| | | | | | UCCA | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| tSJ064_sh | 301 | UGAG | shGF | 404 | GUCUG | 100 | GGGAAACUAU | | |
| GFP7Rz | | GUAG | P7 | | AUAGCA | | ACAAUACGGU | | |
| | | UAGA | | | AUGUCA | | AUAUUACCUG | | |
| | | UUGU | | | ACCUCC | | GUUUUCGAUC | | |
| | | AUAG | | | UTGCAG | | GAAAGAUCGA | | |
| | | UU | | | ATGAAC | | CGAGGUGAAA | | |
| | | | | | TTCAGG | | ACCUCGUGAC | | |
| | | | | | GTCAGC | | AGUCUGAUAG | | |
| | | | | | Tgtgttat | | CAAUGUCAAC | | |
| | | | | | tcttgAGCT | | CUCCUUGCAG | | |
| | | | | | GACCCT | | AUGAACUUCA | | |
| | | | | | GAAGTT | | GGGUCAGCUG | | |
| | | | | | CATCTG | | UGUUAUUCUU | | |
| | | | | | CAagGC | | GAGCUGACCC | | |
| | | | | | GGUUG | | UGAAGUUCAU | | |
| | | | | | ACCAUA | | CUGCAAGGCG | | |
| | | | | | CCGUC | | GUUGACCAUA | | |
| | | | | | CA | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| tSJ065_sh | 302 | UGAG | shGF | 405 | GUCcag | 101 | GGGAAACUAU | | |
| GFP8Rz | | GUAG | P8 | | ccagcuua | | ACAAUACGGU | | |
| | | UAGA | | | uUGcgaa | | AUAUUACCUG | | |
| | | UUGU | | | uCUCga | | GUUUUCGAUC | | |
| | | AUAG | | | TGCAGA | | GAAAGAUCGA | | |
| | | UU | | | TGAACT | | CGAGGUGAAA | | |
| | | | | | TCAGG | | ACCUCGUGAC | | |
| | | | | | GTCAGC | | AGUCCAGCCA | | |
| | | | | | Tgtgttat | | GCUUAUUGCG | | |
| | | | | | tcttgAGCT | | AAUCUCGAUG | | |
| | | | | | GACCCT | | CAGAUGAACU | | |
| | | | | | GAAGTT | | UCAGGGUCAG | | |
| | | | | | CATCTG | | CUGUGUUAUU | | |
| | | | | | CAtcGC | | CUUGAGCUGA | | |
| | | | | | GauucgC | | CCCUGAAGUU | | |
| | | | | | UtattCG | | CAUCUGCAUC | | |
| | | | | | UCuuug | | GCGAUUCGCU | | |
| | | | | | CA | | UAUUCGUCUU | | |
| | | | | | | | UGCAGUCCUG | | |
| | | | | | | | AUUUUCGAAU | | |
| | | | | | | | CAGAGAAGCA | | |
| | | | | | | | CCUACGCCUA | | |
| | | | | | | | AUAAAUCCGC | | |
| | | | | | | | UUUCUGAGAA | | |
| | | | | | | | GUCAACCAGA | | |
| | | | | | | | GAAACACGAC | | |
| | | | | | | | UACUACCUCA | | |
| tSJ066_sh | 303 | UGAG | shGF | 406 | GUCugu | 102 | GGGAAACUAU | | |
| GFP9Rz | | GUAG | P9 | | aucaauu | | ACAAUACGGU | | |
| | | UAGA | | | cuUGugu | | AUAUUACCUG | | |
| | | UUGU | | | auCUCg | | GUUUUCGAUC | | |
| | | AUAG | | | CTGCAG | | GAAAGAUCGA | | |
| | | UU | | | ATGAAC | | CGAGGUGAAA | | |
| | | | | | TTCAGG | | ACCUCGUGAC | | |
| | | | | | GTCAGC | | AGUCUGUAUC | | |
| | | | | | Tgtgttat | | AAUUCUUGUG | | |
| | | | | | tcttgAGCT | | UAUCUCGCUG | | |
| | | | | | GACCCT | | CAGAUGAACU | | |
| | | | | | GAAGTT | | UCAGGGUCAG | | |
| | | | | | CATCTG | | CUGUGUUAUU | | |
| | | | | | CAgcGC | | CUUGAGCUGA | | |
| | | | | | Gguacac | | CCCUGAAGUU | | |
| | | | | | CUtattC | | CAUCUGCAGC | | |
| | | | | | GUCcaa | | GCGGUACACC | | |
| | | | | | uCA | | UUAUUCGUCC | | |
| | | | | | | | AAUCAGUCCU | | |
| | | | | | | | GAUUUUCGAA | | |
| | | | | | | | UCAGAGAAGA | | |
| | | | | | | | UGUUACGCCU | | |
| | | | | | | | AAUAAAGCCU | | |
| | | | | | | | UGCGACAAGA | | |
| | | | | | | | AGUCAACCAG | | |
| | | | | | | | AGAAACACGA | | |
| | | | | | | | CUACUACCUC | | |
| | | | | | | | A | | |
| tSJ067_sh | 304 | UGAG | shGF | 407 | GUCUG | 103 | GGGAAACUAU | | |
| GFP10Rz | | GUAG | P10 | | AUAGCA | | ACAAUACGGU | | |
| | | UAGA | | | AUGUCA | | AUAUUACCUG | | |
| | | UUGU | | | ACCUC | | GUUUUCGAUC | | |
| | | AUAG | | | GCCUT | | GAAAGAUCGA | | |
| | | UU | | | GGTGC | | CGAGGUGAAA | | |
| | | | | | AGATGA | | ACCUCGUGAC | | |
| | | | | | ACTTCA | | AGUCUGAUAG | | |
| | | | | | GGGTgtg | | CAAUGUCAAC | | |
| | | | | | ttattct | | CUCGCCUUGG | | |
| | | | | | tgACCCTG | | UGCAGAUGAA | | |
| | | | | | AAGTTC | | CUUCAGGGUG | | |
| | | | | | ATCTGC | | UGUUAUUCUU | | |
| | | | | | ACCAag | | GACCCUGAAG | | |
| | | | | | GCGCG | | UUCAUCUGCA | | |
| | | | | | GUUGA | | CCAAGGCGCG | | |
| | | | | | CCAUAC | | GUUGACCAUA | | |
| | | | | | CGUCC | | CCGUCCAGUC | | |
| | | | | | A | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| tSJ068_sh | 305 | UGAG | shGF | 408 | GUCcag | 104 | GGGAAACUAU | | |
| GFP11Rz | | GUAG | P11 | | ccagcuua | | ACAAUACGGU | | |
| | | UAGA | | | uUGcgaa | | AUAUUACCUG | | |
| | | UUGU | | | uCUCga | | GUUUUCGAUC | | |
| | | AUAG | | | TGGTGC | | GAAAGAUCGA | | |
| | | UU | | | AGATGA | | CGAGGUGAAA | | |
| | | | | | ACTTCA | | ACCUCGUGAC | | |
| | | | | | GGGTgt | | AGUCCAGCCA | | |
| | | | | | gttattct | | GCUUAUUGCG | | |
| | | | | | tgACCCTG | | AAUCUCGAUG | | |
| | | | | | AAGTTC | | GUGCAGAUGA | | |
| | | | | | ATCTGC | | ACUUCAGGGU | | |
| | | | | | ACCAtc | | GUGUUAUUCU | | |
| | | | | | GCGauu | | UGACCCUGAA | | |
| | | | | | cgCUtatt | | GUUCAUCUGC | | |
| | | | | | CGUCuu | | ACCAUCGCGA | | |
| | | | | | ugCA | | UUCGCUUAUU | | |
| | | | | | | | CGUCUUUGCA | | |
| | | | | | | | GUCCUGAUUU | | |
| | | | | | | | UCGAAUCAGA | | |
| | | | | | | | GAAGCACCUA | | |
| | | | | | | | CGCCUAAUAA | | |
| | | | | | | | AUCCGCUUUC | | |
| | | | | | | | UGAGAAGUCA | | |
| | | | | | | | ACCAGAGAAA | | |
| | | | | | | | CACGACUACU | | |
| | | | | | | | ACCUCA | | |
| tSJ069_sh | 306 | UGAG | shGF | 409 | GUCugu | 105 | GGGAAACUAU | | |
| GFP12Rz | | GUAG | P12 | | aucaauu | | ACAAUACGGU | | |
| | | UAGA | | | cuUGugu | | AUAUUACCUG | | |
| | | UUGU | | | auCUCg | | GUUUUCGAUC | | |
| | | AUAG | | | ccgTGG | | GAAAGAUCGA | | |
| | | UU | | | TGCAGA | | CGAGGUGAAA | | |
| | | | | | TGAACT | | ACCUCGUGAC | | |
| | | | | | TCAGGGT | | AGUCUGUAUC | | |
| | | | | | gtgttat | | AAUUCUUGUG | | |
| | | | | | tcttgAC | | UAUCUCGCCG | | |
| | | | | | CCTGAAG | | UGGUGCAGAU | | |
| | | | | | TTCATC | | GAACUUCAGG | | |
| | | | | | TGCACC | | GUGUGUUAUU | | |
| | | | | | AuggcG | | CUUGACCCUG | | |
| | | | | | CGguaca | | AAGUUCAUCU | | |
| | | | | | cCUtattC | | GCACCAUGGC | | |
| | | | | | GUCcaa | | GCGGUACACC | | |
| | | | | | uCA | | UUAUUCGUCC | | |
| | | | | | | | AAUCAGUCCU | | |
| | | | | | | | GAUUUUCGAA | | |
| | | | | | | | UCAGAGAAGA | | |
| | | | | | | | UGUUACGCCU | | |
| | | | | | | | AAUAAAGCCU | | |
| | | | | | | | UGCGACAAGA | | |
| | | | | | | | AGUCAACCAG | | |
| | | | | | | | AGAAACACGA | | |
| | | | | | | | CUACUACCUC | | |
| | | | | | | | A | | |
| 1sided_T | | | | | | 106 | GGGAAACUAU | | |
| let7f_Csg | | | | | | | ACAAUACGGU | | |
| RNA- | | | | | | | AUAUUACCUG | | |
| GFP_ML | | | | | | | GUUUUCGAUC | | |
| _modA | | | | | | | GAAAGAUCGA | | |
| | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGUAAAG | | |
| | | | | | | | AGGCCAUUGC | | |
| | | | | | | | AAAAGAGAAG | | |
| | | | | | | | UCAACCAGAG | | |
| | | | | | | | AAACACGACU | | |
| | | | | | | | ACUACCUCA | | |
| 1sided_T | | | | | | 107 | GGGAAACUAU | | |
| let7f_Csg | | | | | | | ACAAUACGGU | | |
| RNA- | | | | | | | UUAUUACCUG | | |
| GFP_ML | | | | | | | GUUUUCGAUC | | |
| _modA | | | | | | | GAAAGAUCGA | | |
| C25_A38mut | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGUAAAG | | |
| | | | | | | | AGGCCAUUGC | | |
| | | | | | | | AAAAGAGAAG | | |
| | | | | | | | UCAACCAGAG | | |
| | | | | | | | AAAGACGACU | | |
| | | | | | | | ACUACCUCA | | |
| 1sided_T | | | | | | 108 | GGGAAACUAU | | |
| let7f_Csg | | | | | | | ACAAUACGGU | | |
| RNA- | | | | | | | AUAUUACCUG | | |
| GFP_ML | | | | | | | GUUUUCGAUC | | |
| _modB | | | | | | | GAAAGAUCGA | | |
| | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGGAAAG | | |
| | | | | | | | CGGCCCUUGC | | |
| | | | | | | | CAAAGAGAAG | | |
| | | | | | | | UCAACCAGAG | | |
| | | | | | | | AAACACGACU | | |
| | | | | | | | ACUACCUCA | | |
| 1sided_T | | | | | | 109 | GGGAAACUAU | | |
| let7f_Csg | | | | | | | ACAAUACGGU | | |
| RNA- | | | | | | | UUAUUACCUG | | |
| GFP_ML | | | | | | | GUUUUCGAUC | | |
| _modB | | | | | | | GAAAGAUCGA | | |
| C25_A38_mut | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGGAAAG | | |
| | | | | | | | CGGCCCUUGC | | |
| | | | | | | | CAAAGAGAAG | | |
| | | | | | | | UCAACCAGAG | | |
| | | | | | | | AAAGACGACU | | |
| | | | | | | | ACUACCUCA | | |
| 1sided_T | | | | | | 110 | GGGAAACUAU | | |
| let7f_Csg | | | | | | | ACAAUACGGU | | |
| RNA- | | | | | | | UUAUUACCUG | | |
| GFP_ML | | | | | | | GUUUUCGAUC | | |
| _modC | | | | | | | GAAAGAUCGA | | |
| C25_A38_mut | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGGCAAG | | |
| | | | | | | | CUGCCCGUGC | | |
| | | | | | | | CCAAGAGAAG | | |
| | | | | | | | UCAACCAGAG | | |
| | | | | | | | AAAGACGACU | | |
| | | | | | | | ACUACCUCA | | |
| 1sided_T | | | | | | 111 | GGGAAACUAU | | |
| let7f_Csg | | | | | | | ACAAUACGGU | | |
| RNA- | | | | | | | AUAUUACCUG | | |
| GFP_ML | | | | | | | GUUUUCGAUC | | |
| _modD | | | | | | | GAAAGAUCGA | | |
| | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | AAAAAAAUAU | | |
| | | | | | | | CCCCGGCAAG | | |
| | | | | | | | CUGCCCGUGC | | |
| | | | | | | | CCAAGAGAAG | | |
| | | | | | | | UCAACCAGAG | | |
| | | | | | | | AAACACGACU | | |
| | | | | | | | ACUACCUCA | | |
| 1sided_T | | | | | | 112 | GGGAAACUAU | | |
| let7f_Csg | | | | | | | ACAAUACGGU | | |
| RNA- | | | | | | | UUAUUACCUG | | |
| GFP_ML | | | | | | | GUUUUCGAUC | | |
| _modD | | | | | | | GAAAGAUCGA | | |
| C25_A38_mut | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | AAAAAAAUAU | | |
| | | | | | | | CCCCGGCAAG | | |
| | | | | | | | CUGCCCGUGC | | |
| | | | | | | | CCAAGAGAAG | | |
| | | | | | | | UCAACCAGAG | | |
| | | | | | | | AAAGACGACU | | |
| | | | | | | | ACUACCUCA | | |
| 1sided_T | | | | | | 113 | GGGAAACUAU | | |
| let7f_Csg | | | | | | | ACAAUACGGU | | |
| RNA- | | | | | | | AUAUUACCUG | | |
| GFP_ML | | | | | | | GUUUUCGAUC | | |
| _modC | | | | | | | GAAAGAUCGA | | |
| | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGGCAAG | | |
| | | | | | | | CUGCCCGUGC | | |
| | | | | | | | CCAAGAGAAG | | |
| | | | | | | | UCAACCAGAG | | |
| | | | | | | | AAACACGACU | | |
| | | | | | | | ACUACCUCA | | |
| 1sided_Tlet7f | | | | | | 114 | GGGAAACUAU | | |
| CsgRNA- | | | | | | | ACAAUACGGU | | |
| GFP_ML_mo | | | | | | | AUAUUACCUG | | |
| dC1_8H2_ext | | | | | | | GUUUUCGAUC | | |
| ra_A7_addU | | | | | | | GAAAGAUCGA | | |
| C | | | | | | | CGAGGUGAAA | | |
| | | | | | | | ACCUCGCGUA | | |
| | | | | | | | UGACAGGGCA | | |
| | | | | | | | CGGGCAGCUU | | |
| | | | | | | | GCCGGGUUUU | | |
| | | | | | | | AGAGCUAGAA | | |
| | | | | | | | AUAGCAAGUU | | |
| | | | | | | | AAAAUAAGGC | | |
| | | | | | | | UAGUCCGUUA | | |
| | | | | | | | UCAACUUGAA | | |
| | | | | | | | AAAGUGGCAC | | |
| | | | | | | | CGAGUCGGUG | | |
| | | | | | | | CGUCAGUCCU | | |
| | | | | | | | GAUUUUCGAA | | |
| | | | | | | | UCAGAGAAGA | | |
| | | | | | | | CCCAUGAUAA | | |
| | | | | | | | CUUAUUUUAA | | |
| | | | | | | | CUUUCUAAAA | | |
| | | | | | | | CCCGGCAAGC | | |
| | | | | | | | UGCCCGUGAG | | |
| | | | | | | | AAGUCAUACG | | |
| | | | | | | | ACCAGAGAAA | | |
| | | | | | | | CACGACUACU | | |
| | | | | | | | ACCUCA | | |
| 1sided_T | | | | | | 115 | GGGAAACUAU | | |
| let7f_Csg | | | | | | | ACAAUACGGU | | |
| RNA- | | | | | | | UUAUUACCUG | | |
| GFP_ML | | | | | | | GUUUUCGAUC | | |
| _modC_ | | | | | | | GAAAGAUCGA | | |
| A38mut | | | | | | | CGAGGUGAAA | | |
| | | | | | | | ACCUCGUGAC | | |
| | | | | | | | AGUCCUUGGG | | |
| | | | | | | | CACGGGCAGC | | |
| | | | | | | | UUGCCGGGUU | | |
| | | | | | | | UUAGAGCUAG | | |
| | | | | | | | AAAUAGCAAG | | |
| | | | | | | | UUAAAAUAAG | | |
| | | | | | | | GCUAGUCCGU | | |
| | | | | | | | UAUCAACUUG | | |
| | | | | | | | AAAAAGUGGC | | |
| | | | | | | | ACCGAGUCGG | | |
| | | | | | | | UGCUUUUUUU | | |
| | | | | | | | GUCAGUCCUG | | |
| | | | | | | | AUUUUCGAAU | | |
| | | | | | | | CAGAGAAGAC | | |
| | | | | | | | CCCAACCUAU | | |
| | | | | | | | CCCCGGCAAG | | |
| | | | | | | | CUGCCCGUGC | | |
| | | | | | | | CCAAGAGAAG | | |
| | | | | | | | UCAACCAGAG | | |
| | | | | | | | AAACACGACU | | |
| | | | | | | | ACUACCUCA | | |
| 1sided_Tlet7f | | | | | | 116 | GGGAAACUAU | | |
| CsgRNA- | | | | | | | ACAAUACGGU | | |
| GFP_ML_mo | | | | | | | UUAUUACCUG | | |
| dC1_8H2_ext | | | | | | | GUUUUCGAUC | | |
| ra_A7_addU | | | | | | | GAAAGAUCGA | | |
| C_A38mut | | | | | | | CGAGGUGAAA | | |
| | | | | | | | ACCUCGCGUA | | |
| | | | | | | | UGACAGGGCA | | |
| | | | | | | | CGGGCAGCUU | | |
| | | | | | | | GCCGGGUUUU | | |
| | | | | | | | AGAGCUAGAA | | |
| | | | | | | | AUAGCAAGUU | | |
| | | | | | | | AAAAUAAGGC | | |
| | | | | | | | UAGUCCGUUA | | |
| | | | | | | | UCAACUUGAA | | |
| | | | | | | | AAAGUGGCAC | | |
| | | | | | | | CGAGUCGGUG | | |
| | | | | | | | CGUCAGUCCU | | |
| | | | | | | | GAUUUUCGAA | | |
| | | | | | | | UCAGAGAAGA | | |
| | | | | | | | CCCAUGAUAA | | |
| | | | | | | | CUUAUUUUAA | | |
| | | | | | | | CUUUCUAAAA | | |
| | | | | | | | CCCGGCAAGC | | |
| | | | | | | | UGCCCGUGAG | | |
| | | | | | | | AAGUCAUACG | | |
| | | | | | | | ACCAGAGAAA | | |
| | | | | | | | CACGACUACU | | |
| | | | | | | | ACCUCA | | |
| 1sided_T | | | | | | 117 | GGGAAACUAU | | |
| let7f_Csh | | | | | | | ACAAUACGGU | | |
| RNA6- | | | | | | | AUAUUACCUG | | |
| GFP_ML | | | | | | | GUUUUCGAUC | | |
| | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 118 | GGGAAACUAU | | |
| let7f_Csh | | | | | | | ACAAUACGGU | | |
| RNA- | | | | | | | AUAUUACCUG | | |
| GFP6mut | | | | | | | GUUUUCGAUC | | |
| ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUACUGG | | |
| | | | | | | | UCAGCUUGCG | | |
| | | | | | | | UGUUAUUCUU | | |
| | | | | | | | GGCAAGCUGA | | |
| | | | | | | | CCAGUAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 119 | GGGAAACUAU | | |
| let7f_Csh | | | | | | | ACAAUACGGU | | |
| RNA- | | | | | | | UUAUUACCUG | | |
| GFP6_M | | | | | | | GUUUUCGAUC | | |
| L_A38mu | | | | | | | GAAAGAUCGA | | |
| t | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 120 | GGGAACGUAC | | |
| Egene20_ | | | | | | | CUGUACGGUA | | |
| CshRN | | | | | | | UAUUACCUGG | | |
| A- | | | | | | | UUUUCGAUCG | | |
| GFP6_M | | | | | | | AAAGAUCGAC | | |
| L | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GAAUUGAUGU | | |
| | | | | | | | AAUUUAGCUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 121 | GGGAACGUAC | | |
| Egene20_ | | | | | | | CUGUACGGUA | | |
| CshRN | | | | | | | UAUUACCUGG | | |
| A- | | | | | | | UUUUCGAUCG | | |
| GFP6mut | | | | | | | AAAGAUCGAC | | |
| _ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUACUGGU | | |
| | | | | | | | CAGCUUGCGU | | |
| | | | | | | | GUUAUUCUUG | | |
| | | | | | | | GCAAGCUGAC | | |
| | | | | | | | CAGUAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GAAUUGAUGU | | |
| | | | | | | | AAUUUAGCUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 122 | GGGAACGUAC | | |
| Egene20_ | | | | | | | CUGUACGGUU | | |
| CshRN | | | | | | | UAUUACCUGG | | |
| A- | | | | | | | UUUUCGAUCG | | |
| GFP6_M | | | | | | | AAAGAUCGAC | | |
| L_A38mut | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GAAUUGAUGU | | |
| | | | | | | | AAUUUAGCUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 123 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 1_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | AUUUAGCUCA | | |
| | | | | | | | AGAAGUCAAC | | |
| | | | | | | | CAGAGAAACA | | |
| | | | | | | | CGACUACUAC | | |
| | | | | | | | CUCA | | |
| 1sided_T | | | | | | 124 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 2_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 125 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 3_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGUGCGU | | |
| | | | | | | | UAAUUUCUCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 126 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 4_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | UGACGUGAUG | | |
| | | | | | | | UAAUUUAUAU | | |
| | | | | | | | CCAGAAGUCA | | |
| | | | | | | | ACCAGAGAAA | | |
| | | | | | | | CACGACUACU | | |
| | | | | | | | ACCUCA | | |
| 1sided_T | | | | | | 127 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 5_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | UGACGUGAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 128 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 6_ML_A38mut | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 129 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 1_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGAAUUGAUG | | |
| | | | | | | | AUUUAGCUCA | | |
| | | | | | | | AGAAGUCAAC | | |
| | | | | | | | CAGAGAAACA | | |
| | | | | | | | CGACUACUAC | | |
| | | | | | | | CUCA | | |
| 1sided_T | | | | | | 130 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 131 | GGGAAACUAU | | |
| let7f_Csh | | | | | | | ACAAUACGGU | | |
| RNA- | | | | | | | AUAUUACCUG | | |
| GFP6mut | | | | | | | GUUUUCGAUC | | |
| _D2_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUacUGG | | |
| | | | | | | | UCAGCUUGCG | | |
| | | | | | | | UGUUAUUCUU | | |
| | | | | | | | GGCAAGCUGA | | |
| | | | | | | | CCagUAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 132 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 3_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGUGCGU | | |
| | | | | | | | UAAUUUCUCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 133 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 4_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | UGACGUGAUG | | |
| | | | | | | | UAAUUUAUAU | | |
| | | | | | | | CCAGAAGUCA | | |
| | | | | | | | ACCAGAGAAA | | |
| | | | | | | | CACGACUACU | | |
| | | | | | | | ACCUCA | | |
| 1sided_T | | | | | | 134 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 5_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | UGACGUGAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 135 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 6_ML | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUUAGCU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 136 | GGGAACGUAC | | |
| Egene20_ | | | | | | | CUGUACGGUA | | |
| CshRN | | | | | | | UAUUACCUGG | | |
| A- | | | | | | | UUUUCGAUCG | | |
| GFP6_D | | | | | | | AAAGAUCGAC | | |
| 2_ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 137 | GGGAACGUAC | | |
| Egene20_ | | | | | | | CUGUACGGUU | | |
| CshRN | | | | | | | UAUUACCUGG | | |
| A- | | | | | | | UUUUCGAUCG | | |
| GFP6_D | | | | | | | AAAGAUCGAC | | |
| 2_ML_A38mut | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 138 | GGGAACCCAG | | |
| mir222_ | | | | | | | UAGCACGGUA | | |
| CshRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACAGAUG | | |
| | | | | | | | UAGCU | | |
| 1sided_T | | | | | | 139 | GGGAACCCAG | | |
| mir222_ | | | | | | | UAGCACGGUU | | |
| CshRNA- | | | | | | | UAUUACCUGG | | |
| GFP6_D | | | | | | | UUUUCGAUCG | | |
| 2_ML_A38mut | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACAGAUG | | |
| | | | | | | | UAGCU | | |
| 1sided_T | | | | | | 140 | GGGAACCCAG | | |
| mir222_ | | | | | | | UAGCACGGUA | | |
| CshRNA- | | | | | | | UAUUACCUGG | | |
| GFP6mut | | | | | | | UUUUCGAUCG | | |
| _D2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUacUGGU | | |
| | | | | | | | CAGCUUGCGU | | |
| | | | | | | | GUUAUUCUUG | | |
| | | | | | | | GCAAGCUGAC | | |
| | | | | | | | CagUAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACAGAUG | | |
| | | | | | | | UAGCU | | |
| 1sided_T | | | | | | 141 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_CM1 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGACUACU | | |
| | | | | | | | ACCUCA | | |
| 1sided_T | | | | | | 142 | GGGAAACUAU | | |
| let7f_Csh | | | | | | | ACAAUAUGGU | | |
| RNA- | | | | | | | AUAUUACCUG | | |
| GFP6_D | | | | | | | GUUUUCGAUC | | |
| 2_CM2 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 143 | GGGAAACUAU | | |
| let7f_Csh | | | | | | | ACAAUAUGGU | | |
| RNA- | | | | | | | UUAUUACCUG | | |
| GFP6_D | | | | | | | GUUUUCGAUC | | |
| 2_CM2_ | | | | | | | GAAAGAUCGA | | |
| A38mut | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGACUACU | | |
| | | | | | | | ACCUCA | | |
| 1sided_T | | | | | | 144 | GGGAAACUAU | | |
| let7f_Csh | | | | | | | ACAAUAUGGU | | |
| RNA- | | | | | | | AUAUUACCUG | | |
| GFP6_D | | | | | | | GUUUUCGAUC | | |
| 2_CM3 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGACUACU | | |
| | | | | | | | ACCUCA | | |
| 1sided_T | | | | | | 145 | GGGAAACUAU | | |
| let7f_Csh | | | | | | | ACAAUCGGUA | | |
| RNA- | | | | | | | UAUUACCUGG | | |
| GFP6_D | | | | | | | UUUUCGAUCG | | |
| 2_CM4 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGCUACUAC | | |
| | | | | | | | CUCA | | |
| 1sided_T | | | | | | 146 | GGGAAACUAU | | |
| let7f_Csh | | | | | | | ACAAUUUGGU | | |
| RNA- | | | | | | | AUAUUACCUG | | |
| GFP6_D | | | | | | | GUUUUCGAUC | | |
| 2_CM5 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGGCUACU | | |
| | | | | | | | ACCUCA | | |
| 1sided_T | | | | | | 147 | GGGAAACUAU | | |
| let7f_Csh | | | | | | | ACAAUUAUGU | | |
| RNA- | | | | | | | AUAUUACCUG | | |
| GFP6_D | | | | | | | GUUUUCGAUC | | |
| 2_CM6 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAAUACUACU | | |
| | | | | | | | ACCUCA | | |
| 1sided_T | | | | | | 148 | GGGAAACUAU | | |
| let7f_Csh | | | | | | | ACAAUACUGU | | |
| RNA- | | | | | | | AUAUUACCUG | | |
| GFP6_D | | | | | | | GUUUUCGAUC | | |
| 2_CM7 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAAGACUACU | | |
| | | | | | | | ACCUCA | | |
| 1sided_T | | | | | | 149 | GGGAAACUAU | | |
| let7f_Csh | | | | | | | ACAAUAUGGU | | |
| RNA- | | | | | | | AUAUUACCUG | | |
| GFP6_D | | | | | | | GUUUUCGAUC | | |
| 2_CM8 | | | | | | | GAAAGAUCGA | | |
| | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACAACUACU | | |
| | | | | | | | ACCUCA | | |
| 1sided_T | | | | | | 150 | GGGAAACUAU | | |
| let7f_Csh | | | | | | | ACAAUCUGUA | | |
| RNA- | | | | | | | UAUUACCUGG | | |
| GFP6_D | | | | | | | UUUUCGAUCG | | |
| 2_CM9 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAGCUACUAC | | |
| | | | | | | | CUCA | | |
| 1sided_T | | | | | | 151 | GGGAAACUAU | | |
| let7f_Csh | | | | | | | ACAAUUGGUA | | |
| RNA- | | | | | | | UAUUACCUGG | | |
| GFP6_D | | | | | | | UUUUCGAUCG | | |
| 2_CM10 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACACUACUAC | | |
| | | | | | | | CUCA | | |
| 1sided_T | | | | | | 152 | GGGAACGUAC | | |
| Egene20_ | | | | | | | CUGUACGGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM1 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGACUCUUC | | |
| | | | | | | | CGAA | | |
| 1sided_T | | | | | | 153 | GGGAACGUAC | | |
| Egene20_ | | | | | | | CUGUAUGGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM2 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 154 | GGGAACGUAC | | |
| Egene20_ | | | | | | | CUGUAUGGUU | | |
| CshRN | | | | | | | UAUUACCUGG | | |
| A- | | | | | | | UUUUCGAUCG | | |
| GFP6_D | | | | | | | AAAGAUCGAC | | |
| 2_CM2A38mut | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACUCUUC | | |
| | | | | | | | CGAA | | |
| 1sided_T | | | | | | 155 | GGGAACGUAC | | |
| Egene20_ | | | | | | | CUGUAUGGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM3 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGACUCUUC | | |
| | | | | | | | CGAA | | |
| 1sided_T | | | | | | 156 | GGGAACGUAC | | |
| Egene20_ | | | | | | | CUGUCGGUAU | | |
| CshRN | | | | | | | AUUACCUGGU | | |
| A- | | | | | | | UUUCGAUCGA | | |
| GFP6_D | | | | | | | AAGAUCGACG | | |
| 2_CM4 | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | CGCUCUUCCG | | |
| | | | | | | | AA | | |
| 1sided_T | | | | | | 157 | GGGAACGUAC | | |
| Egene20_ | | | | | | | CUGUUUGGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM5 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGGCUCUUC | | |
| | | | | | | | CGAA | | |
| 1sided_T | | | | | | 158 | GGGAACGUAC | | |
| Egene20_ | | | | | | | CUGUUAUGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM6 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAUACUCUUC | | |
| | | | | | | | CGAA | | |
| 1sided_T | | | | | | 159 | GGGAACGUAC | | |
| Egene20_ | | | | | | | CUGUACUGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM7 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAGACUCUUC | | |
| | | | | | | | CGAA | | |
| 1sided_T | | | | | | 160 | GGGAACGUAC | | |
| Egene20_ | | | | | | | CUGUAUGGUA | | |
| CshRN | | | | | | | UAUUACCUGG | | |
| A- | | | | | | | UUUUCGAUCG | | |
| GFP6_D | | | | | | | AAAGAUCGAC | | |
| 2_CM8 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACAACUCUUC | | |
| | | | | | | | CGAA | | |
| 1sided_T | | | | | | 161 | GGGAACGUAC | | |
| Egene20_ | | | | | | | CUGUCUGUAU | | |
| CshRN | | | | | | | AUUACCUGGU | | |
| A- | | | | | | | UUUCGAUCGA | | |
| GFP6_D | | | | | | | AAGAUCGACG | | |
| 2_CM9 | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | AGCUCUUCCG | | |
| | | | | | | | AA | | |
| 1sided_T | | | | | | 162 | GGGAACGUAC | | |
| Egene20_ | | | | | | | CUGUUGGUAU | | |
| CshRN | | | | | | | AUUACCUGGU | | |
| A- | | | | | | | UUUCGAUCGA | | |
| GFP6_D | | | | | | | AAGAUCGACG | | |
| 2_CM10 | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | CACUCUUCCG | | |
| | | | | | | | AA | | |
| 1sided_T | | | | | | 163 | GGGAACCCAG | | |
| mir222_ | | | | | | | UAGCACGGUA | | |
| CshRNA- | | | | | | | UAUUACCUGG | | |
| GFP6_D | | | | | | | UUUUCGAUCG | | |
| 2_CM1 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGACAGAUG | | |
| | | | | | | | UAGCU | | |
| 1sided_T | | | | | | 164 | GGGAACCCAG | | |
| mir222_ | | | | | | | UAGCAUGGUA | | |
| CshRNA- | | | | | | | UAUUACCUGG | | |
| GFP6_D | | | | | | | UUUUCGAUCG | | |
| 2_CM2 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGACAGAUG | | |
| | | | | | | | UAGCU | | |
| 1sided_T | | | | | | 165 | GGGAACCCAG | | |
| mir222_ | | | | | | | UAGCAUGGUA | | |
| CshRNA- | | | | | | | UAUUACCUGG | | |
| GFP6_D | | | | | | | UUUUCGAUCG | | |
| 2_CM3 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGACAGAUG | | |
| | | | | | | | UAGCU | | |
| 1sided_T | | | | | | 166 | GGGAACCCAG | | |
| mir222_ | | | | | | | UAGCCGGUAU | | |
| CshRNA- | | | | | | | AUUACCUGGU | | |
| GFP6_D | | | | | | | UUUCGAUCGA | | |
| 2_CM4 | | | | | | | AAGAUCGACG | | |
| | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | CGCAGAUGUA | | |
| | | | | | | | GCU | | |
| 1sided_T | | | | | | 167 | GGGAACCCAG | | |
| mir222_ | | | | | | | UAGCUUGGUA | | |
| CshRNA- | | | | | | | UAUUACCUGG | | |
| GFP6_D | | | | | | | UUUUCGAUCG | | |
| 2_CM5 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AUGGCAGAUG | | |
| | | | | | | | UAGCU | | |
| 1sided_T | | | | | | 168 | GGGAACCCAG | | |
| mir222_ | | | | | | | UAGCUAUGUA | | |
| CshRNA- | | | | | | | UAUUACCUGG | | |
| GFP6_D | | | | | | | UUUUCGAUCG | | |
| 2_CM6 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAUACAGAUG | | |
| | | | | | | | UAGCU | | |
| 1sided_T | | | | | | 169 | GGGAACCCAG | | |
| mir222_ | | | | | | | UAGCACUGUA | | |
| CshRNA- | | | | | | | UAUUACCUGG | | |
| GFP6_D | | | | | | | UUUUCGAUCG | | |
| 2_CM7 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAGACAGAUG | | |
| | | | | | | | UAGCU | | |
| 1sided_T | | | | | | 170 | GGGAACCCAG | | |
| mir222_ | | | | | | | UAGCAUGGUA | | |
| CshRNA- | | | | | | | UAUUACCUGG | | |
| GFP6_D | | | | | | | UUUUCGAUCG | | |
| 2_CM8 | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACAACAGAUG | | |
| | | | | | | | UAGCU | | |
| 1sided_T | | | | | | 171 | GGGAACCCAG | | |
| mir222_ | | | | | | | UAGCCUGUAU | | |
| CshRNA- | | | | | | | AUUACCUGGU | | |
| GFP6_D | | | | | | | UUUCGAUCGA | | |
| 2_CM9 | | | | | | | AAGAUCGACG | | |
| | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | AGCAGAUGUA | | |
| | | | | | | | GCU | | |
| 1sided_T | | | | | | 172 | GGGAACCCAG | | |
| mir222_ | | | | | | | UAGCUGGUAU | | |
| CshRNA- | | | | | | | AUUACCUGGU | | |
| GFP6_D | | | | | | | UUUCGAUCGA | | |
| 2_CM10 | | | | | | | AAGAUCGACG | | |
| | | | | | | | AGGUGAAAAC | | |
| | | | | | | | CUCGUGACAG | | |
| | | | | | | | UCUGAUAGCA | | |
| | | | | | | | AUGUCAACCU | | |
| | | | | | | | CGCCUAUGAA | | |
| | | | | | | | CUUCAGGGUC | | |
| | | | | | | | AGCUUGCGUG | | |
| | | | | | | | UUAUUCUUGG | | |
| | | | | | | | CAAGCUGACC | | |
| | | | | | | | CUGAAGUUCA | | |
| | | | | | | | UAGGCGCGGU | | |
| | | | | | | | UGACCAUACC | | |
| | | | | | | | GUCCAGUCCU | | |
| | | | | | | | GAUUUUCGAA | | |
| | | | | | | | UCAGAGAAGG | | |
| | | | | | | | ACGGUAUGUA | | |
| | | | | | | | AUUGCUAUCA | | |
| | | | | | | | AGAAGUCAAC | | |
| | | | | | | | CAGAGAAACA | | |
| | | | | | | | CACAGAUGUA | | |
| | | | | | | | GCU | | |
| 1sided_T | | | | | | 173 | GGGAUCUGAA | | |
| cel-mir- | | | | | | | UGGCAACGGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM1 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGAUCGGA | | |
| | | | | | | | GUACAAA | | |
| 1sided_T | | | | | | 174 | GGGAUCUGAA | | |
| cel-mir- | | | | | | | UGGCAAUGGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM2 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACGAUCGGA | | |
| | | | | | | | GUACAAA | | |
| 1sided_T | | | | | | 175 | GGGAUCUGAA | | |
| cel-mir- | | | | | | | UGGCAAUGGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM3 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGAUCGGA | | |
| | | | | | | | GUACAAA | | |
| 1sided_T | | | | | | 176 | GGGAUCUGAA | | |
| cel-mir- | | | | | | | UGGCACGGUA | | |
| 238_Csh | | | | | | | UAUUACCUGG | | |
| RNA- | | | | | | | UUUUCGAUCG | | |
| GFP6_D | | | | | | | AAAGAUCGAC | | |
| 2_CM4 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGUCGGAGU | | |
| | | | | | | | ACAAA | | |
| 1sided_T | | | | | | 177 | GGGAUCUGAA | | |
| cel-mir- | | | | | | | UGGCAUUGGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM5 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAUGGUCGGA | | |
| | | | | | | | GUACAAA | | |
| 1sided_T | | | | | | 178 | GGGAUCUGAA | | |
| cel-mir- | | | | | | | UGGCAUAUGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM6 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAAUAUCGGA | | |
| | | | | | | | GUACAAA | | |
| 1sided_T | | | | | | 179 | GGGAUCUGAA | | |
| cel-mir- | | | | | | | UGGCAACUGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM7 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CAAGAUCGGA | | |
| | | | | | | | GUACAAA | | |
| 1sided_T | | | | | | 180 | GGGAUCUGAA | | |
| cel-mir- | | | | | | | UGGCAAUGGU | | |
| 238_Csh | | | | | | | AUAUUACCUG | | |
| RNA- | | | | | | | GUUUUCGAUC | | |
| GFP6_D | | | | | | | GAAAGAUCGA | | |
| 2_CM8 | | | | | | | CGAGGUGAAA | | |
| | | | | | | | ACCUCGUGAC | | |
| | | | | | | | AGUCUGAUAG | | |
| | | | | | | | CAAUGUCAAC | | |
| | | | | | | | CUCGCCUAUG | | |
| | | | | | | | AACUUCAGGG | | |
| | | | | | | | UCAGCUUGCG | | |
| | | | | | | | UGUUAUUCUU | | |
| | | | | | | | GGCAAGCUGA | | |
| | | | | | | | CCCUGAAGUU | | |
| | | | | | | | CAUAGGCGCG | | |
| | | | | | | | GUUGACCAUA | | |
| | | | | | | | CCGUCCAGUC | | |
| | | | | | | | CUGAUUUUCG | | |
| | | | | | | | AAUCAGAGAA | | |
| | | | | | | | GGACGGUAUG | | |
| | | | | | | | UAAUUGCUAU | | |
| | | | | | | | CAAGAAGUCA | | |
| | | | | | | | ACCAGAGAAA | | |
| | | | | | | | CACAAUCGGA | | |
| | | | | | | | GUACAAA | | |
| 1sided_T | | | | | | 181 | GGGAUCUGAA | | |
| cel-mir- | | | | | | | UGGCACUGUA | | |
| 238_Csh | | | | | | | UAUUACCUGG | | |
| RNA- | | | | | | | UUUUCGAUCG | | |
| GFP6_D | | | | | | | AAAGAUCGAC | | |
| 2_CM9 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | AAGUCGGAGU | | |
| | | | | | | | ACAAA | | |
| 1sided_T | | | | | | 182 | GGGAUCUGAA | | |
| cel-mir- | | | | | | | UGGCAUGGUA | | |
| 238_Csh | | | | | | | UAUUACCUGG | | |
| RNA- | | | | | | | UUUUCGAUCG | | |
| GFP6_D | | | | | | | AAAGAUCGAC | | |
| 2_CM10 | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACAUCGGAGU | | |
| | | | | | | | ACAAA | | |
| 1sided_T | | | | | | 183 | GGGAGUUAAC | | |
| Egene20_ | | | | | | | AAUAACGGUA | | |
| alt1_Cs | | | | | | | UAUUACCUGG | | |
| hRNA- | | | | | | | UUUUCGAUCG | | |
| GFP6_D | | | | | | | AAAGAUCGAC | | |
| 2_ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAUUGCAG | | |
| | | | | | | | CAGU | | |
| 1sided_T | | | | | | 184 | GGGAGAAGAA | | |
| Egene20_ | | | | | | | UUCAACGGUA | | |
| alt2_Cs | | | | | | | UAUUACCUGG | | |
| hRNA- | | | | | | | UUUUCGAUCG | | |
| GFP6_D | | | | | | | AAAGAUCGAC | | |
| 2_ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAGAUUUU | | |
| | | | | | | | UAAC | | |
| 1sided_T | | | | | | 185 | GGGAUCAGAU | | |
| Egene20_ | | | | | | | UUUAACGGUA | | |
| alt3_Cs | | | | | | | UAUUACCUGG | | |
| hRNA- | | | | | | | UUUUCGAUCG | | |
| GFP6_D | | | | | | | AAAGAUCGAC | | |
| 2_ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAUUGCAG | | |
| | | | | | | | CAGU | | |
| 1sided_T | | | | | | 186 | GGGAAGUAAG | | |
| Egene20_ | | | | | | | GAUGACGGUA | | |
| alt4_Cs | | | | | | | UAUUACCUGG | | |
| hRNA- | | | | | | | UUUUCGAUCG | | |
| GFP6_D | | | | | | | AAAGAUCGAC | | |
| 2_ML | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAGCUAGU | | |
| | | | | | | | GUAA | | |
| 1sided_T | | | | | | 187 | GGGACCGAAA | | |
| Egene20_alt5_Cs | | | | | | | CGAAACGGUA | | |
| hRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAUGAGUA | | |
| | | | | | | | CAUG | | |
| 1sided_T | | | | | | 188 | GGGAAAGUAC | | |
| Egene20alt6_Cs | | | | | | | GCUAACGGUA | | |
| hRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAUUAACU | | |
| | | | | | | | AUUA | | |
| 1sided_T | | | | | | 189 | GGGAAGUGUA | | |
| Egene20_alt7_Cs | | | | | | | ACUAACGGUA | | |
| hRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAGCAAGA | | |
| | | | | | | | AUAC | | |
| 1sided_T | | | | | | 190 | GGGAUACAAG | | |
| Egene20alt8_Cs | | | | | | | ACUCACGGUA | | |
| hRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAACGUUA | | |
| | | | | | | | ACAA | | |
| 1sided_T | | | | | | 191 | GGGAAGUAAA | | |
| Egene20_alt9_Cs | | | | | | | CGUAACGGUA | | |
| hRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAAAAAGA | | |
| | | | | | | | AGGU | | |
| 1sided_T | | | | | | 192 | GGGAGAACUC | | |
| Egene20alt10_C | | | | | | | UAGAACGGUA | | |
| shRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAAGAAUU | | |
| | | | | | | | CAGA | | |
| 1sided_T | | | | | | 193 | GGGAGACCAG | | |
| Egene20_alt11_C | | | | | | | AAGAACGGUA | | |
| shRNA- | | | | | | | UAUUACCUGG | | |
| GFP6_D | | | | | | | UUUUCGAUCG | | |
| 2_ML | | | | | | | AAAGAUCGAC | | |
| | | | | | | | GAGGUGAAAA | | |
| | | | | | | | CCUCGUGACA | | |
| | | | | | | | GUCUGAUAGC | | |
| | | | | | | | AAUGUCAACC | | |
| | | | | | | | UCGCCUAUGA | | |
| | | | | | | | ACUUCAGGGU | | |
| | | | | | | | CAGCUUGCGU | | |
| | | | | | | | GUUAUUCUUG | | |
| | | | | | | | GCAAGCUGAC | | |
| | | | | | | | CCUGAAGUUC | | |
| | | | | | | | AUAGGCGCGG | | |
| | | | | | | | UUGACCAUAC | | |
| | | | | | | | CGUCCAGUCC | | |
| | | | | | | | UGAUUUUCGA | | |
| | | | | | | | AUCAGAGAAG | | |
| | | | | | | | GACGGUAUGU | | |
| | | | | | | | AAUUGCUAUC | | |
| | | | | | | | AAGAAGUCAA | | |
| | | | | | | | CCAGAGAAAC | | |
| | | | | | | | ACGAUCAGGA | | |
| | | | | | | | ACUC | | |
| 1sided_T | | | | | | 194 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_5RH2 | | | | | | | GAAAGAUCGA | | |
| | | | | | | | CGAGGUGAAA | | |
| | | | | | | | ACCUCGAUGA | | |
| | | | | | | | CAGUCUGAUA | | |
| | | | | | | | GCAAUGUCAA | | |
| | | | | | | | CCUCGCCUAU | | |
| | | | | | | | GAACUUCAGG | | |
| | | | | | | | GUCAGCUUGC | | |
| | | | | | | | GUGUUAUUCU | | |
| | | | | | | | UGGCAAGCUG | | |
| | | | | | | | ACCCUGAAGU | | |
| | | | | | | | UCAUAGGCGC | | |
| | | | | | | | GGUUGACCAU | | |
| | | | | | | | ACCGUCCAGU | | |
| | | | | | | | CCUGAUUUUC | | |
| | | | | | | | GAAUCAGAGA | | |
| | | | | | | | AGGACGGUAU | | |
| | | | | | | | GUAAUUGCUA | | |
| | | | | | | | UCAAGAAGUC | | |
| | | | | | | | AUACCAGAGA | | |
| | | | | | | | AACACGACUA | | |
| | | | | | | | CUACCUCA | | |
| 1sided_Tlet7f | | | | | | 195 | GGGAAACUAU | | |
| CshRNA- | | | | | | | ACAAUACGGU | | |
| GFP6_D2_M | | | | | | | AUAUUACCUG | | |
| L_5RH2_left | | | | | | | GUUUUCGAUC | | |
| cleav_site_m | | | | | | | GAAAGAUCGA | | |
| ut_v1 | | | | | | | CGAGGUGAAA | | |
| | | | | | | | ACCUCGAUGA | | |
| | | | | | | | CAGUCUGAUA | | |
| | | | | | | | GCAAUGUCAA | | |
| | | | | | | | CCUCGCCUAU | | |
| | | | | | | | GAACUUCAGG | | |
| | | | | | | | GUCAGCUUGC | | |
| | | | | | | | GUGUUAUUCU | | |
| | | | | | | | UGGCAAGCUG | | |
| | | | | | | | ACCCUGAAGU | | |
| | | | | | | | UCAUAGGCGC | | |
| | | | | | | | GGUUGACCAU | | |
| | | | | | | | ACCGUCCACU | | |
| | | | | | | | CCUGAUUUUC | | |
| | | | | | | | GAAUCAGACA | | |
| | | | | | | | AGGACGGUAU | | |
| | | | | | | | GUAAUUGCUA | | |
| | | | | | | | UCAAGAAGUC | | |
| | | | | | | | AUACCAGAGA | | |
| | | | | | | | AACACGACUA | | |
| | | | | | | | CUACCUCA | | |
| 1sided_T | | | | | | 196 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_7RH2 | | | | | | | GAAAGAUCGA | | |
| | | | | | | | CGAGGUGAAA | | |
| | | | | | | | ACCUCGGUAU | | |
| | | | | | | | GACAGUCUGA | | |
| | | | | | | | UAGCAAUGUC | | |
| | | | | | | | AACCUCGCCU | | |
| | | | | | | | AUGAACUUCA | | |
| | | | | | | | GGGUCAGCUU | | |
| | | | | | | | GCGUGUUAUU | | |
| | | | | | | | CUUGGCAAGC | | |
| | | | | | | | UGACCCUGAA | | |
| | | | | | | | GUUCAUAGGC | | |
| | | | | | | | GCGGUUGACC | | |
| | | | | | | | AUACCGUCCA | | |
| | | | | | | | GUCCUGAUUU | | |
| | | | | | | | UCGAAUCAGA | | |
| | | | | | | | GAAGGACGGU | | |
| | | | | | | | AUGUAAUUGC | | |
| | | | | | | | UAUCAAGAAG | | |
| | | | | | | | UCAUACACCA | | |
| | | | | | | | GAGAAACACG | | |
| | | | | | | | ACUACUACCU | | |
| | | | | | | | CA | | |
| 1sided_Tlet7f | | | | | | 197 | GGGAAACUAU | | |
| CshRNA- | | | | | | | ACAAUACGGU | | |
| GFP6_D2_M | | | | | | | AUAUUACCUG | | |
| L_7RH2_left | | | | | | | GUUUUCGAUC | | |
| cleav_site_ | | | | | | | GAAAGAUCGA | | |
| mut_v1 | | | | | | | CGAGGUGAAA | | |
| | | | | | | | ACCUCGGUAU | | |
| | | | | | | | GACAGUCUGA | | |
| | | | | | | | UAGCAAUGUC | | |
| | | | | | | | AACCUCGCCU | | |
| | | | | | | | AUGAACUUCA | | |
| | | | | | | | GGGUCAGCUU | | |
| | | | | | | | GCGUGUUAUU | | |
| | | | | | | | CUUGGCAAGC | | |
| | | | | | | | UGACCCUGAA | | |
| | | | | | | | GUUCAUAGGC | | |
| | | | | | | | GCGGUUGACC | | |
| | | | | | | | AUACCGUCCA | | |
| | | | | | | | CUCCUGAUUU | | |
| | | | | | | | UCGAAUCAGA | | |
| | | | | | | | CAAGGACGGU | | |
| | | | | | | | AUGUAAUUGC | | |
| | | | | | | | UAUCAAGAAG | | |
| | | | | | | | UCAUACACCA | | |
| | | | | | | | GAGAAACACG | | |
| | | | | | | | ACUACUACCU | | |
| | | | | | | | CA | | |
| 1sided_T | | | | | | 199 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_8RH2 | | | | | | | GAAAGAUCGA | | |
| | | | | | | | CGAGGUGAAA | | |
| | | | | | | | ACCUCGCGUA | | |
| | | | | | | | UGACAGUCUG | | |
| | | | | | | | AUAGCAAUGU | | |
| | | | | | | | CAACCUCGCC | | |
| | | | | | | | UAUGAACUUC | | |
| | | | | | | | AGGGUCAGCU | | |
| | | | | | | | UGCGUGUUAU | | |
| | | | | | | | UCUUGGCAAG | | |
| | | | | | | | CUGACCCUGA | | |
| | | | | | | | AGUUCAUAGG | | |
| | | | | | | | CGCGGUUGAC | | |
| | | | | | | | CAUACCGUCC | | |
| | | | | | | | AGUCCUGAUU | | |
| | | | | | | | UUCGAAUCAG | | |
| | | | | | | | AGAAGGACGG | | |
| | | | | | | | UAUGUAAUUG | | |
| | | | | | | | CUAUCAAGAA | | |
| | | | | | | | GUCAUACGAC | | |
| | | | | | | | CAGAGAAACA | | |
| | | | | | | | CGACUACUAC | | |
| | | | | | | | CUCA | | |
| 1sided_Tlet7f | | | | | | 199 | GGGAAACUAU | | |
| CshRNA- | | | | | | | ACAAUACGGU | | |
| GFP6_D2_M | | | | | | | AUAUUACCUG | | |
| L_8RH2_left_ | | | | | | | GUUUUCGAUC | | |
| cleav_site_m | | | | | | | GAAAGAUCGA | | |
| ut_v1 | | | | | | | CGAGGUGAAA | | |
| | | | | | | | ACCUCGCGUA | | |
| | | | | | | | UGACAGUCUG | | |
| | | | | | | | AUAGCAAUGU | | |
| | | | | | | | CAACCUCGCC | | |
| | | | | | | | UAUGAACUUC | | |
| | | | | | | | AGGGUCAGCU | | |
| | | | | | | | UGCGUGUUAU | | |
| | | | | | | | UCUUGGCAAG | | |
| | | | | | | | CUGACCCUGA | | |
| | | | | | | | AGUUCAUAGG | | |
| | | | | | | | CGCGGUUGAC | | |
| | | | | | | | CAUACCGUCC | | |
| | | | | | | | AcUCCUGAUU | | |
| | | | | | | | UUCGAAUCAG | | |
| | | | | | | | AcAAGGACGG | | |
| | | | | | | | UAUGUAAUUG | | |
| | | | | | | | CUAUCAAGAA | | |
| | | | | | | | GUCAUACGAC | | |
| | | | | | | | CAGAGAAACA | | |
| | | | | | | | CGACUACUAC | | |
| | | | | | | | CUCA | | |
| 1sided_T | | | | | | 200 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_9RH2 | | | | | | | GAAAGAUCGA | | |
| | | | | | | | CGAGGUGAAA | | |
| | | | | | | | ACCUCGACGU | | |
| | | | | | | | AUGACAGUCU | | |
| | | | | | | | GAUAGCAAUG | | |
| | | | | | | | UCAACCUCGC | | |
| | | | | | | | CUAUGAACUU | | |
| | | | | | | | CAGGGUCAGC | | |
| | | | | | | | UUGCGUGUUA | | |
| | | | | | | | UUCUUGGCAA | | |
| | | | | | | | GCUGACCCUG | | |
| | | | | | | | AAGUUCAUAG | | |
| | | | | | | | GCGCGGUUGA | | |
| | | | | | | | CCAUACCGUC | | |
| | | | | | | | CAGUCCUGAU | | |
| | | | | | | | UUUCGAAUCA | | |
| | | | | | | | GAGAAGGACG | | |
| | | | | | | | GUAUGUAAUU | | |
| | | | | | | | GCUAUCAAGA | | |
| | | | | | | | AGUCAUACGU | | |
| | | | | | | | ACCAGAGAAA | | |
| | | | | | | | CACGACUACU | | |
| | | | | | | | ACCUCA | | |
| 1sided_Tlet7f | | | | | | 201 | GGGAAACUAU | | |
| CshRNA- | | | | | | | ACAAUACGGU | | |
| GFP6_D2_M | | | | | | | AUAUUACCUG | | |
| L_9RH2_left_ | | | | | | | GUUUUCGAUC | | |
| cleav_site_m | | | | | | | GAAAGAUCGA | | |
| ut_v1 | | | | | | | CGAGGUGAAA | | |
| | | | | | | | ACCUCGACGU | | |
| | | | | | | | AUGACAGUCU | | |
| | | | | | | | GAUAGCAAUG | | |
| | | | | | | | UCAACCUCGC | | |
| | | | | | | | CUAUGAACUU | | |
| | | | | | | | CAGGGUCAGC | | |
| | | | | | | | UUGCGUGUUA | | |
| | | | | | | | UUCUUGGCAA | | |
| | | | | | | | GCUGACCCUG | | |
| | | | | | | | AAGUUCAUAG | | |
| | | | | | | | GCGCGGUUGA | | |
| | | | | | | | CCAUACCGUC | | |
| | | | | | | | CACUCCUGAU | | |
| | | | | | | | UUUCGAAUCA | | |
| | | | | | | | GACAAGGACG | | |
| | | | | | | | GUAUGUAAUU | | |
| | | | | | | | GCUAUCAAGA | | |
| | | | | | | | AGUCAUACGU | | |
| | | | | | | | ACCAGAGAAA | | |
| | | | | | | | CACGACUACU | | |
| | | | | | | | ACCUCA | | |
| 1sided_T | | | | | | 202 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_10RH2 | | | | | | | GAAAGAUCGA | | |
| | | | | | | | CGAGGUGAAA | | |
| | | | | | | | ACCUCGCACG | | |
| | | | | | | | UAUGACAGUC | | |
| | | | | | | | UGAUAGCAAU | | |
| | | | | | | | GUCAACCUCG | | |
| | | | | | | | CCUAUGAACU | | |
| | | | | | | | UCAGGGUCAG | | |
| | | | | | | | CUUGCGUGUU | | |
| | | | | | | | AUUCUUGGCA | | |
| | | | | | | | AGCUGACCCU | | |
| | | | | | | | GAAGUUCAUA | | |
| | | | | | | | GGCGCGGUU | | |
| | | | | | | | GACCAUACCG | | |
| | | | | | | | UCCAGUCCUG | | |
| | | | | | | | AUUUUCGAAU | | |
| | | | | | | | CAGAGAAGGA | | |
| | | | | | | | CGGUAUGUAA | | |
| | | | | | | | UUGCUAUCAA | | |
| | | | | | | | GAAGUCAUAC | | |
| | | | | | | | GUGACCAGAG | | |
| | | | | | | | AAACACGACU | | |
| | | | | | | | ACUACCUCA | | |
| 1sided_Tlet7f | | | | | | 203 | GGGAAACUAU | | |
| CshRNA- | | | | | | | ACAAUACGGU | | |
| GFP6_D2_M | | | | | | | AUAUUACCUG | | |
| L_10RH2_left | | | | | | | GUUUUCGAUC | | |
| _cleav_site | | | | | | | GAAAGAUCGA | | |
| mut_v1 | | | | | | | CGAGGUGAAA | | |
| | | | | | | | ACCUCGCACG | | |
| | | | | | | | UAUGACAGUC | | |
| | | | | | | | UGAUAGCAAU | | |
| | | | | | | | GUCAACCUCG | | |
| | | | | | | | CCUAUGAACU | | |
| | | | | | | | UCAGGGUCAG | | |
| | | | | | | | CUUGCGUGUU | | |
| | | | | | | | AUUCUUGGCA | | |
| | | | | | | | AGCUGACCCU | | |
| | | | | | | | GAAGUUCAUA | | |
| | | | | | | | GGCGCGGUU | | |
| | | | | | | | GACCAUACCG | | |
| | | | | | | | UCCACUCCUG | | |
| | | | | | | | AUUUUCGAAU | | |
| | | | | | | | CAGACAAGGA | | |
| | | | | | | | CGGUAUGUAA | | |
| | | | | | | | UUGCUAUCAA | | |
| | | | | | | | GAAGUCAUAC | | |
| | | | | | | | GUGACCAGAG | | |
| | | | | | | | AAACACGACU | | |
| | | | | | | | ACUACCUCA | | |
| 1sided_T | | | | | | 204 | GGGAAACUAU | | |
| let7f_Csh | | | | | | | ACAAUACGGU | | |
| RNA- | | | | | | | AUAUUACCUG | | |
| GFP6_D | | | | | | | GUUUUCGAUC | | |
| 2_ML_6RH2 | | | | | | | GAAAGAUCGA | | |
| | | | | | | | CGAGGUGAAA | | |
| | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_Tlet7f | | | | | | 205 | GGGAAACUAU | | |
| CshRNA- | | | | | | | ACAAUACGGU | | |
| GFP6_D2_M | | | | | | | AUAUUACCUG | | |
| L_6RH2_left | | | | | | | GUUUUCGAUC | | |
| cleav_site_m | | | | | | | GAAAGAUCGA | | |
| ut_v1 | | | | | | | CGAGGUGAAA | | |
| | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAC | | |
| | | | | | | | UCCUGAUUUU | | |
| | | | | | | | CGAAUCAGAc | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_Tlet7f | | | | | | 206 | GGGAAACUAU | | |
| CshRNA- | | | | | | | ACAAUACGGU | | |
| GFP6_D2_M | | | | | | | AUAUUACCUG | | |
| L_6RH2_righ | | | | | | | GUUUUCGAUC | | |
| t_cleav_site | | | | | | | GAAAGAUCGA | | |
| mut_v1 | | | | | | | CGAGGUGAAA | | |
| | | | | | | | ACCUCGUAUG | | |
| | | | | | | | ACACUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAACAAGU | | |
| | | | | | | | CAUAACCAGA | | |
| | | | | | | | GAAACACGAC | | |
| | | | | | | | UACUACCUCA | | |
| 1sided_Tlet7f | | | | | | 207 | GGGAAACUAU | | |
| CshRNA- | | | | | | | ACAAUACGGU | | |
| GFP6_D2_M | | | | | | | AUAUUACCUG | | |
| L_6RH2_bot | | | | | | | GUUUUCGAUC | | |
| h_cleav_site | | | | | | | GAAAGAUCGA | | |
| mut_v1 | | | | | | | CGAGGUGAAA | | |
| | | | | | | | ACCUCGUAUG | | |
| | | | | | | | ACACUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAc | | |
| | | | | | | | UCCUGAUUUU | | |
| | | | | | | | CGAAUCAGAC | | |
| | | | | | | | AAGGACGGUA | | |
| | | | | | | | UGUAAUUGCU | | |
| | | | | | | | AUCAACAAGU | | |
| | | | | | | | CAUAACCAGA | | |
| | | | | | | | GAAACACGAC | | |
| | | | | | | | UACUACCUCA | | |
| 1sided_T | | | | | | 208 | GGGAAACUAU | | |
| let7f_Csh | | | | | | | ACAAUACGGU | | |
| RNA- | | | | | | | UUAUUACCUG | | |
| GFP6_D | | | | | | | GUUUUCGAUC | | |
| 2_ML_6RH2_ | | | | | | | GAAAGAUCGA | | |
| A38mut | | | | | | | CGAGGUGAAA | | |
| | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_T | | | | | | 209 | GGGAACGUAC | | |
| Egene20_ | | | | | | | CUGUACGGUA | | |
| CshRN | | | | | | | UAUUACCUGG | | |
| A- | | | | | | | UUUUCGAUCG | | |
| GFP6_D | | | | | | | AAAGAUCGAC | | |
| 2_ML_6RH2 | | | | | | | GAGGUGAAAA | | |
| | | | | | | | CCUCGUAUGA | | |
| | | | | | | | CAGUCUGAUA | | |
| | | | | | | | GCAAUGUCAA | | |
| | | | | | | | CCUCGCCUAU | | |
| | | | | | | | GAACUUCAGG | | |
| | | | | | | | GUCAGCUUGC | | |
| | | | | | | | GUGUUAUUCU | | |
| | | | | | | | UGGCAAGCUG | | |
| | | | | | | | ACCCUGAAGU | | |
| | | | | | | | UCAUAGGCGC | | |
| | | | | | | | GGUUGACCAU | | |
| | | | | | | | ACCGUCCAGU | | |
| | | | | | | | CCUGAUUUUC | | |
| | | | | | | | GAAUCAGAGA | | |
| | | | | | | | AGGACGGUAU | | |
| | | | | | | | GUAAUUGCUA | | |
| | | | | | | | UCAAGAAGUC | | |
| | | | | | | | AUAACCAGAG | | |
| | | | | | | | AAACACGACU | | |
| | | | | | | | CUUCCGAA | | |
| 1sided_T | | | | | | 210 | GGGAACGUAC | | |
| Egene20_ | | | | | | | CUGUACGGUU | | |
| CshRN | | | | | | | UAUUACCUGG | | |
| A- | | | | | | | UUUUCGAUCG | | |
| GFP6_D | | | | | | | AAAGAUCGAC | | |
| 2_ML_6RH2_ | | | | | | | GAGGUGAAAA | | |
| A38mut | | | | | | | CCUCGUAUGA | | |
| | | | | | | | CAGUCUGAUA | | |
| | | | | | | | GCAAUGUCAA | | |
| | | | | | | | CCUCGCCUAU | | |
| | | | | | | | GAACUUCAGG | | |
| | | | | | | | GUCAGCUUGC | | |
| | | | | | | | GUGUUAUUCU | | |
| | | | | | | | UGGCAAGCUG | | |
| | | | | | | | ACCCUGAAGU | | |
| | | | | | | | UCAUAGGCGC | | |
| | | | | | | | GGUUGACCAU | | |
| | | | | | | | ACCGUCCAGU | | |
| | | | | | | | CCUGAUUUUC | | |
| | | | | | | | GAAUCAGAGA | | |
| | | | | | | | AGGACGGUAU | | |
| | | | | | | | GUAAUUGCUA | | |
| | | | | | | | UCAAGAAGUC | | |
| | | | | | | | AUAACCAGAG | | |
| | | | | | | | AAACACGACU | | |
| | | | | | | | CUUCCGAA | | |
| 1sided_T | | | | | | 211 | GGGAAACUAU | | |
| let7f_Csh | | | | | | | ACAAUAUGGU | | |
| RNA- | | | | | | | AUAUUACCUG | | |
| GFP6_D | | | | | | | GUUUUCGAUC | | |
| 2_CM26RH2 | | | | | | | GAAAGAUCGA | | |
| | | | | | | | CGAGGUGAAA | | |
| | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_T | | | | | | 212 | GGGAAACUAU | | |
| let7f_Csh | | | | | | | ACAAUAUGGU | | |
| RNA- | | | | | | | UUAUUACCUG | | |
| GFP6_D | | | | | | | GUUUUCGAUC | | |
| 2_CM2_ | | | | | | | GAAAGAUCGA | | |
| 6RH2_A | | | | | | | CGAGGUGAAA | | |
| 38mut | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_Tlet7f | | | | | | 213 | GGGAAACUAU | | |
| CshRNA- | | | | | | | ACAAUAUGGU | | |
| GFP6_D2_C | | | | | | | AUAUUACCUG | | |
| M2_6RH2_lef | | | | | | | GUUUUCGAUC | | |
| t_cleav_site_ | | | | | | | GAAAGAUCGA | | |
| mut_v1 | | | | | | | CGAGGUGAAA | | |
| | | | | | | | ACCUCGUAUG | | |
| | | | | | | | ACAGUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAC | | |
| | | | | | | | UCCUGAUUUU | | |
| | | | | | | | CGAAUCAGAC | | |
| | | | | | | | AAGGACGGUA | | |
| | | | | | | | UGUAAUUGCU | | |
| | | | | | | | AUCAAGAAGU | | |
| | | | | | | | CAUAACCAGA | | |
| | | | | | | | GAAACACGAC | | |
| | | | | | | | UACUACCUCA | | |
| 1sided_Tlet7f | | | | | | 214 | GGGAAACUAU | | |
| CshRNA- | | | | | | | ACAAUAUGGU | | |
| GFP6_D2_C | | | | | | | AUAUUACCUG | | |
| M2_6RH2_ri | | | | | | | GUUUUCGAUC | | |
| ght_cleav_sit | | | | | | | GAAAGAUCGA | | |
| e_mut_v1 | | | | | | | CGAGGUGAAA | | |
| | | | | | | | ACCUCGUAUG | | |
| | | | | | | | ACACUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAG | | |
| | | | | | | | UCCUGAUUUU | | |
| | | | | | | | CGAAUCAGAG | | |
| | | | | | | | AAGGACGGUA | | |
| | | | | | | | UGUAAUUGCU | | |
| | | | | | | | AUCAACAAGU | | |
| | | | | | | | CAUAACCAGA | | |
| | | | | | | | GAAACACGAC | | |
| | | | | | | | UACUACCUCA | | |
| 1sided_Tlet7f | | | | | | 215 | GGGAAACUAU | | |
| CshRNA- | | | | | | | ACAAUAUGGU | | |
| GFP6_D2_C | | | | | | | AUAUUACCUG | | |
| M2_6RH2_b | | | | | | | GUUUUCGAUC | | |
| oth_cleav_sit | | | | | | | GAAAGAUCGA | | |
| e_mut_v1 | | | | | | | CGAGGUGAAA | | |
| | | | | | | | ACCUCGUAUG | | |
| | | | | | | | ACACUCUGAU | | |
| | | | | | | | AGCAAUGUCA | | |
| | | | | | | | ACCUCGCCUA | | |
| | | | | | | | UGAACUUCAG | | |
| | | | | | | | GGUCAGCUUG | | |
| | | | | | | | CGUGUUAUUC | | |
| | | | | | | | UUGGCAAGCU | | |
| | | | | | | | GACCCUGAAG | | |
| | | | | | | | UUCAUAGGCG | | |
| | | | | | | | CGGUUGACCA | | |
| | | | | | | | UACCGUCCAC | | |
| | | | | | | | UCCUGAUUUU | | |
| | | | | | | | CGAAUCAGAc | | |
| | | | | | | | AAGGACGGUA | | |
| | | | | | | | UGUAAUUGCU | | |
| | | | | | | | AUCAAcAAGU | | |
| | | | | | | | CAUAACCAGA | | |
| | | | | | | | GAAACACGAC | | |
| | | | | | | | UACUACCUCA | | |
| 1sided_T | | | | | | 216 | GGGAAACTAT | | |
| let7f_Csh | | | | | | | ACAATATGGTA | | |
| RNA- | | | | | | | TATTACACTGG | | |
| GFP6_D | | | | | | | TTTTCGATCGA | | |
| 2_CM2_ | | | | | | | AAGATCGACG | | |
| 6RH3 | | | | | | | AGGTGAAAAC | | |
| | | | | | | | CTCGTGACAG | | |
| | | | | | | | TCTGATAGCA | | |
| | | | | | | | ATGTCAACCT | | |
| | | | | | | | CGCCTATGAA | | |
| | | | | | | | CTTCAGGGTC | | |
| | | | | | | | AGCTTGCGTG | | |
| | | | | | | | TTATTCTTGGC | | |
| | | | | | | | AAGCTGACCC | | |
| | | | | | | | TGAAGTTCATA | | |
| | | | | | | | GGCGCGGTTG | | |
| | | | | | | | ACCATACCGT | | |
| | | | | | | | CCAGTCCTGA | | |
| | | | | | | | TTTTCGAATCA | | |
| | | | | | | | GAGAAGGACG | | |
| | | | | | | | GTATGTAATTG | | |
| | | | | | | | CTATCAAGAA | | |
| | | | | | | | GTCAACCAGT | | |
| | | | | | | | AGAAACACGA | | |
| | | | | | | | CTACTACCTCA | | |
| 1sided_T | | | | | | 217 | GGGAAACTAT | | |
| let7f_Csh | | | | | | | ACAATATGGTA | | |
| RNA- | | | | | | | TATTACGACTG | | |
| GFP6_D | | | | | | | GTTTTCGATC | | |
| 2_CM27RH3 | | | | | | | GAAAGATCGA | | |
| | | | | | | | CGAGGTGAAA | | |
| | | | | | | | ACCTCGTGAC | | |
| | | | | | | | AGTCTGATAG | | |
| | | | | | | | CAATGTCAAC | | |
| | | | | | | | CTCGCCTATG | | |
| | | | | | | | AACTTCAGGG | | |
| | | | | | | | TCAGCTTGCG | | |
| | | | | | | | TGTTATTCTTG | | |
| | | | | | | | GCAAGCTGAC | | |
| | | | | | | | CCTGAAGTTC | | |
| | | | | | | | ATAGGCGCGG | | |
| | | | | | | | TTGACCATAC | | |
| | | | | | | | CGTCCAGTCC | | |
| | | | | | | | TGATTTTCGAA | | |
| | | | | | | | TCAGAGAAGG | | |
| | | | | | | | ACGGTATGTA | | |
| | | | | | | | ATTGCTATCAA | | |
| | | | | | | | GAAGTCAACC | | |
| | | | | | | | AGTCAGAAAC | | |
| | | | | | | | ACGACTACTA | | |
| | | | | | | | CCTCA | | |
| 1sided_T | | | | | | 218 | GGGAAACTAT | | |
| let7f_Csh | | | | | | | ACAATATGGTA | | |
| RNA- | | | | | | | TATTACTGACT | | |
| GFP6_D | | | | | | | GGTTTTCGAT | | |
| 2_CM28RH3 | | | | | | | CGAAAGATCG | | |
| | | | | | | | ACGAGGTGAA | | |
| | | | | | | | AACCTCGTGA | | |
| | | | | | | | CAGTCTGATA | | |
| | | | | | | | GCAATGTCAA | | |
| | | | | | | | CCTCGCCTAT | | |
| | | | | | | | GAACTTCAGG | | |
| | | | | | | | GTCAGCTTGC | | |
| | | | | | | | GTGTTATTCTT | | |
| | | | | | | | GGCAAGCTGA | | |
| | | | | | | | CCCTGAAGTT | | |
| | | | | | | | CATAGGCGCG | | |
| | | | | | | | GTTGACCATA | | |
| | | | | | | | CCGTCCAGTC | | |
| | | | | | | | CTGATTTTCGA | | |
| | | | | | | | ATCAGAGAAG | | |
| | | | | | | | GACGGTATGT | | |
| | | | | | | | AATTGCTATCA | | |
| | | | | | | | AGAAGTCAAC | | |
| | | | | | | | CAGTCAAGAA | | |
| | | | | | | | ACACGACTAC | | |
| | | | | | | | TACCTCA | | |
| 1sided_T | | | | | | 219 | GGGAAACTAT | | |
| let7f_Csh | | | | | | | ACAATATGGTA | | |
| RNA- | | | | | | | TATTACCTGAC | | |
| GFP6_D | | | | | | | TGGTTTTCGAT | | |
| 2_CM29RH3 | | | | | | | CGAAAGATCG | | |
| | | | | | | | ACGAGGTGAA | | |
| | | | | | | | AACCTCGTGA | | |
| | | | | | | | CAGTCTGATA | | |
| | | | | | | | GCAATGTCAA | | |
| | | | | | | | CCTCGCCTAT | | |
| | | | | | | | GAACTTCAGG | | |
| | | | | | | | GTCAGCTTGC | | |
| | | | | | | | GTGTTATTCTT | | |
| | | | | | | | GGCAAGCTGA | | |
| | | | | | | | CCCTGAAGTT | | |
| | | | | | | | CATAGGCGCG | | |
| | | | | | | | GTTGACCATA | | |
| | | | | | | | CCGTCCAGTC | | |
| | | | | | | | CTGATTTTCGA | | |
| | | | | | | | ATCAGAGAAG | | |
| | | | | | | | GACGGTATGT | | |
| | | | | | | | AATTGCTATCA | | |
| | | | | | | | AGAAGTCAAC | | |
| | | | | | | | CAGTCAGAGA | | |
| | | | | | | | AACACGACTA | | |
| | | | | | | | CTACCTCA | | |
| 1sided_T | | | | | | 220 | GGGAAACTAT | | |
| let7f_Csh | | | | | | | ACAATATGGTA | | |
| RNA- | | | | | | | TATTACACTGA | | |
| GFP6_D | | | | | | | CTGGTTTTCG | | |
| 2_CM210RH3 | | | | | | | ATCGAAAGAT | | |
| | | | | | | | CGACGAGGTG | | |
| | | | | | | | AAAACCTCGT | | |
| | | | | | | | GACAGTCTGA | | |
| | | | | | | | TAGCAATGTC | | |
| | | | | | | | AACCTCGCCT | | |
| | | | | | | | ATGAACTTCA | | |
| | | | | | | | GGGTCAGCTT | | |
| | | | | | | | GCGTGTTATT | | |
| | | | | | | | CTTGGCAAGC | | |
| | | | | | | | TGACCCTGAA | | |
| | | | | | | | GTTCATAGGC | | |
| | | | | | | | GCGGTTGACC | | |
| | | | | | | | ATACCGTCCA | | |
| | | | | | | | GTCCTGATTTT | | |
| | | | | | | | CGAATCAGAG | | |
| | | | | | | | AAGGACGGTA | | |
| | | | | | | | TGTAATTGCTA | | |
| | | | | | | | TCAAGAAGTC | | |
| | | | | | | | AACCAGTCAG | | |
| | | | | | | | TAGAAACACG | | |
| | | | | | | | ACTACTACCTC | | |
| | | | | | | | A | | |
| 1sided_T | | | | | | 459 | GGGAACGTAC | | |
| Egene20_ | | | | | | | CTGTACGGTA | | |
| CsgRN | | | | | | | TATTACCTGGT | | |
| A- | | | | | | | TTTCGATCGAA | | |
| Stoplight | | | | | | | AGATCGACGA | | |
| _modC | | | | | | | GGTGAAAACC | | |
| 137 | | | | | | | TCGTGACAGT | | |
| | | | | | | | CCTTGGACAG | | |
| | | | | | | | TACTCCGCTC | | |
| | | | | | | | GAGTGTTTTA | | |
| | | | | | | | GAGCTAGAAA | | |
| | | | | | | | TAGCAAGTTAA | | |
| | | | | | | | AATAAGGCTA | | |
| | | | | | | | GTCCGTTATC | | |
| | | | | | | | AACTTGAAAAA | | |
| | | | | | | | GTGGCACCGA | | |
| | | | | | | | GTCGGTGCTT | | |
| | | | | | | | TTTTTGTCAGT | | |
| | | | | | | | CCTGATTTTCG | | |
| | | | | | | | AATCAGAGAA | | |
| | | | | | | | GACCCCAACC | | |
| | | | | | | | TATCCACTCG | | |
| | | | | | | | AGCGGAGTAC | | |
| | | | | | | | TGTCCAAGAG | | |
| | | | | | | | AAGTCAACCA | | |
| | | | | | | | GAGAAACACG | | |
| | | | | | | | ACTCTTCCGA | | |
| | | | | | | | A | | |
| 1sided_T | | | | | | 460 | GGGAACGTAC | | |
| Egene20_ | | | | | | | CTGTACGGTA | | |
| CsgRN | | | | | | | TATTACCTGGT | | |
| A- | | | | | | | TTTCGATCGAA | | |
| Stoplight | | | | | | | AGATCGACGA | | |
| modC | | | | | | | GGTGAAAACC | | |
| 143 | | | | | | | TCGCGTATGA | | |
| | | | | | | | CAGTCGGACA | | |
| | | | | | | | GTACTCCGCT | | |
| | | | | | | | CGAGTGTTTTA | | |
| | | | | | | | GAGCTAGAAA | | |
| | | | | | | | TAGCAAGTTAA | | |
| | | | | | | | AATAAGGCTA | | |
| | | | | | | | GTCCGTTATC | | |
| | | | | | | | AACTTGAAAAA | | |
| | | | | | | | GTGGCACCGA | | |
| | | | | | | | GTCGGTGCGT | | |
| | | | | | | | CAGTCCTGAT | | |
| | | | | | | | TTTCGAATCAG | | |
| | | | | | | | AGAAGACCCA | | |
| | | | | | | | TGATAACTTAC | | |
| | | | | | | | TCGAGCGGAG | | |
| | | | | | | | TACTGTCCAG | | |
| | | | | | | | AAGTCATACG | | |
| | | | | | | | ACCAGAGAAA | | |
| | | | | | | | CACGACTCTT | | |
| | | | | | | | CCGAA | | |

Note that a GGGA is introduced by T7 polymerase at the very 5′ end of each ribozyme sequence; in some examples above this is underlined or presented in lowercase.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to the ribozymes may be made without deviating from the scope of the invention. Example embodiments are not necessarily mutually exclusive as some may be combined with one or more embodiments to form new exemplary embodiments. The example embodiments should not be construed as limiting the scope of the disclosure.

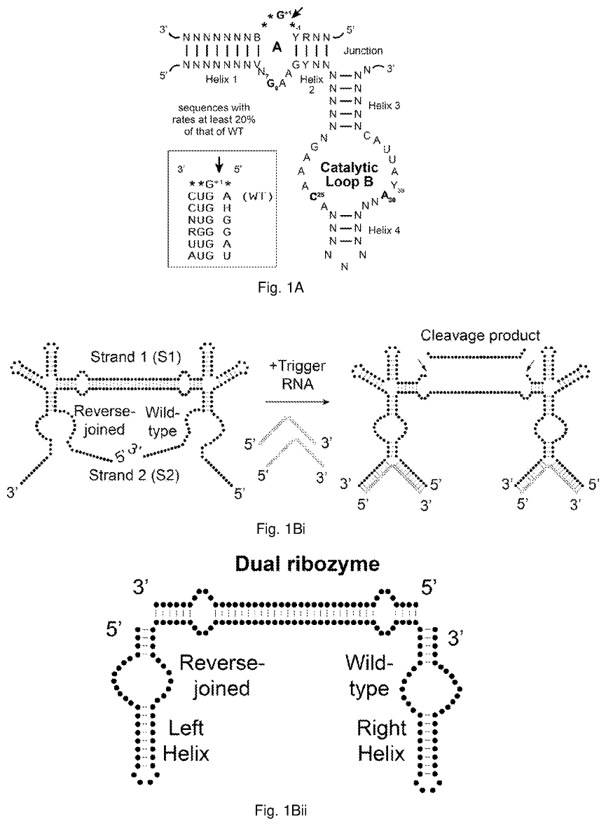

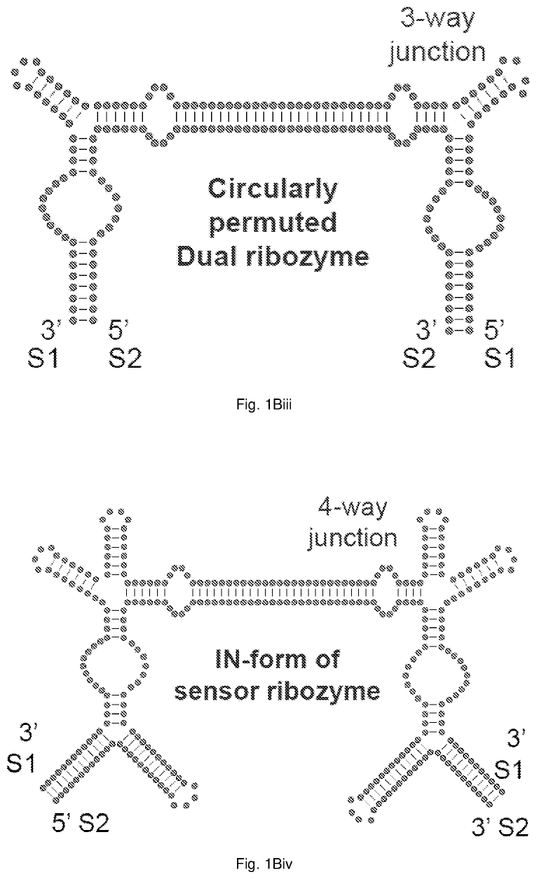

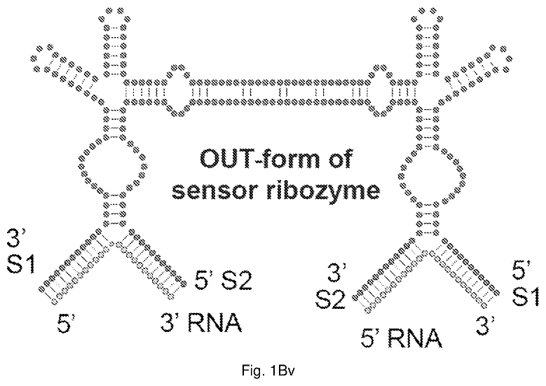

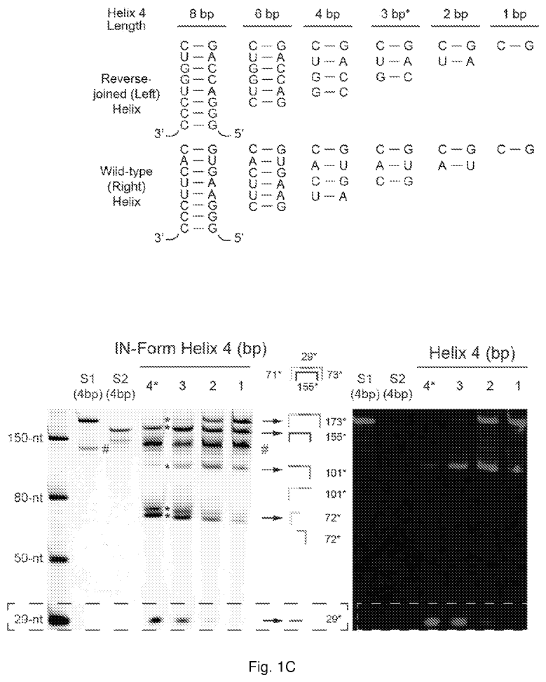

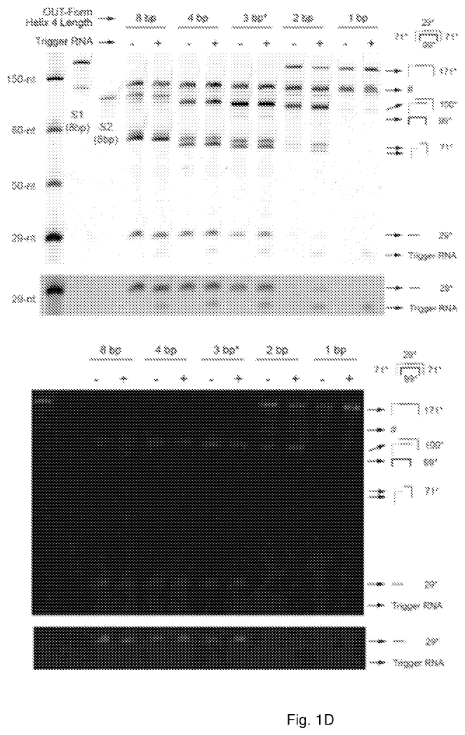

FIG. 1. Development of an RNA trigger-activated dual self-cleaving ribozyme with two trigger-binding catalytic domains.

FIG. 1A. Schematic of a representative hairpin ribozyme, which consists of two Loops A and B, each flanked by two helices. Arrow marks the cleavage site between the N−1 and guanine (G+1) nucleotides in Loop A. Key catalytic nucleotides in catalytic Loop B, A38 and C25, are labelled. 5′-*G**-3′ spans the cleavage site, and the most highly tolerated sequences with cleavage activity of at least 20% of the wildtype ribozyme are shown in the box.

FIG. 1Bi. Design strategy of an RNA trigger-activated self-cleaving dual ribozyme, which releases an embedded RNA product upon trigger-induced cleavage.

FIG. 1Bii. A dual tandem ribozyme.

FIG. 1Biii. Circularly permuted dual ribozyme.

FIG. 1Biv. IN-form of self-cleaving sensor ribozyme, where the RNA-trigger is built into the ribozyme structure.

FIG. 1Bv. OUT-form of the self-cleaving sensor ribozyme, where the trigger RNA is separated from the ribozyme structure.

FIG. 1C. An IN-form of the sensor ribozyme retains self-cleavage activity when Helix 4 is at least 2-3 bp long. Helix 4 configurations tested in FIGS. 1C, D and 9A are shown at top. Asterisks label bands corresponding to predicted cleavage products for the ribozyme with a 4 bp Helix 4. Panel at right shows blot for the same gel probed for the 29-nt cleavage product.

FIG. 1D. Separation of trigger from the ribozyme allows for an RNA trigger-activated dual ribozyme. Asterisks mark bands corresponding to predicted cleavage products for the ribozyme with a 8 bp Helix 4. Panel at bottom shows blot for the same gel probed for the 29-nt cleavage product. Hashtags indicate non-specific products produced in original in vitro transcription that are probe-negative.

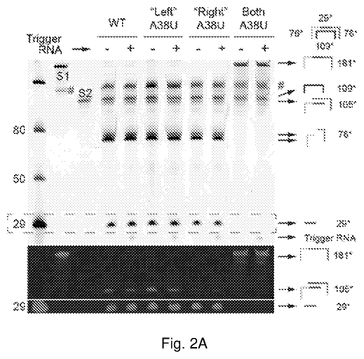

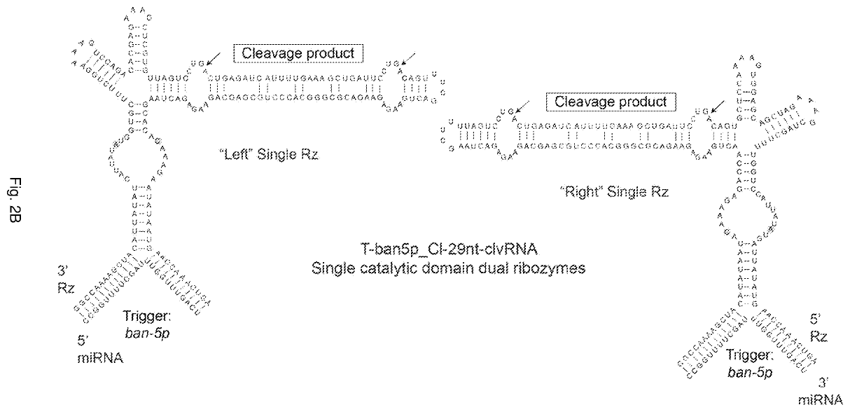

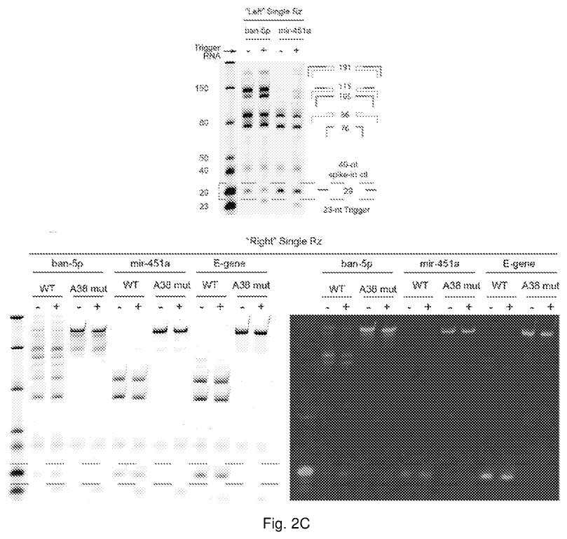

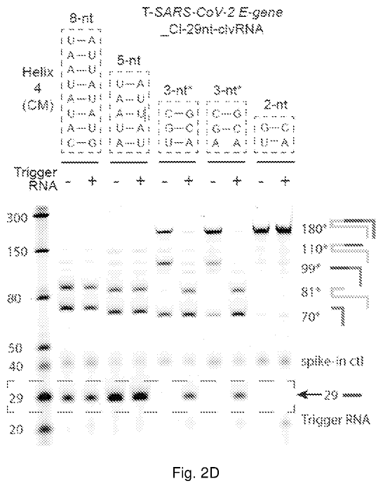

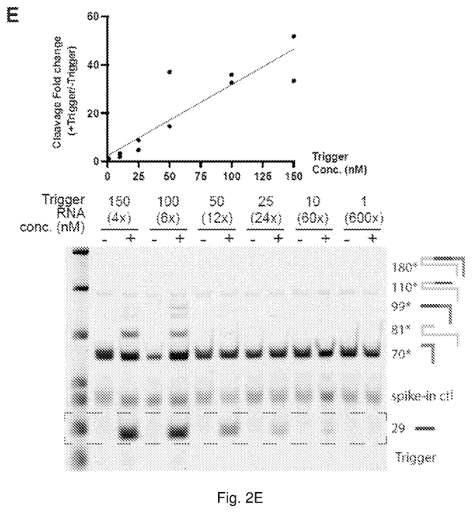

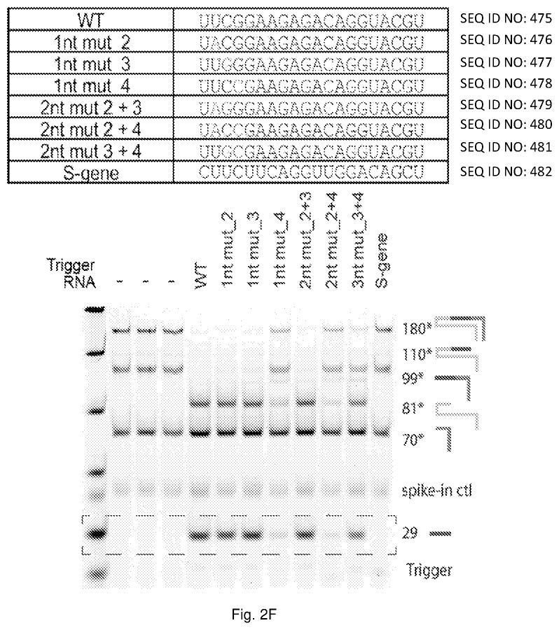

FIG. 2A. Ribozymes with a single catalytic domain exhibit RNA-triggered dual self-cleavage. T-ban5p_Cl-29nt-clvRNA RNA-triggered dual ribozyme with mutations at A38 of either vs both catalytic domains demonstrate that at least one catalytic domain is necessary and sufficient for dual cleavage.

FIG. 2B. Sequence and structure of exemplary dual cleavage site T-ban5p_Cl-29nt-clvRNA ribozymes with either a single “right” wildtype catalytic domain or a single “left” reverse-joined catalytic domain.

FIG. 2C. Single ribozymes with dual cleavage sites and either a single “right” wildtype catalytic domain or a single “left” reverse-joined catalytic domain (structures similar to FIG. 2B) with sensor regions that are triggered by dme-ban-5p, hsa-mir-451 or SARS-CoV-2 E-gene fragment can cleave at two cleavage sites to release an embedded 29-nt RNA cleavage product.

FIG. 2D. Optimisation of the Helix 4 communication module in the T-SARS-CoV-2-E-gene_Cl-29nt-clvRNA ribozyme and identification of 3-nt motifs that improve the signal-to-noise ratio of the ribozyme (Additional motifs have also been identified).

FIG. 2E. Cleavage product release from the T-SARS-CoV-2-E-gene_Cl-29nt-clvRNA ribozyme increases with increasing concentration of E-gene trigger RNA. 600 nM ribozyme was used. Asterisks mark bands corresponding to predicted cleavage products.

FIG. 2F. Testing sequence variants of the E-gene test RNA against the T-SARS-CoV-2-E-gene_Cl-29nt-clvRNA ribozyme shows that the ribozymes can distinguish between closely related trigger RNAs with 1-3 nt differences, while unrelated sequences do not trigger the ribozyme.

FIG. 2G. Ribozyme can detect its trigger from within a complex mixture of RNA, up to at least 1000 fold more non-specific RNA than trigger RNA.

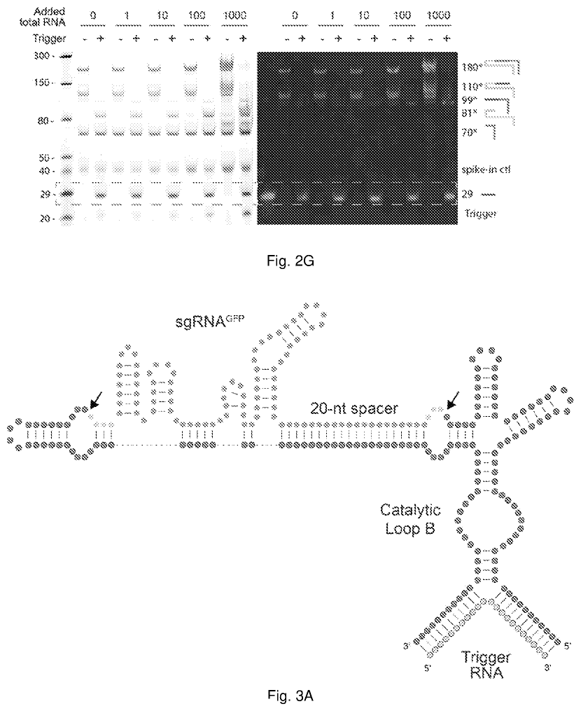

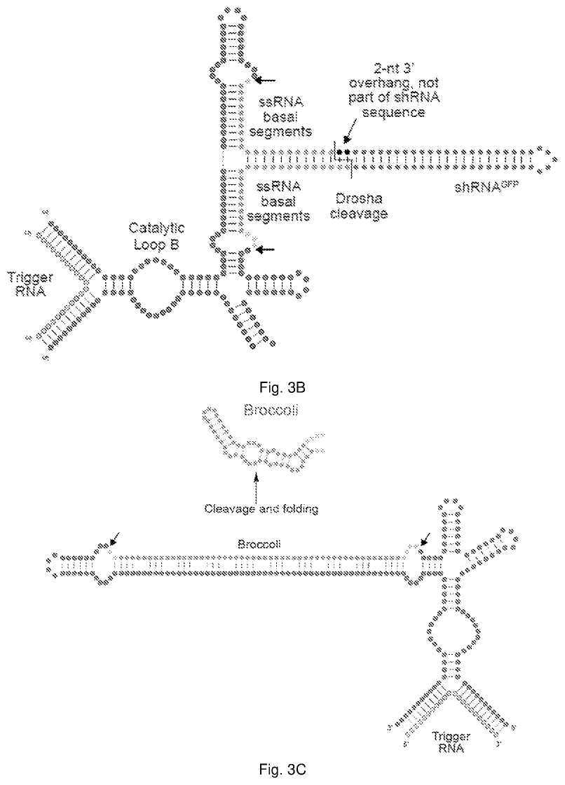

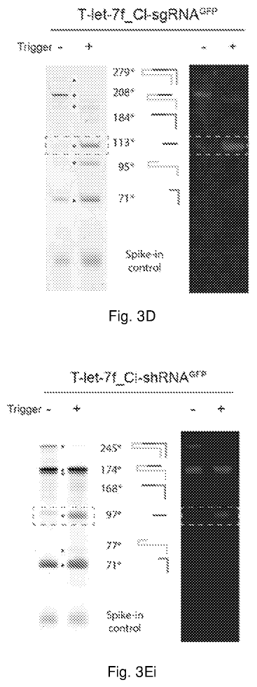

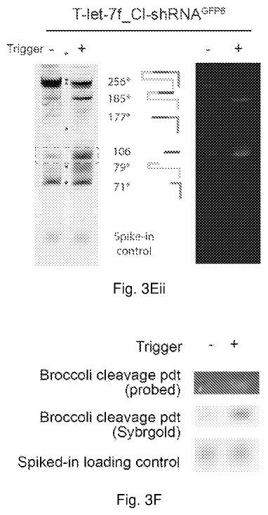

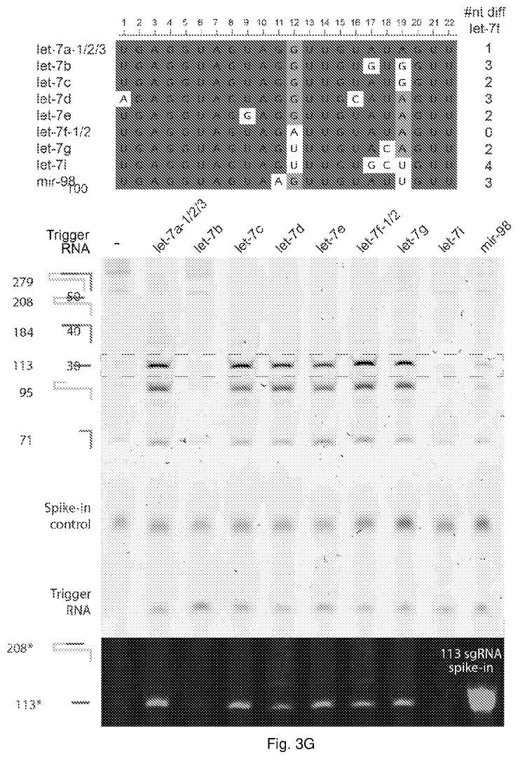

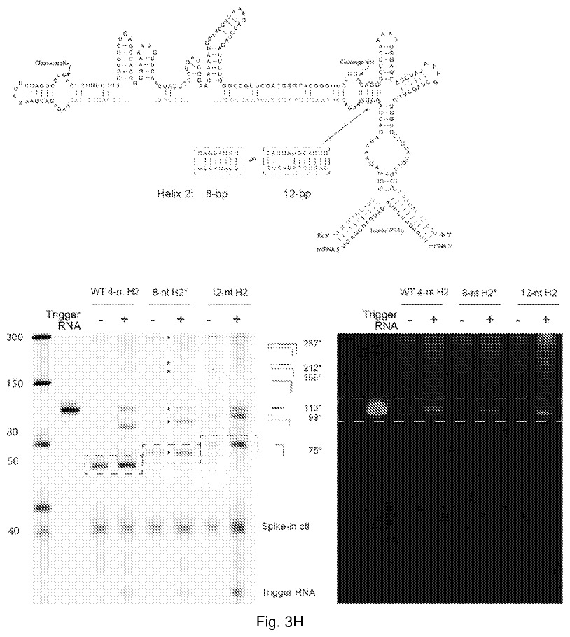

FIG. 3A. Functional RNA can be embedded as cleavage products in ribozymes. Schematic of a ribozyme that comprises an embedded single guide RNA (sgRNA).

FIG. 3B. Schematic of a ribozyme that comprises an embedded short hairpin RNA (shRNA).

FIG. 3C. Schematic of a ribozyme that comprises an embedded RNA aptamer, Broccoli.

FIG. 3D. Cleavage assay for ribozyme T-let-7f_C1-sgRNAGFP. Asterisks mark bands corresponding to predicted cleavage products. Right panel shows blot for the same gel probed for the sgRNA cleavage product.

FIG. 3Ei. Cleavage assays for ribozyme T-let-7f_Cl-shRNAGFP and 3Eii. Ribozyme T-let-7f_C1-shRNAGFP6. Asterisks mark bands corresponding to predicted cleavage products. Right panels show blots for the same gel probed for the shRNA cleavage product.

FIG. 3F. Cleavage assay for ribozyme T-let-7f_Cl-Broccoli aptamer. Top panel shows blot for the same gel probed for the aptamer cleavage product.

FIG. 3G. Members of the human let-7 microRNA family (Top) and cleavage assay for ribozyme T-let-7f_C1-sgRNAGFP when triggered by each member of the let-7 family (Bottom). Bottom-most panel shows blot for the same gel probed for the sgRNA cleavage product. Asterisks mark bands corresponding to predicted cleavage products.

FIG. 3H. Lengthening of Helix 2 on T-let-7f_C1-sgRNAGFP from 4 bp to 8- or 12 bp reduced trigger-independent cleavage at the proximal cleavage site (dashed boxes). Asterisks mark bands corresponding to predicted cleavage products for the ribozyme with a 8-nt Helix 2. Right panel shows blot for the same gel probed for the sgRNA cleavage product.

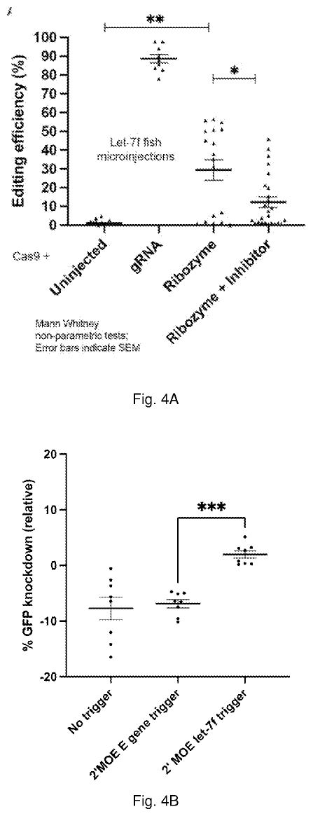

FIG. 4A. Rate of editing in uninjected, positive control gRNA+Cas9-injected, ribozyme (triggered by let-7f to release a gRNA against GFP)+Cas9-injected, and ribozyme+Cas9+let-7f morpholino-injected zebrafish embryos. Mann Whitney non-parametric test was used.

FIG. 4B. Ribozyme (1sided_Tlet7f_CshRNA-GFP6 modified with 50% 2′-fluorinated C and U) detects modified let-7f trigger to increase down-regulation of GFP expression.

FIG. 4C. Ribozyme (1sided_TEgene20_CsgRNA-Stoplight_modC modified with 50% 2′-fluorinated C) detects modified Egene trigger to increase editing of GFP locus.

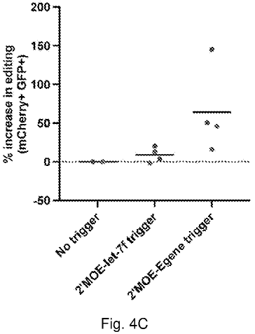

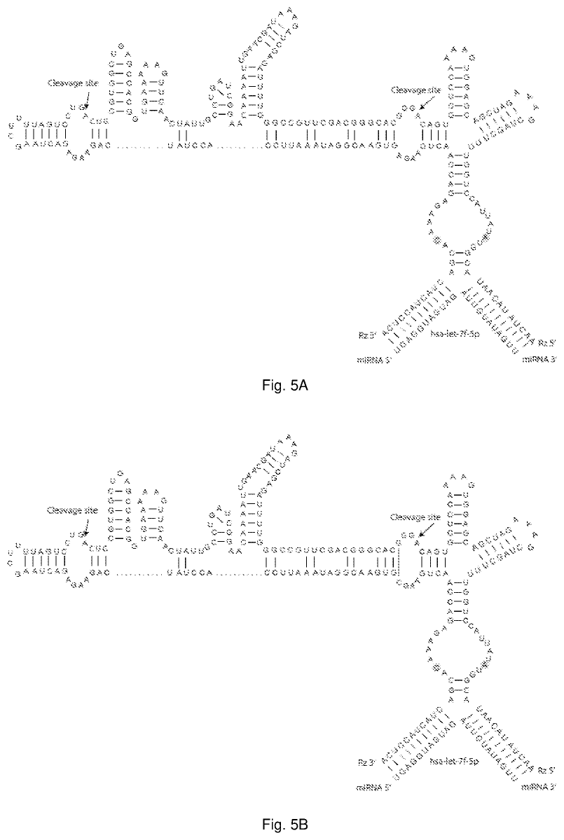

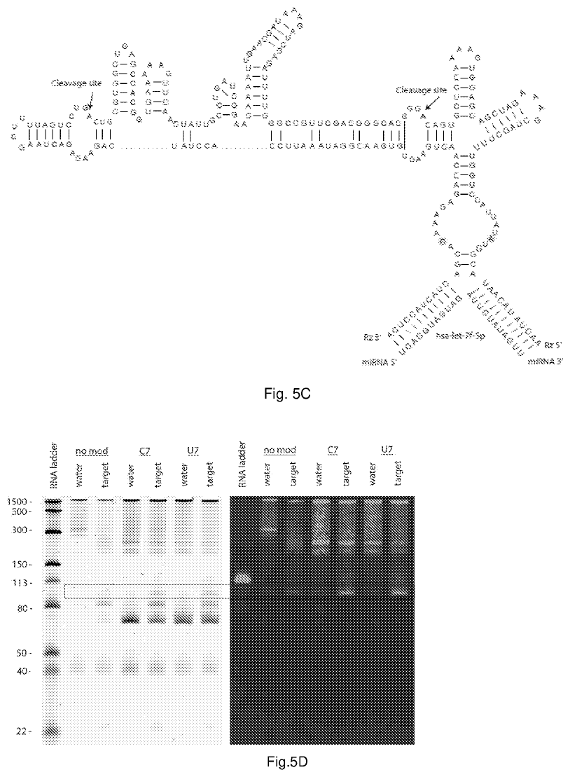

FIG. 5A. Original ribozyme without modifying A7 at the lower strand of the proximal cleavage loop.

FIG. 5B. Mutation of A7 to C7, to be able to pair with opposite strand (bottom C at the lower strand of the proximal cleavage loop).

FIG. 5C: Mutation of A7 to U7, to be able to pair with opposite strand (bottom U at the lower strand of the proximal cleavage loop).

FIG. 5D: Mutation of N7 to pair with N+3 improves cleavage activity of ribozymes with a non-canonical sequence in the cleavage site (cleavage product in outlined box).

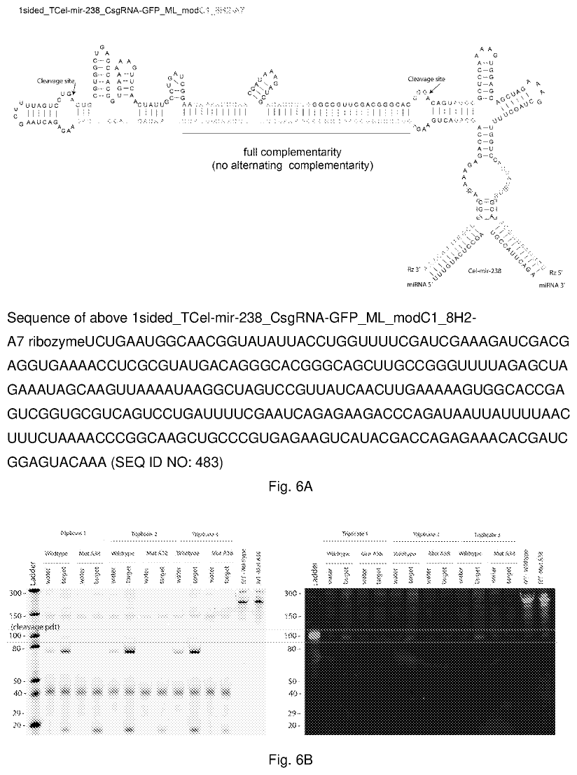

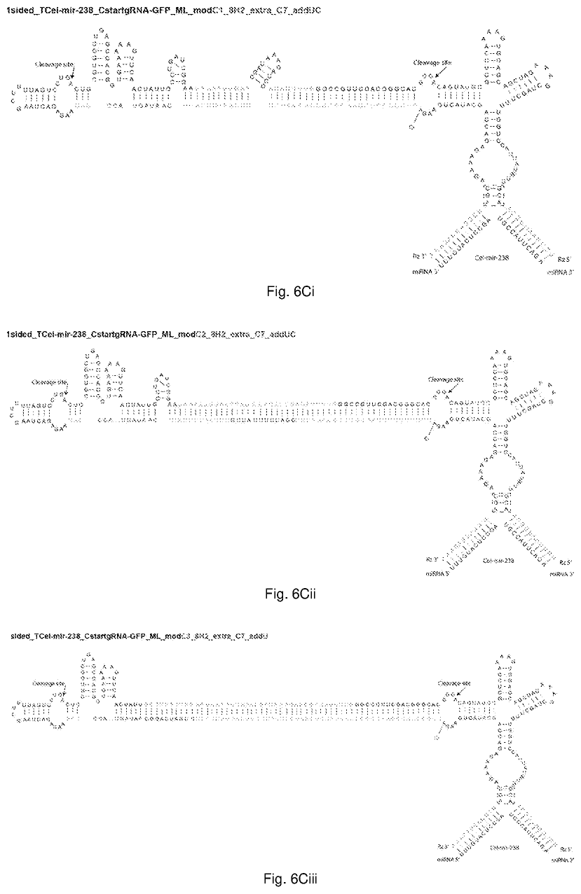

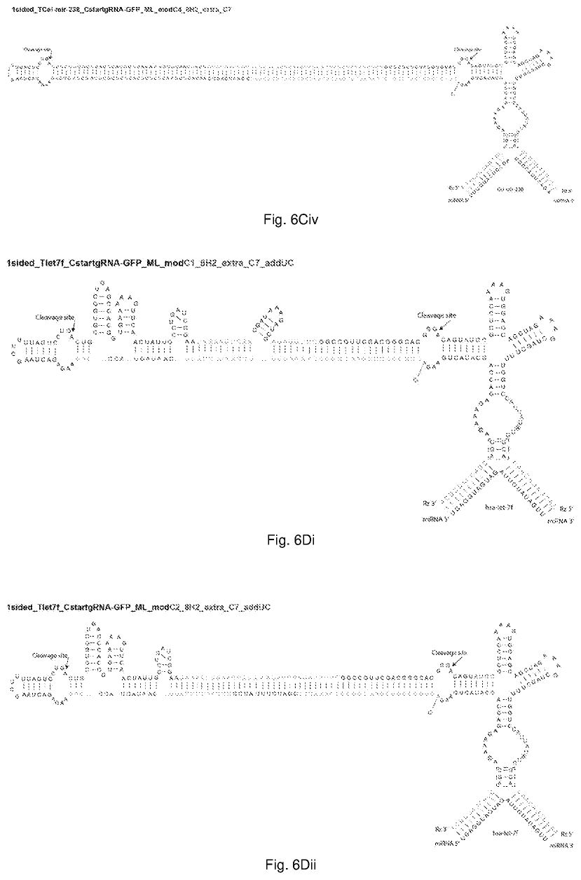

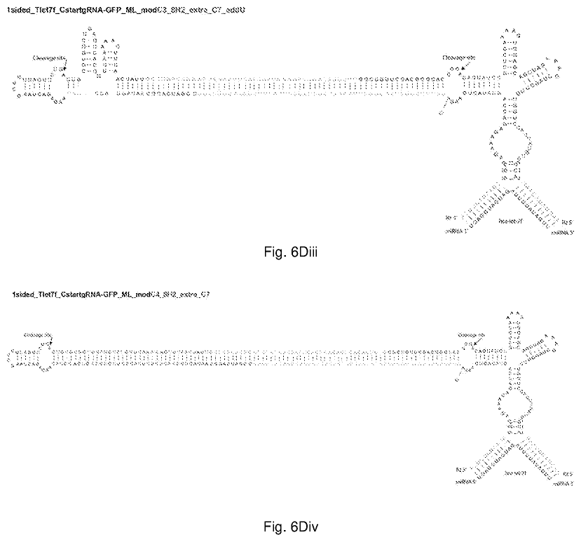

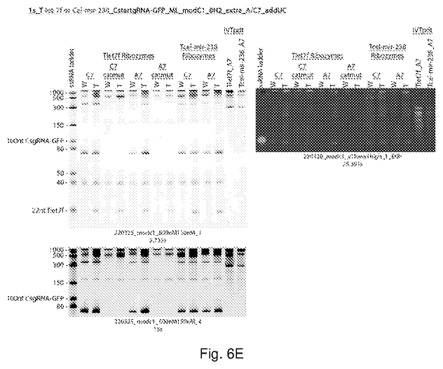

FIG. 6A. 1sided_TCel-mir-238_CsgRNA-GFP_ML_modC1_8H2-A7 ribozyme with full complementary across most of the cleavage product (not alternating complementarity).

FIG. 6B: Cleavage assay for 1sided_TCel-mir-238_CsgRNA-GFP_ML_modC1_8H2-A7 ribozyme.

FIG. 6Ci-iv. 1sided_TCel-mir-238_CsgRNA-GFP_ML_modC1 (i)/or C2 (ii)/or C3 (iii)/or C4 (iv)_8H2-A7 ribozymes with full complementarity across most of the cleavage product (not alternating complementarity).

FIG. 6Di-iv. 1sided_let7f_CsgRNA-GFP_ML_modC1 (i)/or C2 (ii)/or C3 (iii)/or C4 (iv)_8H2-A7 ribozymes with full complementarity across most of the cleavage product (not alternating complementarity).

FIG. 6E: Cleavage assay for 1sided_Tlet-7f or Cel-mir-238_CsgRNA-GFP_ML_modC1_8H2-A7 ribozymes.

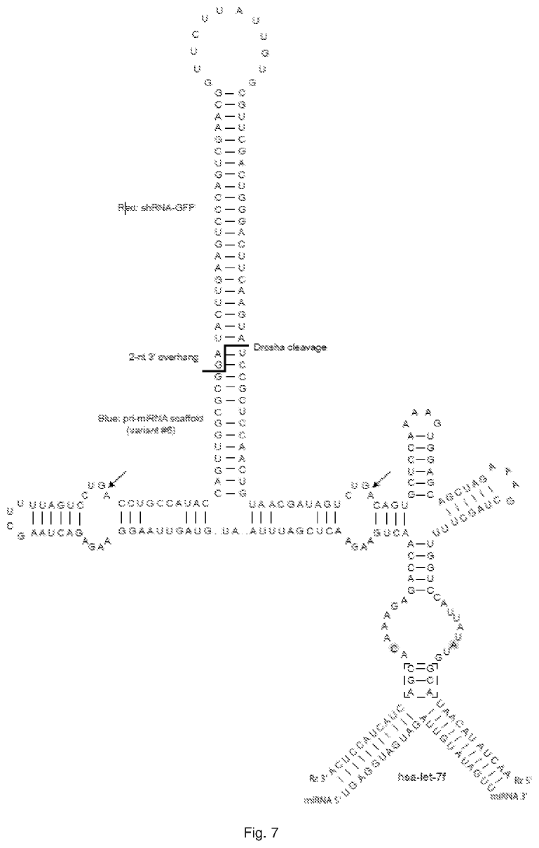

FIG. 7: shRNA ribozymes work in human cells. Structure of 1sided_Tlet7f shRNA-GFP6 ribozyme.

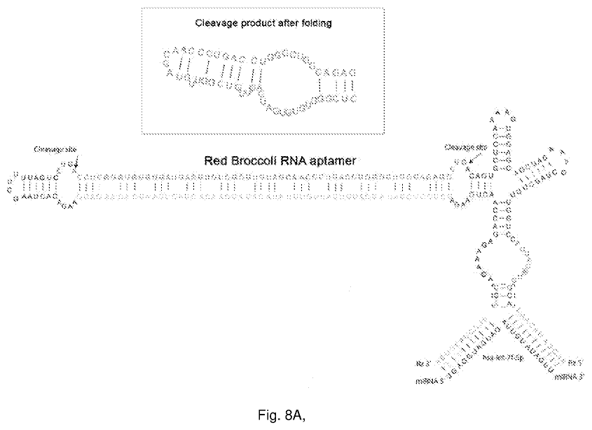

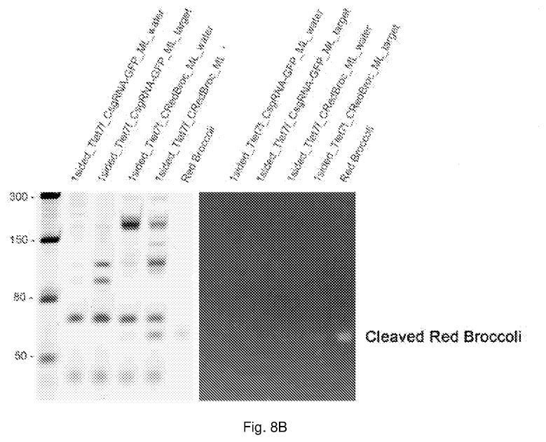

FIG. 8A. Ribozymes can cleave out RNA aptamers. Structure and sequence of ribozyme that cleaves out Red Broccoli fluorescent RNA aptamer.

FIG. 8B. Ribozymes can cleave out RNA aptamers. Trigger-induced cleavage and release of Red Broccoli fluorescent RNA aptamer.

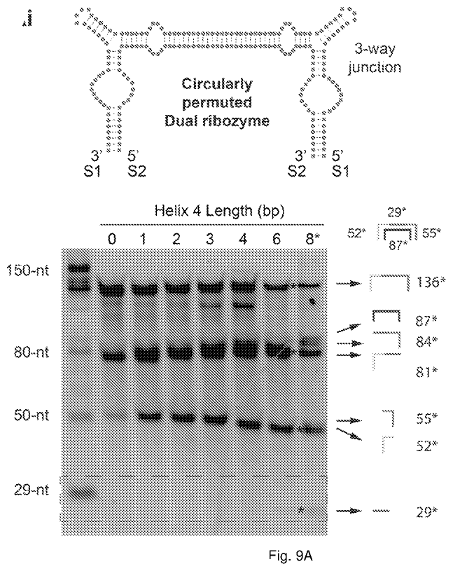

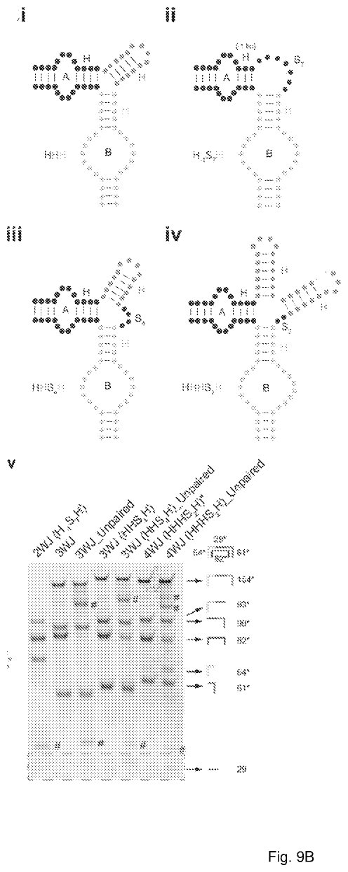

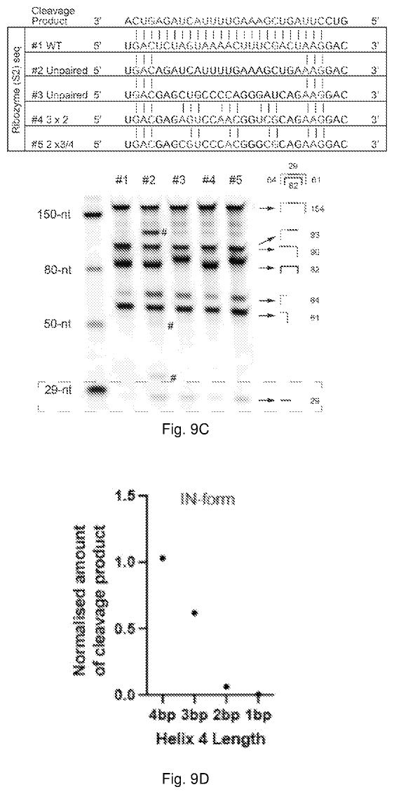

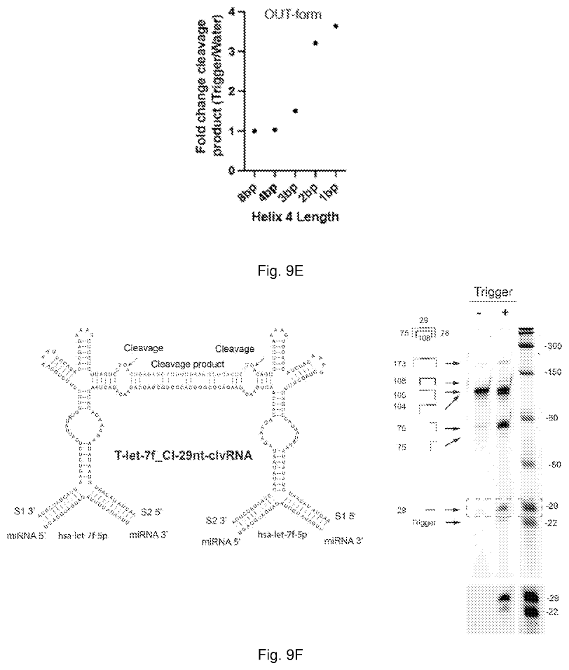

FIG. 9A. A circularly permuted ribozyme with an 8-nt Helix 4 retains self-cleavage activity, while shortening of Helix 4 gradually reduces self-cleavage. Asterisks mark bands corresponding to predicted cleavage products for the 8-bp H2 ribozyme.

FIG. 9B. Schematic of optimal configurations of the hairpin ribozyme junction (i-iv) tested (v). Asterisks mark bands corresponding to predicted cleavage products for the 4WJ paired ribozyme. Hashtags mark unpredicted cleavage products that mostly appear in ribozymes with strong non-complementarity between cleavage product and ribozyme (unpaired).

FIG. 9C. Pairing configurations of Helix 1 tested in 4WJ (HHHS2H) 8-nt Helix 4 ribozymes. Top: Cleavage product sequence in 3′ to 5′ direction. Bottom: Five configurations of pairing on the ribozyme with the cleavage product were tested; sequences are in 5′ to 3′ direction and vertical lines indicate complementary pairing with the cleavage product at top.

FIG. 9D. Graph showing amount (normalised to the S2 strand) of cleavage product released by the IN-Form of the ribozyme when length of Helix 4 is varied from 4 bp to 1 bp (Refers to FIG. 1D).

FIG. 9E. Graph showing fold change in cleavage product released (Trigger Lane/Water Lane) by the OUT-Form of the ribozyme when length of Helix 4 is varied from 8 bp to 1 bp (Refers to FIG. 1E).

FIG. 9F. T-let-7f_Cl-29nt-clvRNA dual ribozyme exhibits let-7f-induced cleavage release of the embedded 29-nt cleavage product.

FIG. 10A. Ribozymes with a single catalytic domain and triggered by dme-mir-184, dme-mir-252, dme-mir-263a, has-let-7f or SARS-CoV-2 Orf1ab gene RNA fragments can self-cleave at dual sites.

FIG. 10B. Mutation of either or both catalytic domains in the ban-5p, mir-451a- or E-gene-triggered dual ribozyme shows that one catalytic domain is sufficient for dual cleavage.

FIG. 10C. Optimisation of the Helix 4 communication module in the T-mir-451a_C1-29nt-clvRNA and T-SARS-CoV-2-S-gene_Cl-29nt-clvRNA ribozyme and identification of a 3-nt v1 and v2 motifs that improve the signal-to-noise ratio of the ribozyme.

FIG. 10D. Testing mutant variants of the E-gene test RNA against the T-SARS-CoV-2-E-gene_Cl-29nt-clvRNA ribozyme shows that the ribozymes can distinguish between closely related trigger RNAs with 1-3 nt differences. Bottom panel shows quantification of the ratio of the intensity of the variant over WT band, each normalised to its 40-nt spike-in control.

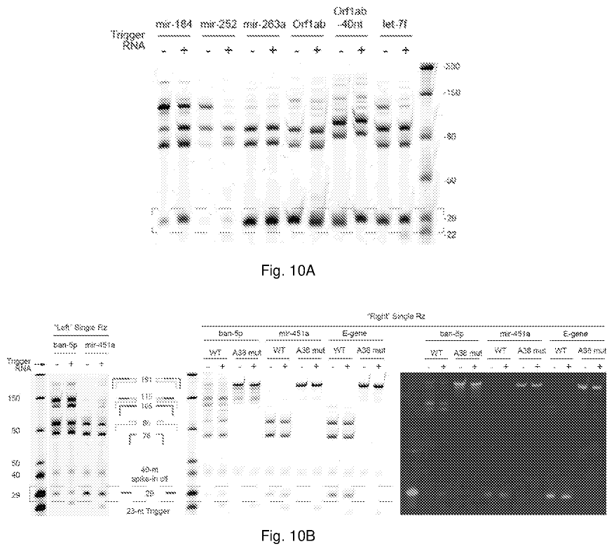

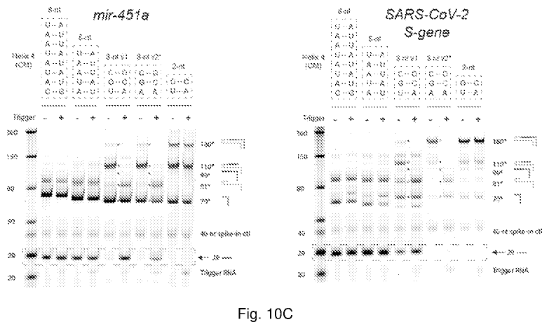

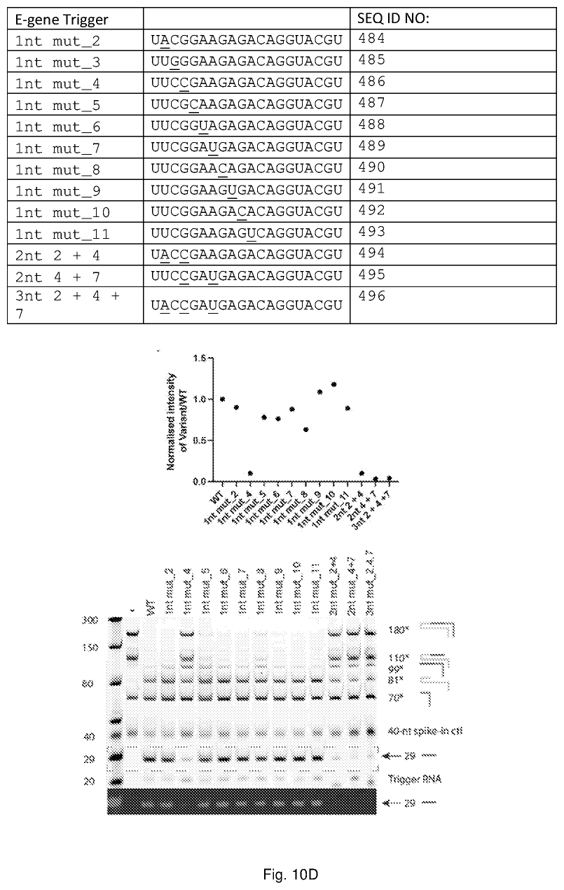

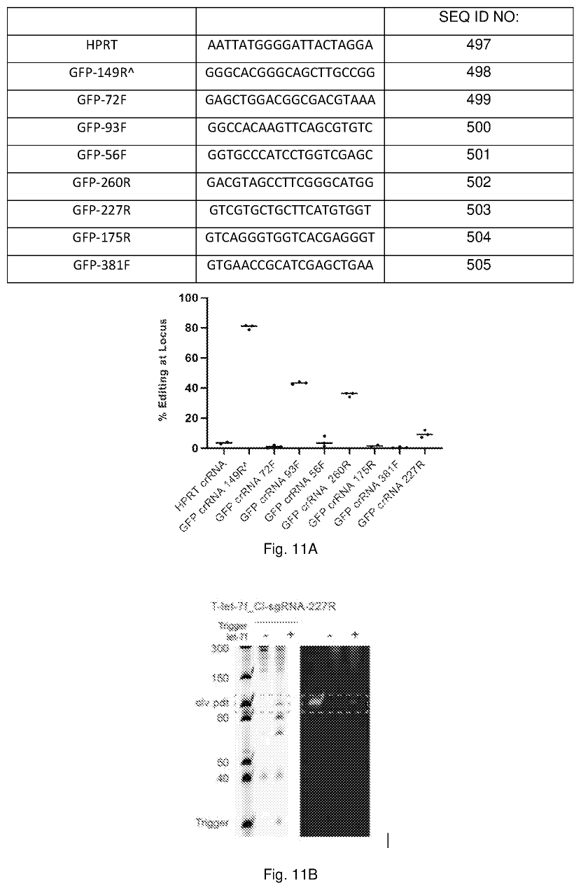

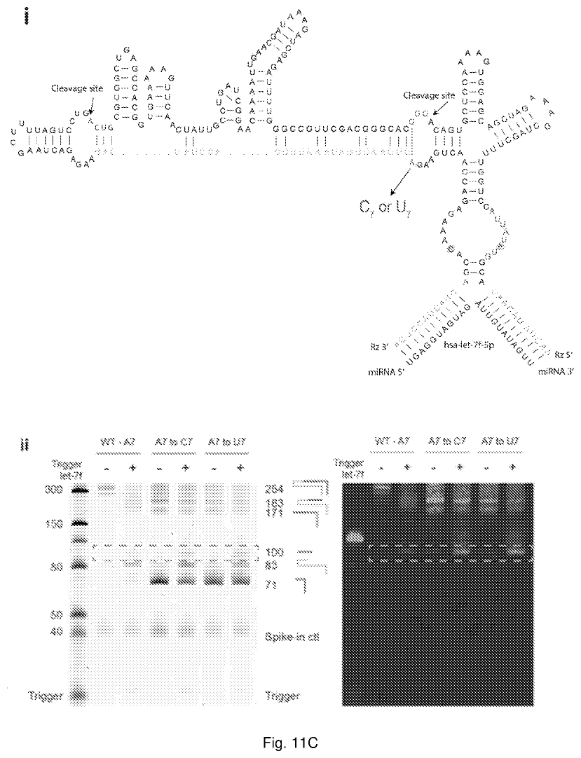

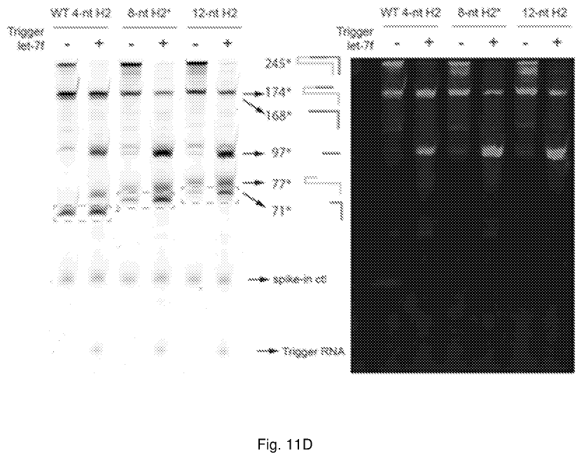

FIG. 11A. Editing efficiency for a range of GFP single guide RNAs tested. \hat{ }indicates the sgRNA selected for encoding within the ribozyme for zebrafish studies.

FIG. 11B. An sgRNA starting with GUC can be cleaved out from a ribozyme in a trigger-dependent maner (227R in previous panel, FIG. 11A).

FIG. 11Ci. Structure of ribozyme where A7 has been changed to C7 or U7 Cii. Mutation of A7 to C7 or U7 restores cleavage to ribozyme comprising the GFP-149R sgRNA, which begins with GGGC. ii) Cleavage assay for ribozyme in Ci. Right panel shows blot for the same gel probed for the sgRNA cleavage product.

FIG. 11D. Lengthening of Helix 2 on T-let-7f_C1-shRNAGFP from 4 bp to 8- or 12 bp reduced trigger-independent cleavage at the proximal cleavage site (dashed boxes). Asterisks mark bands corresponding to predicted cleavage products for the ribozyme with the 8-nt Helix 2. Right panel shows blot for the same gel probed for the shRNA cleavage product.

FIG. 12A. Various Helix 4 motifs tested.

FIG. 12B. Cleavage assay showing that (CM2: 5′-AUG/CGA-3′), (CM7: 5′-ACU/AGA-3′) and (CM8: 5′-AUG/CAA-3′) are additional Helix 4 motifs that perform well.

FIG. 13A. Sequence of shRNA-embedded ribozyme with original right Helix 2 and additional variations in Helix 2 tested.

FIG. 13B. Cleavage assay of ribozymes with varied Helix 2 as shown in FIG. 13A. Thus, FIG. 13B shows extension of Helix 2 beyond 5-nt decreases background cleavage (lower asterisk).

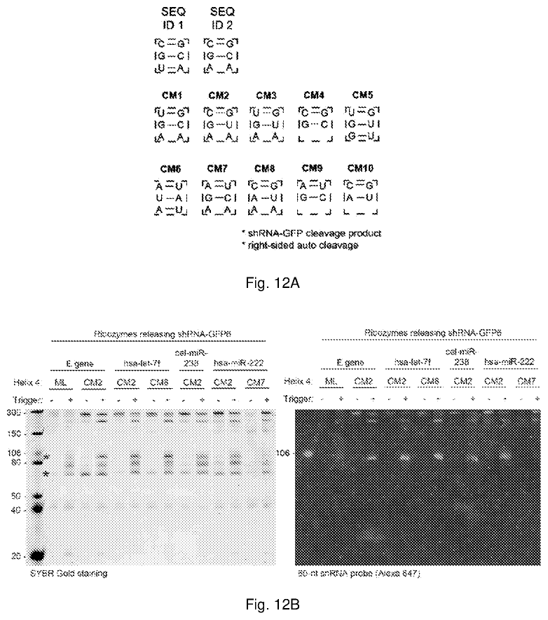

FIG. 14A. Sequence of shRNA-embedded ribozyme with original pairing.

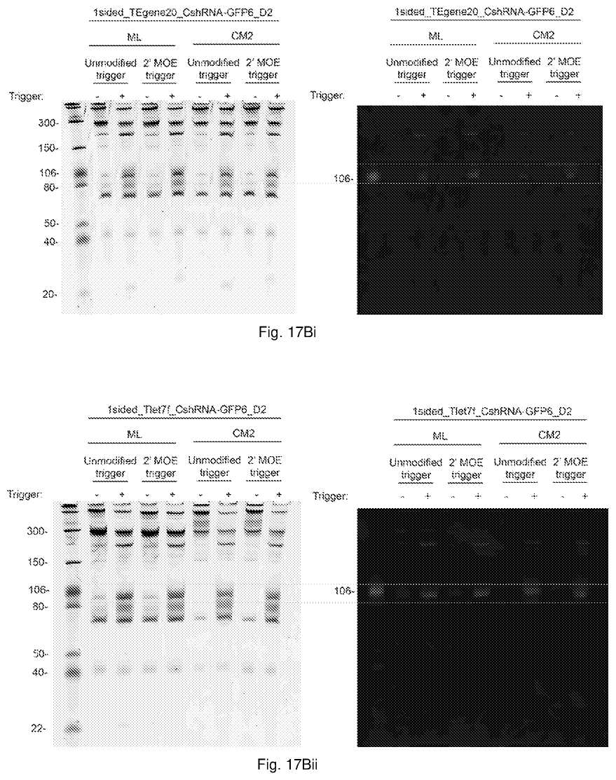

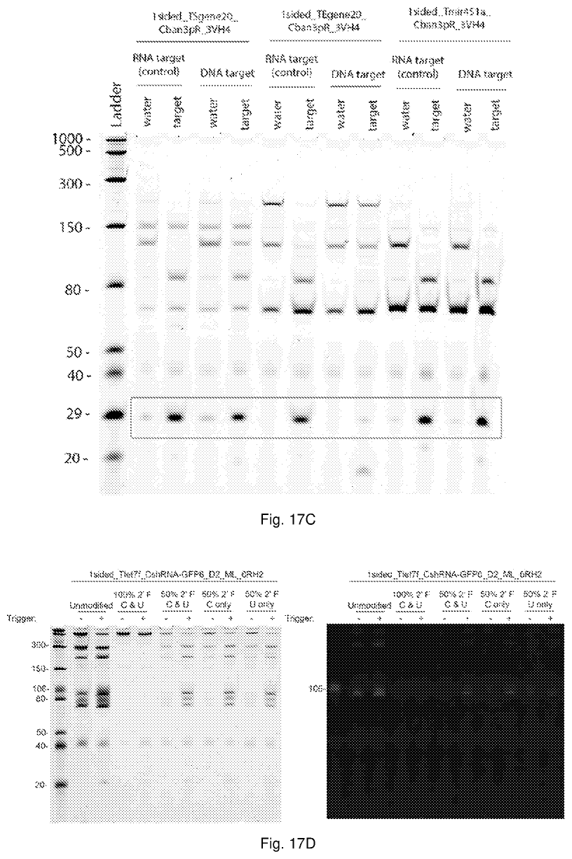

FIG. 14B. Top: Variations in pairing of shRNA ribozyme from FIG. 14A tested. Bottom: Flow cytometry results of shRNA knockdown of GFP fluorescence by ribozymes with varying degree of pairing between embedded shRNA and ribozyme. Thus, FIG. 19B shows that an increase in complementarity between the shRNA cleavage product and ribozyme increases the cleavage dependency of embedded shRNA function.

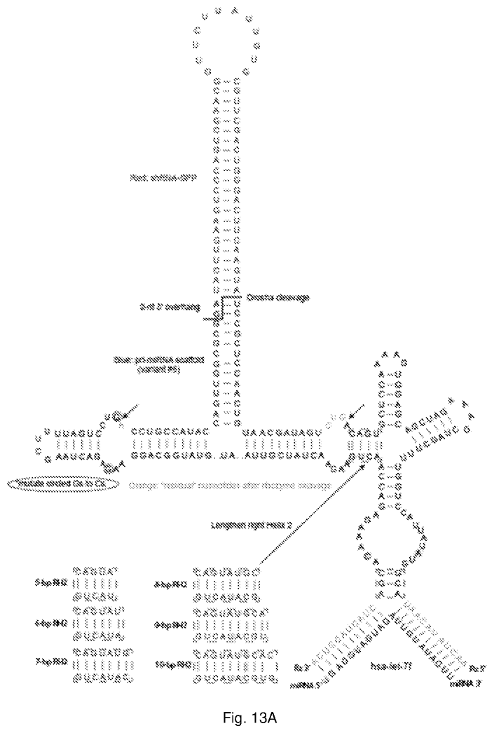

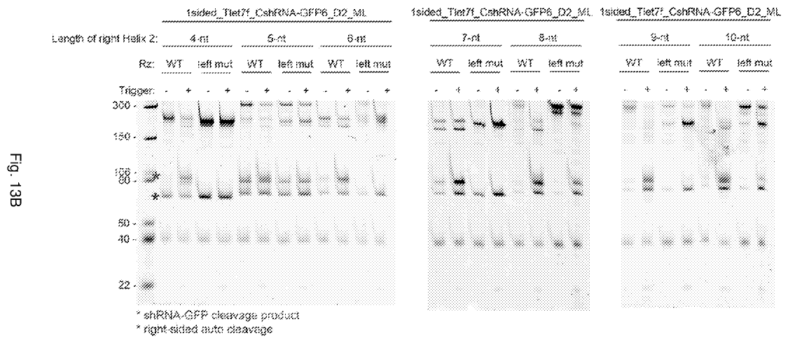

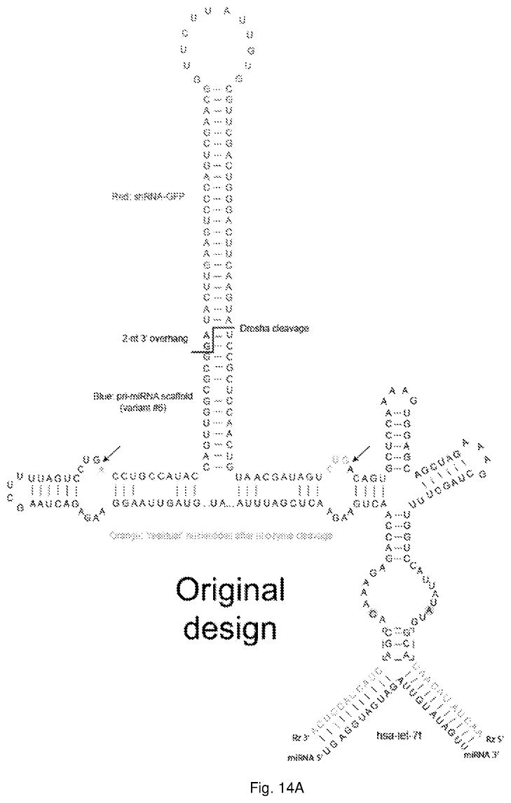

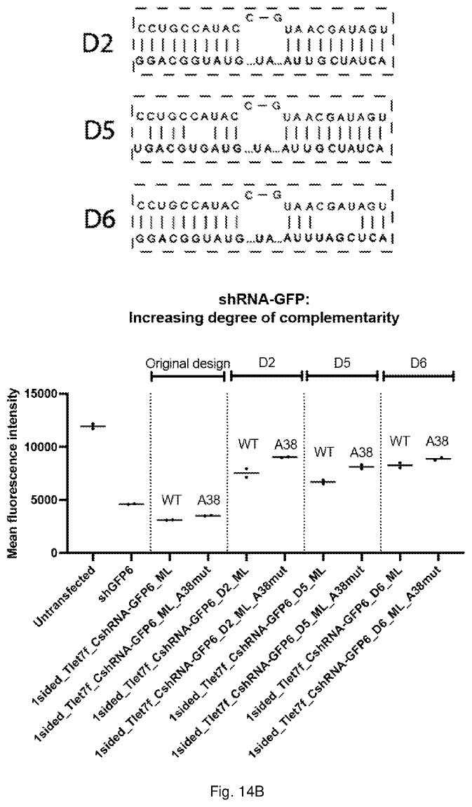

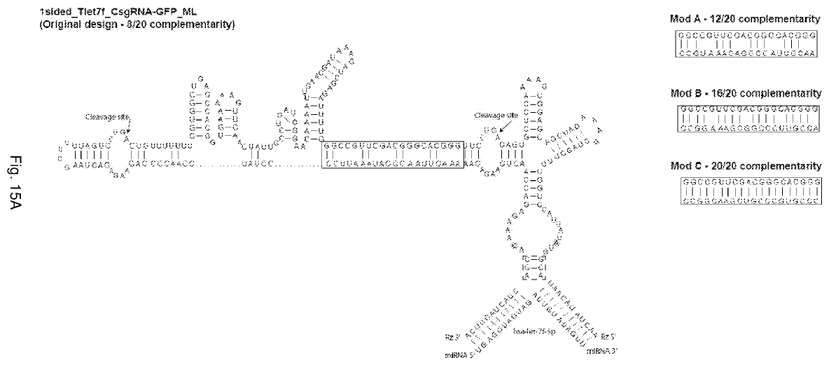

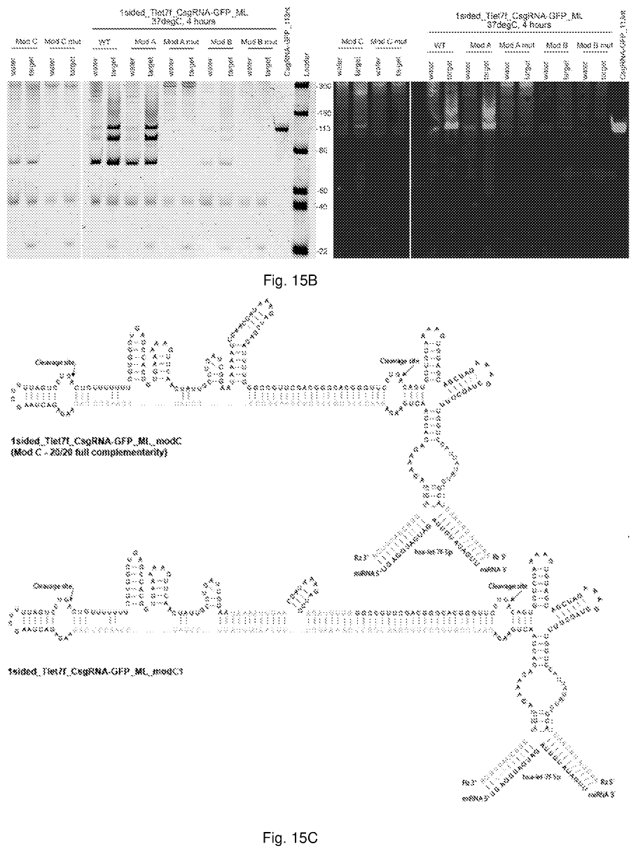

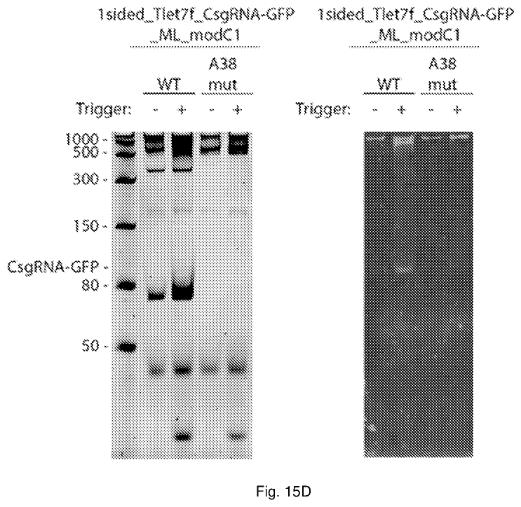

FIG. 15A. Design of original sgRNA-embedded ribozymes with partial complementarity between sgRNA spacer and ribozyme backbone, and changes in spacer complementarity that were tested (mods A, B and C in boxes at right).

FIG. 15B. Cleavage assays of modA, modB and modC sgRNA-embedded ribozymes from (A).

FIG. 15C. Design of sgRNA-embedded ribozymes where part of the first stem loop of the sgRNA is “flattened” to pair with the ribozyme backbone, resulting in increased complementarity between sgRNA and ribozyme backbone (also known as versions modC and modC1).

FIG. 15D. Cleavage assays of modC1 sgRNA-embedded ribozymes from (C).

FIG. 16. A. Structures of ribozymes with lengthened right Helix 3 (RH3). B. Cleavage assay results of ribozymes with lengthened right Helix 3 (RH3).

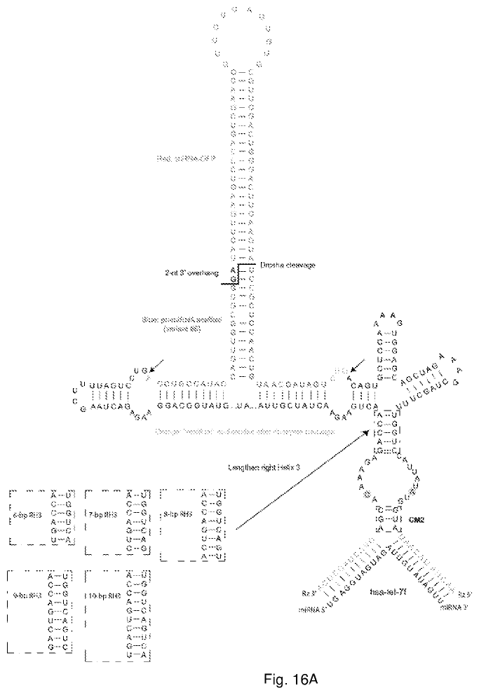

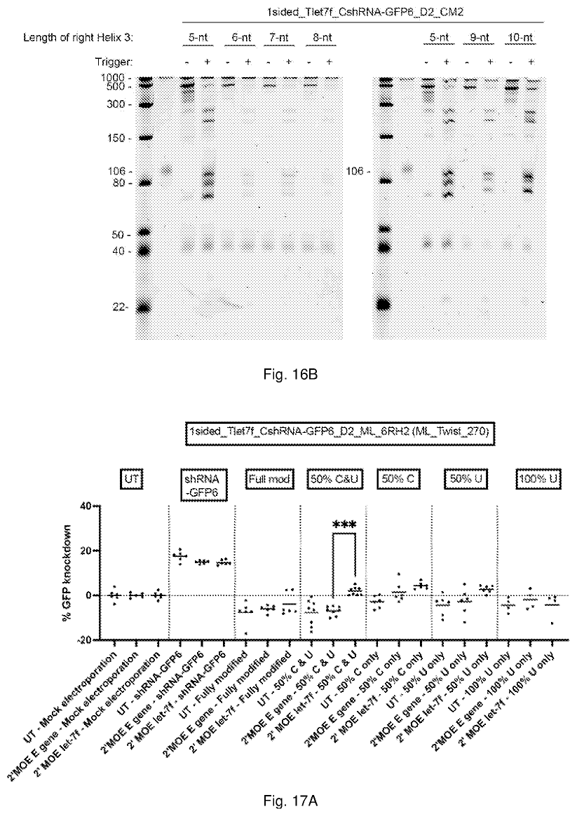

FIG. 17A: Detection of modified RNA triggers in cells.

FIG. 17Bi: Ribozyme can be triggered by 2′ MOE modified synthetic RNA (E gene). Two E-gene-triggered shRNA ribozymes, with ML and CM2 communication modules, can be triggered by modified E-gene trigger to cleave out embedded RNA cleavage product (boxed in gel).
2′MOE let-7f sequence (/52MOErT//i2MOErG//i2MOErA/rGrGrU rArGrU rArGrA rUrUrG rUrArU rA/i2MOErG//i2MOErT//32MOErT/) (SEQ ID NO: 506)
2′MOE E gene sequence (/52MOErT//i2MOErT//i2MOErC/rGrGrA rArGrA rGrArC rArGrG rUrA/i2MOErC//i2MOErG//32MOErT/) (SEQ ID NO: 507)

FIG. 17Bii: Ribozyme can be triggered by 2′ MOE modified synthetic RNA (let7f). Two let-7f-triggered shRNA ribozymes, with ML and CM2 communication modules, can be triggered by modified let-7f trigger to cleave out embedded RNA cleavage product (boxed in gel).

FIG. 17C. Ribozyme can be triggered by DNA. 3 ribozymes, triggered by S-gene fragment, E-gene fragment and mir-451a, respectively, can detect both RNA and DNA triggers to cleave out embedded RNA cleavage product (boxed in gel). E-gene ribozyme potentially shows preference for RNA trigger over DNA trigger.

FIG. 17D. Ribozymes partially modified with 2′-fluoro nucleotides (using Durascribe) can cleave. Fully modified ribozymes (100% 2′F C & U) may not be able to cleave.

### EXPERIMENTAL DATA

### Methods

### PCR Amplification of Template and In Vitro Transcription of RNA

DNA templates and primers were ordered (Integrated DNA Technologies, USA), and PCR amplification was performed using Phusion high fidelity PCR mastermix (#F531L, Thermo Fisher Scientific, USA) according to manufacturer's instructions. PCR products were purified using QIAquick PCR purification kit (#28106, Qiagen, Germany), and used as templates for in vitro transcription using AmpliScribe T7-flash transcription kit (#LGLC-ASF3507, Lucigen, USA) according to manufacturer's instructions. For some templates, DNA gene fragments (Twist Bioscience, USA) were used directly for in vitro transcription. For the experiments using modified ribozymes, Durascribe (#DS010925, Epicentre, USA) was used according to manufacturer's instructions. After the reaction, 280 μL of RNase-free water (#SH30538.02, Hyclone, USA) was added to 20 μL of the in vitro transcription reaction. The RNA was purified by adding 300 μL of acid-phenol:chloroform, pH 4.5 (with IAA, 125:24:1) (#AM9722, Invitrogen, USA), mixed, and centrifuged at 14,000 rpm for 3 min at room temperature. The aqueous phase was transferred to a new tube, and 300 μL of chloroform (#07278-00, Kanto Chemical, Japan) was added. The mixture was centrifuged at 14,000 rpm for 3 min at room temperature. The aqueous phase was transferred to a new tube and the RNA was precipitated with 1/10 volume (25 μL) of 3 M sodium acetate (pH 5.2) and 2.5× volume (625 μL) of absolute ethanol. After overnight precipitation at −20° C., the samples were centrifuged at 13,000 rpm for 20 min at 4° C. The supernatant was discarded, the RNA pellet was washed with cold 75% ethanol, and centrifuged at 13,000 rpm for 20 min at 4° C. After centrifugation, the supernatant was removed completely, and the pellet allowed to air-dry. RNA was re-suspended in 50 μL of RNase-free water (#SH30538.02, Hyclone, USA) and its concentration was measured using a NanoDrop spectrophotometer (Thermo Fisher Scientific, USA).

### Cell-Free Cleavage Assay

Assays were performed with 200 nM ribozyme RNA (in vitro transcribed) and 50 nM trigger RNA (Integrated DNA Technologies, USA) in 1× cleavage buffer (10 mM Tris, 7 mM magnesium chloride, 5 mM spermine, 2 mM sodium chloride, pH 6.4). 200 nM of an inert 40-nt RNA sequence (5′-GGGACAUGGAAGUCACACCUUCGGGAACGUGGUUGACCUA-3′) (SEQ ID NO: 461) was spiked into the reaction as a loading control. Reactions were incubated at 37° C. for 4 hours.

### Detection and Visualization of Cleavage Product

RNA was heat-denatured for 10 min at 70° C., loaded with 2×RNA Loading Dye (#B0363S, New England Biolabs, USA), and separated on a 10% denaturing polyacrylamide gel (#EC-833, National Diagnostics, USA) in 1×TBE (Tris/Boric Acid/EDTA) buffer (#1610770, Bio-Rad, USA) at 200 V for 1 hour, or until the dye front migrated to the bottom of the gel. Low range ssRNA ladder (#N0364S, New England Biolabs, USA) was loaded as a size marker, and 25 ng of a 29 nt oligo (5′-GUCCUUAGUCGAAAGUUUUACUAGAGUCA-3′) (SEQ ID NO: 462) (Integrated DNA Technologies, USA) or an in vitro transcribed RNA sequence, corresponding to the size of the expected cleavage product, was spiked in to the ladder as an additional size marker. Where appropriate, 25 ng of the trigger and spike-in sequence was also added into the ladder as size markers. Gels were stained with SYBR Gold at 1:10,000 dilution (#S11494, Invitrogen, USA), and visualized using ChemiDoc Imaging System (Bio-Rad, USA). Images were analyzed using Image Lab software (Bio-Rad, USA).

Using the Transblot SD semi-dry transfer cell (Bio-Rad, USA), RNA was transferred onto Hybond-N+ membrane (#RPN303B, GE Healthcare, USA) in 1×TBE (#1610770, Bio-Rad, USA) at 10 V and 300 mA for 1 hour in the cold room. The membrane was cross-linked using a UV crosslinker (Analytik Jena, USA), and pre-hybridized in PerfectHyb Plus Hybridization Buffer (#H7033-1L, Merck, USA) at 45° C. for 5 min with rotation. 5′ Alexa Fluor 647 or Cy5 labelled DNA probe against the cleavage product (Integrated DNA Technologies, USA) was added to the pre-hybridization solution and incubated in a hybridization oven at 45° C. for 3 hours with rotation. Following hybridization, membranes were washed progressively with increasing stringency of wash buffer (low stringency wash buffer: 2×SSC, 0.1% SDS; high stringency wash buffer: 0.5×SSC, 0.1% SDS; ultra-high stringency wash buffer: 0.1×SSC, 0.1% SDS). All washing steps were performed at 45° C. in a hybridization oven with rotation (except for low stringency wash, which was performed at room temperature with rotation). Membranes were visualized using ChemiDoc MP Imaging System (Bio-Rad, USA), and analyzed using Image Lab software (Bio-Rad, USA).

TABLE 2

| | | SEQ ID |
|---|---|---|
| Probe name | Sequence | NO: |
| CsgRNA_probe_ | /5Alex647N/GGCAAGCTGCCCGTGCCCAA | 463 |
| Alexa647 | | |
| Cban3pR_probe_ | /5Cy5/ACTCTAGTAAAACTTTCGAC | 464 |
| Cy5 | | |
| Exact_sgRNA_ | /5Alex647N/CCGGCAAGCTGCCCGTGCCC | 465 |
| probe_647 | | |
| CshRNA- | /5Alex647N/TGGACGGTATGGTCAACCGCGCCTATGA | 466 |
| GFP6_60nt_AF | ACTTCAGGGTCAGCTTGCCAAGAATAACACGC | |
| 647 | | |

### Maintenance of Cell Cultures

HEK293 cells were grown in Dulbecco's modified Eagle's medium (DMEM) supplemented with 10% heat-inactivated fetal bovine serum (Gibco #10270106), 1% penicillin/streptomycin (Gibco #15140122). HEK293-GFP cells (#SC001, Amsbio, USA) were grown in Dulbecco's modified Eagle's medium (DMEM) supplemented with 10% heat-inactivated fetal bovine serum (Gibco #10270106), 1% penicillin/streptomycin (Gibco #15140122), 2 mM L-glutamine, 0.1 mM MEM non-essential amino acids (Gibco #11140050), and and 10 μg/ml blasticidin (Gibco #A1113903). All cultures were maintained at 37° C. and 5% CO₂.

### Electroporation

Electroporation of RNA into HEK293-GFP cells was performed using the Neon transfection system (#MPK1096, Invitrogen, USA) according to manufacturer's instructions, with the parameters set as 1500 V, 30 ms pulse width, 1 pulse. The Alt-R CRISPR-Cas9 system (Integrated DNA Technologies, USA) was used for delivery of ribonucleoprotein complexes by electroporation using the Neon kit. After electroporation, cells were seeded on 96-well plates and incubated for 48 hours until analysis.

miRNA Inhibitors

miRNA and control inhibitors were purchased from Integrated DNA Technologies, USA.

hsa-let-7f_inhibitor
(SEQ ID NO: 467)
mA/ZEN/mA mCmUmA mUmAmC mAmAmU mCmUmA mCmUmA
mCmCmU mC/3ZEN/
NC1_neg_ctrl_inhibitor
(SEQ ID NO: 468)
mG/ZEN/mC mGmUmA mUmUmA mUmAmG mCmCmG mAmUmU
mAmAmC mG/3ZEN/

### Flow Cytometry

Cells were washed with 1×PBS (#10010023, Gibco, USA), and dislodged from the cell culture plate with Trypsin-EDTA (0.05%), phenol red (#25300054, Gibco, USA). The trypsin was neutralized by adding complete media and the entire volume from each well was transferred to a U-bottom 96-well plate. The plate was spun down at 300 g for 4 min, and the supernatant discarded. Cells were stained with Live/Dead fixable near-IR dead cell stain (#L34975, Invitrogen, USA) diluted 1:1000 in 1×PBS (#10010023, Gibco, USA) for 15 min at room temperature. After staining, the plate was spun down at 300 g for 4 min, and the supernatant discarded. Cells were re-suspended in 1×PBS (#10010023, Gibco, USA) and analysed on BD FACSymphony High Throughput Sampler (HTS). Data analysis was carried out using FlowJo (BD Biosciences, USA).

### Cloning of Plasmids

pSpCas9 (BB)-2A-Puro V2.0 vector (#62988, Addgene, USA) was used as the base vector for cloning in gRNA or ribozyme sequences. The vector was cut with BbsI (#R0539S, New England Biolabs, USA) and ligated with the desired insert using T4 DNA ligase (#10716359001, Roche, USA). The gRNA insert was made by oligo annealing of sense and antisense strands of the gRNA sequence with BbsI overhangs. For cloning of ribozymes into the same vector, a gblock (Integrated DNA technologies, USA) with the removal of the gRNA scaffold sequence and inclusion of a new BamHI restriction site was inserted between the AflllI and XbaI sites. This modified plasmid was then cut with BbsI and ligated with the desired ribozyme insert. The ribozyme insert was made with PCR amplification using Phusion high fidelity PCR mastermix ((#F531L, Thermo Fisher Scientific, USA) to include BbsI overhangs by creating PaqCI sites, and then cut with PaqCI (#R0745S, New England Biolands, USA), as the ribozyme sequence contains an internal BbsI cut site.

### Fish Micro-Injection

Ribonucleoprotein mixture (RNP) containing 150 ng of in vitro transcribed ribozyme and 0.5 μg of Cas9 protein (Integrated DNA Technologies, USA) was incubated at 37° C. for 10 mins and allowed to cool to room temperature. Each embryo was injected with 0.75 nL of RNP mixture.

Genomic DNA was extracted from single embryos 24 hours after micro-injection. Each embryo was first rinsed with 1×PBS (#10010023, Gibco, USA), followed by addition of 20 μL alkaline lysis solution (25 mM sodium hydroxide, 0.2 mM EDTA) and incubation at 95° C. for 30 min. The tubes were vortexed to check for complete lysis of embryos, and then incubated at 95° C. for another 10 min. 20 μL of neutralization buffer (40 mM Tris-HCl, pH 8.0) was added to each tube, and 2 μL of the extracted genomic DNA was used as template for PCR amplification of GFP using the following primers: SAW907_GFPF2 and SAW908_GFPR2.

SAW907_GFPF2
(SEQ ID NO: 469)
GTGGTGCCCATCCTGGTC
SAW908_GFPR2
(SEQ ID NO: 470)
CTTGTACAGCTCGTCCATGC

### Genome Editing and Detection

sgRNA design was carried out using http://crispor.tefor.net/. Genomic DNA extracted from cells using QuickExtract DNA extraction solution (#QE09050, Lucigen, USA), and used as a template for PCR amplification of GFP. The desired band was gel extracted using ZR-96 Zymoclean gel DNA recovery kit (#D4022, Zymo Research, USA). DNA was eluted in RNase-free water (#SH30538.02, Hyclone, USA), and the concentration was measured using a NanoDrop spectrophotometer (Thermo Fisher Scientific, USA).

50 ng of DNA was used as template for a second PCR using fluorescent labelled primers. Capillary electrophoresis was run on ABI 3730xl DNA Analyzer (Applied Biosystems, USA). The fluorescently labelled DNA fragments were sized by comparison to a size standard (#4322682, Applied Biosystems, USA). Data analysis was performed using GeneMapper (Applied Biosystems, USA).

FW + M13 adaptor
(SEQ ID NO: 471)
TGTAAAACGACGGCCAGTACGTAAACGGCCACAAGT
RV
(SEQ ID NO: 472)
TGAAGAAGATGGTGCGCTC

### Results

### An RNA Trigger-Activated Self-Cleaving Dual Ribozyme

Pandan, a miRNA sensor whose fluorescence is activated upon binding of a specific target miRNA, was previously developed by altering the structure of the fluorescent RNA Spinach2, so that hybridization of a target miRNA to its cognate RNA sensor backbone is required for stable binding of the fluorophore DFHBI (3,5-difluoro-4-hydroxybenzylidene imidazolinone). Pandan was designed by removing part of the structure of Spinach2, replacing it with sequences complementary to a target miRNA, so that stabilisation of the fluorophore DFHBI to the G-quadruplex structure of Spinach2 could only occur when the target RNA was bound. While Pandan sensors exhibited a substantial 50-fold increase in fluorescence in presence of 1 uM of its target miRNA, the Pandan sensor system, unlike other methods for RNA detection, did not comprise an amplification step that could enable more sensitive applications.

It was reasoned that the principle used for designing Pandan could be applied to modify a ribozyme able to amplify and transduce RNA signals: A molecule with ribozyme activity could be assembled from two or more different RNA molecules if the structural elements required for stable RNA folding into the catalytic conformation is only reconstituted upon base-pairing between a ribozyme sensor and its trigger RNA. This can be achieved if part of the ribozyme structure was removed and replaced by sequences complementary to a trigger RNA of interest. In addition, the inventors of the disclosure hypothesised that linkage of two such RNA-activated self-cleaving ribozymes could enable RNA signal amplification and transduction via release of a second RNA molecule from the dual-ribozyme.

After considering the structural characteristics of various ribozymes, the inventors selected as a proof-of-concept the self-cleaving hairpin ribozyme (HpRz), as mutagenesis and crystallography studies suggested that it exhibits the structural elements required. The HpRz consists of two independently folding domains (Domains A and B; FIG. 1A), each consisting of a loop region containing key catalytic nucleotides, flanked by two helices. The phosphodiester cleavage reaction occurs between the N₋₁ and guanine (G₊₁) nucleotides in Loop A (black arrow in FIG. 1A). The sequence requirements across the cleavage site has been systematically studied by many groups; the most highly tolerated sequences with activity of at least 20% of the wildtype ribozyme are shown in FIG. 1A.

FIG. 1Bi schematises our original design approach. Two HpRz, each with a modified Helix 4 containing complementary sequence to a trigger miRNA, would be arranged in tandem, one in a wild-type configuration (“right” side), and the other in a reverse-joined configuration (“left” side). Without the trigger RNA, catalytic Loop B is conformationally unstable, and ribozyme self-cleavage does not occur. Binding of the RNA trigger stabilises Loop B, activating cleavage to release an RNA cleavage product (FIG. 1Bi). This strategy requires three modifications to the original wildtype dual ribozyme (FIG. 1Bii). First, the inventors would circularly permute it so that Helix 4 will no longer be a closed stem loop; instead, the positions of 5′ and 3′ ends will be altered to reside within each Helix 4 (FIG. 1Biii; different junctional configurations are possible, and the 3-way junction is shown). This dual ribozyme would consist of two strands, the first comprising the cleavage sites and cleavage product (Strand 1; S1), and the second a non-cleaved strand (Strand 2; S2). Next, the inventors would introduce a second sequence-variable stem loop that branches off Helix 4 (IN-form; FIG. 1Biv); this mimics the structure that would be formed by binding of a trigger RNA. Finally, the inventors would assemble a functional self-cleaving ribozyme from three RNAs (S1, S2 and Trigger RNA) that are sequence-complementary at the new branched Helix 4 (OUT-form; FIG. 1Bv). The inventors systematically tested the feasibility of this strategy and optimised each feature of the ribozyme.

The inventors first selected an example trigger miRNA and an example cleavage product with which to start development. Since there was a wide range of performance efficacy for Pandan sensors ranging from 4 to 118 fluorescence fold-change, the inventors decided to first select as a trigger RNA the Drosophila microRNA bantam-5p (ban-5p), which was the trigger for the best-performing Pandan sensor. It was reasoned that a trigger RNA that binds well to its cognate Pandan sensor may also bind other similar branched sensor structures effectively. The inventors chose a 29-nt RNA fragment (29nt-clvRNA) as an example cleavage product, long enough to differentiate from the 23-nt ban-5p trigger miRNA on a gel.

The first step was to circularly permute the ribozyme (FIG. 1Biii). The inventors observed that a circularly permuted ribozyme retained self-cleavage activity to release the embedded 29-nt cleavage product (FIG. 9A). In order for the strategy of trigger-activated cleavage to work, the inventors believed that they ought to be able to destabilise Helix 4 by shortening its length; indeed, while ribozymes with an 8 bp Helix 4 showed clear release of cleavage product, shortening of Helix 4 to fewer than 3-4 nucleotides impeded cleavage product release (FIG. 9A). These initial experiments were carried out with the ribozyme in the 3-way junction configuration (FIG. 1Biii), and the inventors observed that the rate of cleavage product formation was low, even when Helix 4 was 8-nt long, and a substantial fraction of S1 strands remained intact (FIG. 9A). Hence, the inventors took two approaches to boost cleavage. First, since re-ligation rates increase when the cleaved product is highly complementary within Helix 1, the inventors decreased base-pairing between the cleavage product and S2 to favour cleavage product release. In addition, it had been shown that HpRzs with a 3-way junction (HHH) configuration (FIG. 1Biii, 9A, 9Bi) cleaved poorly, while those with modified 2-way (H₋₁S₇H) (FIG. 9Bii), modified 3-way (HHS₄H) (FIG. 9Biii) or modified 4-way (HHHS₂H) (FIG. 9Biv) configurations cleaved well. Hence, the inventors tested the cleavage activity of ribozymes with these modified junctions, where the cleavage product was either paired or unpaired with the ribozyme (FIG. 9C). These modifications substantially improved cleavage product release, with the unpaired 3-way (HHS₄H) and 4-way (HHHS₂H) junctions showing the highest rate of cleavage (FIG. 9C). However, the fully unpaired ribozymes exhibited unexpected cleavage bands (FIG. 9C, hashtags), and the inventors hypothesised that non-complementarity of the cleavage product disrupted ribozyme folding to cause off-target cleavage. Hence, the inventors partially restored complementarity between the cleavage product and the ribozyme (FIG. 9C). A configuration of 3/4-unpaired, 2-paired nucleotides, repeated along the cleavage product-ribozyme region, was very effective for cleavage product release (FIG. 9C, configuration #5).

Next, to determine whether the ribozyme could accommodate an RNA-binding trigger region, the inventors introduced branched stems into Helix 4 to mimic such a structure (Trigger-IN form, where the trigger RNA is introduced as part of the ribozyme, FIG. 1Biv). The optimised 4-way junction ribozyme with alternate base-pairing configuration was used (FIG. 9C, configuration #5). As Helix 4 stems longer than 4 bp released cleavage product in absence of trigger (FIG. 9A), the inventors tested Helix 4 stems of 1 to 4 bp. Trigger-IN ribozymes could self-cleave to release substantial 29-nt cleavage product when Helix 4 was at least 3 bp long (FIG. 1C), with increased Helix 4 lengths associated with greater cleavage product release (FIG. 9D). Therefore, circularly permuting the dual ribozyme, reducing complementarity of the cleavage product, introducing a 4-way junction, shortening Helix 4 and altering it to encompass a branched RNA-binding trigger region allowed for the possibility of a trigger-activated dual ribozyme.

### RNA-Triggered Cleavage in Cell-Free Assays

The sequences of both helices branching off Helix 4 could be varied. This allowed the inventors to encode sequences complementary to target RNAs into the ribozyme backbone. Hence, the possibility of reconstituting ribozyme activity from three separate RNAs was looked into by: A dual ribozyme with ban-5p trigger-binding domains (OUT-form, FIG. 1Bv), and a short RNA (ban-5p) complementary to the ribozyme trigger domain (FIG. 1D). This ribozyme was named Trigger RNA-ban-5p_Cleaved product-29nt-clvRNA (T-ban-5p_Cl-29nt-clvRNA). In the OUT-form, when Helix 4 was shorter than 2 bp, the ribozyme released very little cleavage product even in presence of the trigger (most S1 strands remained uncleaved; FIG. 1D). A Helix 4 of 2-3 bp length showed trigger-activated self-cleavage, releasing more cleavage product in presence of the trigger compared to in its absence. Increasing Helix 4 length to 8 bp led to high background cleavage in absence of trigger, and no appreciable trigger-activated cleavage (FIGS. 1D, 9E). A second ribozyme was tested, triggered by a different miRNA let-7f to release the same 29-nt cleavage product (T-let-7f_Cl-29nt-clvRNA). This ribozyme also released the cleavage product in a trigger-dependent manner (FIG. 9F).

Therefore, the inventors have developed a ribozyme with two cleavage sites and two trigger-binding regions, which is activated by a trigger RNA of interest to release an embedded RNA cleavage product.

### a Single Catalytic Domain is Sufficient for RNA-Triggered Dual Self-Cleavage

The observed cleavage patterns hinted at differential cleavage rates between the two sites (FIGS. 1C, D). Hence, each catalytic domain was individually mutated to determine if one was more active. A₃₈ is a critical nucleobase involved in stabilising the transition state during catalysis, and mutation of this nucleotide abolishes cleavage. To the inventors' surprise, mutation of A₃₈→U₃₈, in either the “right” wildtype catalytic or the “left” reverse-joined catalytic domain in the T-ban-5p_Cl-29nt-clvRNA dual ribozyme (FIG. 1Bv, 1D with 8-nt H4), did not abolish cleavage at either cleavage site, nor substantially reduce release of the cleavage product, while mutation of both catalytic domains completely abolished cleavage (FIG. 2A). This suggested that either single catalytic domain can cleave at both cleavage sites.

To test this, dual-cleavage site ribozymes were designed with either a single “right” wildtype catalytic domain or a single “left” reverse-joined catalytic domain (FIG. 2B). These ribozymes can now be encoded by a single strand of RNA. Surprisingly, both single ribozymes were able to cleave at both sites to release the cleavage product (FIG. 2C). Addition of the trigger RNA increased cleavage product release for the ban-5p single “right-sided” ribozyme (FIG. 2C). The inventors chose to proceed with the single “right” wildtype catalytic domain, reasoning that it could be rationally optimised based on the abundance of functional studies that have been carried out on this canonical structure. “Right-sided” single ribozymes, triggered by microRNAs dme-mir-184, dme-mir-252, dme-mir-263a, hsa-let-7f, as well as for fragments of the E-gene and Orf1ab transcripts of the SARS-CoV-2 genome, all could self-cleave at both sites to release the cleavage product (FIGS. 2C, 10A), confirming that one catalytic domain was sufficient for dual cleavage. Mutation of A₃₈→U₃₈ in the single catalytic domain abolished cleavage (FIG. 2C). Several ribozymes showed additional cleavage product release when trigger RNA was added; however, most ribozymes showed cleavage product release even in absence of the trigger (FIGS. 2C, 10A).

To optimise the signal-to-noise ratio in presence vs absence of the trigger, Helix 4 was varied, which acts as a trigger-responsive communication module, to identify an optimal stem length and sequence. Ribozymes with Helix 4 longer than 5-nt cleaved in a trigger-independent manner, while those with lengths of 2-nt were cleavage-resistant even in presence of trigger (FIG. 2D). Two 3-nt motifs, 5′-ACG/CGU-3′ (3-nt v1) and 5′-ACG/CGA-3′ (3-nt v2), greatly improved the signal-to-noise ratio of several ribozymes (FIGS. 2D, 10B). Cleavage product increased with increasing trigger RNA concentration (FIG. 2E). The inventors named this trigger-activated, single-stranded dual ribozyme platform, able to self-cleave and release an embedded RNA product upon trigger RNA-binding, Unlocked By Activating RNA (UNBAR).

The inventors next investigated the ability of the ribozymes to discriminate between unrelated and closely related sequences by introducing sequence changes into the test RNA (FIG. 2F). An unrelated RNA (S-gene fragment) was unable to trigger cleavage. The effect of single nucleotide mismatches depended on the location of the mismatch, with position 4 reducing cleavage product formation by more than 80%. Dual and triple mutants exhibited strong reduction in cleavage activity (FIGS. 2F, 10D). These results suggest that these ribozymes can be optimised to distinguish between similar target sequences.

For these ribozymes to be useful in biological samples, they must identify their triggers in complex mixtures. total RNA from HEK293T cells was prepared and assayed for the ability of the ribozyme to detect trigger RNA spiked into the RNA mixture at a range of concentrations. The ribozyme could detect its trigger in presence of at least 1000-fold excess competing RNA (FIG. 2G).

Functional RNA as Cleavage Products: CRISPR sgRNA and shRNA

To explore whether these ribozymes can be used for RNA context-specific gene regulation in vivo, the inventors engineered ribozymes encoding either a CRISPR single-guide RNA against GFP (sgRNA^{GFP}; FIG. 3A), a short hairpin RNA against GFP (shRNA^{GFP}; FIG. 3B), or an RNA aptamer (Broccoli, FIG. 3C) as the cleavage product. The inventors first carried out a cell-based screen to identify efficient sgRNAs; and found that three sgRNAs beginning with GUC, the canonical sequence at the hairpin ribozyme cleavage site (FIG. 1A), were inefficient at inducing editing (FIG. 11A), although they could be encoded within the ribozyme and released in a trigger-dependent manner (FIG. 11B). Hence, the inventors selected sgRNA GFP-149R, which is efficient (FIG. 11A) and previously characterised. The inventors first retained the conserved hairpin ribozyme sequences at the cleavage sites to test cleavage of these more complex products; hence, at 5′ and 3′ ends of the sgRNA and shRNA, there were one to four nucleotides “leftover” that would be included in the cleavage product. Extra nucleotides at the 3′ end of the gRNA should not alter Cas9 targeting and DNA cleavage. While unpaired nucleotides at the 5′ end of the sgRNA 20-nt protospacer sequence can inhibit DNA cleavage, these effects are context-dependent, and the sgRNA we chose can tolerate unpaired nucleotides at its 5′ end. The inventors later found that this sgRNA can be made to be efficiently cleaved exactly without “leftover” nucleotides at the 5′ end, despite beginning with GGG, by mutating the A7 in the WT cleavage loop to either U₇ or C₇ (FIG. 11C), to pair with G₊₃.

The inventors designed the shRNA ribozyme to resemble a primary microRNA, with single-stranded RNA (ssRNA) basal segments and double stranded (dsRNA) segments predicted to be a substrate for Drosha cleavage, continuous with a GFP^{shRNA}. This was achieved by designing and screening transcripts optimised with features important for microRNA processing⁴⁹, which also included 5′ and 3′ ribozyme scar sequences to enable subsequent embedding within the ribozyme. The inventors identified potential cleavage products that strongly decreased GFP expression. They then designed ribozymes that comprised the most potent cleavage product, shGFP6 (T-let-7f_Cl-shRNA^{GFP6} (FIG. 7). Before cleavage, the ssRNA basal segments are base-paired with the ribozyme (FIG. 3B), and since single-stranded basal strands are essential for Drosha processing, uncleaved shRNA embedded in the ribozyme should not be processed for RNAi. After trigger-induced ribozyme self-cleavage, the cleavage product is released to become a potential substrate for Drosha, and to enter the miRNA maturation pathway. Since Drosha cleavage does not require specific conserved sequences, but rather “counts” from the junction of the ssRNA and dsRNA, the inventors hypothesised that incorporation of 5′ and 3′ scar nucleotides would not affect processing of the shRNA cleavage product. Finally, the inventors also designed and tested ribozymes that cleave out the fluorescent aptamer Broccoli (FIG. 3C).

The inventors chose as the trigger miRNA let-7f, as the let-7 family is highly conserved, with let-7f one of its most abundant members (www.mirbase.org), allowing the inventors to test these ribozymes in both zebrafish embryos and human cell lines. T-let-7f_C1-sgRNA^{GFP}, T-let-7f_Cl-shRNA^{GFP} (two different shRNA for GFP were tested for cleavage) and T-let-7f_Cl-Broccoli were assayed for trigger-activated cleavage in cell-free assays. For all three ribozymes, substantially more cleavage product was released in presence of trigger than without (FIGS. 3D-F). Therefore, these ribozymes can be triggered to release longer functional RNA with secondary structure, including sgRNA, shRNA and aptamers.

The let-7 miRNA family consists of 12 closely related members (FIG. 3G). We asked whether T-let-7f_C1-sgRNA^{GFP} could distinguish between them. T-let-7f_Cl-sgRNA^{GFP} could distinguish between most let-7 family members that differed from let-7f by at least 3-nt (i.e., let-7b, let-7i and mir-98). Let-7d was not distinguishable despite differing by 3-nt, perhaps because the nucleotide at position 1 may have a weaker effect (FIG. 3G).

While cleavage product release from the ribozyme was dependent on presence of the trigger RNA, a substantial fraction of ribozymes cleaved at the site proximal to the catalytic domain in absence of trigger (FIGS. 2, D-F), which could lead to leaky activity of the sgRNA or shRNA cleavage product in vivo. However, the distal cleavage site was not cleaved without trigger, and the inventors hypothesised that this stemmed from its increased distance from catalytic Loop B. To test this, the inventors lengthened Helix 2 of T-let-7f_C1-sgRNA^{GFP} from 4-bp to 8-bp or 12-bp (FIG. 3H). This substantially decreased trigger-independent cleavage at the proximal site (FIG. 3H). To ask if this was generalisable, the inventors similarly altered the T-let-7f_C1-shRNA^{GFP} ribozyme. This also reduced trigger-independent cleavage at the proximal site (FIG. 11D) Therefore, the length of Helix 2 can be tuned to refine cleavage regulation by the trigger RNA.

### In Vivo RNA Signal Transduction

The inventors next asked whether these ribozymes can enable cell RNA context-dependent gene regulation in vivo. CRISPR-Cas9 editing can be carried out in zebrafish embryos by microinjection of RNA-Cas9 complexes. The T-let-7f_C1-sgRNA^{GFP} ribozyme can detect let-7a, c, d, e, f, g (FIG. 3E); let-7a, -c and -f account for 72% of these miRNAs in the early fish embryo (www.mirbase.org). One-cell injection of Cas9 protein with the positive control sgRNA^{GFP} led to high rates of genomic deletions, ˜88% per embryo (FIG. 4A). Co-injection of T-let-7f_C1-sgRNA^{GFP} with Cas9 also led to robust editing, although lower than the gRNA alone, at ˜29% per embryo (p<0.001) (FIG. 4A). This provides evidence that the sgRNA-encoding ribozyme is stable in vivo and able to cause gene editing when delivered as RNA. To determine whether editing was let-7-dependent, the inventors co-expressed with the ribozyme a morpholino antisense inhibitor against let-7f. Co-injection of the inhibitor with the ribozyme reduced ribozyme-induced editing at the GFP locus from ˜29% to ˜12% (p<0.05), providing support for gRNA function being let-7f-dependent (FIG. 4A).

The inventors next asked whether these ribozymes can function in human cells. Expression of Durascribe-modified (50% of Cs and Us modified) T-let-7f_C1-shRNA^{GFP6} ribozymes in HEK293-GFP cells with 2′MOE-modified let-7f led to a ˜9% increase in GFP knockdown, while expression of 2′MOE-modified E-gen had no effect (FIG. 4B).

Similarly, expression of Durascribe-modified (50% of Cs modified) T-Egene20_Cl-Stoplight_modC ribozymes in Stoplight cells (de Jong et al Nature Communications 2020) with 2′MOE-modified E-gene increased gene editing, while expression of 2′MOE-modified let-7f had no effect (FIG. 4C). Therefore regulation by the ribozyme is trigger-dependent in mammalian cells.

Sequence with A₇: (Note: This refers to FIG. 11,
described above)
(SEQ ID NO: 473)
AACUAUACAAUACGGUAUAUUACCUGGUUUUCGAUCGAAAGAUCGACGAG
GUGAAAACCUCGUGACAGGGCACGGGCAGCUUGCCGGGUUUUAGAGCUAG
AAAUAGCAAGUUAAAAUAAGGCUAGUCCGUUAUCAACUUGAAAAAGUGGC
ACCGAGUCGGUGCGUCAGUCCUGAUUUUCGAAUCAGAGAAGACUAUCCAC
CUUAAAUAGGCAAGUGAGAAGUCAACCAGAGAAACACGACUACUACCUCA
Sequence with C₇:
(SEQ ID NO: 474)
AACUAUACAAUACGGUAUAUUACCUGGUUUUCGAUCGAAAGAUCGACGAG
GUGAAAACCUCGUGACAGGGCACGGGCAGCUUGCCGGGUUUUAGAGCUAG
AAAUAGCAAGUUAAAAUAAGGCUAGUCCGUUAUCAACUUGAAAAAGUGGC
ACCGAGUCGGUGCGUCAGUCCUGAUUUUCGAAUCAGAGAAGACUAUCCAC
CUUAAAUAGGCAAGUGCGAAGUCAACCAGAGAAACACGACUACUACCUCA

For clarity, the specific nucleotide sequences are known in the art by numerical positions based on the original hairpin ribozyme. Hence, in one example, A₇ and C₇ as mentioned herein are named based on that original hairpin ribozyme annotation, i.e. it (the location mentioned) does not depend on its specific position in a specific ribozyme, but rather this position refers to a nucleotide located in the bulge opposite the cleavage site in the canonical hairpin ribozyme. Shown in FIG. 1 is a sketch of the canonical hairpin ribozyme with an exemplary N₇ position indicated. One amendment disclosed herein which had an effect on cleavage efficacy is the change of the nucleotide identity of N₇ so that it will pair with nucleotide N₊₃ in the cleavage product. This is useful because it allows variance in the cleavage product sequence. So, exemplarily speaking, if nucleotide N₊₃ is a G in the cleavage product, then N₇ can be changed to a C (to pair with this G). But if N₊₃ is changed to an A, for example, then N₇ can be changed to a U (which pairs with A) to increase cleavage activity.

Disclosed herein are features of the ribozyme, whereby these features are shown to improve its signal to noise ratio, i.e. to decrease its background cleavage in absence of the target/trigger RNA, and increase its cleavage rate in presence of the target/trigger RNA. Preferably, any such modification should be applicable over a wide series of ribozymes. Towards this, the inventors tested several motifs for the communication module of the ribozyme, i.e. the region between the catalytic domain and the trigger arms (FIG. 1A). The inventors found that a 2-nt communication module was too short (FIG. 12B), while a 3-nt motif was generally preferred over the other tested lengths. Specifically, several 3-nt communication module dsRNA motifs, 5′-ACG/CGU-3′ (SEQ ID NO: 1) and 5′-ACG/CGA-3′ (SEQ ID NO: 2) were highly effective (FIGS. 2D, 10C), as well as several other variants shown in FIGS. 12A and 12B, especially (CM2: 5′-AUG/CGA-3′) (SEQ ID NO: 450), (CM7: 5′-ACU/AGA-3′) (SEQ ID NO: 455) and (CM8: 5′-AUG/CAA-3′) (SEQ ID NO: 456), 5′-UAU/AUA-3′ (SEQ ID NO: 454), and 5′-CU/AG-3′ (SEQ ID NO: 457) (FIGS. 12A, B). Therefore, changing the communication module to these variants could greatly improve the signal to noise ratio of the ribozyme.

Next, using these optimised communication modules, the inventors developed ribozymes that were triggered by a short RNA to release either an shRNA or a sgRNA against Green Fluorescent Protein (GFP), or a fluorescent aptamer (Figs. A-C). All of these ribozymes released the embedded shRNA, sgRNA or aptamer when triggered by RNA let-7f, at a high signal-to-noise ratio (FIGS. 3D-F).

However, the inventors noticed that there was substantial background cleavage at the cleavage site that was proximal to the catalytic domain, with little to no background cleavage at the distal cleavage site. The inventors wondered whether the lower background cleavage at the distal site was due to its greater distance from the catalytic domain. Hence, the inventors asked whether extending the length of Helix 2 could decrease the level of background cleavage at the proximal site. Hence, the inventors extended Helix 2 from its normal 4-bp configuration to either 8-bp or 12-bp. This significantly decreased the background cleavage at the proximal site in absence of the trigger (FIG. 3H). Lengthening of Helix 2 to lengths 5-10 bp also had similar benefits (FIG. 13A). Therefore, lengthening of Helix 2 can decrease background cleavage in absence of the trigger.

The inventors also asked whether extension of Helix 3 could affect cleavage. They found that alteration of Helix 3 also dramatically decreased background cleavage in absence of trigger (FIG. 16).

### Optimisations and Other Experimental Data

1) A new sequence motif in Helix 4 of the ribozyme that results in Rz with very high signal-to-noise ratio in presence of the trigger RNA, generalizable over many Rz

FIG. 12A exemplified 10 additional motifs in Helix 4 of the ribozymes that has very high signal-to-noise ratio in the presence of the trigger RNA. These additional motifs include, but are not limited to (5′-ACG/UGA-3′) (SEQ ID NO: 449), (5′-AUG/CGA-3′) (SEQ ID NO: 450), (5′-AUG/UGA-3′) (SEQ ID NO: 451), (5′-CG/CG-3′) (SEQ ID NO: 452), (5′-UUG/UGG-3′) (SEQ ID NO: 453), (5′-UAU/AUA-3′) (SEQ ID NO 454), (5′-ACU/AGA-3′) (SEQ ID NO: 455), (5′-AUG/CAA-3′) (SEQ ID NO: 456), (5′-CU/AG-3′) (SEQ ID NO: 457), and (5′-UG/CA-3′) (SEQ ID NO: 458).

Cleavage assays showed that at least 3 of these work as well as or better than SEQ ID1 and SEQ ID2 motifs (FIG. 12B) (CM2: 5′-AUG/CGA-3′) (SEQ ID NO 450), (CM7: 5′-ACU/AGA-3′) (SEQ ID NO: 455) and (CM8: 5′-AUG/CAA-3′) (SEQ ID NO: 456). Also, marginally, 5′-UAU/AUA-3′ (SEQ ID NO: 454), and 5′-CU/AG-3′ (SEQ ID NO: 457), which shows that a 2-nt Helix 4 can also work in some ribozyme contexts.
2) A new, lengthened Helix 2 (we previously tested 8-nt and 12-nt), which reduces the background cleavage of the cleavage site proximal to the catalytic domain in absence of the trigger

FIG. 13 shows the testing of additional modified Helix 2 lengths, from 5-10-nt, some of which also exhibit reduced background cleavage, e.g. 6-nt to 10-nt.
3) Ribozymes with full complementarity exhibit increased cleavage-dependency of some functional ribozymes

The inventors of the present disclosure made shRNA-releasing ribozymes that have increased complementarity between the cleavage product and ribozyme, including full complementarity (FIG. 14A,B, D2 design). These greatly improve the mild cleavage dependency of the original design (FIG. 14B). sgRNA-releasing ribozymes with increased complementarity between the cleavage product and ribozyme were also made and found to function (FIG. 15).
4) Rz embedded with CRISPR sgRNA, shRNA and aptamers, that release them in presence of trigger RNA

The inventors present examples of ribozymes embedded with shRNA and sgRNA. For shRNA, see FIGS. 3, 12, 13, and 14. New sgRNA examples are in FIGS. 3 and 15.
5) Use of sgRNA and shRNA-releasing ribozymes in vivo and in vitro, in zebrafish and mammalian cells, respectively.

Additional examples of shRNA and sgRNA-releasing ribozymes are listed in the Table 1 above. The functional efficacy of shRNA and sgRNA ribozymes in cells and in vivo are demonstrated in FIGS. 4A-C.

### Applications

Embodiments of the methods disclosed herein provide a sensitive, low to no background, specific, and functional ribozyme.

Advantageously, ribozymes as disclosed herein includes one or more surprising improvements including: 1) new sequence motifs in the Helix 4 communication module of the ribozyme that result in ribozymes with very low background cleavage in absence of the target/trigger RNA, and high cleavage rates in presence of the target/trigger RNA, applicable over a series of ribozymes, 2) new lengthened Helix 2 domains, which reduce the background cleavage of the cleavage site proximal to the catalytic domain, 3) ribozymes with full complementarity between the releasable cleavage product and its complementary strand, to reduce background cleavage product release, 4) a mutation of nucleotide N₇ to pair with nucleotide N₊₃, to increase cleavage when the sequence at the cleavage site deviates from canonical cleavage site sequences, and 5) new lengthened Helix 3 domains, which reduce the background cleavage of the cleavage site proximal to the catalytic domain.

Even more advantageously, the ribozymes as disclosed herein have very low background cleavage and leakiness that enable them to be useful for various cell-free, in vitro and in vivo applications.

The ribozymes as disclosed herein may be advantagenously designed to comprise functional RNA such as, but not limited to, single guide RNA (sgRNA), short hairpin RNA (shRNA), or an RNA aptamer, and the like. Therefore, the ribozymes as disclosed herein are capable of releasing functional RNA in the presence of trigger RNA.

The ribozymes as disclosed herein are also advantageously capable of releasing functional RNA with secondary structure, such as, but not limited to, single guide RNA (sgRNA), short hairpin RNA (shRNA), an RNA aptamer, and the like.

The ribozymes as disclosed herein may be used in vitro and in vivo (such as as exemplified in zebrafish and mammalian cells) to elicit gene knockdown (when in the case of shRNA ribozyme) or gene editing (in the case of sgRNA ribozyme) when in the presence of trigger RNA. Therefore, the ribozymes as disclosed herein may also be used for gene regulation, with the output dependent on the type of functional RNA encoded as the releasable cleavage product.

Disclosed herein are features of the ribozyme, whereby these features are shown to improve its signal-to-noise ratio, i.e. to decrease its background cleavage in absence of the target/trigger RNA, and increase its cleavage rate in presence of the target/trigger RNA. Preferably, any such modification should be applicable over a wide series of ribozymes.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A ribozyme comprising:
a) one or more catalytic domains capable of switching between an active state and an inactive state;
b) one or more releasable RNA segments, wherein each of said releasable RNA segments is flanked by two ribozyme cleavage sites, wherein cleavage at each cleavage site is catalysed by at least one of the one or more catalytic domains in an active state;
c) one or more trigger-binding domains, each of which is for the binding of a trigger nucleic acid molecule; wherein each of the one or more catalytic domains is linked to one of the one or more trigger-binding domains;
wherein the catalytic domain is in an inactive state when the trigger-binding domain linked to said catalytic domain is not bound by the trigger nucleic acid molecule, and wherein the catalytic domain is in an active state when the trigger-binding domain linked to said catalytic domain is bound by the trigger nucleic acid molecule; and
wherein when both cleavage sites flanking a releasable RNA segment are cleaved, the one or more releasable RNA segment is released from the ribozyme,
wherein the ribozyme comprises an RNA strand with
motifs [A] and [a], wherein motifs [A] and [a] constitute the trigger-binding domain for binding the trigger nucleic acid molecule;
motifs [B] and [b], wherein motifs [B] and [b] constitute a linker that functions as a communication module to stabilise the catalytic domain when the trigger nucleic acid is bound, wherein motif [B] and [b] are independently at least 1 nucleotide in length;
motifs [C] and [c], wherein motifs [C] and [c] constitute the catalytic domain;
motif [D], wherein motif [D] comprises the first cleavage site capable of being cleaved when catalysed by the catalytic domain;
motif [D′], wherein motif [D′] comprises the second cleavage site capable of being cleaved when catalysed by the catalytic domain;
motif [E], wherein motif [E] comprises the releasable RNA segment;
motif [e], wherein motif [e] comprises a sequence that is partially or fully complementary to the sequence of motif [E];
and wherein the motifs are connected by one or more optional linker region.

2. The ribozyme complex of claim 1, wherein the linker between motifs [C] and [D] is selected from the group consisting of two-way junction, three-way junction, four-way junction, a stem, single-nucleotide bulges, two-nucleotide bulges, three-nucleotide bulges, multi-nucleotide bulges and combinations thereof.

3. The ribozyme complex of claim 2, wherein the linker between motifs [C] and [D] comprises a three-way junction and a stem.

4. The ribozyme complex of claim 3, wherein the stem sequence connecting the junction to motif [D] is 4 to 12 nucleotides in length.

5. The ribozyme complex of claim 3, wherein the stem sequence connecting motif [C] and [c] to the junction is 4 to 12 nucleotides in length.

6. The ribozyme of claim 1, wherein the trigger nucleic acid molecule comprises a region that is complementary to the trigger-binding domain, wherein said region is more than 10 nucleotides in length, optionally the one or more trigger-binding domains are for binding the same trigger nucleic acid molecule.

7. The ribozyme of claim 1, wherein the releasable RNA segment is 6 to 150 nucleotides in length.

8. The ribozyme of claim 1, wherein the releasable RNA segment comprises a sequence that is identical to at least one of the one or more trigger RNA molecules.

9. The ribozyme of claim 1, wherein the releasable RNA segment is a functional RNA selected from the group consisting of single-guide RNA (sgRNA), guide RNA (gRNA), short hairpin RNA (shRNA), and RNA aptamer.

10. The ribozyme of claim 1, wherein motifs [B] and [b] are independently 1 or more nucleotides in length, optionally 3 or more nucleotides in length.

11. The ribozyme of claim 1, wherein motifs [B] and [b] has a sequence selected from the group consisting of SEQ ID NO: 1 (5′-ACG/CGU-3′), SEQ ID NO: 2 (5′-ACG/CGA-3′), SEQ ID NO: 449 (5′-ACG/UGA-3′), SEQ ID NO: 450 (5′-AUG/CGA-3′), SEQ ID NO: 451 (5′-AUG/UGA-3′), SEQ ID NO: 452 (5′-CG/CG-3′), SEQ ID NO: 453 (5′-UUG/UGG-3′), SEQ ID NO: 454 (5′-UAU/AUA-3′), SEQ ID NO: 455 (5′-ACU/AGA-3′), SEQ ID NO: 456 (5′-AUG/CAA-3′), SEQ ID NO: 457 (5′-CU/AG-3′), and SEQ ID NO: 458 (5′-UG/CA-3′).

12. The ribozyme of claim 1, wherein if motifs [e] and [E] are partially complementary to each other, the complementarity between motif [e] and [E] is characterised by alternating regions of complementarity and regions of non-complementarity.

13. The ribozyme of claim 1, wherein motif [D] comprises a mutation of nucleotide N₇ to pair with nucleotide N₊₃.

14. The ribozyme of claim 1, wherein the optional linker regions individually or collectively form one or more secondary structures, optionally the one or more secondary structures are selected from the group consisting of: single-nucleotide bulges, two-nucleotide bulges, three-nucleotide bulges, multi-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junctions), two-stem junctions (2-way junctions) or coaxial stacks or combinations thereof.

15. The ribozyme of claim 1, wherein the ribozyme complex comprises the sequences of any one or more of SEQ ID NOs: 3 to SEQ ID NOs: 448.

16. The ribozyme of claim 1, wherein the ribozyme further comprises one or more modification.

17. The ribozyme of claim 1, wherein the trigger nucleic acid molecule comprises one or more modified nucleotide; optionally wherein the trigger nucleic acid molecule is a genome of a virus, or a fragment thereof.

18. A method of detecting presence of a target/trigger nucleic acid molecule in a sample, wherein the method comprises:
incubating the sample with a ribozyme according to claim 1 at temperature T1 which allows the binding of the target/trigger nucleic acid molecule with one or more target/trigger-binding domains comprised in the ribozyme;
incubating the sample at temperature T2 which allows the nucleic acid molecule and the RNA segment to be released from the ribozyme;
detecting the release of the releasable RNA segment from the ribozyme;
optionally wherein the target/trigger nucleic acid molecule is a genome of a virus, or a fragment thereof.

19. A method of detecting presence of a sequence or mutation of interest on a nucleic acid molecule of interest in a sample, wherein the method comprises:
incubating the sample with a ribozyme according to claim 1, thereby allowing binding of the nucleic acid molecule of interest with one or more target/trigger-binding domains comprised in the ribozyme;
incubating the sample which allows the nucleic acid molecule and a releasable RNA segment to be released from the ribozyme;
detecting the release of the releasable RNA segment from the ribozyme;
wherein the releasable RNA segment is an sgRNA or shRNA; wherein detection of the sequence or mutation of interest in the sample results in a signal being generated;
optionally wherein the nucleic acid of interest is a genome of a virus, or a fragment thereof.

20.-21. (canceled)


SEQUENCE LISTING (507 sequences)

SEQ ID NO: 1-2 - skipped (2 entries)

SEQ ID NO: 3 - RNA, 136 nt, synthetic construct
  misc_feature 1..136: note = Synthetic sequence
  source 1..136: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacctggtccc

SEQ ID NO: 4 - RNA, 134 nt, synthetic construct
  misc_feature 1..134: note = Synthetic sequence
  source 1..134: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacctggtt

SEQ ID NO: 5 - RNA, 132 nt, synthetic construct
  misc_feature 1..132: note = Synthetic sequence
  source 1..132: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacctgg

SEQ ID NO: 6 - RNA, 131 nt, synthetic construct
  misc_feature 1..131: note = Synthetic sequence
  source 1..131: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacctg

SEQ ID NO: 7 - RNA, 130 nt, synthetic construct
  misc_feature 1..130: note = Synthetic sequence
  source 1..130: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacct

SEQ ID NO: 8 - RNA, 129 nt, synthetic construct
  misc_feature 1..129: note = Synthetic sequence
  source 1..129: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattacc

SEQ ID NO: 9 - RNA, 128 nt, synthetic construct
  misc_feature 1..128: note = Synthetic sequence
  source 1..128: mol_type = other RNA
gggaagtggtatattacctggtacgcgttcacggttcgccgtgaacgcgttgacagtccttagtcgaaagttttactagagtcagtcctgattgaggtcgtgagtccacgacctccgtggtatattac

SEQ ID NO: 10 - RNA, 173 nt, synthetic construct
  misc_feature 1..173: note = Synthetic sequence
  source 1..173: mol_type = other RNA
gggaagtcaaaccaaagtggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctggatcgaaaaccgg

SEQ ID NO: 11 - RNA, 171 nt, synthetic construct
  misc_feature 1..171: note = Synthetic sequence
  source 1..171: mol_type = other RNA
gggaagtcaaaccaaagtgtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctgatcgaaaaccgg

SEQ ID NO: 12 - RNA, 169 nt, synthetic construct
  misc_feature 1..169: note = Synthetic sequence
  source 1..169: mol_type = other RNA
gggaagtcaaaccaaaggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctatcgaaaaccgg

SEQ ID NO: 13 - RNA, 167 nt, synthetic construct
  misc_feature 1..167: note = Synthetic sequence
  source 1..167: mol_type = other RNA
gggaagtcaaaccaaagtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattaccatcgaaaaccgg

SEQ ID NO: 14 - RNA, 181 nt, synthetic construct
  misc_feature 1..181: note = Synthetic sequence
  source 1..181: mol_type = other RNA
gggaagtcaaaccaagggaagtggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctggtcccatcgaaaaccgg

SEQ ID NO: 15 - RNA, 173 nt, synthetic construct; identical to SEQ ID NO: 10
  misc_feature 1..173: note = Synthetic sequence
  source 1..173: mol_type = other RNA

SEQ ID NO: 16 - RNA, 171 nt, synthetic construct
  misc_feature 1..171: note = Synthetic sequence
  source 1..171: mol_type = other RNA
gggaagtcaaaccaagtggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctgatcgaaaaccgg

SEQ ID NO: 17 - RNA, 169 nt, synthetic construct
  misc_feature 1..169: note = Synthetic sequence
  source 1..169: mol_type = other RNA
gggaagtcaaaccaatggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctatcgaaaaccgg

SEQ ID NO: 18 - RNA, 167 nt, synthetic construct
  misc_feature 1..167: note = Synthetic sequence
  source 1..167: mol_type = other RNA
gggaagtcaaaccaaggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattaccatcgaaaaccgg

SEQ ID NO: 19 - DNA, 98 bp, synthetic construct
  source 1..98: mol_type = other DNA
  misc_RNA 1..98
gggaagtggtatattacctggttctttctgacagtccttagtcgaaagttttactagagtcagtcctgattctttctcgtggtatattacctggtccc

SEQ ID NO: 20 - DNA, 136 bp, synthetic construct
  misc_RNA 1..136
  source 1..136: mol_type = other DNA
gggaagtggtatattacctggtacgcgttcacggttcgccgtgaacgcgttgacagtccttagtcgaaagttttactagagtcagtcctgattgaggtcgtgagtccacgacctccgtggtatattacctggtccc

SEQ ID NO: 21 - DNA, 136 bp, synthetic construct; identical to SEQ ID NO: 20
  misc_RNA 1..136
  source 1..136: mol_type = other DNA

SEQ ID NO: 22 - DNA, 144 bp, synthetic construct
  misc_RNA 1..144
  source 1..144: mol_type = other DNA
gggaagtggtatattacctggtctttacgcgttcacggttcgccgtgaacgcgttgacagtccttagtcgaaagttttactagagtcagtcctgattgaggtcgtgagtccacgacctcctttcgtggtatattacctggtccc

SEQ ID NO: 23 - DNA, 144 bp, synthetic construct; identical to SEQ ID NO: 22
  misc_RNA 1..144
  source 1..144: mol_type = other DNA

SEQ ID NO: 24 - DNA, 154 bp, synthetic construct
  misc_RNA 1..154
  source 1..154: mol_type = other DNA
gggaagtggtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattacctggtccc

SEQ ID NO: 25 - DNA, 154 bp, synthetic construct; identical to SEQ ID NO: 24
  misc_RNA 1..154
  source 1..154: mol_type = other DNA

SEQ ID NO: 26 - DNA, 154 bp, synthetic construct; identical to SEQ ID NO: 24
  misc_RNA 1..154
  source 1..154: mol_type = other DNA

SEQ ID NO: 27 - DNA, 154 bp, synthetic construct; identical to SEQ ID NO: 24
  misc_RNA 1..154
  source 1..154: mol_type = other DNA

SEQ ID NO: 28 - DNA, 154 bp, synthetic construct; identical to SEQ ID NO: 24
  misc_RNA 1..154
  source 1..154: mol_type = other DNA

SEQ ID NO: 29 - RNA, 180 nt, synthetic construct
  source 1..180: mol_type = other RNA
gggaaactatacaatgtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaactactacctca

SEQ ID NO: 30 - RNA, 181 nt, synthetic construct
  misc_feature 1..181: note = Synthetic sequence
  source 1..181: mol_type = other RNA
gggaagtcaaaccaagtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaaatcgaaaaccgg

SEQ ID NO: 31 - RNA, 181 nt, synthetic construct
  misc_feature 1..181: note = Synthetic sequence
  source 1..181: mol_type = other RNA
gggaagtcaaaccaagtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaaatcgaaaaccgg

SEQ ID NO: 32 - RNA, 181 nt, synthetic construct
  misc_feature 1..181: note = Synthetic sequence
  source 1..181: mol_type = other RNA
gggaagtcaaaccaagtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaaatcgaaaaccgg

SEQ ID NO: 33 - RNA, 181 nt, synthetic construct
  misc_feature 1..181: note = Synthetic sequence
  source 1..181: mol_type = other RNA
gggaagtcaaaccaagtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaaatcgaaaaccgg

SEQ ID NO: 34 - RNA, 180 nt, synthetic construct
  misc_feature 1..180: note = Synthetic sequence
  source 1..180: mol_type = other RNA
gggatttggcaatgggtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaataatgactcaa

SEQ ID NO: 35 - RNA, 180 nt, synthetic construct
  misc_feature 1..180: note = Synthetic sequence
  source 1..180: mol_type = other RNA
gggatttggcaatgggtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaataatgactcaa

SEQ ID NO: 36 - RNA, 180 nt, synthetic construct
  misc_feature 1..180: note = Synthetic sequence
  source 1..180: mol_type = other RNA
gggatttggcaatgggtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaataatgactcaa

SEQ ID NO: 37 - RNA, 180 nt, synthetic construct
  misc_feature 1..180: note = Synthetic sequence
  source 1..180: mol_type = other RNA
gggatttggcaatgggtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaataatgactcaa

SEQ ID NO: 38 - RNA, 180 nt, synthetic construct; identical to SEQ ID NO: 29
  misc_feature 1..180: note = Synthetic sequence
  source 1..180: mol_type = other RNA

SEQ ID NO: 39 - RNA, 180 nt, synthetic construct
  misc_feature 1..180: note = Synthetic sequence
  source 1..180: mol_type = other RNA
gggaaactatacaatgtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaactactacctca

SEQ ID NO: 40 - RNA, 180 nt, synthetic construct
  misc_feature 1..180: note = Synthetic sequence
  source 1..180: mol_type = other RNA
gggaaactatacaatgtatattagtatattacctggttttcgatcgaaagatcgacgaggtgaaaacctcgtgacagtccttagtcgaaagttttactagagtcagtcctgattgtgctcgaaagagcacagacctgaaaaggtctttcgtggtttattactctctgaactactacctca

SEQ ID NO: 41 - RNA, 180 nt, synthetic construct
  misc_feature 1..180: note = Synthetic sequence
  source 1..180: mol_type = other RNA
gggaaactatacaatgtatattagtttattacctggttttcgatcgaaagatcgacgaggtgaaaacctcgtgacagtccttagtcgaaagttttactagagtcagtcctgattgtgctcgaaagagcacagacctgaaaaggtctttcgtggtatattactctctgaactactacctca

SEQ ID NO: 42 - RNA, 193 nt, synthetic construct
  misc_feature 1..193: note = Synthetic sequence
  source 1..193: mol_type = other RNA
gggaagtcaaaccaagtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacatcgaaaaccgg

SEQ ID NO: 43 - RNA, 192 nt, synthetic construct
  misc_feature 1..192: note = Synthetic sequence
  source 1..192: mol_type = other RNA
gggaaactcagtaatgtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacggtaacggttt

SEQ ID NO: 44 - RNA, 190 nt, synthetic construct
  misc_feature 1..190: note = Synthetic sequence
  source 1..190: mol_type = other RNA
gggaacgtacctgtgtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacctcttccgaa

SEQ ID NO: 45 - RNA, 192 nt, synthetic construct
  misc_feature 1..192: note = Synthetic sequence
  source 1..192: mol_type = other RNA
gggagcccttatcaggtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacttctccgtcca

SEQ ID NO: 46 - RNA, 192 nt, synthetic construct
  misc_feature 1..192: note = Synthetic sequence
  source 1..192: mol_type = other RNA
gggactcctgcggcagtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacctagtacttag

SEQ ID NO: 47 - RNA, 194 nt, synthetic construct
  misc_feature 1..194: note = Synthetic sequence
  source 1..194: mol_type = other RNA
gggacccgtgaattctgtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatactccagtgccatt

SEQ ID NO: 48 - RNA, 190 nt, synthetic construct
  misc_feature 1..190: note = Synthetic sequence
  source 1..190: mol_type = other RNA
gggagcagccattagtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacgatctgtccc

SEQ ID NO: 49 - RNA, 210 nt, synthetic construct
  misc_feature 1..210: note = Synthetic sequence
  source 1..210: mol_type = other RNA
gggaattaggtttcttaatagtaagtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacgactagaattgtctactccc

SEQ ID NO: 50 - RNA, 192 nt, synthetic construct
  misc_feature 1..192: note = Synthetic sequence
  source 1..192: mol_type = other RNA
gggaaactatacaatgtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatatacctactacctca

SEQ ID NO: 51 - RNA, 190 nt, synthetic construct; identical to SEQ ID NO: 44
  misc_feature 1..190: note = Synthetic sequence
  source 1..190: mol_type = other RNA

SEQ ID NO: 52 - RNA, 184 nt, synthetic construct
  misc_feature 1..184: note = Synthetic sequence
  source 1..184: mol_type = other RNA
gggaacgtacctgttattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatactcttccgaa

SEQ ID NO: 53 - RNA, 180 nt, synthetic construct
  misc_feature 1..180: note = Synthetic sequence
  source 1..180: mol_type = other RNA
gggaacgtacctgtacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgtctcttccgaa

SEQ ID NO: 54 - RNA, 180 nt, synthetic construct
  misc_feature 1..180: note = Synthetic sequence
  source 1..180: mol_type = other RNA
gggaacgtacctgtacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgactcttccgaa

SEQ ID NO: 55 - RNA, 178 nt, synthetic construct
  misc_feature 1..178: note = Synthetic sequence
  source 1..178: mol_type = other RNA
gggaacgtacctgtacgtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacagtctcttccgaa

SEQ ID NO: 56 - RNA, 192 nt, synthetic construct; identical to SEQ ID NO: 43
  misc_feature 1..192: note = Synthetic sequence
  source 1..192: mol_type = other RNA

SEQ ID NO: 57 - RNA, 186 nt, synthetic construct
  misc_feature 1..186: note = Synthetic sequence
  source 1..186: mol_type = other RNA
gggaaactcagtaattattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataataggtaacggttt

SEQ ID NO: 58 - RNA, 182 nt, synthetic construct
  misc_feature 1..182: note = Synthetic sequence
  source 1..182: mol_type = other RNA
gggaaactcagtaatacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgtggtaacggttt

SEQ ID NO: 59 - RNA, 182 nt, synthetic construct
  misc_feature 1..182: note = Synthetic sequence
  source 1..182: mol_type = other RNA
gggaaactcagtaatacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgaggtaacggttt

SEQ ID NO: 60 - RNA, 180 nt, synthetic construct
  misc_feature 1..180: note = Synthetic sequence
  source 1..180: mol_type = other RNA
gggaaactcagtaatacgtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacagtggtaacggttt

SEQ ID NO: 61 - RNA, 190 nt, synthetic construct
  misc_feature 1..190: note = Synthetic sequence
  source 1..190: mol_type = other RNA
gggaagctgtccaagtatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatataccctgaagaag

SEQ ID NO: 62 - RNA, 184 nt, synthetic construct
  misc_feature 1..184: note = Synthetic sequence
  source 1..184: mol_type = other RNA
gggaagctgtccaatattagtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacataatacctgaagaag

SEQ ID NO: 63 - RNA, 180 nt, synthetic construct
  misc_feature 1..180: note = Synthetic sequence
  source 1..180: mol_type = other RNA
gggaagctgtccaaacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgtcctgaagaag

SEQ ID NO: 64 - RNA, 180 nt, synthetic construct
  misc_feature 1..180: note = Synthetic sequence
  source 1..180: mol_type = other RNA
gggaagctgtccaaacggtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacacgacctgaagaag

SEQ ID NO: 65 - RNA, 178 nt, synthetic construct
  misc_feature 1..178: note = Synthetic sequence
  source 1..178: mol_type = other RNA
gggaagctgtccaaacgtatattacctggttttcgatcgaaagatcgacgaggtgaaaacctcgtgacagtccttagtcgaaagttttactagagtcagtcctgattttcgaatcagagaagacgagcgtcccacgggcgcagaagagaagtcaaccagagaaacagtcctgaagaag

SEQ ID NO: 66 - RNA, 279 nt, synthetic construct
  misc_feature 1..279: note = Synthetic sequence
  source 1..279: mol_type = other RNA
gggaaactatacaatacggtatattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccttaaataggcaattgaaaaagagaagtcaaccagagaaacacgactactacctca

SEQ ID NO: 67 - RNA, 254 nt, synthetic construct
  misc_feature 1..254: note = Synthetic sequence
  source 1..254: mol_type = other RNA
gggaaactatacaatacggtatattacctggttttcgatcgaaagatcgacgaggtgaaaacctcgtgacagggcacgggcagcttgccgggttttagagctagaaatagcaagttaaaataaggctagtccgttatcaacttgaaaaagtggcaccgagtcggtgcgtcagtcctgattttcgaatcagagaagactatccaccttaaataggcaagtgagaagtcaaccagagaaacacgactactacctca

SEQ ID NO: 68 - RNA, 254 nt, synthetic construct; identical to SEQ ID NO: 67
  misc_feature 1..254: note = Synthetic sequence
  source 1..254: mol_type = other RNA

SEQ ID NO: 69 - RNA, 286 nt, synthetic construct
  misc_feature 1..286: note = Synthetic sequence
  source 1..286: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacttattttaactttctaaaacccggcaagctgcccgtgagaagtcatacgaccagagaaacacgactactacctca

SEQ ID NO: 70 - RNA, 298 nt, synthetic construct
  misc_feature 1..298: note = Synthetic sequence
  source 1..298: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacttattttaacttgctatttctagctctaaaacccggcaagctgcccgtgagaagtcatacgaccagagaaacacgactactacctca

SEQ ID NO: 71 - RNA, 307 nt, synthetic construct
  misc_feature 1..307: note = Synthetic sequence
  source 1..307: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacggactagccttattttaacttgctatttctagctctaaaacccggcaagctgcccgtgagaagtcatacgaccagagaaacacgactactacctca

SEQ ID NO: 72 - RNA, 332 nt, synthetic construct
  misc_feature 1..332: note = Synthetic sequence
  source 1..332: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacgcaccgactcggtgccactttttcaagttgataacggactagccttattttaacttgctatttctagctctaaaacccggcaagctgcccgtgagaagtcatacgaccagagaaacacgactactacctca

SEQ ID NO: 73 - RNA, 287 nt, synthetic construct
  misc_feature 1..287: note = Synthetic sequence
  source 1..287: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacttattttaactttctaaaacccggcaagctgcccgtgagaagtcatacgaccagagaaacacgatcggagtacaaa

SEQ ID NO: 74 - RNA, 299 nt, synthetic construct
  misc_feature 1..299: note = Synthetic sequence
  source 1..299: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacttattttaacttgctatttctagctctaaaacccggcaagctgcccgtgagaagtcatacgaccagagaaacacgatcggagtacaaa

SEQ ID NO: 75 - RNA, 308 nt, synthetic construct
  misc_feature 1..308: note = Synthetic sequence
  source 1..308: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacggactagccttattttaacttgctatttctagctctaaaacccggcaagctgcccgtgagaagtcatacgaccagagaaacacgatcggagtacaaa

SEQ ID NO: 76 - RNA, 333 nt, synthetic construct
  misc_feature 1..333: note = Synthetic sequence
  source 1..333: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacgcaccgactcggtgccactttttcaagttgataacggactagccttattttaacttgctatttctagctctaaaacccggcaagctgcccgtgagaagtcatacgaccagagaaacacgatcggagtacaaa

SEQ ID NO: 77 - RNA, 286 nt, synthetic construct
  misc_feature 1..286: note = Synthetic sequence
  source 1..286: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagcgaagacccatgataacttattttaactttctaaaacccggcaagctgcccgtgagaagtcatacgaccagagaaacacgactactacctca

SEQ ID NO: 78 - RNA, 298 nt, synthetic construct
  misc_feature 1..298: note = Synthetic sequence
  source 1..298: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagcgaagacccatgataacttattttaacttgctatttctagctctaaaacccggcaagctgcccgtgagaagtcatacgaccagagaaacacgactactacctca

SEQ ID NO: 79 - RNA, 286 nt, synthetic construct
  misc_feature 1..286: note = Synthetic sequence
  source 1..286: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagggaagacccatgataacttattttaactttctaaaacccggcaagctgcccgtgagaagtcatacgaccagagaaacacgactactacctca

SEQ ID NO: 80 - RNA, 298 nt, synthetic construct
  misc_feature 1..298: note = Synthetic sequence
  source 1..298: mol_type = other RNA
gggaaactatacaatacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagggaagacccatgataacttattttaacttgctatttctagctctaaaacccggcaagctgcccgtgagaagtcatacgaccagagaaacacgactactacctca

SEQ ID NO: 81 - RNA, 287 nt, synthetic construct
  misc_feature 1..287: note = Synthetic sequence
  source 1..287: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagcgaagacccatgataacttattttaactttctaaaacccggcaagctgcccgtgagaagtcatacgaccagagaaacacgatcggagtacaaa

SEQ ID NO: 82 - RNA, 299 nt, synthetic construct
  misc_feature 1..299: note = Synthetic sequence
  source 1..299: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagcgaagacccatgataacttattttaacttgctatttctagctctaaaacccggcaagctgcccgtgagaagtcatacgaccagagaaacacgatcggagtacaaa

SEQ ID NO: 83 - RNA, 287 nt, synthetic construct
  misc_feature 1..287: note = Synthetic sequence
  source 1..287: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagggaagacccatgataacttattttaactttctaaaacccggcaagctgcccgtgagaagtcatacgaccagagaaacacgatcggagtacaaa

SEQ ID NO: 84 - RNA, 299 nt, synthetic construct
  misc_feature 1..299: note = Synthetic sequence
  source 1..299: mol_type = other RNA
gggatctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagggaagacccatgataacttattttaacttgctatttctagctctaaaacccggcaagctgcccgtgagaagtcatacgaccagagaaacacgatcggagtacaaa

SEQ ID NO: 85 - RNA, 245 nt, synthetic construct
  misc_feature 1..245: note = Synthetic sequence
  source 1..245: mol_type = other RNA
gggaaactatacaatacggtatattacctggttttcgatcgaaagatcgacgaggtgaaaacctcgtgacagtctgatagcaatgtcagcagtgcctgcaagctgaccctgaagttcaccacctgaacttcagggtcagcttgcaagtaaggttgaccatactctacagtcctgattttcgaatcagagaagtatctgatgatttagctcaagaagtcaaccagagaaacacgactactacctca

SEQ ID NO: 86 - RNA, 253 nt, synthetic construct
  misc_feature 1..253: note = Synthetic sequence
  source 1..253: mol_type = other RNA
gggaaactatacaatacggtatattacctggttttcgatcgaaagatcgacgaggtgaaaacctcgcgtatgacagtctgatagcaatgtcagcagtgcctgcaagctgaccctgaagttcaccacctgaacttcagggtcagcttgcaagtaaggttgaccatactctacagtcctgattttcgaatcagagaagtatctgatgatttagctcaagaagtcatacgaccagagaaacacgactactacctca

SEQ ID NO: 87 - RNA, 245 nt, synthetic construct
  misc_feature 1..245: note = Synthetic sequence
  source 1..245: mol_type = other RNA
gggaaactatacaatacggtttattacctggttttcgatcgaaagatcgacgaggtgaaaacctcgtgacagtctgatagcaatgtcagcagtgcctgcaagctgaccctgaagttcaccacctgaacttcagggtcagcttgcaagtaaggttgaccatactctacagtcctgattttcgaatcagagaagtatctgatgatttagctcaagaagtcaaccagagaaacacgactactacctca

SEQ ID NO: 88 - RNA, 245 nt, synthetic construct
  misc_feature 1..245: note = Synthetic sequence
  source 1..245: mol_type = other RNA
gggaaactatacaatacggtatattacctggttttcgatcgaaagatcgacgaggtgaaaacctcgtgacagtctgatagcaatgtcagcagtgcctgcaagctgcaactgaagttcaccacctgaacttcagttgcagcttgcaagtaaggttgaccatactctacagtcctgattttcgaatcagagaagtatctgatgatttagctcaagaagtcaaccagagaaacacgactactacctca

SEQ ID NO: 89 - RNA, 246 nt, synthetic construct
  misc_feature 1..246: note = Synthetic sequence
  source 1..246: mol_type = other RNA
gggatctgaatggcaacggtatattacctggttttcgatcgaaagatcgacgaggtgaaaacctcgtgacagtctgatagcaatgtcagcagtgcctgcaagctgaccctgaagttcaccacctgaacttcagggtcagcttgcaagtaaggttgaccatactctacagtcctgattttcgaatcagagaagtatctgatgatttagctcaagaagtcaaccagagaaacacgatcggagtacaaa

SEQ ID NO: 90 - RNA, 246 nt, synthetic construct
  misc_feature 1..246: note = Synthetic sequence
  source 1..246: mol_type = other RNA
gggatctgaatggcaacggtttattacctggttttcgatcgaaagatcgacgaggtgaaaacctcgtgacagtctgatagcaatgtcagcagtgcctgcaagctgaccctgaagttcaccacctgaacttcagggtcagcttgcaagtaaggttgaccatactctacagtcctgattttcgaatcagagaagtatctgatgatttagctcaagaagtcaaccagagaaacacgatcggagtacaaa

SEQ ID NO: 91 - RNA, 246 nt, synthetic construct
  misc_feature 1..246: note = Synthetic sequence
  source 1..246: mol_type = other RNA
gggatctgaatggcaacggtatattacctggttttcgatcgaaagatcgacgaggtgaaaacctcgtgacagtctgatagcaatgtcagcagtgcctgcaagctgcaactgaagttcaccacctgaacttcagttgcagcttgcaagtaaggttgaccatactctacagtcctgattttcgaatcagagaagtatctgatgatttagctcaagaagtcaaccagagaaacacgatcggagtacaaa

SEQ ID NO: 92 - RNA, 226 nt, synthetic construct
  misc_feature 1..226: note = Synthetic sequence
  source 1..226: mol_type = other RNA
gggaaactatacaatacggtatattacctggttttcgatcgaaagatcgacgaggtgaaaacctcgtgacagtccgagacggtcgggtccagtcccaacgatgttggctgttgagtagtgtgtgggctccagtcctgattttcgaatcagagaagaccccaacctatccccttaaataggcaattgaaaaagagaagtcaaccagagaaacacgactactacctca

SEQ ID NO: 93 - RNA, 250 nt, synthetic construct
  misc_feature 1..250: note = Synthetic sequence
  source 1..250: mol_type = other RNA
gggaaactatacaatacggtatattacctggttttcgatcgaaagatcgacgaggtgaaaacctcgtgacagtctgatagcaatgtcagcagtgcctgcaagctgaccctgaagttcatctgcagatgaacttcagggtcagcttgcaagtaaggttgaccatactctacagtcctgattttcgaatcagagaagtatctgatgtaatttagctcaagaagtcaaccagagaaacacgactactacctca

SEQ ID NO: 94 - RNA, 247 nt, synthetic construct
  misc_feature 1..247: note = Synthetic sequence
  source 1..247: mol_type = other RNA
gggaaactatacaatacggtatattacctggttttcgatcgaaagatcgacgaggtgaaaacctcgtgacagtctgatagcaatgtcagcagtgcctgcaagctgaccctgaagttcaccacctgaacttcagggtcagcttgcaagtaaggttgaccatactctacagtcctgattttcgaatcagagaagtatctgatgtaatttagctcaagaagtcaaccagagaaacacgactactacctca

SEQ ID NO: 95 - RNA, 247 nt, synthetic construct
  misc_feature 1..247: note = Synthetic sequence
  source 1..247: mol_type = other RNA
gggaaactatacaatacggtatattacctggttttcgatcgaaagatcgacgaggtgaaaacctcgtgacagtctgatagcaatgtcaacctcgcctgcaagctgaccctgaagttcaccacctgaacttcagggtcagcttgcaggcgcggttgaccataccgtccagtcctgattttcgaatcagagaaggaattgatgtaatttagctcaagaagtcaaccagagaaacacgactactacctca

SEQ ID NO: 96 - RNA, 247 nt, synthetic construct
  misc_feature 1..247: note = Synthetic sequence
  source 1..247: mol_type = other RNA
gggaaactatacaatacggtatattacctggttttcgatcgaaagatcgacgaggtgaaaacctcgtgacagtctgatagcaatgtcaacctcgcctgcaagctgcaactgaagttcaccacctgaacttcagggtcagcttgcaggcgcggttgaccataccgtccagtcctgattttcgaatcagagaaggaattgatgtaatttagctcaagaagtcaaccagagaaacacgactactacctca

SEQ ID NO: 97 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgctagcaatgtcaacctcgcctgcaagctgaccctgaagttcatgtgccacctctaatgaacttcagggtcagcttgcaggcgcggttgaccataccgtccagtcctgattttcgaatcagagaaggaattgatgtaatttagcgcaagaagtcaaccagagaaacacgactactacctca

SEQ ID NO: 98 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgctagcaatgtcaacctcgcctgcaagctgaccctgaagttcatgtgttattcttgatgaacttcagggtcagcttgcaggcgcggttgaccataccgtccagtcctgattttcgaatcagagaaggaattgatgtaatttagcgcaagaagtcaaccagagaaacacgactactacctca

SEQ ID NO: 99 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgtaatttagctcaagaagtcaaccagagaaacacgactactacctca

SEQ ID NO: 100 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctccttgcagatgaacttcagggtcagctgtgttattcttgagctgaccctgaagttcatctgcaaggcggttgaccataccgtccagtcctgattttcgaatcagagaaggaattgatgtaatttagctcaagaagtcaaccagagaaacacgactactacctca

SEQ ID NO: 101 - RNA, 270 nt, synthetic construct
  misc_feature 1..270: note = Synthetic sequence
  source 1..270: mol_type = other RNA
gggaaactatacaatacggtatattacctggttttcgatcgaaagatcgacgaggtgaaaacctcgtgacagtccagccagcttattgcgaatctcgatgcagatgaacttcagggtcagctgtgttattcttgagctgaccctgaagttcatctgcatcgcgattcgcttattcgtctttgcagtcctgattttcgaatcagagaagcacctacgcctaataaatccgctttctgagaagtcaaccagagaaacacgactactacctca

SEQ ID NO: 102 - RNA, 271 nt, synthetic construct
  misc_feature 1..271: note = Synthetic sequence
  source 1..271: mol_type = other RNA
gggaaactatacaatacggtatattacctggttttcgatcgaaagatcgacgaggtgaaaacctcgtgacagtctgtatcaattcttgtgtatctcgctgcagatgaacttcagggtcagctgtgttattcttgagctgaccctgaagttcatctgcagcgcggtacaccttattcgtccaatcagtcctgattttcgaatcagagaagatgttacgcctaataaagccttgcgacaagaagtcaaccagagaaacacgactactacctca

SEQ ID NO: 103 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgccttggtgcagatgaacttcagggtgtgttattcttgaccctgaagttcatctgcaccaaggcgcggttgaccataccgtccagtcctgattttcgaatcagagaaggaattgatgtaatttagctcaagaagtcaaccagagaaacacgactactacctca

SEQ ID NO: 104 - RNA, 266 nt, synthetic construct
  misc_feature 1..266: note = Synthetic sequence
  source 1..266: mol_type = other RNA
gggaaactatacaatacggtatattacctggttttcgatcgaaagatcgacgaggtgaaaacctcgtgacagtccagccagcttattgcgaatctcgatggtgcagatgaacttcagggtgtgttattcttgaccctgaagttcatctgcaccatcgcgattcgcttattcgtctttgcagtcctgattttcgaatcagagaagcacctacgcctaataaatccgctttctgagaagtcaaccagagaaacacgactactacctca

SEQ ID NO: 105 - RNA, 271 nt, synthetic construct
  misc_feature 1..271: note = Synthetic sequence
  source 1..271: mol_type = other RNA
gggaaactatacaatacggtatattacctggttttcgatcgaaagatcgacgaggtgaaaacctcgtgacagtctgtatcaattcttgtgtatctcgccgtggtgcagatgaacttcagggtgtgttattcttgaccctgaagttcatctgcaccatggcgcggtacaccttattcgtccaatcagtcctgattttcgaatcagagaagatgttacgcctaataaagccttgcgacaagaagtcaaccagagaaacacgactactacctca

SEQ ID NO: 106 - RNA, 279 nt, synthetic construct
  misc_feature 1..279: note = Synthetic sequence
  source 1..279: mol_type = other RNA
gggaaactatacaatacggtatattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccgtaaagaggccattgcaaaagagaagtcaaccagagaaacacgactactacctca

SEQ ID NO: 107 - RNA, 279 nt, synthetic construct
  misc_feature 1..279: note = Synthetic sequence
  source 1..279: mol_type = other RNA
gggaaactatacaatacggtttattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccgtaaagaggccattgcaaaagagaagtcaaccagagaaagacgactactacctca

SEQ ID NO: 108 - RNA, 279 nt, synthetic construct
  misc_feature 1..279: note = Synthetic sequence
  source 1..279: mol_type = other RNA
gggaaactatacaatacggtatattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccggaaagcggcccttgccaaagagaagtcaaccagagaaacacgactactacctca

SEQ ID NO: 109 - RNA, 279 nt, synthetic construct
  misc_feature 1..279: note = Synthetic sequence
  source 1..279: mol_type = other RNA
gggaaactatacaatacggtttattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccggaaagcggcccttgccaaagagaagtcaaccagagaaagacgactactacctca

SEQ ID NO: 110 - RNA, 279 nt, synthetic construct
  misc_feature 1..279: note = Synthetic sequence
  source 1..279: mol_type = other RNA
gggaaactatacaatacggtttattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccggcaagctgcccgtgcccaagagaagtcaaccagagaaagacgactactacctca

SEQ ID NO: 111 - RNA, 279 nt, synthetic construct
  misc_feature 1..279: note = Synthetic sequence
  source 1..279: mol_type = other RNA
gggaaactatacaatacggtatattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagacaaaaaaatatccccggcaagctgcccgtgcccaagagaagtcaaccagagaaacacgactactacctca

SEQ ID NO: 112 - RNA, 279 nt, synthetic construct
  misc_feature 1..279: note = Synthetic sequence
  source 1..279: mol_type = other RNA
gggaaactatacaatacggtttattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagacaaaaaaatatccccggcaagctgcccgtgcccaagagaagtcaaccagagaaagacgactactacctca

SEQ ID NO: 113 - RNA, 279 nt, synthetic construct
  misc_feature 1..279: note = Synthetic sequence
  source 1..279: mol_type = other RNA
gggaaactatacaatacggtatattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccggcaagctgcccgtgcccaagagaagtcaaccagagaaacacgactactacctca

SEQ ID NO: 114 - RNA, 286 nt, synthetic construct; identical to SEQ ID NO: 69
  misc_feature 1..286: note = Synthetic sequence
  source 1..286: mol_type = other RNA

SEQ ID NO: 115 - RNA, 279 nt, synthetic construct
  misc_feature 1..279: note = Synthetic sequence
  source 1..279: mol_type = other RNA
gggaaactatacaatacggtttattacctggttttcgatcgaaagatcgacgaggtgaaaacctcgtgacagtccttgggcacgggcagcttgccgggttttagagctagaaatagcaagttaaaataaggctagtccgttatcaacttgaaaaagtggcaccgagtcggtgctttttttgtcagtcctgattttcgaatcagagaagaccccaacctatccccggcaagctgcccgtgcccaagagaagtcaaccagagaaacacgactactacctca

SEQ ID NO: 116 - RNA, 286 nt, synthetic construct
  misc_feature 1..286: note = Synthetic sequence
  source 1..286: mol_type = other RNA
gggaaactatacaatacggtttattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccatgataacttattttaactttctaaaacccggcaagctgcccgtgagaagtcatacgaccagagaaacacgactactacctca

SEQ ID NO: 117 - RNA, 256 nt, synthetic construct; identical to SEQ ID NO: 99
  misc_feature 1..256: note = Synthetic sequence
  source 1..256: mol_type = other RNA

SEQ ID NO: 118 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttactggtcagcttgcgtgttattcttggcaagctgaccagtaagttcataggcgcggttgaccataccgtccagtcctgattttcgaatcagagaaggaattgatgtaatttagctcaagaagtcaaccagagaaacacgactactacctca

SEQ ID NO: 119 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgtaatttagctcaagaagtcaaccagagaaacacgactactacctca

SEQ ID NO: 120 - RNA, 254 nt, synthetic construct
  misc_feature 1..254: note = Synthetic sequence
  source 1..254: mol_type = other RNA
gggaacgtacctgtacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgtaatttagctcaagaagtcaaccagagaaacacgactcttccgaa

SEQ ID NO: 121 - RNA, 254 nt, synthetic construct
  misc_feature 1..254: note = Synthetic sequence
  source 1..254: mol_type = other RNA
gggaacgtacctgtacggtatattacctggttttcgatcgaaagatcgacgaggtgaaaacctcgtgacagtctgatagcaatgtcaacctcgcctatgaacttactggtcagcttgcgtgttattcttggcaagctgaccagtaagttcataggcgcggttgaccataccgtccagtcctgattttcgaatcagagaaggaattgatgtaatttagctcaagaagtcaaccagagaaacacgactcttccgaa

SEQ ID NO: 122 - RNA, 254 nt, synthetic construct
  misc_feature 1..254: note = Synthetic sequence
  source 1..254: mol_type = other RNA
gggaacgtacctgtacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgtaatttagctcaagaagtcaaccagagaaacacgactcttccgaa

SEQ ID NO: 123 - RNA, 254 nt, synthetic construct
  misc_feature 1..254: note = Synthetic sequence
  source 1..254: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgatttagctcaagaagtcaaccagagaaacacgactactacctca

SEQ ID NO: 124 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactactacctca

SEQ ID NO: 125 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacgtgcgttaatttctctcaagaagtcaaccagagaaacacgactactacctca

SEQ ID NO: 126 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaatgacgtgatgtaatttatatccagaagtcaaccagagaaacacgactactacctca

SEQ ID NO: 127 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaatgacgtgatgtaattgctatcaagaagtcaaccagagaaacacgactactacctca

SEQ ID NO: 128 - RNA, 256 nt, synthetic construct
  misc_feature 1..256: note = Synthetic sequence
  source 1..256: mol_type = other RNA
gggaaactatacaatacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaatttagctcaagaagtcaaccagagaaacacgactactacctca

SEQ ID NO: 129 - RNA, 254 nt, synthetic construct
  misc_feature 1..254: note = Synthetic sequence
  source 1..254: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggaattgatgatttagctcaagaagtcaaccagagaaacacgactactacctca

SEQ ID NO: 130 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactactacctca

SEQ ID NO: 131 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttactggtcagcttgcgtgttattcttggcaagctgaccagtaagttcataggcgcggttgaccataccgtccagtcctgattttcgaatcagagaaggacggtatgtaattgctatcaagaagtcaaccagagaaacacgactactacctca

SEQ ID NO: 132 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacgtgcgttaatttctctcaagaagtcaaccagagaaacacgactactacctca

SEQ ID NO: 133 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaatgacgtgatgtaatttatatccagaagtcaaccagagaaacacgactactacctca

SEQ ID NO: 134 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaatgacgtgatgtaattgctatcaagaagtcaaccagagaaacacgactactacctca

SEQ ID NO: 135 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaatttagctcaagaagtcaaccagagaaacacgactactacctca

SEQ ID NO: 136 - RNA, 254 nt, synthetic construct
  misc_feature 1..254: note = Synthetic sequence
  source 1..254: mol_type = other RNA
gggaacgtacctgtacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactcttccgaa

SEQ ID NO: 137 - RNA, 254 nt, synthetic construct
  misc_feature 1..254: note = Synthetic sequence
  source 1..254: mol_type = other RNA
gggaacgtacctgtacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactcttccgaa

SEQ ID NO: 138 - RNA, 255 nt, synthetic construct
  misc_feature 1..255: note = Synthetic sequence
  source 1..255: mol_type = other RNA
gggaacccagtagcacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgacagatgtagct

SEQ ID NO: 139 - RNA, 255 nt, synthetic construct
  misc_feature 1..255: note = Synthetic sequence
  source 1..255: mol_type = other RNA
gggaacccagtagcacggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgacagatgtagct

SEQ ID NO: 140 - RNA, 255 nt, synthetic construct
  misc_feature 1..255: note = Synthetic sequence
  source 1..255: mol_type = other RNA
gggaacccagtagcacggtatattacctggttttcgatcgaaagatcgacgaggtgaaaacctcgtgacagtctgatagcaatgtcaacctcgcctatgaacttactggtcagcttgcgtgttattcttggcaagctgaccagtaagttcataggcgcggttgaccataccgtccagtcctgattttcgaatcagagaaggacggtatgtaattgctatcaagaagtcaaccagagaaacacgacagatgtagct

SEQ ID NO: 141 - RNA, 256 nt, synthetic construct
  misc_feature 1..256: note = Synthetic sequence
  source 1..256: mol_type = other RNA
gggaaactatacaatacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgactactacctca

SEQ ID NO: 142 - RNA, 256 nt, synthetic construct
  misc_feature 1..256: note = Synthetic sequence
  source 1..256: mol_type = other RNA
gggaaactatacaatatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactactacctca

SEQ ID NO: 143 - RNA, 256 nt, synthetic construct
  misc_feature 1..256: note = Synthetic sequence
  source 1..256: mol_type = other RNA
gggaaactatacaatatggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactactacctca

SEQ ID NO: 144 - RNA, 256 nt, synthetic construct
  misc_feature 1..256: note = Synthetic sequence
  source 1..256: mol_type = other RNA
gggaaactatacaatatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgactactacctca

SEQ ID NO: 145 - RNA, 254 nt, synthetic construct
  misc_feature 1..254: note = Synthetic sequence
  source 1..254: mol_type = other RNA
gggaaactatacaatcggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgctactacctca

SEQ ID NO: 146 - RNA, 256 nt, synthetic construct
  misc_feature 1..256: note = Synthetic sequence
  source 1..256: mol_type = other RNA
gggaaactatacaatttggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatggctactacctca

SEQ ID NO: 147 - RNA, 256 nt, synthetic construct
  misc_feature 1..256: note = Synthetic sequence
  source 1..256: mol_type = other RNA
gggaaactatacaattatgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaatactactacctca

SEQ ID NO: 148 - RNA, 256 nt, synthetic construct
  misc_feature 1..256: note = Synthetic sequence
  source 1..256: mol_type = other RNA
gggaaactatacaatactgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagactactacctca

SEQ ID NO: 149 - RNA, 256 nt, synthetic construct
  misc_feature 1..256: note = Synthetic sequence
  source 1..256: mol_type = other RNA
gggaaactatacaatatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacaactactacctca

SEQ ID NO: 150 - RNA, 254 nt, synthetic construct
  misc_feature 1..254: note = Synthetic sequence
  source 1..254: mol_type = other RNA
gggaaactatacaatctgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagctactacctca

SEQ ID NO: 151 - RNA, 254 nt, synthetic construct
  misc_feature 1..254: note = Synthetic sequence
  source 1..254: mol_type = other RNA
gggaaactatacaattggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacactactacctca

SEQ ID NO: 152 - RNA, 254 nt, synthetic construct
  misc_feature 1..254: note = Synthetic sequence
  source 1..254: mol_type = other RNA
gggaacgtacctgtacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgactcttccgaa

SEQ ID NO: 153 - RNA, 254 nt, synthetic construct
  misc_feature 1..254: note = Synthetic sequence
  source 1..254: mol_type = other RNA
gggaacgtacctgtatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactcttccgaa

SEQ ID NO: 154 - RNA, 254 nt, synthetic construct
  misc_feature 1..254: note = Synthetic sequence
  source 1..254: mol_type = other RNA
gggaacgtacctgtatggtttattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgactcttccgaa

SEQ ID NO: 155 - RNA, 254 nt, synthetic construct
  misc_feature 1..254: note = Synthetic sequence
  source 1..254: mol_type = other RNA
gggaacgtacctgtatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgactcttccgaa

SEQ ID NO: 156 - RNA, 252 nt, synthetic construct
  misc_feature 1..252: note = Synthetic sequence
  source 1..252: mol_type = other RNA
gggaacgtacctgtcggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgctcttccgaa

SEQ ID NO: 157 - RNA, 254 nt, synthetic construct
  misc_feature 1..254: note = Synthetic sequence
  source 1..254: mol_type = other RNA
gggaacgtacctgtttggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatggctcttccgaa

SEQ ID NO: 158 - RNA, 254 nt, synthetic construct
  misc_feature 1..254: note = Synthetic sequence
  source 1..254: mol_type = other RNA
gggaacgtacctgttatgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaatactcttccgaa

SEQ ID NO: 159 - RNA, 254 nt, synthetic construct
  misc_feature 1..254: note = Synthetic sequence
  source 1..254: mol_type = other RNA
gggaacgtacctgtactgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagactcttccgaa

SEQ ID NO: 160 - RNA, 254 nt, synthetic construct
  misc_feature 1..254: note = Synthetic sequence
  source 1..254: mol_type = other RNA
gggaacgtacctgtatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacaactcttccgaa

SEQ ID NO: 161 - RNA, 252 nt, synthetic construct
  misc_feature 1..252: note = Synthetic sequence
  source 1..252: mol_type = other RNA
gggaacgtacctgtctgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagctcttccgaa

SEQ ID NO: 162 - RNA, 252 nt, synthetic construct
  misc_feature 1..252: note = Synthetic sequence
  source 1..252: mol_type = other RNA
gggaacgtacctgttggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacactcttccgaa

SEQ ID NO: 163 - RNA, 255 nt, synthetic construct
  misc_feature 1..255: note = Synthetic sequence
  source 1..255: mol_type = other RNA
gggaacccagtagcacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgacagatgtagct

SEQ ID NO: 164 - RNA, 255 nt, synthetic construct
  misc_feature 1..255: note = Synthetic sequence
  source 1..255: mol_type = other RNA
gggaacccagtagcatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgacagatgtagct

SEQ ID NO: 165 - RNA, 255 nt, synthetic construct
  misc_feature 1..255: note = Synthetic sequence
  source 1..255: mol_type = other RNA
gggaacccagtagcatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgacagatgtagct

SEQ ID NO: 166 - RNA, 253 nt, synthetic construct
  misc_feature 1..253: note = Synthetic sequence
  source 1..253: mol_type = other RNA
gggaacccagtagccggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgcagatgtagct

SEQ ID NO: 167 - RNA, 255 nt, synthetic construct
  misc_feature 1..255: note = Synthetic sequence
  source 1..255: mol_type = other RNA
gggaacccagtagcttggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatggcagatgtagct

SEQ ID NO: 168 - RNA, 255 nt, synthetic construct
  misc_feature 1..255: note = Synthetic sequence
  source 1..255: mol_type = other RNA
gggaacccagtagctatgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaatacagatgtagct

SEQ ID NO: 169 - RNA, 255 nt, synthetic construct
  misc_feature 1..255: note = Synthetic sequence
  source 1..255: mol_type = other RNA
gggaacccagtagcactgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagacagatgtagct

SEQ ID NO: 170 - RNA, 255 nt, synthetic construct
  misc_feature 1..255: note = Synthetic sequence
  source 1..255: mol_type = other RNA
gggaacccagtagcatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacaacagatgtagct

SEQ ID NO: 171 - RNA, 253 nt, synthetic construct
  misc_feature 1..253: note = Synthetic sequence
  source 1..253: mol_type = other RNA
gggaacccagtagcctgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagcagatgtagct

SEQ ID NO: 172 - RNA, 253 nt, synthetic construct
  misc_feature 1..253: note = Synthetic sequence
  source 1..253: mol_type = other RNA
gggaacccagtagctggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacacagatgtagct

SEQ ID NO: 173 - RNA, 257 nt, synthetic construct
  misc_feature 1..257: note = Synthetic sequence
  source 1..257: mol_type = other RNA
gggatctgaatggcaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgatcggagtacaaa

SEQ ID NO: 174 - RNA, 257 nt, synthetic construct
  misc_feature 1..257: note = Synthetic sequence
  source 1..257: mol_type = other RNA
gggatctgaatggcaatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgatcggagtacaaa

SEQ ID NO: 175 - RNA, 257 nt, synthetic construct
  misc_feature 1..257: note = Synthetic sequence
  source 1..257: mol_type = other RNA
gggatctgaatggcaatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatgatcggagtacaaa

SEQ ID NO: 176 - RNA, 255 nt, synthetic construct
  misc_feature 1..255: note = Synthetic sequence
  source 1..255: mol_type = other RNA
gggatctgaatggcacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgtcggagtacaaa

SEQ ID NO: 177 - RNA, 257 nt, synthetic construct
  misc_feature 1..257: note = Synthetic sequence
  source 1..257: mol_type = other RNA
gggatctgaatggcattggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacatggtcggagtacaaa

SEQ ID NO: 178 - RNA, 257 nt, synthetic construct
  misc_feature 1..257: note = Synthetic sequence
  source 1..257: mol_type = other RNA
gggatctgaatggcatatgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaatatcggagtacaaa

SEQ ID NO: 179 - RNA, 257 nt, synthetic construct
  misc_feature 1..257: note = Synthetic sequ ence
  source 1..257: mol_type = other RNA
gggatctgaatggcaactgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagatcggagtacaaa

SEQ ID NO: 180 - RNA, 257 nt, synthetic construct
  misc_feature 1..257: note = Synthetic sequence
  source 1..257: mol_type = other RNA
gggatctgaatggcaatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacaatcggagtacaaa

SEQ ID NO: 181 - RNA, 255 nt, synthetic construct
  misc_feature 1..255: note = Synthetic sequence
  source 1..255: mol_type = other RNA
gggatctgaatggcactgtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacaagtcggagtacaaa

SEQ ID NO: 182 - RNA, 255 nt, synthetic construct
  misc_feature 1..255: note = Synthetic sequence
  source 1..255: mol_type = other RNA
gggatctgaatggcatggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacatcggagtacaaa

SEQ ID NO: 183 - RNA, 254 nt, synthetic construct
  misc_feature 1..254: note = Synthetic sequence
  source 1..254: mol_type = other RNA
gggagttaacaataacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgattgcagcagt

SEQ ID NO: 184 - RNA, 254 nt, synthetic construct
  misc_feature 1..254: note = Synthetic sequence
  source 1..254: mol_type = other RNA
gggagaagaattcaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgagatttttaac

SEQ ID NO: 185 - RNA, 254 nt, synthetic construct
  misc_feature 1..254: note = Synthetic sequence
  source 1..254: mol_type = other RNA
gggatcagattttaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgattgcagcagt

SEQ ID NO: 186 - RNA, 254 nt, synthetic construct
  misc_feature 1..254: note = Synthetic sequence
  source 1..254: mol_type = other RNA
gggaagtaaggatgacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgagctagtgtaa

SEQ ID NO: 187 - RNA, 254 nt, synthetic construct
  misc_feature 1..254: note = Synthetic sequence
  source 1..254: mol_type = other RNA
gggaccgaaacgaaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgatgagtacatg

SEQ ID NO: 188 - RNA, 254 nt, synthetic construct
  misc_feature 1..254: note = Synthetic sequence
  source 1..254: mol_type = other RNA
gggaaagtacgctaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgattaactatta

SEQ ID NO: 189 - RNA, 254 nt, synthetic construct
  misc_feature 1..254: note = Synthetic sequence
  source 1..254: mol_type = other RNA
gggaagtgtaactaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgagcaagaatac

SEQ ID NO: 190 - RNA, 254 nt, synthetic construct
  misc_feature 1..254: note = Synthetic sequence
  source 1..254: mol_type = other RNA
gggatacaagactcacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgaacgttaacaa

SEQ ID NO: 191 - RNA, 254 nt, synthetic construct
  misc_feature 1..254: note = Synthetic sequence
  source 1..254: mol_type = other RNA
gggaagtaaacgtaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgaaaaagaaggt

SEQ ID NO: 192 - RNA, 254 nt, synthetic construct
  misc_feature 1..254: note = Synthetic sequence
  source 1..254: mol_type = other RNA
gggagaactctagaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgaagaattcaga

SEQ ID NO: 193 - RNA, 254 nt, synthetic construct
  misc_feature 1..254: note = Synthetic sequence
  source 1..254: mol_type = other RNA
gggagaccagaagaacggtatattacctggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagagaaacacgatcaggaactc

SEQ ID NO: 194 - RNA, 258 nt, synthetic construct
  misc_feature 1..258: note = Synthetic sequence
  source 1..258: mol_type = other RNA
gggaaactatacaatacggtatattacctggttttcgatcgaaagatcgacgaggtgaaaacctcgatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataccagagaaacacgactactacctca

SEQ ID NO: 195 - RNA, 258 nt, synthetic construct
  misc_feature 1..258: note = Synthetic sequence
  source 1..258: mol_type = other RNA
gggaaactatacaatacggtatattacctggttttcgatcgaaagatcgacgaggtgaaaacctcgatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcataccagagaaacacgactactacctca

SEQ ID NO: 196 - RNA, 262 nt, synthetic construct
  misc_feature 1..262: note = Synthetic sequence
  source 1..262: mol_type = other RNA
gggaaactatacaatacggtatattacctggttttcgatcgaaagatcgacgaggtgaaaacctcggtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcatacaccagagaaacacgactactacctca

SEQ ID NO: 197 - RNA, 262 nt, synthetic construct
  misc_feature 1..262: note = Synthetic sequence
  source 1..262: mol_type = other RNA
gggaaactatacaatacggtatattacctggttttcgatcgaaagatcgacgaggtgaaaacctcggtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcatacaccagagaaacacgactactacctca

SEQ ID NO: 198 - RNA, 264 nt, synthetic construct
  misc_feature 1..264: note = Synthetic sequence
  source 1..264: mol_type = other RNA
gggaaactatacaatacggtatattacctggttttcgatcgaaagatcgacgaggtgaaaacctcgcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcatacgaccagagaaacacgactactacctca

SEQ ID NO: 199 - RNA, 264 nt, synthetic construct
  misc_feature 1..264: note = Synthetic sequence
  source 1..264: mol_type = other RNA
gggaaactatacaatacggtatattacctggttttcgatcgaaagatcgacgaggtgaaaacctcgcgtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcatacgaccagagaaacacgactactacctca

SEQ ID NO: 200 - RNA, 266 nt, synthetic construct
  misc_feature 1..266: note = Synthetic sequence
  source 1..266: mol_type = other RNA
gggaaactatacaatacggtatattacctggttttcgatcgaaagatcgacgaggtgaaaacctcgacgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcatacgtaccagagaaacacgactactacctca

SEQ ID NO: 201 - RNA, 266 nt, synthetic construct
  misc_feature 1..266: note = Synthetic sequence
  source 1..266: mol_type = other RNA
gggaaactatacaatacggtatattacctggttttcgatcgaaagatcgacgaggtgaaaacctcgacgtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcatacgtaccagagaaacacgactactacctca

SEQ ID NO: 202 - RNA, 268 nt, synthetic construct
  misc_feature 1..268: note = Synthetic sequence
  source 1..268: mol_type = other RNA
gggaaactatacaatacggtatattacctggttttcgatcgaaagatcgacgaggtgaaaacctcgcacgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcatacgtgaccagagaaacacgactactacctca

SEQ ID NO: 203 - RNA, 268 nt, synthetic construct
  misc_feature 1..268: note = Synthetic sequence
  source 1..268: mol_type = other RNA
gggaaactatacaatacggtatattacctggttttcgatcgaaagatcgacgaggtgaaaacctcgcacgtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcatacgtgaccagagaaacacgactactacctca

SEQ ID NO: 204 - RNA, 260 nt, synthetic construct
  misc_feature 1..260: note = Synthetic sequence
  source 1..260: mol_type = other RNA
gggaaactatacaatacggtatattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactactacctca

SEQ ID NO: 205 - RNA, 260 nt, synthetic construct
  misc_feature 1..260: note = Synthetic sequence
  source 1..260: mol_type = other RNA
gggaaactatacaatacggtatattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcataaccagagaaacacgactactacctca

SEQ ID NO: 206 - RNA, 260 nt, synthetic construct
  misc_feature 1..260: note = Synthetic sequence
  source 1..260: mol_type = other RNA
gggaaactatacaatacggtatattacctggttttcgatcgaaagatcgacgaggtgaaaacctcgtatgacactctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaacaagtcataaccagagaaacacgactactacctca

SEQ ID NO: 207 - RNA, 260 nt, synthetic construct
  misc_feature 1..260: note = Synthetic sequence
  source 1..260: mol_type = other RNA
gggaaactatacaatacggtatattacctggttttcgatcgaaagatcgacgaggtgaaaacctcgtatgacactctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaacaagtcataaccagagaaacacgactactacctca

SEQ ID NO: 208 - RNA, 260 nt, synthetic construct
  misc_feature 1..260: note = Synthetic sequence
  source 1..260: mol_type = other RNA
gggaaactatacaatacggtttattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactactacctca

SEQ ID NO: 209 - RNA, 258 nt, synthetic construct
  misc_feature 1..258: note = Synthetic sequence
  source 1..258: mol_type = other RNA
gggaacgtacctgtacggtatattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactcttccgaa

SEQ ID NO: 210 - RNA, 258 nt, synthetic construct
  misc_feature 1..258: note = Synthetic sequence
  source 1..258: mol_type = other RNA
gggaacgtacctgtacggtttattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactcttccgaa

SEQ ID NO: 211 - RNA, 260 nt, synthetic construct
  misc_feature 1..260: note = Synthetic sequence
  source 1..260: mol_type = other RNA
gggaaactatacaatatggtatattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactactacctca

SEQ ID NO: 212 - RNA, 260 nt, synthetic construct
  misc_feature 1..260: note = Synthetic seqyuence
  source 1..260: mol_type = other RNA
gggaaactatacaatatggtttattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcataaccagagaaacacgactactacctca

SEQ ID NO: 213 - RNA, 260 nt, synthetic construct
  misc_feature 1..260: note = Synthetic sequence
  source 1..260: mol_type = other RNA
gggaaactatacaatatggtatattacctggttttcgatcgaaagatcgacgaggtgaaaacctcgtatgacagtctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaagaagtcataaccagagaaacacgactactacctca

SEQ ID NO: 214 - RNA, 260 nt, synthetic construct
  misc_feature 1..260: note = Synthetic sequence
  source 1..260: mol_type = other RNA
gggaaactatacaatatggtatattacctggttttcgatcgaaagatcgacgaggtgaaaacctcgtatgacactctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaacaagtcataaccagagaaacacgactactacctca

SEQ ID NO: 215 - RNA, 260 nt, synthetic construct
  misc_feature 1..260: note = Synthetic sequence
  source 1..260: mol_type = other RNA
gggaaactatacaatatggtatattacctggttttcgatcgaaagatcgacgaggtgaaaacctcgtatgacactctgatagcaatgtcaacctcgcctatgaacttcagggtcagcttgcgtgttattcttggcaagctgaccctgaagttcataggcgcggttgaccataccgtccactcctgattttcgaatcagacaaggacggtatgtaattgctatcaacaagtcataaccagagaaacacgactactacctca

SEQ ID NO: 216 - DNA, 258 bp, synthetic construct
  misc_RNA 1..258
  source 1..258: mol_type = other DNA
gggaaactatacaatatggtatattacactggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagtagaaacacgactactacctca

SEQ ID NO: 217 - DNA, 260 bp, synthetic construct
  misc_RNA 1..260
  source 1..260: mol_type = other DNA
gggaaactatacaatatggtatattacgactggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagtcagaaacacgactactacctca

SEQ ID NO: 218 - DNA, 262 bp, synthetic construct
  misc_RNA 1..262
  source 1..262: mol_type = other DNA
gggaaactatacaatatggtatattactgactggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagtcaagaaacacgactactacctca

SEQ ID NO: 219 - DNA, 264 bp, synthetic construct
  misc_RNA 1..264
  source 1..264: mol_type = other DNA
gggaaactatacaatatggtatattacctgactggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagtcagagaaacacgactactacctca

SEQ ID NO: 220 - DNA, 266 bp, synthetic construct
  misc_RNA 1..266
  source 1..266: mol_type = other DNA
gggaaactatacaatatggtatattacactgactggttttcgatcgaaagatcgacgaggtgaaaacctcgtgacagtctgatagcaatgtcaacctcgcctatgaacttcagggtcagcttgcgtgttattcttggcaagctgaccctgaagttcataggcgcggttgaccataccgtccagtcctgattttcgaatcagagaaggacggtatgtaattgctatcaagaagtcaaccagtcagtagaaacacgactactacctca

SEQ ID NO: 221 - RNA, 23 nt, synthetic construct
  misc_feature 1..23: note = Synthetic sequence
  source 1..23: mol_type = other RNA
ccggttttcgatttggtttgact

SEQ ID NO: 222 - RNA, 23 nt, synthetic construct
  misc_feature 1..23: note = Synthetic sequence
  source 1..23: mol_type = other RNA
ccggttttcgatttggtttgact

SEQ ID NO: 223 - RNA, 23 nt, synthetic construct
  misc_feature 1..23: note = Synthetic sequence
  source 1..23: mol_type = other RNA
ccggttttcgatttggtttgact

SEQ ID NO: 224 - RNA, 23 nt, synthetic construct
  misc_feature 1..23: note = Synthetic sequence
  source 1..23: mol_type = other RNA
ccggttttcgatttggtttgact

SEQ ID NO: 225 - RNA, 23 nt, synthetic construct
  misc_feature 1..23: note = Synthetic sequence
  source 1..23: mol_type = other RNA
ccggttttcgatttggtttgact

SEQ ID NO: 226 - RNA, 23 nt, synthetic construct
  misc_feature 1..23: note = Synthetic sequence
  source 1..23: mol_type = other RNA
ccggttttcgatttggtttgact

SEQ ID NO: 227 - RNA, 23 nt, synthetic construct
  misc_feature 1..23: note = Synthetic sequence
  source 1..23: mol_type = other RNA
ccggttttcgatttggtttgact

SEQ ID NO: 228 - RNA, 23 nt, synthetic construct
  misc_feature 1..23: note = Synthetic sequence
  source 1..23: mol_type = other RNA
ccggttttcgatttggtttgact

SEQ ID NO: 229 - RNA, 23 nt, synthetic construct
  misc_feature 1..23: note = Synthetic sequence
  source 1..23: mol_type = other RNA
ccggttttcgatttggtttgact

SEQ ID NO: 230 - RNA, 22 nt, synthetic construct
  misc_feature 1..22: note = Synthetic sequence
  source 1..22: mol_type = other RNA
tgaggtagtagattgtatagtt

SEQ ID NO: 231 - RNA, 23 nt, synthetic construct
  misc_feature 1..23: note = Synthetic sequence
  source 1..23: mol_type = other RNA
ccggttttcgatttggtttgact

SEQ ID NO: 232 - RNA, 23 nt, synthetic construct
  misc_feature 1..23: note = Synthetic sequence
  source 1..23: mol_type = other RNA
ccggttttcgatttggtttgact

SEQ ID NO: 233 - RNA, 23 nt, synthetic construct
  misc_feature 1..23: note = Synthetic sequence
  source 1..23: mol_type = other RNA
ccggttttcgatttggtttgact

SEQ ID NO: 234 - RNA, 23 nt, synthetic construct
  misc_feature 1..23: note = Synthetic sequence
  source 1..23: mol_type = other RNA
ccggttttcgatttggtttgact

SEQ ID NO: 235 - RNA, 22 nt, synthetic construct
  misc_feature 1..22: note = Synthetic sequence
  source 1..22: mol_type = other RNA
aaaccgttaccattactgagtt

SEQ ID NO: 236 - RNA, 22 nt, synthetic construct
  misc_feature 1..22: note = Synthetic sequence
  source 1..22: mol_type = other RNA
aaaccgttaccattactgagtt

SEQ ID NO: 237 - RNA, 22 nt, synthetic construct
  misc_feature 1..22: note = Synthetic sequence
  source 1..22: mol_type = other RNA
aaaccgttaccattactgagtt

SEQ ID NO: 238 - RNA, 22 nt, synthetic construct
  misc_feature 1..22: note = Synthetic sequence
  source 1..22: mol_type = other RNA
aaaccgttaccattactgagtt

SEQ ID NO: 239 - RNA, 22 nt, synthetic construct
  misc_feature 1..22: note = Synthetic sequence
  source 1..22: mol_type = other RNA
tgaggtagtagattgtatagtt

SEQ ID NO: 240 - RNA, 22 nt, synthetic construct
  misc_feature 1..22: note = Synthetic sequence
  source 1..22: mol_type = other RNA
tgaggtagtagattgtatagtt

SEQ ID NO: 241 - RNA, 22 nt, synthetic construct
  misc_feature 1..22: note = Synthetic sequence
  source 1..22: mol_type = other RNA
tgaggtagtagattgtatagtt

SEQ ID NO: 242 - RNA, 22 nt, synthetic construct
  misc_feature 1..22: note = Synthetic sequence
  source 1..22: mol_type = other RNA
tgaggtagtagattgtatagtt

SEQ ID NO: 243 - RNA, 23 nt, synthetic construct
  misc_feature 1..23: note = Synthetic sequence
  source 1..23: mol_type = other RNA
ccggttttcgatttggtttgact

SEQ ID NO: 244 - RNA, 22 nt, synthetic construct
  misc_feature 1..22: note = Synthetic sequence
  source 1..22: mol_type = other RNA
aaaccgttaccattactgagtt

SEQ ID NO: 245 - RNA, 20 nt, synthetic construct
  misc_feature 1..20: note = Synthetic sequence
  source 1..20: mol_type = other RNA
ttcggaagagacaggtacgt

SEQ ID NO: 246 - RNA, 22 nt, synthetic construct
  misc_feature 1..22: note = Synthetic sequence
  source 1..22: mol_type = other RNA
tggacggagaactgataagggc

SEQ ID NO: 247 - RNA, 22 nt, synthetic construct
  misc_feature 1..22: note = Synthetic sequence
  source 1..22: mol_type = other RNA
ctaagtactagtgccgcaggag

SEQ ID NO: 248 - RNA, 24 nt, synthetic construct
  misc_feature 1..24: note = Synthetic sequence
  source 1..24: mol_type = other RNA
aatggcactggaagaattcacggg

SEQ ID NO: 249 - DNA, 20 bp, synthetic construct
  misc_RNA 1..20
  source 1..20: mol_type = other DNA
gggacagatctaatggctgc

SEQ ID NO: 250 - DNA, 40 bp, synthetic construct
  misc_RNA 1..40
  source 1..40: mol_type = other DNA
gggagtagacaattctagtcttactattaagaaacctaat

SEQ ID NO: 251 - RNA, 22 nt, synthetic construct
  misc_feature 1..22: note = Synthetic sequence
  source 1..22: mol_type = other RNA
tgaggtagtagattgtatagtt

SEQ ID NO: 252 - RNA, 20 nt, synthetic construct
  misc_feature 1..20: note = Synthetic sequence
  source 1..20: mol_type = other RNA
ttcggaagagacaggtacgt

SEQ ID NO: 253 - RNA, 20 nt, synthetic construct
  misc_feature 1..20: note = Synthetic sequence
  source 1..20: mol_type = other RNA
ttcggaagagacaggtacgt

SEQ ID NO: 254 - RNA, 20 nt, synthetic construct
  misc_feature 1..20: note = Synthetic sequence
  source 1..20: mol_type = other RNA
ttcggaagagacaggtacgt

SEQ ID NO: 255 - RNA, 20 nt, synthetic construct
  misc_feature 1..20: note = Synthetic sequence
  source 1..20: mol_type = other RNA
ttcggaagagacaggtacgt

SEQ ID NO: 256 - RNA, 20 nt, synthetic construct
  misc_feature 1..20: note = Synthetic sequence
  source 1..20: mol_type = other RNA
ttcggaagagacaggtacgt

SEQ ID NO: 257 - RNA, 22 nt, synthetic construct
  misc_feature 1..22: note = Synthetic sequence
  source 1..22: mol_type = other RNA
aaaccgttaccattactgagtt

SEQ ID NO: 258 - RNA, 22 nt, synthetic construct
  misc_feature 1..22: note = Synthetic sequence
  source 1..22: mol_type = other RNA
aaaccgttaccattactgagtt

SEQ ID NO: 259 - RNA, 22 nt, synthetic construct
  misc_feature 1..22: note = Synthetic sequence
  source 1..22: mol_type = other RNA
aaaccgttaccattactgagtt

SEQ ID NO: 260 - RNA, 22 nt, synthetic construct
  misc_feature 1..22: note = Synthetic sequence
  source 1..22: mol_type = other RNA
aaaccgttaccattactgagtt

SEQ ID NO: 261 - RNA, 22 nt, synthetic construct
  misc_feature 1..22: note = Synthetic sequence
  source 1..22: mol_type = other RNA
aaaccgttaccattactgagtt

SEQ ID NO: 262 - RNA, 20 nt, synthetic construct
  misc_feature 1..20: note = Synthetic sequence
  source 1..20: mol_type = other RNA
cttcttcaggttggacagct

SEQ ID NO: 263 - RNA, 20 nt, synthetic construct
  misc_feature 1..20: note = Synthetic sequence
  source 1..20: mol_type = other RNA
cttcttcaggttggacagct

SEQ ID NO: 264 - RNA, 20 nt, synthetic construct
  misc_feature 1..20: note = Synthetic sequence
  source 1..20: mol_type = other RNA
cttcttcaggttggacagct

SEQ ID NO: 265 - RNA, 20 nt, synthetic construct
  misc_feature 1..20: note = Synthetic sequence
  source 1..20: mol_type = other RNA
cttcttcaggttggacagct

SEQ ID NO: 266 - RNA, 20 nt, synthetic construct
  misc_feature 1..20: note = Synthetic sequence
  source 1..20: mol_type = other RNA
cttcttcaggttggacagct

SEQ ID NO: 267 - RNA, 22 nt, synthetic construct
  misc_feature 1..22: note = Synthetic sequence
  source 1..22: mol_type = other RNA
tgaggtagtagattgtatagtt

SEQ ID NO: 268 - RNA, 22 nt, synthetic construct
  misc_feature 1..22: note = Synthetic sequence
  source 1..22: mol_type = other RNA
tgaggtagtagattgtatagtt

SEQ ID NO: 269 - RNA, 22 nt, synthetic construct
  misc_feature 1..22: note = Synthetic sequence
  source 1..22: mol_type = other RNA
tgaggtagtagattgtatagtt

SEQ ID NO: 270 - RNA, 22 nt, synthetic construct
  misc_feature 1..22: note = Synthetic sequence
  source 1..22: mol_type = other RNA
tgaggtagtagattgtatagtt

SEQ ID NO: 271 - RNA, 22 nt, synthetic construct
  misc_feature 1..22: note = Synthetic sequence
  source 1..22: mol_type = other RNA
tgaggtagtagattgtatagtt

SEQ ID NO: 272 - RNA, 22 nt, synthetic construct
  misc_feature 1..22: note = Synthetic sequence
  source 1..22: mol_type = other RNA
tgaggtagtagattgtatagtt

SEQ ID NO: 273 - RNA, 22 nt, synthetic construct
  misc_feature 1..22: note = Synthetic sequence
  source 1..22: mol_type = other RNA
tgaggtagtagattgtatagtt

SEQ ID NO: 274 - RNA, 23 nt, synthetic construct
  misc_feature 1..23: note = synthetic sequence
  source 1..23: mol_type = other RNA
tttgtactccgatgccattcaga

SEQ ID NO: 275 - RNA, 23 nt, synthetic construct
  misc_feature 1..23: note = Synthetic sequence
  source 1..23: mol_type = other RNA
tttgtactccgatgccattcaga

SEQ ID NO: 276 - RNA, 23 nt, synthetic construct
  misc_feature 1..23: note = Synthetic sequence
  source 1..23: mol_type = other RNA
tttgtactccgatgccattcaga

SEQ ID NO: 277 - RNA, 23 nt, synthetic construct
  misc_feature 1..23: note = Synthetic sequence
  source 1..23: mol_type = other RNA
tttgtactccgatgccattcaga

SEQ ID NO: 278 - RNA, 22 nt, synthetic construct
  misc_feature 1..22: note = Synthetic sequence
  source 1..22: mol_type = other RNA
tgaggtagtagattgtatagtt

SEQ ID NO: 279 - RNA, 22 nt, synthetic construct
  misc_feature 1..22: note = Synthetic sequence
  source 1..22: mol_type = other RNA
tgaggtagtagattgtatagtt

SEQ ID NO: 280 - RNA, 22 nt, synthetic construct
  misc_feature 1..22: note = Synthetic sequence
  source 1..22: mol_type = other RNA
tgaggtagtagattgtatagtt

SEQ ID NO: 281 - RNA, 22 nt, synthetic construct
  misc_feature 1..22: note = Synthetic sequence
  source 1..22: mol_type = other RNA
tgaggtagtagattgtatagtt

SEQ ID NO: 282 - RNA, 23 nt, synthetic construct
  misc_feature 1..23: note = Synthetic sequence
  source 1..23: mol_type = other RNA
tttgtactccgatgccattcaga

SEQ ID NO: 283 - RNA, 23 nt, synthetic construct
  misc_feature 1..23: note = Synthetic sequence
  source 1..23: mol_type = other RNA
tttgtactccgatgccattcaga

SEQ ID NO: 284 - RNA, 23 nt, synthetic construct
  misc_feature 1..23: note = Synthetic sequence
  source 1..23: mol_type = other RNA
tttgtactccgatgccattcaga

SEQ ID NO: 285 - RNA, 23 nt, synthetic construct
  misc_feature 1..23: note = Synthetic sequence
  source 1..23: mol_type = other RNA
tttgtactccgatgccattcaga

SEQ ID NO: 286 - RNA, 22 nt, synthetic construct
  misc_feature 1..22: note = Synthetic sequence
  source 1..22: mol_type = other RNA
tgaggtagtagattgtatagtt

SEQ ID NO: 287 - RNA, 22 nt, synthetic construct
  misc_feature 1..22: note = Synthetic sequence
  source 1..22: mol_type = other RNA
tgaggtagtagattgtatagtt

SEQ ID NO: 288 - RNA, 22 nt, synthetic construct
  misc_feature 1..22: note = Synthetic sequence
  source 1..22: mol_type = other RNA
tgaggtagtagattgtatagtt

SEQ ID NO: 289 - RNA, 22 nt, synthetic construct
  misc_feature 1..22: note = Synthetic sequence
  source 1..22: mol_type = other RNA
tgaggtagtagattgtatagtt

SEQ ID NO: 290 - RNA, 23 nt, synthetic construct
  misc_feature 1..23: note = Synthetic sequence
  source 1..23: mol_type = other RNA
tttgtactccgatgccattcaga

SEQ ID NO: 291 - RNA, 23 nt, synthetic construct
  misc_feature 1..23: note = Synthetic sequence
  source 1..23: mol_type = other RNA
tttgtactccgatgccattcaga

SEQ ID NO: 292 - RNA, 23 nt, synthetic construct
  misc_feature 1..23: note = Synthetic sequence
  source 1..23: mol_type = other RNA
tttgtactccgatgccattcaga

SEQ ID NO: 293 - RNA, 22 nt, synthetic construct
  misc_feature 1..22: note = Synthetic sequence
  source 1..22: mol_type = other RNA
tgaggtagtagattgtatagtt

SEQ ID NO: 294 - RNA, 22 nt, synthetic construct
  misc_feature 1..22: note = Synthetic sequence
  source 1..22: mol_type = other RNA
tgaggtagtagattgtatagtt

SEQ ID NO: 295 - RNA, 22 nt, synthetic construct
  misc_feature 1..22: note = Synthetic sequence
  source 1..22: mol_type = other RNA
tgaggtagtagattgtatagtt

SEQ ID NO: 296 - RNA, 22 nt, synthetic construct
  misc_feature 1..22: note = Synthetic sequence
  source 1..22: mol_type = other RNA
tgaggtagtagattgtatagtt

SEQ ID NO: 297 - RNA, 22 nt, synthetic construct
  misc_feature 1..22: note = Synthetic sequence
  source 1..22: mol_type = other RNA
tgaggtagtagattgtatagtt

SEQ ID NO: 298 - RNA, 22 nt, synthetic construct
  misc_feature 1..22: note = Synthetic sequence
  source 1..22: mol_type = other RNA
tgaggtagtagattgtatagtt

SEQ ID NO: 299 - RNA, 22 nt, synthetic construct
  misc_feature 1..22: note = Synthetic sequence
  source 1..22: mol_type = other RNA
tgaggtagtagattgtatagtt

SEQ ID NO: 300 - RNA, 22 nt, synthetic construct
  misc_feature 1..22: note = Synthetic sequence
  source 1..22: mol_type = other RNA
tgaggtagtagattgtatagtt

SEQ ID NO: 301 - RNA, 22 nt, synthetic construct
  misc_feature 1..22: note = Synthetic sequence
  source 1..22: mol_type = other RNA
tgaggtagtagattgtatagtt

SEQ ID NO: 302 - RNA, 22 nt, synthetic construct
  misc_feature 1..22: note = Synthetic sequence
  source 1..22: mol_type = other RNA
tgaggtagtagattgtatagtt

SEQ ID NO: 303 - RNA, 22 nt, synthetic construct
  misc_feature 1..22: note = Synthetic sequence
  source 1..22: mol_type = other RNA
tgaggtagtagattgtatagtt

SEQ ID NO: 304 - RNA, 22 nt, synthetic construct
  misc_feature 1..22: note = Synthetic sequence
  source 1..22: mol_type = other RNA
tgaggtagtagattgtatagtt

SEQ ID NO: 305 - RNA, 22 nt, synthetic construct
  misc_feature 1..22: note = Synthetic sequence
  source 1..22: mol_type = other RNA
tgaggtagtagattgtatagtt

SEQ ID NO: 306 - RNA, 22 nt, synthetic construct
  misc_feature 1..22: note = Synthetic sequence
  source 1..22: mol_type = other RNA
tgaggtagtagattgtatagtt

SEQ ID NO: 307 - RNA, 29 nt, synthetic construct
  misc_feature 1..29: note = Synthetic sequence
  source 1..29: mol_type = other RNA
gtccttagtcgaaagttttactagagtca

SEQ ID NO: 308 - RNA, 29 nt, synthetic construct
  misc_feature 1..29: note = Synthetic sequence
  source 1..29: mol_type = other RNA
gtccttagtcgaaagttttactagagtca

SEQ ID NO: 309 - RNA, 29 nt, synthetic construct
  misc_feature 1..29: note = Synthetic sequence
  source 1..29: mol_type = other RNA
gtccttagtcgaaagttttactagagtca

SEQ ID NO: 310 - RNA, 29 nt, synthetic construct
  misc_feature 1..29: note = Synthetic sequence
  source 1..29: mol_type = other RNA
gtccttagtcgaaagttttactagagtca

SEQ ID NO: 311 - RNA, 29 nt, synthetic construct
  misc_feature 1..29: note = Synthetic sequence
  source 1..29: mol_type = other RNA
gtccttagtcgaaagttttactagagtca

SEQ ID NO: 312 - RNA, 29 nt, synthetic construct
  misc_feature 1..29: note = Synthetic sequence
  source 1..29: mol_type = other RNA
gtccttagtcgaaagttttactagagtca

SEQ ID NO: 313 - RNA, 29 nt, synthetic construct
  misc_feature 1..29: note = Synthetic sequence
  source 1..29: mol_type = other RNA
gtccttagtcgaaagttttactagagtca

SEQ ID NO: 314 - RNA, 29 nt, synthetic construct
  misc_feature 1..29: note = Synthetic sequence
  source 1..29: mol_type = other RNA
gtccttagtcgaaagttttactagagtca

SEQ ID NO: 315 - RNA, 29 nt, synthetic construct
  misc_feature 1..29: note = Synthetic sequence
  source 1..29: mol_type = other RNA
gtccttagtcgaaagttttactagagtca

SEQ ID NO: 316 - RNA, 29 nt, synthetic construct
  misc_feature 1..29: note = Synthetic sequence
  source 1..29: mol_type = other RNA
gtccttagtcgaaagttttactagagtca

SEQ ID NO: 317 - RNA, 29 nt, synthetic construct
  misc_feature 1..29: note = Synthetic sequence
  source 1..29: mol_type = other RNA
gtccttagtcgaaagttttactagagtca

SEQ ID NO: 318 - RNA, 29 nt, synthetic construct
  misc_feature 1..29: note = Synthetic sequence
  source 1..29: mol_type = other RNA
gtccttagtcgaaagttttactagagtca

SEQ ID NO: 319 - RNA, 29 nt, synthetic construct
  misc_feature 1..29: note = Synthetic sequence
  source 1..29: mol_type = other RNA
gtccttagtcgaaagttttactagagtca

SEQ ID NO: 320 - RNA, 29 nt, synthetic construct
  misc_feature 1..29: note = Synthetic sequence
  source 1..29: mol_type = other RNA
gtccttagtcgaaagttttactagagtca

SEQ ID NO: 321 - RNA, 29 nt, synthetic construct
  misc_feature 1..29: note = Synthetic sequence
  source 1..29: mol_type = other RNA
gtccttagtcgaaagttttactagagtca

SEQ ID NO: 322 - RNA, 29 nt, synthetic construct
  misc_feature 1..29: note = Synthetic sequence
  source 1..29: mol_type = other RNA
gtccttagtcgaaagttttactagagtca

SEQ ID NO: 323 - RNA, 29 nt, synthetic construct
  misc_feature 1..29: note = Synthetic sequence
  source 1..29: mol_type = other RNA
gtccttagtcgaaagttttactagagtca

SEQ ID NO: 324 - RNA, 29 nt, synthetic construct
  misc_feature 1..29: note = Synthetic sequence
  source 1..29: mol_type = other RNA
gtccttagtcgaaagttttactagagtca

SEQ ID NO: 325 - RNA, 29 nt, synthetic construct
  misc_feature 1..29: note = Synthetic sequence
  source 1..29: mol_type = other RNA
gtccttagtcgaaagttttactagagtca

SEQ ID NO: 326 - RNA, 29 nt, synthetic construct
  misc_feature 1..29: note = Synthetic sequence
  source 1..29: mol_type = other RNA
gtccttagtcgaaagttttactagagtca

SEQ ID NO: 327 - RNA, 29 nt, synthetic construct
  misc_feature 1..29: note = Synthetic sequence
  source 1..29: mol_type = other RNA
gtccttagtcgaaagttttactagagtca

SEQ ID NO: 328 - RNA, 29 nt, synthetic construct
  misc_feature 1..29: note = Synthetic sequence
  source 1..29: mol_type = other RNA
gtccttagtcgaaagttttactagagtca

SEQ ID NO: 329 - RNA, 29 nt, synthetic construct
  misc_feature 1..29: note = Synthetic sequence
  source 1..29: mol_type = other RNA
gtccttagtcgaaagttttactagagtca

SEQ ID NO: 330 - RNA, 29 nt, synthetic construct
  misc_feature 1..29: note = Synthetic sequence
  source 1..29: mol_type = other RNA
gtccttagtcgaaagttttactagagtca

SEQ ID NO: 331 - RNA, 29 nt, synthetic construct
  misc_feature 1..29: note = Synthetic sequence
  source 1..29: mol_type = other RNA
gtccttagtcgaaagttttactagagtca

SEQ ID NO: 332 - RNA, 29 nt, synthetic construct
  misc_feature 1..29: note = Synthetic sequence
  source 1..29: mol_type = other RNA
gtccttagtcgaaagttttactagagtca

SEQ ID NO: 333 - RNA, 29 nt, synthetic construct
  misc_feature 1..29: note = Synthetic sequence
  source 1..29: mol_type = other RNA
gtccttagtcgaaagttttactagagtca

SEQ ID NO: 334 - RNA, 29 nt, synthetic construct
  misc_feature 1..29: note = Synthetic sequence
  source 1..29: mol_type = other RNA
gtccttagtcgaaagttttactagagtca

SEQ ID NO: 335 - RNA, 29 nt, synthetic construct
  misc_feature 1..29: note = Synthetic sequence
  source 1..29: mol_type = other RNA
gtccttagtcgaaagttttactagagtca

SEQ ID NO: 336 - RNA, 29 nt, synthetic construct
  misc_feature 1..29: note = Synthetic sequence
  source 1..29: mol_type = other RNA
gtccttagtcgaaagttttactagagtca

SEQ ID NO: 337 - RNA, 29 nt, synthetic construct
  misc_feature 1..29: note = Synthetic sequence
  source 1..29: mol_type = other RNA
gtccttagtcgaaagttttactagagtca

SEQ ID NO: 338 - RNA, 29 nt, synthetic construct
  misc_feature 1..29: note = Synthetic sequence
  source 1..29: mol_type = other RNA
gtccttagtcgaaagttttactagagtca

SEQ ID NO: 339 - RNA, 29 nt, synthetic construct
  misc_feature 1..29: note = Synthetic sequence
  source 1..29: mol_type = other RNA
gtccttagtcgaaagttttactagagtca

SEQ ID NO: 340 - RNA, 29 nt, synthetic construct
  misc_feature 1..29: note = Synthetic sequence
  source 1..29: mol_type = other RNA
gtccttagtcgaaagttttactagagtca

SEQ ID NO: 341 - RNA, 29 nt, synthetic construct
  misc_feature 1..29: note = Synthetic sequence
  source 1..29: mol_type = other RNA
gtccttagtcgaaagttttactagagtca

SEQ ID NO: 342 - RNA, 29 nt, synthetic construct
  misc_feature 1..29: note = Synthetic sequence
  source 1..29: mol_type = other RNA
gtccttagtcgaaagttttactagagtca

SEQ ID NO: 343 - RNA, 29 nt, synthetic construct
  misc_feature 1..29: note = Synthetic sequence
  source 1..29: mol_type = other RNA
gtccttagtcgaaagttttactagagtca

SEQ ID NO: 344 - RNA, 29 nt, synthetic construct
  misc_feature 1..29: note = Synthetic sequence
  source 1..29: mol_type = other RNA
gtccttagtcgaaagttttactagagtca

SEQ ID NO: 345 - RNA, 29 nt, synthetic construct
  misc_feature 1..29: note = Synthetic sequence
  source 1..29: mol_type = other RNA
gtccttagtcgaaagttttactagagtca

SEQ ID NO: 346 - RNA, 29 nt, synthetic construct
  misc_feature 1..29: note = Synthetic sequence
  source 1..29: mol_type = other RNA
gtccttagtcgaaagttttactagagtca

SEQ ID NO: 347 - RNA, 29 nt, synthetic construct
  misc_feature 1..29: note = Synthetic sequence
  source 1..29: mol_type = other RNA
gtccttagtcgaaagttttactagagtca

SEQ ID NO: 348 - RNA, 29 nt, synthetic construct
  misc_feature 1..29: note = Synthetic sequence
  source 1..29: mol_type = other RNA
gtccttagtcgaaagttttactagagtca

SEQ ID NO: 349 - RNA, 29 nt, synthetic construct
  misc_feature 1..29: note = Synthetic sequence
  source 1..29: mol_type = other RNA
gtccttagtcgaaagttttactagagtca

SEQ ID NO: 350 - RNA, 29 nt, synthetic construct
  misc_feature 1..29: note = Synthetic sequence
  source 1..29: mol_type = other RNA
gtccttagtcgaaagttttactagagtca

SEQ ID NO: 351 - RNA, 29 nt, synthetic construct
  misc_feature 1..29: note = Synthetic sequence
  source 1..29: mol_type = other RNA
gtccttagtcgaaagttttactagagtca

SEQ ID NO: 352 - RNA, 29 nt, synthetic construct
  misc_feature 1..29: note = Synthetic sequence
  source 1..29: mol_type = other RNA
gtccttagtcgaaagttttactagagtca

SEQ ID NO: 353 - RNA, 29 nt, synthetic construct
  misc_feature 1..29: note = Synthetic sequence
  source 1..29: mol_type = other RNA
gtccttagtcgaaagttttactagagtca

SEQ ID NO: 354 - RNA, 29 nt, synthetic construct
  misc_feature 1..29: note = Synthetic sequence
  source 1..29: mol_type = other RNA
gtccttagtcgaaagttttactagagtca

SEQ ID NO: 355 - RNA, 29 nt, synthetic construct
  misc_feature 1..29: note = Synthetic sequence
  source 1..29: mol_type = other RNA
gtccttagtcgaaagttttactagagtca

SEQ ID NO: 356 - RNA, 29 nt, synthetic construct
  misc_feature 1..29: note = Synthetic sequence
  source 1..29: mol_type = other RNA
gtccttagtcgaaagttttactagagtca

SEQ ID NO: 357 - RNA, 29 nt, synthetic construct
  misc_feature 1..29: note = Synthetic sequence
  source 1..29: mol_type = other RNA
gtccttagtcgaaagttttactagagtca

SEQ ID NO: 358 - RNA, 29 nt, synthetic construct
  misc_feature 1..29: note = Synthetic sequence
  source 1..29: mol_type = other RNA
gtccttagtcgaaagttttactagagtca

SEQ ID NO: 359 - RNA, 29 nt, synthetic construct
  misc_feature 1..29: note = Synthetic sequence
  source 1..29: mol_type = other RNA
gtccttagtcgaaagttttactagagtca

SEQ ID NO: 360 - RNA, 29 nt, synthetic construct
  misc_feature 1..29: note = Synthetic sequence
  source 1..29: mol_type = other RNA
gtccttagtcgaaagttttactagagtca

SEQ ID NO: 361 - RNA, 29 nt, synthetic construct
  misc_feature 1..29: note = Synthetic sequence
  source 1..29: mol_type = other RNA
gtccttagtcgaaagttttactagagtca

SEQ ID NO: 362 - RNA, 29 nt, synthetic construct
  misc_feature 1..29: note = Synthetic sequence
  source 1..29: mol_type = other RNA
gtccttagtcgaaagttttactagagtca

SEQ ID NO: 363 - RNA, 29 nt, synthetic construct
  misc_feature 1..29: note = Synthetic sequence
  source 1..29: mol_type = other RNA
gtccttagtcgaaagttttactagagtca

SEQ ID NO: 364 - RNA, 29 nt, synthetic construct
  misc_feature 1..29: note = Synthetic sequence
  source 1..29: mol_type = other RNA
gtccttagtcgaaagttttactagagtca

SEQ ID NO: 365 - RNA, 29 nt, synthetic construct
  misc_feature 1..29: note = Synthetic sequence
  source 1..29: mol_type = other RNA
gtccttagtcgaaagttttactagagtca

SEQ ID NO: 366 - RNA, 29 nt, synthetic construct
  misc_feature 1..29: note = Synthetic sequence
  source 1..29: mol_type = other RNA
gtccttagtcgaaagttttactagagtca

SEQ ID NO: 367 - RNA, 29 nt, synthetic construct
  misc_feature 1..29: note = Synthetic sequence
  source 1..29: mol_type = other RNA
gtccttagtcgaaagttttactagagtca

SEQ ID NO: 368 - RNA, 29 nt, synthetic construct
  misc_feature 1..29: note = Synthetic sequence
  source 1..29: mol_type = other RNA
gtccttagtcgaaagttttactagagtca

SEQ ID NO: 369 - RNA, 29 nt, synthetic construct
  misc_feature 1..29: note = Synthetic sequence
  source 1..29: mol_type = other RNA
gtccttagtcgaaagttttactagagtca

SEQ ID NO: 370 - RNA, 113 nt, synthetic construct
  misc_feature 1..113: note = Synthetic sequence
  source 1..113: mol_type = other RNA
gtccttgggcacgggcagcttgccgggttttagagctagaaatagcaagttaaaataaggctagtccgttatcaacttgaaaaagtggcaccgagtcggtgctttttttgtca

SEQ ID NO: 371 - RNA, 100 nt, synthetic construct
  misc_feature 1..100: note = Synthetic sequence
  source 1..100: mol_type = other RNA
gggcacgggcagcttgccgggttttagagctagaaatagcaagttaaaataaggctagtccgttatcaacttgaaaaagtggcaccgagtcggtgcgtca

SEQ ID NO: 372 - RNA, 100 nt, synthetic construct; identical to SEQ ID NO: 371
  misc_feature 1..100: note = Synthetic sequence
  source 1..100: mol_type = other RNA

SEQ ID NO: 373 - RNA, 100 nt, synthetic construct; identical to SEQ ID NO: 371
  misc_feature 1..100: note = Synthetic sequence
  source 1..100: mol_type = other RNA

SEQ ID NO: 374 - RNA, 100 nt, synthetic construct; identical to SEQ ID NO: 371
  misc_feature 1..100: note = Synthetic sequenc
  source 1..100: mol_type = other RNA

SEQ ID NO: 375 - RNA, 100 nt, synthetic construct; identical to SEQ ID NO: 371
  misc_feature 1..100: note = Synthetic sequence
  source 1..100: mol_type = other RNA

SEQ ID NO: 376 - RNA, 100 nt, synthetic construct; identical to SEQ ID NO: 371
  misc_feature 1..100: note = Synthetic sequence
  source 1..100: mol_type = other RNA

SEQ ID NO: 377 - RNA, 100 nt, synthetic construct; identical to SEQ ID NO: 371
  misc_feature 1..100: note = Synthetic sequence
  source 1..100: mol_type = other RNA

SEQ ID NO: 378 - RNA, 100 nt, synthetic construct; identical to SEQ ID NO: 371
  misc_feature 1..100: note = Synthetic sequence
  source 1..100: mol_type = other RNA

SEQ ID NO: 379 - RNA, 100 nt, synthetic construct; identical to SEQ ID NO: 371
  misc_feature 1..100: note = Synthetic sequence
  source 1..100: mol_type = other RNA

SEQ ID NO: 380 - RNA, 100 nt, synthetic construct; identical to SEQ ID NO: 371
  misc_feature 1..100: note = Synthetic sequence
  source 1..100: mol_type = other RNA

SEQ ID NO: 381 - RNA, 100 nt, synthetic construct; identical to SEQ ID NO: 371
  misc_feature 1..100: note = Synthetic sequence
  source 1..100: mol_type = other RNA

SEQ ID NO: 382 - RNA, 100 nt, synthetic construct; identical to SEQ ID NO: 371
  misc_feature 1..100: note = Synthetic sequence
  source 1..100: mol_type = other RNA

SEQ ID NO: 383 - RNA, 100 nt, synthetic construct; identical to SEQ ID NO: 371
  misc_feature 1..100: note = Synthetic sequence
  source 1..100: mol_type = other RNA

SEQ ID NO: 384 - RNA, 100 nt, synthetic construct; identical to SEQ ID NO: 371
  misc_feature 1..100: note = Synthetic sequence
  source 1..100: mol_type = other RNA

SEQ ID NO: 385 - RNA, 100 nt, synthetic construct; identical to SEQ ID NO: 371
  misc_feature 1..100: note = Synthetic sequence
  source 1..100: mol_type = other RNA

SEQ ID NO: 386 - RNA, 100 nt, synthetic construct; identical to SEQ ID NO: 371
  misc_feature 1..100: note = Synthetic sequence
  source 1..100: mol_type = other RNA

SEQ ID NO: 387 - RNA, 100 nt, synthetic construct; identical to SEQ ID NO: 371
  misc_feature 1..100: note = Synthetic sequence
  source 1..100: mol_type = other RNA

SEQ ID NO: 388 - RNA, 100 nt, synthetic construct; identical to SEQ ID NO: 371
  misc_feature 1..100: note = Synthetic sequence
  source 1..100: mol_type = other RNA

SEQ ID NO: 389 - RNA, 97 nt, synthetic construct
  misc_feature 1..97: note = Synthetic sequence
  source 1..97: mol_type = other RNA
gtctgatagcaatgtcagcagtgcctgcaagctgaccctgaagttcaccacctgaacttcagggtcagcttgcaagtaaggttgaccatactctaca

SEQ ID NO: 390 - RNA, 97 nt, synthetic construct; identical to SEQ ID NO: 389
  misc_feature 1..97: note = Synthetic sequence
  source 1..97: mol_type = other RNA

SEQ ID NO: 391 - RNA, 97 nt, synthetic construct; identical to SEQ ID NO: 389
  misc_feature 1..97: note = Synthetic sequence
  source 1..97: mol_type = other RNA

SEQ ID NO: 392 - RNA, 97 nt, synthetic construct
  misc_feature 1..97: note = Synthetic sequence
  source 1..97: mol_type = other RNA
gtctgatagcaatgtcagcagtgcctgcaagctgcaactgaagttcaccacctgaacttcagttgcagcttgcaagtaaggttgaccatactctaca

SEQ ID NO: 393 - RNA, 97 nt, synthetic construct; identical to SEQ ID NO: 389
  misc_feature 1..97: note = Synthetic sequence
  source 1..97: mol_type = other RNA

SEQ ID NO: 394 - RNA, 97 nt, synthetic construct; identical to SEQ ID NO: 389
  misc_feature 1..97: note = Synthetic sequence
  source 1..97: mol_type = other RNA

SEQ ID NO: 395 - RNA, 97 nt, synthetic construct; identical to SEQ ID NO: 392
  misc_feature 1..97: note = Synthetic sequence
  source 1..97: mol_type = other RNA

SEQ ID NO: 396 - RNA, 60 nt, synthetic construct
  misc_feature 1..60: note = Synthetic sequence
  source 1..60: mol_type = other RNA
gtccgagacggtcgggtccagtcccaacgatgttggctgttgagtagtgtgtgggctcca

SEQ ID NO: 397 - RNA, 100 nt, synthetic construct
  misc_feature 1..100: note = Synthetic sequence
  source 1..100: mol_type = other RNA
gtctgatagcaatgtcagcagtgcctgcaagctgaccctgaagttcatctgcagatgaacttcagggtcagcttgcaagtaaggttgaccatactctaca

SEQ ID NO: 398 - RNA, 97 nt, synthetic construct; identical to SEQ ID NO: 389
  misc_feature 1..97: note = Synthetic sequence
  source 1..97: mol_type = other RNA

SEQ ID NO: 399 - RNA, 97 nt, synthetic construct
  misc_feature 1..97: note = Synthetic sequence
  source 1..97: mol_type = other RNA
gtctgatagcaatgtcaacctcgcctgcaagctgaccctgaagttcaccacctgaacttcagggtcagcttgcaggcgcggttgaccataccgtcca

SEQ ID NO: 400 - RNA, 97 nt, synthetic construct
  misc_feature 1..97: note = Synthetic sequence
  source 1..97: mol_type = other RNA
gtctgatagcaatgtcaacctcgcctgcaagctgcaactgaagttcaccacctgaacttcagggtcagcttgcaggcgcggttgaccataccgtcca

SEQ ID NO: 401 - RNA, 106 nt, synthetic construct
  misc_feature 1..106: note = Synthetic sequence
  source 1..106: mol_type = other RNA
gtctgctagcaatgtcaacctcgcctgcaagctgaccctgaagttcatgtgccacctctaatgaacttcagggtcagcttgcaggcgcggttgaccataccgtcca

SEQ ID NO: 402 - RNA, 106 nt, synthetic construct
  misc_feature 1..106: note = Synthetic sequence
  source 1..106: mol_type = other RNA
gtctgctagcaatgtcaacctcgcctgcaagctgaccctgaagttcatgtgttattcttgatgaacttcagggtcagcttgcaggcgcggttgaccataccgtcca

SEQ ID NO: 403 - RNA, 106 nt, synthetic construct
  misc_feature 1..106: note = Synthetic sequence
  source 1..106: mol_type = other RNA
gtctgatagcaatgtcaacctcgcctatgaacttcagggtcagcttgcgtgttattcttggcaagctgaccctgaagttcataggcgcggttgaccataccgtcca

SEQ ID NO: 404 - RNA, 106 nt, synthetic construct
  misc_feature 1..106: note = Synthetic sequence
  source 1..106: mol_type = other RNA
gtctgatagcaatgtcaacctccttgcagatgaacttcagggtcagctgtgttattcttgagctgaccctgaagttcatctgcaaggcggttgaccataccgtcca

SEQ ID NO: 405 - RNA, 113 nt, synthetic construct
  misc_feature 1..113: note = Synthetic sequence
  source 1..113: mol_type = other RNA
gtccagccagcttattgcgaatctcgatgcagatgaacttcagggtcagctgtgttattcttgagctgaccctgaagttcatctgcatcgcgattcgcttattcgtctttgca

SEQ ID NO: 406 - RNA, 114 nt, synthetic construct
  misc_feature 1..114: note = Synthetic sequence
  source 1..114: mol_type = other RNA
gtctgtatcaattcttgtgtatctcgctgcagatgaacttcagggtcagctgtgttattcttgagctgaccctgaagttcatctgcagcgcggtacaccttattcgtccaatca

SEQ ID NO: 407 - RNA, 106 nt, synthetic construct
  misc_feature 1..106: note = Synthetic sequence
  source 1..106: mol_type = other RNA
gtctgatagcaatgtcaacctcgccttggtgcagatgaacttcagggtgtgttattcttgaccctgaagttcatctgcaccaaggcgcggttgaccataccgtcca

SEQ ID NO: 408 - RNA, 109 nt, synthetic construct
  misc_feature 1..109: note = Synthetic sequence
  source 1..109: mol_type = other RNA
gtccagccagcttattgcgaatctcgatggtgcagatgaacttcagggtgtgttattcttgaccctgaagttcatctgcaccatcgcgattcgcttattcgtctttgca

SEQ ID NO: 409 - RNA, 114 nt, synthetic construct
  misc_feature 1..114: note = Synthetic sequence
  source 1..114: mol_type = other RNA
gtctgtatcaattcttgtgtatctcgccgtggtgcagatgaacttcagggtgtgttattcttgaccctgaagttcatctgcaccatggcgcggtacaccttattcgtccaatca

SEQ ID NO: 410 - RNA, 82 nt, synthetic construct
  misc_feature 1..82: note = Synthetic sequence
  source 1..82: mol_type = other RNA
gggaccagagaaacacacgaatcagagaagactctagtaaaactttcgactaagagaagtcaaccagagaaacacacttccc

SEQ ID NO: 411 - RNA, 80 nt, synthetic construct
  misc_feature 1..80: note = Synthetic sequence
  source 1..80: mol_type = other RNA
gggaccagagaaacacacgaatcagagaagactctagtaaaactttcgactaagagaagtcaaccagagaaacacacttc

SEQ ID NO: 412 - RNA, 78 nt, synthetic construct
  misc_feature 1..78: note = Synthetic sequence
  source 1..78: mol_type = other RNA
gggaccagagaaacacacgaatcagagaagactctagtaaaactttcgactaagagaagtcaaccagagaaacacact

SEQ ID NO: 413 - RNA, 77 nt, synthetic construct
  misc_feature 1..77: note = Synthetic sequence
  source 1..77: mol_type = other RNA
gggaccagagaaacacacgaatcagagaagactctagtaaaactttcgactaagagaagtcaaccagagaaacacac

SEQ ID NO: 414 - RNA, 77 nt, synthetic construct
  misc_feature 1..77: note = Synthetic sequence
  source 1..77: mol_type = other RNA
gggaaccagagaaacacacgaatcagagaagactctagtaaaactttcgactaagagaagtcaaccagagaaacaca

SEQ ID NO: 415 - RNA, 75 nt, synthetic construct
  misc_feature 1..75: note = Synthetic sequence
  source 1..75: mol_type = other RNA
gggaccagagaaacacacgaatcagagaagactctagtaaaactttcgactaagagaagtcaaccagagaaacac

SEQ ID NO: 416 - RNA, 74 nt, synthetic construct
  misc_feature 1..74: note = Synthetic sequence
  source 1..74: mol_type = other RNA
gggaccagagaaacacacgaatcagagaagactctagtaaaactttcgactaagagaagtcaaccagagaaaca

SEQ ID NO: 417 - RNA, 155 nt, synthetic construct
  misc_feature 1..155: note = Synthetic sequence
  source 1..155: mol_type = other RNA
gggaccggttttcgatttggtttgactttcgagtcaaaccaaccagagaaacacacgaatcagagaagacgagcgtcccacgggcgcagaagagaagtcaaccagagaaacacactatcgaaaaccggttcgccggttttcgatttggtttgact

SEQ ID NO: 418 - RNA, 153 nt, synthetic construct
  misc_feature 1..153: note = Synthetic sequence
  source 1..153: mol_type = other RNA
gggaccggttttcgatttggtttgactttcgagtcaaaccaacagagaaacacacgaatcagagaagacgagcgtcccacgggcgcagaagagaagtcaaccagagaaacaactatcgaaaaccggttcgccggttttcgatttggtttgact

SEQ ID NO: 419 - RNA, 151 nt, synthetic construct
  misc_feature 1..151: note = Synthetic sequence
  source 1..151: mol_type = other RNA
gggaccggttttcgatttggtttgactttcgagtcaaaccaaagagaaacacacgaatcagagaagacgagcgtcccacgggcgcagaagagaagtcaaccagagaaacactatcgaaaaccggttcgccggttttcgatttggtttgact

SEQ ID NO: 420 - RNA, 149 nt, synthetic construct
  misc_feature 1..149: note = Synthetic sequence
  source 1..149: mol_type = other RNA
gggaccggttttcgatttggtttgactttcgagtcaaaccaagagaaacacacgaatcagagaagacgagcgtcccacgggcgcagaagagaagtcaaccagagaaacatatcgaaaaccggttcgccggttttcgatttggtttgact

SEQ ID NO: 421 - RNA, 109 nt, synthetic construct
  misc_feature 1..109: note = Synthetic sequence
  source 1..109: mol_type = other RNA
gggaagtcaaaccaagggaccagagaaacacacgaatcagagaagacgagcgtcccacgggcgcagaagagaagtcaaccagagaaacacacttcccatcgaaaaccgg

SEQ ID NO: 422 - RNA, 101 nt, synthetic construct
  misc_feature 1..101: note = Synthetic sequence
  source 1..101: mol_type = other RNA
gggaagtcaaaccaaccagagaaacacacgaatcagagaagacgagcgtcccacgggcgcagaagagaagtcaaccagagaaacacactatcgaaaaccgg

SEQ ID NO: 423 - RNA, 99 nt, synthetic construct
  misc_feature 1..99: note = Synthetic sequence
  source 1..99: mol_type = other RNA
gggaagtcaaaccaacagagaaacacacgaatcagagaagacgagcgtcccacgggcgcagaagagaagtcaaccagagaaacacacatcgaaaaccgg

SEQ ID NO: 424 - RNA, 97 nt, synthetic construct
  misc_feature 1..97: note = Synthetic sequence
  source 1..97: mol_type = other RNA
gggaagtcaaaccaaagagaaacacacgaatcagagaagacgagcgtcccacgggcgcagaagagaagtcaaccagagaaacacaatcgaaaaccgg

SEQ ID NO: 425 - RNA, 95 nt, synthetic construct
  misc_feature 1..95: note = Synthetic sequence
  source 1..95: mol_type = other RNA
gggaagtcaaaccaagagaaacacacgaatcagagaagacgagcgtcccacgggcgcagaagagaagtcaaccagagaaacacatcgaaaaccgg

SEQ ID NO: 426 - DNA, 80 bp, synthetic construct
  misc_RNA 1..80
  source 1..80: mol_type = other DNA
gggaccagagaaacacacgatcagagaagactctagtaaaactttcgactaagagaagtcaccagagaaacacacttccc

SEQ ID NO: 427 - DNA, 82 bp, synthetic construct
  misc_RNA 1..82
  source 1..82: mol_type = other DNA
gggaccagagaaacacacgaatcagagaagactctagtaaaactttcgactaagagaagtcaaccagagaaacacacttccc

SEQ ID NO: 428 - DNA, 82 bp, synthetic construct
  misc_RNA 1..82
  source 1..82: mol_type = other DNA
gggaccagagaaacacacgaatcagagaagacagatcattttgaaagctgaaagagaagtcaaccagagaaacacacttccc

SEQ ID NO: 429 - DNA, 82 bp, synthetic construct; identical to SEQ ID NO: 427
  misc_RNA 1..82
  source 1..82: mol_type = other DNA

SEQ ID NO: 430 - DNA, 82 bp, synthetic construct; identical to SEQ ID NO: 428
  misc_RNA 1..82
  source 1..82: mol_type = other DNA

SEQ ID NO: 431 - DNA, 82 bp, synthetic construct; identical to SEQ ID NO: 427
  misc_RNA 1..82
  source 1..82: mol_type = other DNA

SEQ ID NO: 432 - DNA, 82 bp, synthetic construct; identical to SEQ ID NO: 428
  misc_RNA 1..82
  source 1..82: mol_type = other DNA

SEQ ID NO: 433 - DNA, 82 bp, synthetic construct
  misc_RNA 1..82
  source 1..82: mol_type = other DNA
gggaccagagaaacacacgaatcagagaagacgagctgccccagggatcagaagagaagtcaaccagagaaacacacttccc

SEQ ID NO: 434 - DNA, 82 bp, synthetic construct
  misc_RNA 1..82
  source 1..82: mol_type = other DNA
gggaccagagaaacacacgaatcagagaagacgagagtccaacggtcgcagaagagaagtcaaccagagaaacacacttccc

SEQ ID NO: 435 - DNA, 82 bp, synthetic construct
  misc_RNA 1..82
  source 1..82: mol_type = other DNA
gggaccagagaaacacacgaatcagagaagacgagcgtcccacgggcgcagaagagaagtcaaccagagaaacacacttccc

SEQ ID NO: 436 - RNA, 108 nt, synthetic construct
  misc_feature 1..108: note = Synthetic sequence
  source 1..108: mol_type = other RNA
gggaaactatacaatgtaatataagaaacacacgaatcagagaagacgagcgtcccacgggcgcagaagagaagtcaaccagagaaacataatatacctactacctca

SEQ ID NO: 437 - RNA, 109 nt, synthetic construct
  misc_feature 1..109: note = Synthetic sequence
  source 1..109: mol_type = other RNA
gggaagtcaaaccaagtaatataagaaacacacgaatcagagaagacgagcgtcccacgggcgcagaagagaagtcaaccagagaaacataatatacatcgaaaaccgg

SEQ ID NO: 438 - RNA, 109 nt, synthetic construct
  misc_feature 1..109: note = Synthetic sequence
  source 1..109: mol_type = other RNA
gggaagtcaaaccaagtaatataagaaagacacgaatcagagaagacgagcgtcccacgggcgcagaagagaagtcaaccagagaaagataatatacatcgaaaaccgg

SEQ ID NO: 439 - RNA, 109 nt, synthetic construct
  misc_feature 1..109: note = Synthetic sequence
  source 1..109: mol_type = other RNA
gggaagtcaaaccaagtaatataagaaagacacgaatcagagaagacgagcgtcccacgggcgcagaagagaagtcaaccagagaaacataatatacatcgaaaaccgg

SEQ ID NO: 440 - RNA, 109 nt, synthetic construct
  misc_feature 1..109: note = Synthetic sequence
  source 1..109: mol_type = other RNA
gggaagtcaaaccaagtaatataagaaacacacgaatcagagaagacgagcgtcccacgggcgcagaagagaagtcaaccagagaaagataatatacatcgaaaaccgg

SEQ ID NO: 441 - RNA, 108 nt, synthetic construct
  misc_feature 1..108: note = Synthetic sequence
  source 1..108: mol_type = other RNA
gggatttggcaatgggtaatataagaaacacacgaatcagagaagacgagcgtcccacgggcgcagaagagaagtcaaccagagaaacataatatactaatgactcaa

SEQ ID NO: 442 - RNA, 108 nt, synthetic construct
  misc_feature 1..108: note = Synthetic sequence
  source 1..108: mol_type = other RNA
gggatttggcaatgggtaatataagaaagacacgaatcagagaagacgagcgtcccacgggcgcagaagagaagtcaaccagagaaagataatatactaatgactcaa

SEQ ID NO: 443 - RNA, 108 nt, synthetic construct
  misc_feature 1..108: note = Synthetic sequence
  source 1..108: mol_type = other RNA
gggatttggcaatgggtaatataagaaagacacgaatcagagaagacgagcgtcccacgggcgcagaagagaagtcaaccagagaaacataatatactaatgactcaa

SEQ ID NO: 444 - RNA, 108 nt, synthetic construct
  misc_feature 1..108: note = Synthetic sequence
  source 1..108: mol_type = other RNA
gggatttggcaatgggtaatataagaaacacacgaatcagagaagacgagcgtcccacgggcgcagaagagaagtcaaccagagaaagataatatactaatgactcaa

SEQ ID NO: 445 - RNA, 108 nt, synthetic construct; identical to SEQ ID NO: 436
  misc_feature 1..108: note = Synthetic sequence
  source 1..108: mol_type = other RNA

SEQ ID NO: 446 - RNA, 108 nt, synthetic construct
  misc_feature 1..108: note = Synthetic sequence
  source 1..108: mol_type = other RNA
gggaaactatacaatgtaatataagaaagacacgaatcagagaagacgagcgtcccacgggcgcagaagagaagtcaaccagagaaagataatatacctactacctca

SEQ ID NO: 447 - RNA, 108 nt, synthetic construct
  misc_feature 1..108: note = Synthetic sequence
  source 1..108: mol_type = other RNA
gggaaactatacaatgtaatataagaaagacacgaatcagagaagacgagcgtcccacgggcgcagaagagaagtcaaccagagaaacataatatacctactacctca

SEQ ID NO: 448 - RNA, 108 nt, synthetic construct
  misc_feature 1..108: note = Synthetic sequence
  source 1..108: mol_type = other RNA
gggaaactatacaatgtaatataagaaacacacgaatcagagaagacgagcgtcccacgggcgcagaagagaagtcaaccagagaaagataatatacctactacctca

SEQ ID NO: 449-458 - skipped (10 entries)

SEQ ID NO: 459 - DNA, 277 bp, synthetic construct
  misc_RNA 1..277
  source 1..277: mol_type = other DNA
gggaacgtacctgtacggtatattacctggttttcgatcgaaagatcgacgaggtgaaaacctcgtgacagtccttggacagtactccgctcgagtgttttagagctagaaatagcaagttaaaataaggctagtccgttatcaacttgaaaaagtggcaccgagtcggtgctttttttgtcagtcctgattttcgaatcagagaagaccccaacctatccactcgagcggagtactgtccaagagaagtcaaccagagaaacacgactcttccgaa

SEQ ID NO: 460 - DNA, 272 bp, synthetic construct
  misc_RNA 1..272
  source 1..272: mol_type = other DNA
gggaacgtacctgtacggtatattacctggttttcgatcgaaagatcgacgaggtgaaaacctcgcgtatgacagtcggacagtactccgctcgagtgttttagagctagaaatagcaagttaaaataaggctagtccgttatcaacttgaaaaagtggcaccgagtcggtgcgtcagtcctgattttcgaatcagagaagacccatgataacttactcgagcggagtactgtccagaagtcatacgaccagagaaacacgactcttccgaa

SEQ ID NO: 461 - RNA, 40 nt, synthetic construct
  misc_feature 1..40: note = Synthetic sequence
  source 1..40: mol_type = other RNA
gggacatggaagtcacaccttcgggaacgtggttgaccta

SEQ ID NO: 462 - RNA, 29 nt, synthetic construct
  misc_feature 1..29: note = Synthetic sequence
  source 1..29: mol_type = other RNA
gtccttagtcgaaagttttactagagtca

SEQ ID NO: 463 - DNA, 20 bp, synthetic construct
  misc_RNA 1..20
  source 1..20: mol_type = other DNA
ggcaagctgcccgtgcccaa

SEQ ID NO: 464 - DNA, 20 bp, synthetic construct
  misc_RNA 1..20
  source 1..20: mol_type = other DNA
actctagtaaaactttcgac

SEQ ID NO: 465 - DNA, 20 bp, synthetic construct
  misc_RNA 1..20
  source 1..20: mol_type = other DNA
ccggcaagctgcccgtgccc

SEQ ID NO: 466 - DNA, 60 bp, synthetic construct
  misc_RNA 1..60
  source 1..60: mol_type = other DNA
tggacggtatggtcaaccgcgcctatgaacttcagggtcagcttgccaagaataacacgc

SEQ ID NO: 467 - RNA, 21 nt, synthetic construct
  misc_feature 1..21: note = Synthetic sequence
  source 1..21: mol_type = other RNA
aactatacaatctactacctc

SEQ ID NO: 468 - RNA, 21 nt, synthetic construct
  misc_feature 1..21: note = Synthetic sequence
  source 1..21: mol_type = other RNA
gcgtattatagccgattaacg

SEQ ID NO: 469 - DNA, 18 bp, synthetic construct
  misc_RNA 1..18
  source 1..18: mol_type = other DNA
gtggtgcccatcctggtc

SEQ ID NO: 470 - DNA, 20 bp, synthetic construct
  misc_RNA 1..20
  source 1..20: mol_type = other DNA
cttgtacagctcgtccatgc

SEQ ID NO: 471 - DNA, 36 bp, synthetic construct
  misc_RNA 1..36
  source 1..36: mol_type = other DNA
tgtaaaacgacggccagtacgtaaacggccacaagt

SEQ ID NO: 472 - DNA, 19 bp, synthetic construct
  misc_RNA 1..19
  source 1..19: mol_type = other DNA
tgaagaagatggtgcgctc

SEQ ID NO: 473 - RNA, 250 nt, synthetic construct
  misc_feature 1..250: note = Synthetic sequence
  source 1..250: mol_type = other RNA
aactatacaatacggtatattacctggttttcgatcgaaagatcgacgaggtgaaaacctcgtgacagggcacgggcagcttgccgggttttagagctagaaatagcaagttaaaataaggctagtccgttatcaacttgaaaaagtggcaccgagtcggtgcgtcagtcctgattttcgaatcagagaagactatccaccttaaataggcaagtgagaagtcaaccagagaaacacgactactacctca

SEQ ID NO: 474 - RNA, 250 nt, synthetic construct
  misc_feature 1..250: note = Synthetic sequence
  source 1..250: mol_type = other RNA
aactatacaatacggtatattacctggttttcgatcgaaagatcgacgaggtgaaaacctcgtgacagggcacgggcagcttgccgggttttagagctagaaatagcaagttaaaataaggctagtccgttatcaacttgaaaaagtggcaccgagtcggtgcgtcagtcctgattttcgaatcagagaagactatccaccttaaataggcaagtgcgaagtcaaccagagaaacacgactactacctca

SEQ ID NO: 475 - RNA, 20 nt, synthetic construct
  misc_feature 1..20: note = Synthetic sequence
  source 1..20: mol_type = other RNA
ttcggaagagacaggtacgt

SEQ ID NO: 476 - RNA, 20 nt, synthetic construct
  misc_feature 1..20: note = Synthetic sequence
  source 1..20: mol_type = other RNA
tacggaagagacaggtacgt

SEQ ID NO: 477 - RNA, 20 nt, synthetic construct
  misc_feature 1..20: note = Synthetic sequence
  source 1..20: mol_type = other RNA
ttgggaagagacaggtacgt

SEQ ID NO: 478 - RNA, 20 nt, synthetic construct
  misc_feature 1..20: note = Synthetic sequence
  source 1..20: mol_type = other RNA
ttccgaagagacaggtacgt

SEQ ID NO: 479 - RNA, 20 nt, synthetic construct
  misc_feature 1..20: note = Synthetic sequence
  source 1..20: mol_type = other RNA
tagggaagagacaggtacgt

SEQ ID NO: 480 - RNA, 20 nt, synthetic construct
  misc_feature 1..20: note = Synthetic sequence
  source 1..20: mol_type = other RNA
taccgaagagacaggtacgt

SEQ ID NO: 481 - RNA, 20 nt, synthetic construct
  misc_feature 1..20: note = Synthetic sequence
  source 1..20: mol_type = other RNA
ttgcgaagagacaggtacgt

SEQ ID NO: 482 - RNA, 20 nt, synthetic construct
  misc_feature 1..20: note = Synthetic sequence
  source 1..20: mol_type = other RNA
cttcttcaggttggacagct

SEQ ID NO: 483 - RNA, 281 nt, synthetic construct
  misc_feature 1..281: note = Synthetic sequence
  source 1..281: mol_type = other RNA
tctgaatggcaacggtatattacctggttttcgatcgaaagatcgacgaggtgaaaacctcgcgtatgacagggcacgggcagcttgccgggttttagagctagaaatagcaagttaaaataaggctagtccgttatcaacttgaaaaagtggcaccgagtcggtgcgtcagtcctgattttcgaatcagagaagacccagataattattttaactttctaaaacccggcaagctgcccgtgagaagtcatacgaccagagaaacacgatcggagtacaaa

SEQ ID NO: 484 - RNA, 20 nt, synthetic construct
  misc_feature 1..20: note = Synthetic sequence
  source 1..20: mol_type = other RNA
tacggaagagacaggtacgt

SEQ ID NO: 485 - RNA, 20 nt, synthetic construct
  misc_feature 1..20: note = Synthetic sequence
  source 1..20: mol_type = other RNA
ttgggaagagacaggtacgt

SEQ ID NO: 486 - RNA, 20 nt, synthetic construct
  misc_feature 1..20: note = Synthetic sequence
  source 1..20: mol_type = other RNA
ttccgaagagacaggtacgt

SEQ ID NO: 487 - RNA, 20 nt, synthetic construct
  misc_feature 1..20: note = Synthetic sequence
  source 1..20: mol_type = other RNA
ttcgcaagagacaggtacgt

SEQ ID NO: 488 - RNA, 20 nt, synthetic construct
  misc_feature 1..20: note = Synthetic sequence
  source 1..20: mol_type = other RNA
ttcggtagagacaggtacgt

SEQ ID NO: 489 - RNA, 20 nt, synthetic construct
  misc_feature 1..20: note = Synthetic sequence
  source 1..20: mol_type = other RNA
ttcggatgagacaggtacgt

SEQ ID NO: 490 - RNA, 20 nt, synthetic construct
  misc_feature 1..20: note = Synthetic sequence
  source 1..20: mol_type = other RNA
ttcggaacagacaggtacgt

SEQ ID NO: 491 - RNA, 20 nt, synthetic construct
  misc_feature 1..20: note = Synthetic sequence
  source 1..20: mol_type = other RNA
ttcggaagtgacaggtacgt

SEQ ID NO: 492 - RNA, 20 nt, synthetic construct
  misc_feature 1..20: note = Synthetic sequence
  source 1..20: mol_type = other RNA
ttcggaagacacaggtacgt

SEQ ID NO: 493 - RNA, 20 nt, synthetic construct
  misc_feature 1..20: note = Synthetic sequence
  source 1..20: mol_type = other RNA
ttcggaagagtcaggtacgt

SEQ ID NO: 494 - RNA, 20 nt, synthetic construct
  misc_feature 1..20: note = Synthetic sequence
  source 1..20: mol_type = other RNA
taccgaagagacaggtacgt

SEQ ID NO: 495 - RNA, 20 nt, synthetic construct
  misc_feature 1..20: note = Synthetic sequence
  source 1..20: mol_type = other RNA
ttccgatgagacaggtacgt

SEQ ID NO: 496 - RNA, 20 nt, synthetic construct
  misc_feature 1..20: note = Synthetic sequence
  source 1..20: mol_type = other RNA
taccgatgagacaggtacgt

SEQ ID NO: 497 - DNA, 20 bp, synthetic construct
  misc_RNA 1..20
  source 1..20: mol_type = other DNA
aattatggggattactagga

SEQ ID NO: 498 - DNA, 20 bp, synthetic construct
  misc_RNA 1..20
  source 1..20: mol_type = other DNA
gggcacgggcagcttgccgg

SEQ ID NO: 499 - DNA, 20 bp, synthetic construct
  misc_RNA 1..20
  source 1..20: mol_type = other DNA
gagctggacggcgacgtaaa

SEQ ID NO: 500 - DNA, 20 bp, synthetic construct
  misc_RNA 1..20
  source 1..20: mol_type = other DNA
ggccacaagttcagcgtgtc

SEQ ID NO: 501 - DNA, 20 bp, synthetic construct
  misc_RNA 1..20
  source 1..20: mol_type = other DNA
ggtgcccatcctggtcgagc

SEQ ID NO: 502 - DNA, 20 bp, synthetic construct
  misc_RNA 1..20
  source 1..20: mol_type = other DNA
gacgtagccttcgggcatgg

SEQ ID NO: 503 - DNA, 20 bp, synthetic construct
  misc_RNA 1..20
  source 1..20: mol_type = other DNA
gtcgtgctgcttcatgtggt

SEQ ID NO: 504 - DNA, 20 bp, synthetic construct
  misc_RNA 1..20
  source 1..20: mol_type = other DNA
gtcagggtggtcacgagggt

SEQ ID NO: 505 - DNA, 20 bp, synthetic construct
  misc_RNA 1..20
  source 1..20: mol_type = other DNA
gtgaaccgcatcgagctgaa

SEQ ID NO: 506 - DNA, 44 bp, synthetic construct
  misc_RNA 1..44
  source 1..44: mol_type = other DNA
tgaggtagtagattgtatagtttgaggtagtagattgtatagtt

SEQ ID NO: 507 - DNA, 40 bp, synthetic construct
  misc_RNA 1..40
  source 1..40: mol_type = other DNA
ttcggaagagacaggtacgtttcggaagagacaggtacgt